SEQUENCE LISTING (1143 sequences)

SEQ ID NO: 1 - protein, 9 aa, Homo sapiens
TLDDAVHGV

SEQ ID NO: 2 - protein, 9 aa, Homo sapiens
YQIGQVQGV

SEQ ID NO: 3 - protein, 10 aa, Homo sapiens
NEYSNITRLY

SEQ ID NO: 4 - protein, 9 aa, Homo sapiens
RLLADLTGA

SEQ ID NO: 5 - protein, 9 aa, Homo sapiens
TPLSPDPTL

SEQ ID NO: 6 - protein, 9 aa, Homo sapiens
STYYWPRPR

SEQ ID NO: 7 - protein, 9 aa, Homo sapiens
DSFSATETR

SEQ ID NO: 8 - protein, 9 aa, Homo sapiens
ETSYVKVLH

SEQ ID NO: 9 - protein, 8 aa, Homo sapiens
AEMLGSVV

SEQ ID NO: 10 - protein, 9 aa, Homo sapiens
EELSVLEVF

SEQ ID NO: 11 - protein, 9 aa, Homo sapiens
FVQENYLEY

SEQ ID NO: 12 - protein, 9 aa, Homo sapiens
IVQQPTEEK

SEQ ID NO: 13 - protein, 9 aa, Homo sapiens
SVFAHPRKL

SEQ ID NO: 14 - protein, 10 aa, Homo sapiens
ASSLPTTMNY

SEQ ID NO: 15 - protein, 10 aa, Homo sapiens
IVQQPTEEKR

SEQ ID NO: 16 - protein, 9 aa, Homo sapiens
LPTTMNYPL

SEQ ID NO: 17 - protein, 9 aa, Homo sapiens
GSNPARYEF

SEQ ID NO: 18 - protein, 8 aa, Homo sapiens
TADDATVM

SEQ ID NO: 19 - protein, 8 aa, Homo sapiens
VLEHVVRV

SEQ ID NO: 20 - protein, 9 aa, Homo sapiens
EVDPIGHLY

SEQ ID NO: 21 - protein, 8 aa, Homo sapiens
QIMPKAGL

SEQ ID NO: 22 - protein, 9 aa, Homo sapiens
KVLEHVVRV

SEQ ID NO: 23 - protein, 9 aa, Homo sapiens
QVPGSNPAR

SEQ ID NO: 24 - protein, 9 aa, Homo sapiens
FVYGEPREL

SEQ ID NO: 25 - protein, 10 aa, Homo sapiens
TLPTFDPTKV

SEQ ID NO: 26 - protein, 9 aa, Homo sapiens
NQIMPKTGF

SEQ ID NO: 27 - protein, 9 aa, Homo sapiens
EVDPIGHVY

SEQ ID NO: 28 - protein, 9 aa, Homo sapiens
ELVRRILSR

SEQ ID NO: 29 - protein, 10 aa, Homo sapiens
MEVDPIGHLY

SEQ ID NO: 30 - protein, 9 aa, Homo sapiens
SVMSSNVSF

SEQ ID NO: 31 - protein, 9 aa, Homo sapiens
TVPSSFPSW

SEQ ID NO: 32 - protein, 9 aa, Homo sapiens
KYKDYFPVI

SEQ ID NO: 33 - protein, 10 aa, Homo sapiens
MEVDPIGHVY

SEQ ID NO: 34 - protein, 9 aa, Homo sapiens
TVYGEPRKL

SEQ ID NO: 35 - protein, 9 aa, Homo sapiens
GPDVREGTL

SEQ ID NO: 36 - protein, 9 aa, Homo sapiens
VYDGREHTV

SEQ ID NO: 37 - protein, 11 aa, Homo sapiens
LMEVDPIGHLY

SEQ ID NO: 38 - protein, 11 aa, Homo sapiens
KYKDYFPVILK

SEQ ID NO: 39 - protein, 11 aa, Homo sapiens
EVFEGREDSIF

SEQ ID NO: 40 - protein, 10 aa, Homo sapiens
GVYDGREHTV

SEQ ID NO: 41 - protein, 9 aa, Homo sapiens
EVVPISHLY

SEQ ID NO: 42 - protein, 9 aa, Homo sapiens
NAVGVYAGR

SEQ ID NO: 43 - protein, 9 aa, Homo sapiens
RPDSRLLEL

SEQ ID NO: 44 - protein, 11 aa, Homo sapiens
SVFAHPRKLLM

SEQ ID NO: 45 - protein, 11 aa, Homo sapiens
EVFEGREDSVF

SEQ ID NO: 46 - protein, 9 aa, Homo sapiens
RVSHSHASF

SEQ ID NO: 47 - protein, 10 aa, Homo sapiens
ATFLQRKVLR

SEQ ID NO: 48 - protein, 9 aa, Homo sapiens
SYVKVLHHL

SEQ ID NO: 49 - protein, 9 aa, Homo sapiens
FAFGEPREL

SEQ ID NO: 50 - protein, 9 aa, Homo sapiens
KMAELVHFL

SEQ ID NO: 51 - protein, 10 aa, Homo sapiens
FAHGHPLYTR

SEQ ID NO: 52 - protein, 10 aa, Homo sapiens
GVYAGREHFV

SEQ ID NO: 53 - protein, 11 aa, Homo sapiens
NTAIATFLQRK

SEQ ID NO: 54 - protein, 9 aa, Homo sapiens
TLDEKVAEL

SEQ ID NO: 55 - protein, 9 aa, Homo sapiens
RLLELHITM

SEQ ID NO: 56 - protein, 9 aa, Homo sapiens
EVPNSSPPR

SEQ ID NO: 57 - protein, 8 aa, Homo sapiens
VEVTLREV

SEQ ID NO: 58 - protein, 9 aa, Homo sapiens
ISGGPRISY

SEQ ID NO: 59 - protein, 11 aa, Homo sapiens
GVPGPLLILPR

SEQ ID NO: 60 - protein, 10 aa, Homo sapiens
GVYDGEEHSV

SEQ ID NO: 61 - protein, 10 aa, Homo sapiens
KISGGPRISY

SEQ ID NO: 62 - protein, 10 aa, Homo sapiens
FLYNQTNIRM

SEQ ID NO: 63 - protein, 9 aa, Homo sapiens
GQYQLVVSL

SEQ ID NO: 64 - protein, 9 aa, Homo sapiens
GVDDLVFRR

SEQ ID NO: 65 - protein, 9 aa, Homo sapiens
KVLEYVIKV

SEQ ID NO: 66 - protein, 9 aa, Homo sapiens
HTLPALSRR

SEQ ID NO: 67 - protein, 9 aa, Homo sapiens
ALREEEEGV

SEQ ID NO: 68 - protein, 9 aa, Homo sapiens
TTINFTRQR

SEQ ID NO: 69 - protein, 9 aa, Homo sapiens
SAYGEPRKL

SEQ ID NO: 70 - protein, 9 aa, Homo sapiens
STANVTRAH

SEQ ID NO: 71 - protein, 9 aa, Homo sapiens
SAFPTTINF

SEQ ID NO: 72 - protein, 9 aa, Homo sapiens
FRAVITKKV

SEQ ID NO: 73 - protein, 9 aa, Homo sapiens
AAAGVSSTK

SEQ ID NO: 74 - protein, 9 aa, Homo sapiens
FPTTINFTR

SEQ ID NO: 75 - protein, 9 aa, Homo sapiens
TTSEKIHER

SEQ ID NO: 76 - protein, 9 aa, Homo sapiens
GLYDGMEHL

SEQ ID NO: 77 - protein, 11 aa, Homo sapiens
STANVTRAHGR

SEQ ID NO: 78 - protein, 10 aa, Homo sapiens
IEDPKAGLHV

SEQ ID NO: 79 - protein, 9 aa, Homo sapiens
GVVGPQGLR

SEQ ID NO: 80 - protein, 11 aa, Homo sapiens
NVFAHATGESR

SEQ ID NO: 81 - protein, 9 aa, Homo sapiens
FLFGEPKRL

SEQ ID NO: 82 - protein, 11 aa, Homo sapiens
SAFPTTINFTR

SEQ ID NO: 83 - protein, 9 aa, Homo sapiens
RLFSEDIHV

SEQ ID NO: 84 - protein, 9 aa, Homo sapiens
VYMPANTTF

SEQ ID NO: 85 - protein, 9 aa, Homo sapiens
EAVGFQPPR

SEQ ID NO: 86 - protein, 9 aa, Homo sapiens
EVDPTSHSY

SEQ ID NO: 87 - protein, 10 aa, Homo sapiens
LLDPAQRNLY

SEQ ID NO: 88 - protein, 9 aa, Homo sapiens
SGISQVFQR

SEQ ID NO: 89 - protein, 10 aa, Homo sapiens
RLFSEDIHVA

SEQ ID NO: 90 - protein, 9 aa, Homo sapiens
VFDVGKGNM

SEQ ID NO: 91 - protein, 9 aa, Homo sapiens
KASEKIFYV

SEQ ID NO: 92 - protein, 10 aa, Homo sapiens
EAAAAFRFPR

SEQ ID NO: 93 - protein, 9 aa, Homo sapiens
MSISMSLLK

SEQ ID NO: 94 - protein, 9 aa, Homo sapiens
KASPSSLPH

SEQ ID NO: 95 - protein, 11 aa, Homo sapiens
RTLQQGVLQDW

SEQ ID NO: 96 - protein, 9 aa, Homo sapiens
TTATTFRAR

SEQ ID NO: 97 - protein, 9 aa, Homo sapiens
NPSHLITPR

SEQ ID NO: 98 - protein, 10 aa, Homo sapiens
ETMETLRTSR

SEQ ID NO: 99 - protein, 11 aa, Homo sapiens
MVWEGHPRERR

SEQ ID NO: 100 - protein, 9 aa, Homo sapiens
LPDGSRVEL

SEQ ID NO: 101 - protein, 10 aa, Homo sapiens
DVVTGNVPLK

SEQ ID NO: 102 - protein, 10 aa, Homo sapiens
ENPSHLITPR

SEQ ID NO: 103 - protein, 9 aa, Homo sapiens
SLARTILHL

SEQ ID NO: 104 - protein, 9 aa, Homo sapiens
KPHYGLRAI

SEQ ID NO: 105 - protein, 9 aa, Homo sapiens
AFLDIHTAL

SEQ ID NO: 106 - protein, 9 aa, Homo sapiens
KMSEFVYRF

SEQ ID NO: 107 - protein, 9 aa, Homo sapiens
FLVTKTVTM

SEQ ID NO: 108 - protein, 9 aa, Homo sapiens
HTTSVMENK

SEQ ID NO: 109 - protein, 9 aa, Homo sapiens
LVSIPIAVR

SEQ ID NO: 110 - protein, 9 aa, Homo sapiens
ITSGVKVKK

SEQ ID NO: 111 - protein, 9 aa, Homo sapiens
VSIPIAVRR

SEQ ID NO: 112 - protein, 10 aa, Homo sapiens
DIIELQEGLR

SEQ ID NO: 113 - protein, 9 aa, Homo sapiens
HLKPPELGF

SEQ ID NO: 114 - protein, 9 aa, Homo sapiens
QVIVKIIGH

SEQ ID NO: 115 - protein, 9 aa, Homo sapiens
AEQEIMKKV

SEQ ID NO: 116 - protein, 9 aa, Homo sapiens
EVPLKILAH

SEQ ID NO: 117 - protein, 9 aa, Homo sapiens
IVKIIGHFY

SEQ ID NO: 118 - protein, 10 aa, Homo sapiens
SLVSIPIAVR

SEQ ID NO: 119 - protein, 10 aa, Homo sapiens
MVSPGVSLFK

SEQ ID NO: 120 - protein, 9 aa, Homo sapiens
VLMMKLEDL

SEQ ID NO: 121 - protein, 10 aa, Homo sapiens
YSDLHTPGRY

SEQ ID NO: 122 - protein, 9 aa, Homo sapiens
EVARFLTYY

SEQ ID NO: 123 - protein, 9 aa, Homo sapiens
SVGDLMAPR

SEQ ID NO: 124 - protein, 9 aa, Homo sapiens
VLWRGDSPL

SEQ ID NO: 125 - protein, 10 aa, Homo sapiens
QPRQGSPVAA

SEQ ID NO: 126 - protein, 9 aa, Homo sapiens
KQTGIRHSY

SEQ ID NO: 127 - protein, 9 aa, Homo sapiens
RPRQDAISV

SEQ ID NO: 128 - protein, 9 aa, Homo sapiens
KLITQIRTA

SEQ ID NO: 129 - protein, 11 aa, Homo sapiens
SYLSEHSAAIF

SEQ ID NO: 130 - protein, 10 aa, Homo sapiens
TSVGDLMAPR

SEQ ID NO: 131 - protein, 9 aa, Homo sapiens
EIPTGVAKR

SEQ ID NO: 132 - protein, 9 aa, Homo sapiens
SQINLSQIY

SEQ ID NO: 133 - protein, 9 aa, Homo sapiens
RRPSGGFRL

SEQ ID NO: 134 - protein, 11 aa, Homo sapiens
EVFNVKNTFPK

SEQ ID NO: 135 - protein, 9 aa, Homo sapiens
KVLGGGWTV

SEQ ID NO: 136 - protein, 10 aa, Homo sapiens
HEAPGQHSEV

SEQ ID NO: 137 - protein, 10 aa, Homo sapiens
EVSHPNMSGR

SEQ ID NO: 138 - protein, 9 aa, Homo sapiens
DTAPATFLR

SEQ ID NO: 139 - protein, 9 aa, Homo sapiens
KPQKDLHFI

SEQ ID NO: 140 - protein, 9 aa, Homo sapiens
RVRGAGTHL

SEQ ID NO: 141 - protein, 9 aa, Homo sapiens
TAPIIVAQR

SEQ ID NO: 142 - protein, 10 aa, Homo sapiens
TSWDTKFYEK

SEQ ID NO: 143 - protein, 10 aa, Homo sapiens
KVFAEHKISY

SEQ ID NO: 144 - protein, 9 aa, Homo sapiens
DAFQGIANR

SEQ ID NO: 145 - protein, 9 aa, Homo sapiens
IVADPRVPR

SEQ ID NO: 146 - protein, 9 aa, Homo sapiens
TGFSTSLGR

SEQ ID NO: 147 - protein, 10 aa, Homo sapiens
ATAPIIVAQR

SEQ ID NO: 148 - protein, 9 aa, Homo sapiens
GQFLVKSGY

SEQ ID NO: 149 - protein, 9 aa, Homo sapiens
WTSRTVQNK

SEQ ID NO: 150 - protein, 10 aa, Homo sapiens
ATYGGSDHYI

SEQ ID NO: 151 - protein, 9 aa, Homo sapiens
VYIAELEKI

SEQ ID NO: 152 - protein, 9 aa, Homo sapiens
VFLDRAVSI

SEQ ID NO: 153 - protein, 9 aa, Homo sapiens
MPSPIDHPR

SEQ ID NO: 154 - protein, 11 aa, Homo sapiens
EAVRHILHGSR

SEQ ID NO: 155 - protein, 9 aa, Homo sapiens
YPAGNVKEL

SEQ ID NO: 156 - protein, 9 aa, Homo sapiens
SVPRSLASR

SEQ ID NO: 157 - protein, 9 aa, Homo sapiens
ALIKHVANA

SEQ ID NO: 158 - protein, 9 aa, Homo sapiens
YTANLVPVR

SEQ ID NO: 159 - protein, 9 aa, Homo sapiens
LSSSLVTKL

SEQ ID NO: 160 - protein, 9 aa, Homo sapiens
QNSNVLAVR

SEQ ID NO: 161 - protein, 9 aa, Homo sapiens
TTTKNTLNK

SEQ ID NO: 162 - protein, 9 aa, Homo sapiens
HNYINLHSL

SEQ ID NO: 163 - protein, 9 aa, Homo sapiens
HLKDYFEEY

SEQ ID NO: 164 - protein, 9 aa, Homo sapiens
SPEYFTSYL

SEQ ID NO: 165 - protein, 9 aa, Homo sapiens
ATDVLDHLY

SEQ ID NO: 166 - protein, 9 aa, Homo sapiens
QTFEESLGR

SEQ ID NO: 167 - protein, 9 aa, Homo sapiens
HEQPELQKV

SEQ ID NO: 168 - protein, 10 aa, Homo sapiens
MATDVLDHLY

SEQ ID NO: 169 - protein, 9 aa, Homo sapiens
TSGSLLFPR

SEQ ID NO: 170 - protein, 9 aa, Homo sapiens
KLGINNVSF

SEQ ID NO: 171 - protein, 9 aa, Homo sapiens
KYISQFQVF

SEQ ID NO: 172 - protein, 9 aa, Homo sapiens
QTAAVSGVK

SEQ ID NO: 173 - protein, 10 aa, Homo sapiens
LTPLNHPFSY

SEQ ID NO: 174 - protein, 9 aa, Homo sapiens
VFINKKYVF

SEQ ID NO: 175 - protein, 9 aa, Homo sapiens
NLITEEHPV

SEQ ID NO: 176 - protein, 11 aa, Homo sapiens
ELVKVIDGSSR

SEQ ID NO: 177 - protein, 9 aa, Homo sapiens
FHSLPIHLF

SEQ ID NO: 178 - protein, 8 aa, Homo sapiens
YSHDYSYY

SEQ ID NO: 179 - protein, 9 aa, Homo sapiens
FQIEKLTPL

SEQ ID NO: 180 - protein, 9 aa, Homo sapiens
EHSVENGSI

SEQ ID NO: 181 - protein, 9 aa, Homo sapiens
ETIQNTHSF

SEQ ID NO: 182 - protein, 9 aa, Homo sapiens
YYSIWKTQI

SEQ ID NO: 183 - protein, 9 aa, Homo sapiens
SYYFHGLSF

SEQ ID NO: 184 - protein, 10 aa, Homo sapiens
ESFQQPYIPR

SEQ ID NO: 185 - protein, 9 aa, Homo sapiens
KQVPSRVLY

SEQ ID NO: 186 - protein, 10 aa, Homo sapiens
MLFNKDLDAA

SEQ ID NO: 187 - protein, 10 aa, Homo sapiens
APRSAEPRAT

SEQ ID NO: 188 - protein, 11 aa, Homo sapiens
YLDPGAVHAVF

SEQ ID NO: 189 - protein, 8 aa, Homo sapiens
TLKKHKIV

SEQ ID NO: 190 - protein, 9 aa, Homo sapiens
VGVPISTLY

SEQ ID NO: 191 - protein, 8 aa, Homo sapiens
SPRPQPEL

SEQ ID NO: 192 - protein, 9 aa, Homo sapiens
KLSFPDSKF

SEQ ID NO: 193 - protein, 10 aa, Homo sapiens
KNKDEYIHFY

SEQ ID NO: 194 - protein, 10 aa, Homo sapiens
RAFEGRRRGW

SEQ ID NO: 195 - protein, 9 aa, Homo sapiens
RLKARPSTY

SEQ ID NO: 196 - protein, 8 aa, Homo sapiens
APSLHATL

SEQ ID NO: 197 - protein, 9 aa, Homo sapiens
AQKEPTGNY

SEQ ID NO: 198 - protein, 9 aa, Homo sapiens
LSLGTTACR

SEQ ID NO: 199 - protein, 9 aa, Homo sapiens
SRNPTSYKY

SEQ ID NO: 200 - protein, 10 aa, Homo sapiens
SLNNDDHQIY

SEQ ID NO: 201 - protein, 9 aa, Homo sapiens
YVDPMITEI

SEQ ID NO: 202 - protein, 9 aa, Homo sapiens
PSFSPYAKR

SEQ ID NO: 203 - protein, 10 aa, Homo sapiens
HVNGSRPHAR

SEQ ID NO: 204 - protein, 9 aa, Homo sapiens
ITVRLAPPL

SEQ ID NO: 205 - protein, 9 aa, Homo sapiens
SANPLSHRY

SEQ ID NO: 206 - protein, 9 aa, Homo sapiens
VPLAHSSSA

SEQ ID NO: 207 - protein, 9 aa, Homo sapiens
GQVPTTEVV

SEQ ID NO: 208 - protein, 9 aa, Homo sapiens
KTWGQYWQV

SEQ ID NO: 209 - protein, 9 aa, Homo sapiens
ALNFPGSQK

SEQ ID NO: 210 - protein, 9 aa, Homo sapiens
YVWKTWGQY

SEQ ID NO: 211 - protein, 9 aa, Homo sapiens
MPTAESTGM

SEQ ID NO: 212 - protein, 11 aa, Homo sapiens
HTMEVTVYHRR

SEQ ID NO: 213 - protein, 9 aa, Homo sapiens
EEAAGIGIL

SEQ ID NO: 214 - protein, 9 aa, Homo sapiens
EGFDHRDSK

SEQ ID NO: 215 - protein, 10 aa, Homo sapiens
AEEAAGIGIL

SEQ ID NO: 216 - protein, 9 aa, Homo sapiens
LTSSMVTPR

SEQ ID NO: 217 - protein, 9 aa, Homo sapiens
TSSMVTPRR

SEQ ID NO: 218 - protein, 9 aa, Homo sapiens
GLSNLTHVL

SEQ ID NO: 219 - protein, 9 aa, Homo sapiens
QLLALLPSL

SEQ ID NO: 220 - protein, 9 aa, Homo sapiens
EAAQPMTKK

SEQ ID NO: 221 - protein, 9 aa, Homo sapiens
RVLGTKPSW

SEQ ID NO: 222 - protein, 10 aa, Homo sapiens
VLYPVPLESY

SEQ ID NO: 223 - protein, 9 aa, Homo sapiens
PYLGQMINL

SEQ ID NO: 224 - protein, 9 aa, Homo sapiens
ELFSYLIEK

SEQ ID NO: 225 - protein, 8 aa, Homo sapiens
EEQYIAQF

SEQ ID NO: 226 - protein, 10 aa, Homo sapiens
EAAQPMTKKR

SEQ ID NO: 227 - protein, 9 aa, Homo sapiens
MPMQDIKMI

SEQ ID NO: 228 - protein, 10 aa, Homo sapiens
RLSEGDVMHL

SEQ ID NO: 229 - protein, 10 aa, Homo sapiens
HASSYISPEK

SEQ ID NO: 230 - protein, 10 aa, Homo sapiens
MPMQDIKMIL

SEQ ID NO: 231 - protein, 10 aa, Homo sapiens
SPSVSQLSVL

SEQ ID NO: 232 - protein, 9 aa, Homo sapiens
RLDQLLRHV

SEQ ID NO: 233 - protein, 10 aa, Homo sapiens
ALLERASATL

SEQ ID NO: 234 - protein, 10 aa, Homo sapiens
TVAPTPATAR

SEQ ID NO: 235 - protein, 9 aa, Homo sapiens
SQSPSVSQL

SEQ ID NO: 236 - protein, 11 aa, Homo sapiens
STVAPTPATAR

SEQ ID NO: 237 - protein, 10 aa, Homo sapiens
QYIAQFTSQF

SEQ ID NO: 238 - protein, 9 aa, Homo sapiens
GQHLHLETF

SEQ ID NO: 239 - protein, 9 aa, Homo sapiens
TIASVLVAR

SEQ ID NO: 240 - protein, 9 aa, Homo sapiens
SLLQHLIGL

SEQ ID NO: 241 - protein, 9 aa, Homo sapiens
TASPRSPVM

SEQ ID NO: 242 - protein, 11 aa, Homo sapiens
AAFDGRHSQTL

SEQ ID NO: 243 - protein, 8 aa, Homo sapiens
SPRRLVEL

SEQ ID NO: 244 - protein, 9 aa, Homo sapiens
LYVDSLFFL

SEQ ID NO: 245 - protein, 9 aa, Homo sapiens
VFLREFPNF

SEQ ID NO: 246 - protein, 11 aa, Homo sapiens
RPRFSTATEIA

SEQ ID NO: 247 - protein, 9 aa, Homo sapiens
ALIANPRRK

SEQ ID NO: 248 - protein, 9 aa, Homo sapiens
DFMGQIVYR

SEQ ID NO: 249 - protein, 9 aa, Homo sapiens
EIPDTVMGL

SEQ ID NO: 250 - protein, 9 aa, Homo sapiens
VMSSTLYTV

SEQ ID NO: 251 - protein, 8 aa, Homo sapiens
SPQHKPRL

SEQ ID NO: 252 - protein, 9 aa, Homo sapiens
EPKPPTTGL

SEQ ID NO: 253 - protein, 9 aa, Homo sapiens
IPDTIASVL

SEQ ID NO: 254 - protein, 10 aa, Homo sapiens
TDFMGQIVYR

SEQ ID NO: 255 - protein, 9 aa, Homo sapiens
GEDDEHRKI

SEQ ID NO: 256 - protein, 10 aa, Homo sapiens
RLRGSIQSRY

SEQ ID NO: 257 - protein, 9 aa, Homo sapiens
HLDESPAYL

SEQ ID NO: 258 - protein, 9 aa, Homo sapiens
KMEETTVTY

SEQ ID NO: 259 - protein, 9 aa, Homo sapiens
LPTDLFNSV

SEQ ID NO: 260 - protein, 8 aa, Homo sapiens
RPQGLAVL

SEQ ID NO: 261 - protein, 9 aa, Homo sapiens
SLSEKSPEV

SEQ ID NO: 262 - protein, 9 aa, Homo sapiens
GAFREGPPY

SEQ ID NO: 263 - protein, 9 aa, Homo sapiens
YLNGATVVA

SEQ ID NO: 264 - protein, 9 aa, Homo sapiens
SMYLHTEGF

SEQ ID NO: 265 - protein, 9 aa, Homo sapiens
HILSHLHEV

SEQ ID NO: 266 - protein, 10 aa, Homo sapiens
SISEAPLTEV

SEQ ID NO: 267 - protein, 9 aa, Homo sapiens
HQINSEATF

SEQ ID NO: 268 - protein, 10 aa, Homo sapiens
DIKQEGVGAF

SEQ ID NO: 269 - protein, 9 aa, Homo sapiens
SALGVTITK

SEQ ID NO: 270 - protein, 10 aa, Homo sapiens
TVNDFTQNPR

SEQ ID NO: 271 - protein, 9 aa, Homo sapiens
SFIQAGSSY

SEQ ID NO: 272 - protein, 9 aa, Homo sapiens
GVTITKTLK

SEQ ID NO: 273 - protein, 9 aa, Homo sapiens
AERPQGLAV

SEQ ID NO: 274 - protein, 9 aa, Homo sapiens
REKGPVKEV

SEQ ID NO: 275 - protein, 9 aa, Homo sapiens
TSVPPFNLR

SEQ ID NO: 276 - protein, 9 aa, Homo sapiens
LSRLPSIAF

SEQ ID NO: 277 - protein, 9 aa, Homo sapiens
FPVHAAEKF

SEQ ID NO: 278 - protein, 8 aa, Homo sapiens
GEISASHV

SEQ ID NO: 279 - protein, 10 aa, Homo sapiens
AADGGQHWTY

SEQ ID NO: 280 - protein, 9 aa, Homo sapiens
EISASHVSR

SEQ ID NO: 281 - protein, 9 aa, Homo sapiens
SPRIPFSTF

SEQ ID NO: 282 - protein, 11 aa, Homo sapiens
KLMDPGSLPPL

SEQ ID NO: 283 - protein, 9 aa, Homo sapiens
ETKPEFERR

SEQ ID NO: 284 - protein, 9 aa, Homo sapiens
ETIMKFLKM

SEQ ID NO: 285 - protein, 9 aa, Homo sapiens
RRAHLWSTF

SEQ ID NO: 286 - protein, 9 aa, Homo sapiens
HLQEKLQSL

SEQ ID NO: 287 - protein, 9 aa, Homo sapiens
ELSVVTSER

SEQ ID NO: 288 - protein, 10 aa, Homo sapiens
YLDQQVPYTF

SEQ ID NO: 289 - protein, 9 aa, Homo sapiens
EAIPRIPRL

SEQ ID NO: 290 - protein, 11 aa, Homo sapiens
GYLDQQVPYTF

SEQ ID NO: 291 - protein, 9 aa, Homo sapiens
SPKLGGIGF

SEQ ID NO: 292 - protein, 9 aa, Homo sapiens
YPEKSRGCL

SEQ ID NO: 293 - protein, 11 aa, Homo sapiens
TTIPFSTLRQR

SEQ ID NO: 294 - protein, 10 aa, Homo sapiens
SLGAHSPALY

SEQ ID NO: 295 - protein, 9 aa, Homo sapiens
SQNGQLIHY

SEQ ID NO: 296 - protein, 11 aa, Homo sapiens
KAKEHTANISW

SEQ ID NO: 297 - protein, 9 aa, Homo sapiens
RVSKSDSTF

SEQ ID NO: 298 - protein, 9 aa, Homo sapiens
SLNSQVTGL

SEQ ID NO: 299 - protein, 10 aa, Homo sapiens
NSDLSEKLEY

SEQ ID NO: 300 - protein, 9 aa, Homo sapiens
DSSTVGLQR

SEQ ID NO: 301 - protein, 9 aa, Homo sapiens
RASAIIIQR

SEQ ID NO: 302 - protein, 9 aa, Homo sapiens
SQETLPLKI

SEQ ID NO: 303 - protein, 9 aa, Homo sapiens
STFDGLDRR

SEQ ID NO: 304 - protein, 9 aa, Homo sapiens
RVKEIVINK

SEQ ID NO: 305 - protein, 9 aa, Homo sapiens
AQPHHGVPY

SEQ ID NO: 306 - protein, 11 aa, Homo sapiens
EVIQLKERDRK

SEQ ID NO: 307 - protein, 8 aa, Homo sapiens
EVKEKTEL

SEQ ID NO: 308 - protein, 8 aa, Homo sapiens
TDPETLHY

SEQ ID NO: 309 - protein, 9 aa, Homo sapiens
KMKEKTQEL

SEQ ID NO: 310 - protein, 10 aa, Homo sapiens
STFDGLDRRR

SEQ ID NO: 311 - protein, 9 aa, Homo sapiens
KMSELQTYV

SEQ ID NO: 312 - protein, 9 aa, Homo sapiens
GTDPETLHY

SEQ ID NO: 313 - protein, 9 aa, Homo sapiens
TEEKGSILI

SEQ ID NO: 314 - protein, 9 aa, Homo sapiens
DTAMPAHSR

SEQ ID NO: 315 - protein, 9 aa, Homo sapiens
KLQEALKTI

SEQ ID NO: 316 - protein, 10 aa, Homo sapiens
GTDPETLHYF

SEQ ID NO: 317 - protein, 9 aa, Homo sapiens
SLKNRIESV

SEQ ID NO: 318 - protein, 9 aa, Homo sapiens
NLKDPVGVY

SEQ ID NO: 319 - protein, 9 aa, Homo sapiens
FTETNPQVY

SEQ ID NO: 320 - protein, 11 aa, Homo sapiens
ETQEAEVAVSR

SEQ ID NO: 321 - protein, 9 aa, Homo sapiens
SPRPESGTV

SEQ ID NO: 322 - protein, 9 aa, Homo sapiens
SEFAKDPSL

SEQ ID NO: 323 - protein, 9 aa, Homo sapiens
KVIKKIGHY

SEQ ID NO: 324 - protein, 9 aa, Homo sapiens
RTWQTRKQF

SEQ ID NO: 325 - protein, 9 aa, Homo sapiens
AMAHKVIKA

SEQ ID NO: 326 - protein, 9 aa, Homo sapiens
KLKQFQKEY

SEQ ID NO: 327 - protein, 9 aa, Homo sapiens
SPLEDEESL

SEQ ID NO: 328 - protein, 9 aa, Homo sapiens
NSTDFTTDR

SEQ ID NO: 329 - protein, 10 aa, Homo sapiens
ETKDTKGRYF

SEQ ID NO: 330 - protein, 9 aa, Homo sapiens
RPRVSSTEM

SEQ ID NO: 331 - protein, 9 aa, Homo sapiens
LMDKSSTIY

SEQ ID NO: 332 - protein, 10 aa, Homo sapiens
GTLRPLKVRR

SEQ ID NO: 333 - protein, 9 aa, Homo sapiens
ILSEGSVPL

SEQ ID NO: 334 - protein, 9 aa, Homo sapiens
TVYNFRTEL

SEQ ID NO: 335 - protein, 9 aa, Homo sapiens
LPSSADVEF

SEQ ID NO: 336 - protein, 10 aa, Homo sapiens
RLPSSADVEF

SEQ ID NO: 337 - protein, 9 aa, Homo sapiens
ALMDKSLHV

SEQ ID NO: 338 - protein, 9 aa, Homo sapiens
MHNALHIYM

SEQ ID NO: 339 - protein, 9 aa, Homo sapiens
MEKEDYHSL

SEQ ID NO: 340 - protein, 10 aa, Homo sapiens
MEKEDYHSLY

SEQ ID NO: 341 - protein, 9 aa, Homo sapiens
YHSLYQSHL

SEQ ID NO: 342 - protein, 9 aa, Homo sapiens
YMVPFIPLY

SEQ ID NO: 343 - protein, 10 aa, Homo sapiens
LMEKEDYHSL

SEQ ID NO: 344 - protein, 9 aa, Homo sapiens
FAHEAPAFL

SEQ ID NO: 345 - protein, 9 aa, Homo sapiens
FASPLTGIA

SEQ ID NO: 346 - protein, 9 aa, Homo sapiens
HEAPAFLPW

SEQ ID NO: 347 - protein, 9 aa, Homo sapiens
VLFKKFIQK

SEQ ID NO: 348 - protein, 9 aa, Homo sapiens
APAFLPWHR

SEQ ID NO: 349 - protein, 11 aa, Homo sapiens
LLMEKEDYHSL

SEQ ID NO: 350 - protein, 9 aa, Homo sapiens
HYYVSMDAL

SEQ ID NO: 351 - protein, 8 aa, Homo sapiens
WEQEIQKL

SEQ ID NO: 352 - protein, 11 aa, Homo sapiens
LMEKEDYHSLY

SEQ ID NO: 353 - protein, 10 aa, Homo sapiens
LEQASRIWSW

SEQ ID NO: 354 - protein, 10 aa, Homo sapiens
NIFDLSAPEK

SEQ ID NO: 355 - protein, 10 aa, Homo sapiens
EAPAFLPWHR

SEQ ID NO: 356 - protein, 9 aa, Homo sapiens
SEIWRDIDF

SEQ ID NO: 357 - protein, 8 aa, Homo sapiens
AAMVGAVL

SEQ ID NO: 358 - protein, 10 aa, Homo sapiens
TFLGVGQESF

SEQ ID NO: 359 - protein, 10 aa, Homo sapiens
ESWPSVFYNR

SEQ ID NO: 360 - protein, 9 aa, Homo sapiens
ISQDTTVGR

SEQ ID NO: 361 - protein, 9 aa, Homo sapiens
RPWSGPYIL

SEQ ID NO: 362 - protein, 11 aa, Homo sapiens
SSDYVIPIGTY

SEQ ID NO: 363 - protein, 10 aa, Homo sapiens
DSDPDSFQDY

SEQ ID NO: 364 - protein, 9 aa, Homo sapiens
DTRPWSGPY

SEQ ID NO: 365 - protein, 9 aa, Homo sapiens
HYYSVKKTF

SEQ ID NO: 366 - protein, 9 aa, Homo sapiens
NVCGSQQGR

SEQ ID NO: 367 - protein, 9 aa, Homo sapiens
YMNGTMSQV

SEQ ID NO: 368 - protein, 9 aa, Homo sapiens
MAKRTTHPL

SEQ ID NO: 369 - protein, 10 aa, Homo sapiens
RPWSGPYILR

SEQ ID NO: 370 - protein, 9 aa, Homo sapiens
MTVDSLVNK

SEQ ID NO: 371 - protein, 10 aa, Homo sapiens
TRPWSGPYIL

SEQ ID NO: 372 - protein, 11 aa, Homo sapiens
TTHPLFVIATR

SEQ ID NO: 373 - protein, 9 aa, Homo sapiens
FVWTHYYSV

SEQ ID NO: 374 - protein, 9 aa, Homo sapiens
LSKEEKNHF

SEQ ID NO: 375 - protein, 9 aa, Homo sapiens
RAANQVFVL

SEQ ID NO: 376 - protein, 9 aa, Homo sapiens
MVRSATLIK

SEQ ID NO: 377 - protein, 9 aa, Homo sapiens
NQVFVLHSY

SEQ ID NO: 378 - protein, 8 aa, Homo sapiens
HNSTEFLI

SEQ ID NO: 379 - protein, 9 aa, Homo sapiens
SQDVAGTTF

SEQ ID NO: 380 - protein, 9 aa, Homo sapiens
AHNSTEFLI

SEQ ID NO: 381 - protein, 9 aa, Homo sapiens
STEFLIYER

SEQ ID NO: 382 - protein, 9 aa, Homo sapiens
EGFFTRQER

SEQ ID NO: 383 - protein, 8 aa, Homo sapiens
FQKVLVSY

SEQ ID NO: 384 - protein, 10 aa, Homo sapiens
FTDFMGQIVY

SEQ ID NO: 385 - protein, 9 aa, Homo sapiens
YFQKVLVSY

SEQ ID NO: 386 - protein, 10 aa, Homo sapiens
ALVNMPPHYR

SEQ ID NO: 387 - protein, 10 aa, Homo sapiens
GVMSSTLYTV

SEQ ID NO: 388 - protein, 9 aa, Homo sapiens
LLSNTVSTL

SEQ ID NO: 389 - protein, 10 aa, Homo sapiens
RVFHETGAQR

SEQ ID NO: 390 - protein, 9 aa, Homo sapiens
SASDHCRSR

SEQ ID NO: 391 - protein, 9 aa, Homo sapiens
RRDGGIIIY

SEQ ID NO: 392 - protein, 9 aa, Homo sapiens
VAALIANPR

SEQ ID NO: 393 - protein, 9 aa, Homo sapiens
RLLGTEFQV

SEQ ID NO: 394 - protein, 9 aa, Homo sapiens
FSIIGVSRR

SEQ ID NO: 395 - protein, 9 aa, Homo sapiens
LSSDGMYEY

SEQ ID NO: 396 - protein, 8 aa, Homo sapiens
DEYFLPSL

SEQ ID NO: 397 - protein, 10 aa, Homo sapiens
SYIGLKGLYF

SEQ ID NO: 398 - protein, 10 aa, Homo sapiens
FTGFGGALGY

SEQ ID NO: 399 - protein, 10 aa, Homo sapiens
KTWDQVPFSV

SEQ ID NO: 400 - protein, 9 aa, Homo sapiens
RVADIPIKP

SEQ ID NO: 401 - protein, 9 aa, Homo sapiens
SVAKHTQSY

SEQ ID NO: 402 - protein, 10 aa, Homo sapiens
QTKGGQTWAR

SEQ ID NO: 403 - protein, 9 aa, Homo sapiens
ETGSSFMSF

SEQ ID NO: 404 - protein, 9 aa, Homo sapiens
SLYSYFQKV

SEQ ID NO: 405 - protein, 11 aa, Homo sapiens
QTKGGQTWARR

SEQ ID NO: 406 - protein, 11 aa, Homo sapiens
TVVAALIANPR

SEQ ID NO: 407 - protein, 9 aa, Homo sapiens
HLMVKDWQL

SEQ ID NO: 408 - protein, 9 aa, Homo sapiens
NPLSKLSVL

SEQ ID NO: 409 - protein, 11 aa, Homo sapiens
FVIPSTKDSNR

SEQ ID NO: 410 - protein, 8 aa, Homo sapiens
HYQVQTHL

SEQ ID NO: 411 - protein, 11 aa, Homo sapiens
ASDILSFAHKY

SEQ ID NO: 412 - protein, 9 aa, Homo sapiens
RLGQGVPLV

SEQ ID NO: 413 - protein, 10 aa, Homo sapiens
SVPDHVQPSK

SEQ ID NO: 414 - protein, 9 aa, Homo sapiens
RLLEKHISL

SEQ ID NO: 415 - protein, 9 aa, Homo sapiens
SLFGVSERL

SEQ ID NO: 416 - protein, 9 aa, Homo sapiens
NHYQVQTHL

SEQ ID NO: 417 - protein, 11 aa, Homo sapiens
DLVGKDVTRVY

SEQ ID NO: 418 - protein, 9 aa, Homo sapiens
SLSPPTLPR

SEQ ID NO: 419 - protein, 10 aa, Homo sapiens
LADNGTLGKY

SEQ ID NO: 420 - protein, 9 aa, Homo sapiens
KAMYIRVSY

SEQ ID NO: 421 - protein, 9 aa, Homo sapiens
MQPKLKQVF

SEQ ID NO: 422 - protein, 9 aa, Homo sapiens
AIIQDGILL

SEQ ID NO: 423 - protein, 8 aa, Homo sapiens
AFDRAVHF

SEQ ID NO: 424 - protein, 9 aa, Homo sapiens
NTPNIVSLR

SEQ ID NO: 425 - protein, 10 aa, Homo sapiens
RVADIPIKPW

SEQ ID NO: 426 - protein, 9 aa, Homo sapiens
HYIIPLQTI

SEQ ID NO: 427 - protein, 9 aa, Homo sapiens
VMKGINQGY

SEQ ID NO: 428 - protein, 9 aa, Homo sapiens
ATSPPASVR

SEQ ID NO: 429 - protein, 9 aa, Homo sapiens
ALEKDTPRY

SEQ ID NO: 430 - protein, 10 aa, Homo sapiens
QVMKGINQGY

SEQ ID NO: 431 - protein, 9 aa, Homo sapiens
AAFDRAVHF

SEQ ID NO: 432 - protein, 9 aa, Homo sapiens
RPLRIKGVF

SEQ ID NO: 433 - protein, 11 aa, Homo sapiens
RQVMKGINQGY

SEQ ID NO: 434 - protein, 9 aa, Homo sapiens
KLDTTVKNI

SEQ ID NO: 435 - protein, 9 aa, Homo sapiens
LPRTPRPDL

SEQ ID NO: 436 - protein, 9 aa, Homo sapiens
ITYTESNSR

SEQ ID NO: 437 - protein, 9 aa, Homo sapiens
RLQGSVTLV

SEQ ID NO: 438 - protein, 11 aa, Homo sapiens
SQFPSKNLPAF

SEQ ID NO: 439 - protein, 11 aa, Homo sapiens
LPRTPRPDLIL

SEQ ID NO: 440 - protein, 9 aa, Homo sapiens
DTAPQSHQV

SEQ ID NO: 441 - protein, 9 aa, Homo sapiens
MAILGTAGR

SEQ ID NO: 442 - protein, 9 aa, Homo sapiens
SVSSHLIHR

SEQ ID NO: 443 - protein, 10 aa, Homo sapiens
STSPGTGVRK

SEQ ID NO: 444 - protein, 10 aa, Homo sapiens
EVAEVLGSVK

SEQ ID NO: 445 - protein, 10 aa, Homo sapiens
EIPRTPSEGR

SEQ ID NO: 446 - protein, 9 aa, Homo sapiens
VFLKFSRRF

SEQ ID NO: 447 - protein, 10 aa, Homo sapiens
SVSSHLIHRY

SEQ ID NO: 448 - protein, 9 aa, Homo sapiens
THLEDPHLM

SEQ ID NO: 449 - protein, 9 aa, Homo sapiens
SVFPRTSSR

SEQ ID NO: 450 - protein, 11 aa, Homo sapiens
GQYGQSKPWTF

SEQ ID NO: 451 - protein, 10 aa, Homo sapiens
VPRPTSTVGL

SEQ ID NO: 452 - protein, 11 aa, Homo sapiens
NSVSSHLIHRY

SEQ ID NO: 453 - protein, 10 aa, Homo sapiens
QEEDNLLRYF

SEQ ID NO: 454 - protein, 10 aa, Homo sapiens
THLEDPHLML

SEQ ID NO: 455 - protein, 9 aa, Homo sapiens
ATTNILEHV

SEQ ID NO: 456 - protein, 9 aa, Homo sapiens
MTLRPTQPR

SEQ ID NO: 457 - protein, 9 aa, Homo sapiens
ETRPNNSKF

SEQ ID NO: 458 - protein, 9 aa, Homo sapiens
HSITEPLLY

SEQ ID NO: 459 - protein, 10 aa, Homo sapiens
HLFLQPPSKR

SEQ ID NO: 460 - protein, 9 aa, Homo sapiens
ETVRFTSSF

SEQ ID NO: 461 - protein, 10 aa, Homo sapiens
SPMPIPPLYL

SEQ ID NO: 462 - protein, 9 aa, Homo sapiens
ESYGRYTFY

SEQ ID NO: 463 - protein, 9 aa, Homo sapiens
KSQGSFISF

SEQ ID NO: 464 - protein, 9 aa, Homo sapiens
GTILHIGVF

SEQ ID NO: 465 - protein, 9 aa, Homo sapiens
KVFSGSSKR

SEQ ID NO: 466 - protein, 11 aa, Homo sapiens
SSFSTLASLDK

SEQ ID NO: 467 - protein, 9 aa, Homo sapiens
TTQGAAPQR

SEQ ID NO: 468 - protein, 10 aa, Homo sapiens
HTTQGAAPQR

SEQ ID NO: 469 - protein, 9 aa, Homo sapiens
KIFQERAAF

SEQ ID NO: 470 - protein, 9 aa, Homo sapiens
GQITHNTSF

SEQ ID NO: 471 - protein, 10 aa, Homo sapiens
ISLKPLIARY

SEQ ID NO: 472 - protein, 9 aa, Homo sapiens
KHSDLILFW

SEQ ID NO: 473 - protein, 10 aa, Homo sapiens
MVWEGHPRER

SEQ ID NO: 474 - protein, 9 aa, Homo sapiens
STDLPILLK

SEQ ID NO: 475 - protein, 10 aa, Homo sapiens
EVISINTGHF

SEQ ID NO: 476 - protein, 11 aa, Homo sapiens
ETERVLEKSSY

SEQ ID NO: 477 - protein, 9 aa, Homo sapiens
KIKTINLRY

SEQ ID NO: 478 - protein, 9 aa, Homo sapiens
YVSQKVSDF

SEQ ID NO: 479 - protein, 9 aa, Homo sapiens
DAGIPVYHR

SEQ ID NO: 480 - protein, 11 aa, Homo sapiens
TILEAEILHQA

SEQ ID NO: 481 - protein, 9 aa, Homo sapiens
EYAQKTPLF

SEQ ID NO: 482 - protein, 9 aa, Homo sapiens
TWTPHGIRI

SEQ ID NO: 483 - protein, 10 aa, Homo sapiens
EVHEGGVARF

SEQ ID NO: 484 - protein, 9 aa, Homo sapiens
REIEIEGRL

SEQ ID NO: 485 - protein, 9 aa, Homo sapiens
SWLPIPAKY

SEQ ID NO: 486 - protein, 9 aa, Homo sapiens
RYSERVYTI

SEQ ID NO: 487 - protein, 10 aa, Homo sapiens
FTDSQGNDIK

SEQ ID NO: 488 - protein, 10 aa, Homo sapiens
SVPSDFLSLR

SEQ ID NO: 489 - protein, 9 aa, Homo sapiens
RQMSLEFRA

SEQ ID NO: 490 - protein, 9 aa, Homo sapiens
ETISPRPSI

SEQ ID NO: 491 - protein, 9 aa, Homo sapiens
GPAGGRGVI

SEQ ID NO: 492 - protein, 9 aa, Homo sapiens
LTDHHIKEY

SEQ ID NO: 493 - protein, 11 aa, Homo sapiens
EAVPYNGSLFK

SEQ ID NO: 494 - protein, 9 aa, Homo sapiens
NLLAEIHGV

SEQ ID NO: 495 - protein, 9 aa, Homo sapiens
KEGPVTRGA

SEQ ID NO: 496 - protein, 9 aa, Homo sapiens
VHVDLNLGF

SEQ ID NO: 497 - protein, 9 aa, Homo sapiens
HTVQGLAGF

SEQ ID NO: 498 - protein, 10 aa, Homo sapiens
MRIEDSHTKL

SEQ ID NO: 499 - protein, 9 aa, Homo sapiens
SALSSAQPR

SEQ ID NO: 500 - protein, 10 aa, Homo sapiens
SPSIKSLLVY

SEQ ID NO: 501 - protein, 11 aa, Homo sapiens
PTDATLNRLLY

SEQ ID NO: 502 - protein, 10 aa, Homo sapiens
WTSQPPNYEK

SEQ ID NO: 503 - protein, 10 aa, Homo sapiens
PAPRPVAPTL

SEQ ID NO: 504 - protein, 9 aa, Homo sapiens
VYLHSNSTL

SEQ ID NO: 505 - protein, 9 aa, Homo sapiens
SLYAWTQSH

SEQ ID NO: 506 - DNA, 27 bp, Homo sapiens
  source 1..27: mol_type = other DNA
actctcgatgacgccgtccacggcgtt

SEQ ID NO: 507 - DNA, 27 bp, Homo sapiens
  source 1..27: mol_type = other DNA
taccaaattggccaagttcaaggtgta

SEQ ID NO: 508 - DNA, 30 bp, Homo sapiens
  source 1..30: mol_type = other DNA
aatgaatattctaacattacccggctttat

SEQ ID NO: 509 - DNA, 27 bp, Homo sapiens
  source 1..27: mol_type = other DNA
cggctcctggctgatctcactggggcc

SEQ ID NO: 510 - DNA, 27 bp, Homo sapiens
  source 1..27: mol_type = other DNA
actccattgtctcctgaccccaccctc

SEQ ID NO: 511 - DNA, 27 bp, Homo sapiens
  source 1..27: mol_type = other DNA
tcgacctattattggcctagaccaagg

SEQ ID NO: 512 - DNA, 27 bp, Homo sapiens
  source 1..27: mol_type = other DNA
gattctttctccgctactgagacacgg

SEQ ID NO: 513 - DNA, 27 bp, Homo sapiens
  source 1..27: mol_type = other DNA
gaaaccagctatgtgaaagtcctgcac

SEQ ID NO: 514 - DNA, 24 bp, Homo sapiens
  source 1..24: mol_type = other DNA
gcagaaatgctggggagtgtcgtc

SEQ ID NO: 515 - DNA, 27 bp, Homo sapiens
  source 1..27: mol_type = other DNA
gaggagctgagtgtgttagaggtgttt

SEQ ID NO: 516 - DNA, 27 bp, Homo sapiens
  source 1..27: mol_type = other DNA
ttcgtgcaggaaaactacctggagtac

SEQ ID NO: 517 - DNA, 27 bp, Homo sapiens
  source 1..27: mol_type = other DNA
attgtccagcagcccactgaggaaaaa

SEQ ID NO: 518 - DNA, 27 bp, Homo sapiens
  source 1..27: mol_type = other DNA
agtgtctttgcgcatcccaggaagctg

SEQ ID NO: 519 - DNA, 27 bp, Homo sapiens
  source 1..27: mol_type = other DNA
agtgtcttcgcacatcccaggaagctg

SEQ ID NO: 520 - DNA, 30 bp, Homo sapiens
  source 1..30: mol_type = other DNA
gcctccagcctccccactaccatgaactac

SEQ ID NO: 521 - DNA, 30 bp, Homo sapiens
  source 1..30: mol_type = other DNA
attgtccagcagcccactgaggaaaaacgt

SEQ ID NO: 522 - DNA, 27 bp, Homo sapiens
  source 1..27: mol_type = other DNA
ctccccactaccatgaactaccctctc

SEQ ID NO: 523 - DNA, 27 bp, Homo sapiens
  source 1..27: mol_type = other DNA
ggcagtaatcctgcgcgctatgagttc

SEQ ID NO: 524 - DNA, 24 bp, Homo sapiens
  source 1..24: mol_type = other DNA
accgcagatgatgccactgtcatg

SEQ ID NO: 525 - DNA, 24 bp, Homo sapiens
  source 1..24: mol_type = other DNA
gtcctggagcatgtggtcagggtc

SEQ ID NO: 526 - DNA, 27 bp, Homo sapiens
  source 1..27: mol_type = other DNA
gaagtggaccccatcggccacttgtac

SEQ ID NO: 527 - DNA, 24 bp, Homo sapiens
  source 1..24: mol_type = other DNA
cagatcatgcccaaggcaggcctc

SEQ ID NO: 528 - DNA, 27 bp, Homo sapiens
  source 1..27: mol_type = other DNA
aaagtcctggagcatgtggtcagggtc

SEQ ID NO: 529 - DNA, 27 bp, Homo sapiens
  source 1..27: mol_type = other DNA
caggtacccggcagtaatcctgcgcgc

SEQ ID NO: 530 - DNA, 27 bp, Homo sapiens
  source 1..27: mol_type = other DNA
ttcgtctatggggagcctagggagctc

SEQ ID NO: 531 - DNA, 30 bp, Homo sapiens
  source 1..30: mol_type = other DNA
actctgcccacttttgatcccactaaagtg

SEQ ID NO: 532 - DNA, 27 bp, Homo sapiens
  source 1..27: mol_type = other DNA
aatcagatcatgcccaagacaggcttc

SEQ ID NO: 533 - DNA, 27 bp, Homo sapiens
  source 1..27: mol_type = other DNA
gaagtggaccccatcggccacgtgtac

SEQ ID NO: 534 - DNA, 27 bp, Homo sapiens
  source 1..27: mol_type = other DNA
gagctggtccgcaggatcctgtcccgg

SEQ ID NO: 535 - DNA, 30 bp, Homo sapiens
  source 1..30: mol_type = other DNA
atggaagtggaccccatcggccacttgtac

SEQ ID NO: 536 - DNA, 27 bp, Homo sapiens
  source 1..27: mol_type = other DNA
agtgtcatgtccagcaacgtctccttt

SEQ ID NO: 537 - DNA, 27 bp, Homo sapiens
  source 1..27: mol_type = other DNA
actgttcctagttcctttccatcctgg

SEQ ID NO: 538 - DNA, 27 bp, Homo sapiens
  source 1..27: mol_type = other DNA
aagtacaaagattactttcctgtgata

SEQ ID NO: 539 - DNA, 30 bp, Homo sapiens
  source 1..30: mol_type = other DNA
atggaagtggaccccatcggccacgtgtac

SEQ ID NO: 540 - DNA, 27 bp, Homo sapiens
  source 1..27: mol_type = other DNA
actgtctatggggagcccaggaaactg

SEQ ID NO: 541 - DNA, 27 bp, Homo sapiens
  source 1..27: mol_type = other DNA
ggtcctgatgtcagggaggggactctg

SEQ ID NO: 542 - DNA, 27 bp, Homo sapiens
  source 1..27: mol_type = other DNA
gtgtatgatgggagggagcacactgtc

SEQ ID NO: 543 - DNA, 33 bp, Homo sapiens
  source 1..33: mol_type = other DNA
ctgatggaagtggaccccatcggccacttgtac

SEQ ID NO: 544 - DNA, 33 bp, Homo sapiens
  source 1..33: mol_type = other DNA
aagtacaaagattactttcctgtgatactcaag

SEQ ID NO: 545 - DNA, 33 bp, Homo sapiens
  source 1..33: mol_type = other DNA
gaggtgtttgaggggagggaagacagtatcttc

SEQ ID NO: 546 - DNA, 30 bp, Homo sapiens
  source 1..30: mol_type = other DNA
ggggtgtatgatgggagggagcacactgtc

SEQ ID NO: 547 - DNA, 27 bp, Homo sapiens
  source 1..27: mol_type = other DNA
gaagtggtccccatcagccacttgtac

SEQ ID NO: 548 - DNA, 27 bp, Homo sapiens
  source 1..27: mol_type = other DNA
aatgcagtaggggtatatgctgggagg

SEQ ID NO: 549 - DNA, 27 bp, Homo sapiens
  source 1..27: mol_type = other DNA
aggccggacagccgcctgcttgagttg

SEQ ID NO: 550 - DNA, 33 bp, Homo sapiens
  source 1..33: mol_type = other DNA
agtgtcttcgcacatcccaggaagctgctcatg

SEQ ID NO: 551 - DNA, 33 bp, Homo sapiens
  source 1..33: mol_type = other DNA
gaggtgtttgaggggagggaggacagtgtcttc

SEQ ID NO: 552 - DNA, 27 bp, Homo sapiens
  source 1..27: mol_type = other DNA
cgagtatctcactcacatgcttctttt

SEQ ID NO: 553 - DNA, 30 bp, Homo sapiens
  source 1..30: mol_type = other DNA
gccacctttttgcagaggaaggtactgagg

SEQ ID NO: 554 - DNA, 27 bp, Homo sapiens
  source 1..27: mol_type = other DNA
agctatgtgaaagtcctgcaccatttg

SEQ ID NO: 555 - DNA, 27 bp, Homo sapiens
  source 1..27: mol_type = other DNA
tttgcctttggggagcccagggagctc

SEQ ID NO: 556 - DNA, 27 bp, Homo sapiens
  source 1..27: mol_type = other DNA
aagatggctgagttggttcattttctg

SEQ ID NO: 557 - DNA, 30 bp, Homo sapiens
  source 1..30: mol_type = other DNA
tttgcccacgggcacccactgtacacacgc

SEQ ID NO: 558 - DNA, 30 bp, Homo sapiens
  source 1..30: mol_type = other DNA
ggggtatatgctgggagggagcacttcgtc

SEQ ID NO: 559 - DNA, 33 bp, Homo sapiens
  source 1..33: mol_type = other DNA
aacactgctattgccacctttttgcagaggaag

SEQ ID NO: 560 - DNA, 27 bp, Homo sapiens
  source 1..27: mol_type = other DNA
acactagatgaaaaggtggccgagtta

SEQ ID NO: 561 - DNA, 27 bp, Homo sapiens
  source 1..27: mol_type = other DNA
cgcctgcttgagttgcacatcacgatg

SEQ ID NO: 562 - DNA, 27 bp, Homo sapiens
  source 1..27: mol_type = other DNA
gaggtgcccaactcttctcctcctcgt

SEQ ID NO: 563 - DNA, 24 bp, Homo sapiens
  source 1..24: mol_type = other DNA
gtggaagtcaccctgcgggaggtg

SEQ ID NO: 564 - DNA, 27 bp, Homo sapiens
  source 1..27: mol_type = other DNA
atcagtggaggacctcgcatttcctac

SEQ ID NO: 565 - DNA, 33 bp, Homo sapiens
  source 1..33: mol_type = other DNA
ggagtccccggacccctcctcatccttcccagg

SEQ ID NO: 566 - DNA, 30 bp, Homo sapiens
  source 1..30: mol_type = other DNA
ggagtctatgatggagaggagcactcagtc

SEQ ID NO: 567 - DNA, 30 bp, Homo sapiens
  source 1..30: mol_type = other DNA
aagatcagtggaggacctcgcatttcctac

SEQ ID NO: 568 - DNA, 30 bp, Homo sapiens
  source 1..30: mol_type = other DNA
tttctatataatcagacaaatattaggatg

SEQ ID NO: 569 - DNA, 27 bp, Homo sapiens
  source 1..27: mol_type = other DNA
ggccagtaccagctggtggtgagcctg

SEQ ID NO: 570 - DNA, 27 bp, Homo sapiens
  source 1..27: mol_type = other DNA
ggagttgatgaccttgttttcagaagg

SEQ ID NO: 571 - DNA, 27 bp, Homo sapiens
  source 1..27: mol_type = other DNA
aaagtccttgagtatgtgatcaaggtc

SEQ ID NO: 572 - DNA, 27 bp, Homo sapiens
  source 1..27: mol_type = other DNA
cacactttaccagccctttctagaagg

SEQ ID NO: 573 - DNA, 27 bp, Homo sapiens
  source 1..27: mol_type = other DNA
gctttgagagaggaggaagagggagtc

SEQ ID NO: 574 - DNA, 27 bp, Homo sapiens
  source 1..27: mol_type = other DNA
actaccatcaacttcactcgacagagg

SEQ ID NO: 575 - DNA, 27 bp, Homo sapiens
  source 1..27: mol_type = other DNA
agtgcctatggggagcccaggaagctg

SEQ ID NO: 576 - DNA, 27 bp, Homo sapiens
  source 1..27: mol_type = other DNA
agcactgccaatgtgacgcgggcccac

SEQ ID NO: 577 - DNA, 27 bp, Homo sapiens
  source 1..27: mol_type = other DNA
tccgcctttcccactaccatcaacttc

SEQ ID NO: 578 - DNA, 27 bp, Homo sapiens
  source 1..27: mol_type = other DNA
ttccgagcagtaatcactaagaaggtg

SEQ ID NO: 579 - DNA, 27 bp, Homo sapiens
  source 1..27: mol_type = other DNA
gcggctgcgggtgtttcatccacaaaa

SEQ ID NO: 580 - DNA, 27 bp, Homo sapiens
  source 1..27: mol_type = other DNA
tttcccactaccatcaacttcactcga

SEQ ID NO: 581 - DNA, 27 bp, Homo sapiens
  source 1..27: mol_type = other DNA
actacctctgagaagattcacgagaga

SEQ ID NO: 582 - DNA, 27 bp, Homo sapiens
  source 1..27: mol_type = other DNA
gggctgtatgatgggatggagcacctc

SEQ ID NO: 583 - DNA, 33 bp, Homo sapiens
  source 1..33: mol_type = other DNA
agcactgccaatgtgacgcgggcccacggccgc

SEQ ID NO: 584 - DNA, 30 bp, Homo sapiens
  source 1..30: mol_type = other DNA
atagaggacccaaaggcagggctgcatgtg

SEQ ID NO: 585 - DNA, 27 bp, Homo sapiens
  source 1..27: mol_type = other DNA
ggtgtggtgggcccccagggcctgagg

SEQ ID NO: 586 - DNA, 33 bp, Homo sapiens
  source 1..33: mol_type = other DNA
aatgtatttgctcatgcaactggggaatcaagg

SEQ ID NO: 587 - DNA, 27 bp, Homo sapiens
  source 1..27: mol_type = other DNA
ttcctctttggggagcccaagaggctc

SEQ ID NO: 588 - DNA, 33 bp, Homo sapiens
  source 1..33: mol_type = other DNA
tccgcctttcccactaccatcaacttcactcga

SEQ ID NO: 589 - DNA, 27 bp, Homo sapiens
  source 1..27: mol_type = other DNA
aggctcttctcagaagacatacatgtc

SEQ ID NO: 590 - DNA, 27 bp, Homo sapiens
  source 1..27: mol_type = other DNA
gtttacatgcctgctaatacaacattt

SEQ ID NO: 591 - DNA, 27 bp, Homo sapiens
  source 1..27: mol_type = other DNA
gaggctgtgggctttcaaccaccacga

SEQ ID NO: 592 - DNA, 27 bp, Homo sapiens
  source 1..27: mol_type = other DNA
gaagtggaccccactagccactcctat

SEQ ID NO: 593 - DNA, 30 bp, Homo sapiens
  source 1..30: mol_type = other DNA
ttactggacccagctcagagaaacctgtac

SEQ ID NO: 594 - DNA, 27 bp, Homo sapiens
  source 1..27: mol_type = other DNA
tctggaatctcgcaagtcttccagagg

SEQ ID NO: 595 - DNA, 30 bp, Homo sapiens
  source 1..30: mol_type = other DNA
aggctcttctcagaagacatacatgtcgcc

SEQ ID NO: 596 - DNA, 27 bp, Homo sapiens
  source 1..27: mol_type = other DNA
gtgtttgatgttggcaaaggaaacatg

SEQ ID NO: 597 - DNA, 27 bp, Homo sapiens
  source 1..27: mol_type = other DNA
aaagcctcggagaaaatcttctatgtg

SEQ ID NO: 598 - DNA, 30 bp, Homo sapiens
  source 1..30: mol_type = other DNA
gaggccgccgctgccttcaggtttccgaga

SEQ ID NO: 599 - DNA, 27 bp, Homo sapiens
  source 1..27: mol_type = other DNA
atgagtatctccatgtctttattgaaa

SEQ ID NO: 600 - DNA, 27 bp, Homo sapiens
  source 1..27: mol_type = other DNA
aaggcatcccccagctctctgcctcat

SEQ ID NO: 601 - DNA, 33 bp, Homo sapiens
  source 1..33: mol_type = other DNA
agaaccttgcagcaaggagttctccaagactgg

SEQ ID NO: 602 - DNA, 27 bp, Homo sapiens
  source 1..27: mol_type = other DNA
actactgccacgacttttagagcgcgt

SEQ ID NO: 603 - DNA, 27 bp, Homo sapiens
  source 1..27: mol_type = other DNA
aatcctagccatctcatcacacccaga

SEQ ID NO: 604 - DNA, 30 bp, Homo sapiens
  source 1..30: mol_type = other DNA
gagacaatggagactctgagaacatccaga

SEQ ID NO: 605 - DNA, 33 bp, Homo sapiens
  source 1..33: mol_type = other DNA
atggtatgggaggggcatcccagagaaagaaga

SEQ ID NO: 606 - DNA, 27 bp, Homo sapiens
  source 1..27: mol_type = other DNA
ctcccagacggctcccgcgtggagctg

SEQ ID NO: 607 - DNA, 30 bp, Homo sapiens
  source 1..30: mol_type = other DNA
gatgttgtgactggcaacgtgcctctaaaa

SEQ ID NO: 608 - DNA, 30 bp, Homo sapiens
  source 1..30: mol_type = other DNA
gagaatcctagccatctcatcacacccaga

SEQ ID NO: 609 - DNA, 27 bp, Homo sapiens
  source 1..27: mol_type = other DNA
tcactggccaggactatactccatctc

SEQ ID NO: 610 - DNA, 27 bp, Homo sapiens
  source 1..27: mol_type = other DNA
aaaccacattatggattgagagcaatc

SEQ ID NO: 611 - DNA, 27 bp, Homo sapiens
  source 1..27: mol_type = other DNA
gcctttctggacatccacacagccctg

SEQ ID NO: 612 - DNA, 27 bp, Homo sapiens
  source 1..27: mol_type = other DNA
aagatgtcagagtttgtctacagattt

SEQ ID NO: 613 - DNA, 27 bp, Homo sapiens
  source 1..27: mol_type = other DNA
tttttagtgacaaagactgtgactatg

SEQ ID NO: 614 - DNA, 27 bp, Homo sapiens
  source 1..27: mol_type = other DNA
cacaccacctctgtgatggagaacaag

SEQ ID NO: 615 - DNA, 27 bp, Homo sapiens
  source 1..27: mol_type = other DNA
cttgtgagcattcccattgcagtaaga

SEQ ID NO: 616 - DNA, 27 bp, Homo sapiens
  source 1..27: mol_type = other DNA
attacatcaggagtaaaagtgaagaaa

SEQ ID NO: 617 - DNA, 27 bp, Homo sapiens
  source 1..27: mol_type = other DNA
gtgagcattcccattgcagtaagaaga

SEQ ID NO: 618 - DNA, 30 bp, Homo sapiens
  source 1..30: mol_type = other DNA
gacatcattgagctccaggaggggctgaga

SEQ ID NO: 619 - DNA, 27 bp, Homo sapiens
  source 1..27: mol_type = other DNA
catctcaagcctccagagctgggcttc

SEQ ID NO: 620 - DNA, 27 bp, Homo sapiens
  source 1..27: mol_type = other DNA
caggtcatcgtgaaaatcatcggacat

SEQ ID NO: 621 - DNA, 27 bp, Homo sapiens
  source 1..27: mol_type = other DNA
gccgagcaggaaataatgaagaaagtt

SEQ ID NO: 622 - DNA, 27 bp, Homo sapiens
  source 1..27: mol_type = other DNA
gaggttcccctgaagatcctggcccat

SEQ ID NO: 623 - DNA, 27 bp, Homo sapiens
  source 1..27: mol_type = other DNA
atcgtgaaaatcatcggacatttctat

SEQ ID NO: 624 - DNA, 30 bp, Homo sapiens
  source 1..30: mol_type = other DNA
tctcttgtgagcattcccattgcagtaaga

SEQ ID NO: 625 - DNA, 30 bp, Homo sapiens
  source 1..30: mol_type = other DNA
atggtttctcctggagtttctctattcaaa

SEQ ID NO: 626 - DNA, 27 bp, Homo sapiens
  source 1..27: mol_type = other DNA
gtgttgatgatgaagttggaagatttg

SEQ ID NO: 627 - DNA, 30 bp, Homo sapiens
  source 1..30: mol_type = other DNA
tattcggacttacacacccctgggaggtat

SEQ ID NO: 628 - DNA, 27 bp, Homo sapiens
  source 1..27: mol_type = other DNA
gaagtggcccggttcctgacctactac

SEQ ID NO: 629 - DNA, 27 bp, Homo sapiens
  source 1..27: mol_type = other DNA
agtgtcggagacctcatggctccccgg

SEQ ID NO: 630 - DNA, 27 bp, Homo sapiens
  source 1..27: mol_type = other DNA
gtgctctggagaggtgactctcctcta

SEQ ID NO: 631 - DNA, 30 bp, Homo sapiens
  source 1..30: mol_type = other DNA
cagccccgccagggctcacctgtggcagcg

SEQ ID NO: 632 - DNA, 27 bp, Homo sapiens
  source 1..27: mol_type = other DNA
aaacaaacaggtattaggcacagttat

SEQ ID NO: 633 - DNA, 27 bp, Homo sapiens
  source 1..27: mol_type = other DNA
agacccagacaagatgccatttcagta

SEQ ID NO: 634 - DNA, 27 bp, Homo sapiens
  source 1..27: mol_type = other DNA
aaactgataacacagatacgtacagca

SEQ ID NO: 635 - DNA, 33 bp, Homo sapiens
  source 1..33: mol_type = other DNA
tcttatttgtctgagcactctgcagctattttc

SEQ ID NO: 636 - DNA, 30 bp, Homo sapiens
  source 1..30: mol_type = other DNA
accagtgtcggagacctcatggctccccgg

SEQ ID NO: 637 - DNA, 27 bp, Homo sapiens
  source 1..27: mol_type = other DNA
gagataccaactggggtggccaagcgg

SEQ ID NO: 638 - DNA, 27 bp, Homo sapiens
  source 1..27: mol_type = other DNA
agtcaaatcaaccttagccaaatttat

SEQ ID NO: 639 - DNA, 27 bp, Homo sapiens
  source 1..27: mol_type = other DNA
cgaagaccctcaggcgggttccggctc

SEQ ID NO: 640 - DNA, 33 bp, Homo sapiens
  source 1..33: mol_type = other DNA
gaagtatttaatgtaaaaaatacatttccaaag

SEQ ID NO: 641 - DNA, 27 bp, Homo sapiens
  source 1..27: mol_type = other DNA
aaggtcttaggaggtggatggactgtc

SEQ ID NO: 642 - DNA, 30 bp, Homo sapiens
  source 1..30: mol_type = other DNA
catgaggctcctggccagcacagtgaagtt

SEQ ID NO: 643 - DNA, 30 bp, Homo sapiens
  source 1..30: mol_type = other DNA
gaagtaagccaccctaacatgagtggcaga

SEQ ID NO: 644 - DNA, 27 bp, Homo sapiens
  source 1..27: mol_type = other DNA
gacacagccccagccaccttcctacgg

SEQ ID NO: 645 - DNA, 27 bp, Homo sapiens
  source 1..27: mol_type = other DNA
aaaccccaaaaagacctgcacttcata

SEQ ID NO: 646 - DNA, 27 bp, Homo sapiens
  source 1..27: mol_type = other DNA
cgagtgcgtggggcaggaacacacctg

SEQ ID NO: 647 - DNA, 27 bp, Homo sapiens
  source 1..27: mol_type = other DNA
acggctcccattattgtggctcagaga

SEQ ID NO: 648 - DNA, 30 bp, Homo sapiens
  source 1..30: mol_type = other DNA
acgagctgggataccaagttctatgaaaag

SEQ ID NO: 649 - DNA, 30 bp, Homo sapiens
  source 1..30: mol_type = other DNA
aaagtgtttgcggagcacaagatctcctac

SEQ ID NO: 650 - DNA, 27 bp, Homo sapiens
  source 1..27: mol_type = other DNA
gacgccttccagggcatcgccaacagg

SEQ ID NO: 651 - DNA, 27 bp, Homo sapiens
  source 1..27: mol_type = other DNA
attgtggccgacccgcgggtgccccgt

SEQ ID NO: 652 - DNA, 27 bp, Homo sapiens
  source 1..27: mol_type = other DNA
acaggcttctccacgagcctgggcaga

SEQ ID NO: 653 - DNA, 30 bp, Homo sapiens
  source 1..30: mol_type = other DNA
gccacggctcccattattgtggctcagaga

SEQ ID NO: 654 - DNA, 27 bp, Homo sapiens
  source 1..27: mol_type = other DNA
ggccagttcttggtcaaatccggctac

SEQ ID NO: 655 - DNA, 27 bp, Homo sapiens
  source 1..27: mol_type = other DNA
tggacttccagaacggtgcaaaataaa

SEQ ID NO: 656 - DNA, 30 bp, Homo sapiens
  source 1..30: mol_type = other DNA
gccacatatgggggttcagatcattacatt

SEQ ID NO: 657 - DNA, 27 bp, Homo sapiens
  source 1..27: mol_type = other DNA
gtttacattgcagagttggaaaagata

SEQ ID NO: 658 - DNA, 27 bp, Homo sapiens
  source 1..27: mol_type = other DNA
gtgttcctggaccgcgccgtgtccatc

SEQ ID NO: 659 - DNA, 27 bp, Homo sapiens
  source 1..27: mol_type = other DNA
atgcccagtcccatcgaccacccaagg

SEQ ID NO: 660 - DNA, 33 bp, Homo sapiens
  source 1..33: mol_type = other DNA
gaagcagtaaggcacatcttacatggcagcagg

SEQ ID NO: 661 - DNA, 27 bp, Homo sapiens
  source 1..27: mol_type = other DNA
tacccagcaggcaacgtcaaagagcta

SEQ ID NO: 662 - DNA, 27 bp, Homo sapiens
  source 1..27: mol_type = other DNA
agcgtacccagaagcctggcatcccgg

SEQ ID NO: 663 - DNA, 27 bp, Homo sapiens
  source 1..27: mol_type = other DNA
gcgctaattaagcacgttgcgaacgca

SEQ ID NO: 664 - DNA, 27 bp, Homo sapiens
  source 1..27: mol_type = other DNA
tacacggcaaatctggttccagttaga

SEQ ID NO: 665 - DNA, 27 bp, Homo sapiens
  source 1..27: mol_type = other DNA
ttgtcctcttcccttgtcaccaaactt

SEQ ID NO: 666 - DNA, 27 bp, Homo sapiens
  source 1..27: mol_type = other DNA
cagaacagtaatgttttagcagtgagg

SEQ ID NO: 667 - DNA, 27 bp, Homo sapiens
  source 1..27: mol_type = other DNA
acgacaacaaaaaacaccttgaacaaa

SEQ ID NO: 668 - DNA, 27 bp, Homo sapiens
  source 1..27: mol_type = other DNA
cacaattatattaatttgcattctctg

SEQ ID NO: 669 - DNA, 27 bp, Homo sapiens
  source 1..27: mol_type = other DNA
caccttaaagattactttgaggaatat

SEQ ID NO: 670 - DNA, 27 bp, Homo sapiens
  source 1..27: mol_type = other DNA
tctccagaatattttaccagctatctg

SEQ ID NO: 671 - DNA, 27 bp, Homo sapiens
  source 1..27: mol_type = other DNA
gccacagatgttttggaccacttatat

SEQ ID NO: 672 - DNA, 27 bp, Homo sapiens
  source 1..27: mol_type = other DNA
cagacctttgaggaatcactgggaaga

SEQ ID NO: 673 - DNA, 27 bp, Homo sapiens
  source 1..27: mol_type = other DNA
catgaacagccagaactgcagaaggta

SEQ ID NO: 674 - DNA, 30 bp, Homo sapiens
  source 1..30: mol_type = other DNA
atggccacagatgttttggaccacttatat

SEQ ID NO: 675 - DNA, 27 bp, Homo sapiens
  source 1..27: mol_type = other DNA
acttctggcagtctcctgtttcccaga

SEQ ID NO: 676 - DNA, 27 bp, Homo sapiens
  source 1..27: mol_type = other DNA
aagcttggaataaataacgtttccttt

SEQ ID NO: 677 - DNA, 27 bp, Homo sapiens
  source 1..27: mol_type = other DNA
aaatatatttcacaatttcaagtattc

SEQ ID NO: 678 - DNA, 27 bp, Homo sapiens
  source 1..27: mol_type = other DNA
cagactgcggctgtgtcgggagtaaag

SEQ ID NO: 679 - DNA, 30 bp, Homo sapiens
  source 1..30: mol_type = other DNA
ttgacaccattaaaccatcctttttcctac

SEQ ID NO: 680 - DNA, 27 bp, Homo sapiens
  source 1..27: mol_type = other DNA
gtatttataaataaaaagtacgttttt

SEQ ID NO: 681 - DNA, 27 bp, Homo sapiens
  source 1..27: mol_type = other DNA
aacctcatcactgaggagcatcctgtc

SEQ ID NO: 682 - DNA, 33 bp, Homo sapiens
  source 1..33: mol_type = other DNA
gagttagtgaaggtcattgatggaagctccagg

SEQ ID NO: 683 - DNA, 27 bp, Homo sapiens
  source 1..27: mol_type = other DNA
tttcactcacttccaattcatttgttc

SEQ ID NO: 684 - DNA, 24 bp, Homo sapiens
  source 1..24: mol_type = other DNA
tattctcatgattactcttactat

SEQ ID NO: 685 - DNA, 27 bp, Homo sapiens
  source 1..27: mol_type = other DNA
ttccagatagaaaaattgacacccctg

SEQ ID NO: 686 - DNA, 27 bp, Homo sapiens
  source 1..27: mol_type = other DNA
gagcactctgttgaaaatggcagtatt

SEQ ID NO: 687 - DNA, 27 bp, Homo sapiens
  source 1..27: mol_type = other DNA
gagacaattcaaaacacacattcattt

SEQ ID NO: 688 - DNA, 27 bp, Homo sapiens
  source 1..27: mol_type = other DNA
tactacagtatttggaagacacaaatt

SEQ ID NO: 689 - DNA, 27 bp, Homo sapiens
  source 1..27: mol_type = other DNA
tcttattattttcacggcctgtcattt

SEQ ID NO: 690 - DNA, 30 bp, Homo sapiens
  source 1..30: mol_type = other DNA
gagtcttttcaacaaccttatattcccagg

SEQ ID NO: 691 - DNA, 27 bp, Homo sapiens
  source 1..27: mol_type = other DNA
aaacaggtaccctctagggtcctctac

SEQ ID NO: 692 - DNA, 30 bp, Homo sapiens
  source 1..30: mol_type = other DNA
atgttatttaataaagatttggatgcagca

SEQ ID NO: 693 - DNA, 30 bp, Homo sapiens
  source 1..30: mol_type = other DNA
gccccacggtcagcagagccacgggccacc

SEQ ID NO: 694 - DNA, 33 bp, Homo sapiens
  source 1..33: mol_type = other DNA
tatctggatcctggggcagtgcatgcagtgttt

SEQ ID NO: 695 - DNA, 24 bp, Homo sapiens
  source 1..24: mol_type = other DNA
acccttaagaaacataagatagtt

SEQ ID NO: 696 - DNA, 27 bp, Homo sapiens
  source 1..27: mol_type = other DNA
gtgggtgtgccaatcagcactctgtat

SEQ ID NO: 697 - DNA, 24 bp, Homo sapiens
  source 1..24: mol_type = other DNA
agcccccgcccccagccagagctc

SEQ ID NO: 698 - DNA, 27 bp, Homo sapiens
  source 1..27: mol_type = other DNA
aagctttccttccctgattcaaaattt

SEQ ID NO: 699 - DNA, 30 bp, Homo sapiens
  source 1..30: mol_type = other DNA
aaaaacaaagatgaatatatccatttttac

SEQ ID NO: 700 - DNA, 30 bp, Homo sapiens
  source 1..30: mol_type = other DNA
agggcatttgaggggagaagaagaggatgg

SEQ ID NO: 701 - DNA, 27 bp, Homo sapiens
  source 1..27: mol_type = other DNA
cgtctcaaggccaggccttccacctac

SEQ ID NO: 702 - DNA, 24 bp, Homo sapiens
  source 1..24: mol_type = other DNA
gccccctctcttcacgccacactc

SEQ ID NO: 703 - DNA, 27 bp, Homo sapiens
  source 1..27: mol_type = other DNA
gcgcagaaagaacccactggcaattac

SEQ ID NO: 704 - DNA, 27 bp, Homo sapiens
  source 1..27: mol_type = other DNA
ctctcactgggtaccacagcttgcagg

SEQ ID NO: 705 - DNA, 27 bp, Homo sapiens
  source 1..27: mol_type = other DNA
agcagaaaccctacaagctacaagtat

SEQ ID NO: 706 - DNA, 30 bp, Homo sapiens
  source 1..30: mol_type = other DNA
agcctaaataatgacgatcaccagatatac

SEQ ID NO: 707 - DNA, 27 bp, Homo sapiens
  source 1..27: mol_type = other DNA
tatgttgatcccatgattacagagata

SEQ ID NO: 708 - DNA, 27 bp, Homo sapiens
  source 1..27: mol_type = other DNA
cccagcttttctccatatgcaaaaagg

SEQ ID NO: 709 - DNA, 30 bp, Homo sapiens
  source 1..30: mol_type = other DNA
cacgtcaacggctcccgcccccatgcacgg

SEQ ID NO: 710 - DNA, 27 bp, Homo sapiens
  source 1..27: mol_type = other DNA
ataacagtgagattggctcctccactc

SEQ ID NO: 711 - DNA, 27 bp, Homo sapiens
  source 1..27: mol_type = other DNA
tcagccaatcccctaagtcatagatac

SEQ ID NO: 712 - DNA, 27 bp, Homo sapiens
  source 1..27: mol_type = other DNA
gtgcctcttgctcattccagctcagcc

SEQ ID NO: 713 - DNA, 27 bp, Homo sapiens
  source 1..27: mol_type = other DNA
ggccaagtgcctactacagaagttgtg

SEQ ID NO: 714 - DNA, 27 bp, Homo sapiens
  source 1..27: mol_type = other DNA
aagacctggggccaatactggcaagtt

SEQ ID NO: 715 - DNA, 27 bp, Homo sapiens
  source 1..27: mol_type = other DNA
gccttgaacttccctggaagccaaaag

SEQ ID NO: 716 - DNA, 27 bp, Homo sapiens
  source 1..27: mol_type = other DNA
tatgtctggaagacctggggccaatac

SEQ ID NO: 717 - DNA, 27 bp, Homo sapiens
  source 1..27: mol_type = other DNA
atgccaactgcagagagcacaggtatg

SEQ ID NO: 718 - DNA, 33 bp, Homo sapiens
  source 1..33: mol_type = other DNA
cacaccatggaagtgactgtctaccatcgccgg

SEQ ID NO: 719 - DNA, 27 bp, Homo sapiens
  source 1..27: mol_type = other DNA
gaagaggccgctgggatcggcatcctg

SEQ ID NO: 720 - DNA, 27 bp, Homo sapiens
  source 1..27: mol_type = other DNA
gaagggtttgatcatcgggacagcaaa

SEQ ID NO: 721 - DNA, 30 bp, Homo sapiens
  source 1..30: mol_type = other DNA
gctgaagaggccgctgggatcggcatcctg

SEQ ID NO: 722 - DNA, 27 bp, Homo sapiens
  source 1..27: mol_type = other DNA
ctcacttcatctatggttaccccaaga

SEQ ID NO: 723 - DNA, 27 bp, Homo sapiens
  source 1..27: mol_type = other DNA
acttcatctatggttaccccaagaagg

SEQ ID NO: 724 - DNA, 27 bp, Homo sapiens
  source 1..27: mol_type = other DNA
gggctgagcaatctgacccacgtgctg

SEQ ID NO: 725 - DNA, 27 bp, Homo sapiens
  source 1..27: mol_type = other DNA
cagctccttgccctcctgccttccctg

SEQ ID NO: 726 - DNA, 27 bp, Homo sapiens
  source 1..27: mol_type = other DNA
gaagcagctcagcccatgacaaagaag

SEQ ID NO: 727 - DNA, 27 bp, Homo sapiens
  source 1..27: mol_type = other DNA
agggttttaggaacaaagccttcctgg

SEQ ID NO: 728 - DNA, 30 bp, Homo sapiens
  source 1..30: mol_type = other DNA
gtgctgtatcctgtccccctggagagttat

SEQ ID NO: 729 - DNA, 27 bp, Homo sapiens
  source 1..27: mol_type = other DNA
ccttacctgggccagatgattaatctg

SEQ ID NO: 730 - DNA, 27 bp, Homo sapiens
  source 1..27: mol_type = other DNA
gaattgttctcctacctcattgagaaa

SEQ ID NO: 731 - DNA, 24 bp, Homo sapiens
  source 1..24: mol_type = other DNA
gaagagcagtatatcgcccagttc

SEQ ID NO: 732 - DNA, 30 bp, Homo sapiens
  source 1..30: mol_type = other DNA
gaagcagctcagcccatgacaaagaagcga

SEQ ID NO: 733 - DNA, 27 bp, Homo sapiens
  source 1..27: mol_type = other DNA
atgcccatgcaggatatcaagatgatc

SEQ ID NO: 734 - DNA, 30 bp, Homo sapiens
  source 1..30: mol_type = other DNA
cggctttcggaaggggatgtgatgcatctg

SEQ ID NO: 735 - DNA, 30 bp, Homo sapiens
  source 1..30: mol_type = other DNA
catgcatcttcctacatttccccggagaag

SEQ ID NO: 736 - DNA, 30 bp, Homo sapiens
  source 1..30: mol_type = other DNA
atgcccatgcaggatatcaagatgatcctg

SEQ ID NO: 737 - DNA, 30 bp, Homo sapiens
  source 1..30: mol_type = other DNA
agtcccagcgtcagtcagctaagtgtcctg

SEQ ID NO: 738 - DNA, 27 bp, Homo sapiens
  source 1..27: mol_type = other DNA
cgcctggatcagttgctcaggcacgtg

SEQ ID NO: 739 - DNA, 30 bp, Homo sapiens
  source 1..30: mol_type = other DNA
gctctgctggagagagcctctgccaccctc

SEQ ID NO: 740 - DNA, 30 bp, Homo sapiens
  source 1..30: mol_type = other DNA
accgtcgctcctaccccggccaccgctcgg

SEQ ID NO: 741 - DNA, 27 bp, Homo sapiens
  source 1..27: mol_type = other DNA
tcccagagtcccagcgtcagtcagcta

SEQ ID NO: 742 - DNA, 33 bp, Homo sapiens
  source 1..33: mol_type = other DNA
tcgaccgtcgctcctaccccggccaccgctcgg

SEQ ID NO: 743 - DNA, 30 bp, Homo sapiens
  source 1..30: mol_type = other DNA
cagtatatcgcccagttcacctctcagttc

SEQ ID NO: 744 - DNA, 27 bp, Homo sapiens
  source 1..27: mol_type = other DNA
ggacaacatcttcacctggagaccttc

SEQ ID NO: 745 - DNA, 27 bp, Homo sapiens
  source 1..27: mol_type = other DNA
acaattgcaagtgtgttggttgcaaga

SEQ ID NO: 746 - DNA, 27 bp, Homo sapiens
  source 1..27: mol_type = other DNA
agtctcctgcagcacctcatcgggctg

SEQ ID NO: 747 - DNA, 27 bp, Homo sapiens
  source 1..27: mol_type = other DNA
acagccagtcccaggagcccagtaatg

SEQ ID NO: 748 - DNA, 33 bp, Homo sapiens
  source 1..33: mol_type = other DNA
gcagcctttgacgggagacacagccagaccctg

SEQ ID NO: 749 - DNA, 24 bp, Homo sapiens
  source 1..24: mol_type = other DNA
agcccacggagacttgtggagctg

SEQ ID NO: 750 - DNA, 27 bp, Homo sapiens
  source 1..27: mol_type = other DNA
ctctatgtggactctttatttttcctt

SEQ ID NO: 751 - DNA, 27 bp, Homo sapiens
  source 1..27: mol_type = other DNA
gtcttcctgagagaatttcctaatttt

SEQ ID NO: 752 - DNA, 33 bp, Homo sapiens
  source 1..33: mol_type = other DNA
aggcccagattctccactgctaccgagatcgcc

SEQ ID NO: 753 - DNA, 27 bp, Homo sapiens
  source 1..27: mol_type = other DNA
gctttgattgctaacccaaggaggaag

SEQ ID NO: 754 - DNA, 27 bp, Homo sapiens
  source 1..27: mol_type = other DNA
gatttcatgggccagattgtgtaccgc

SEQ ID NO: 755 - DNA, 27 bp, Homo sapiens
  source 1..27: mol_type = other DNA
gaaattcccgatacagtaatgggcctt

SEQ ID NO: 756 - DNA, 27 bp, Homo sapiens
  source 1..27: mol_type = other DNA
gtaatgtccagcaccctgtacactgtg

SEQ ID NO: 757 - DNA, 24 bp, Homo sapiens
  source 1..24: mol_type = other DNA
tcacctcagcacaagccacgactg

SEQ ID NO: 758 - DNA, 27 bp, Homo sapiens
  source 1..27: mol_type = other DNA
gagcctaagccacccaccacagggctt

SEQ ID NO: 759 - DNA, 27 bp, Homo sapiens
  source 1..27: mol_type = other DNA
ataccagacacaattgcaagtgtgttg

SEQ ID NO: 760 - DNA, 30 bp, Homo sapiens
  source 1..30: mol_type = other DNA
acagatttcatgggccagattgtgtaccgc

SEQ ID NO: 761 - DNA, 27 bp, Homo sapiens
  source 1..27: mol_type = other DNA
ggagaagatgatgaacacagaaaaatt

SEQ ID NO: 762 - DNA, 30 bp, Homo sapiens
  source 1..30: mol_type = other DNA
cgtttgcggggttccattcagagccgatac

SEQ ID NO: 763 - DNA, 27 bp, Homo sapiens
  source 1..27: mol_type = other DNA
cacttggatgagagccccgcctacctc

SEQ ID NO: 764 - DNA, 27 bp, Homo sapiens
  source 1..27: mol_type = other DNA
aagatggaagaaactacggtcacatat

SEQ ID NO: 765 - DNA, 27 bp, Homo sapiens
  source 1..27: mol_type = other DNA
ctcccaacagatctgtttaatagtgtg

SEQ ID NO: 766 - DNA, 24 bp, Homo sapiens
  source 1..24: mol_type = other DNA
aggcctcagggcctggctgtcctg

SEQ ID NO: 767 - DNA, 27 bp, Homo sapiens
  source 1..27: mol_type = other DNA
agtctatctgaaaagtctccagaagtc

SEQ ID NO: 768 - DNA, 27 bp, Homo sapiens
  source 1..27: mol_type = other DNA
ggtgcatttcgagaggggccgccctac

SEQ ID NO: 769 - DNA, 27 bp, Homo sapiens
  source 1..27: mol_type = other DNA
tacctcaatggggctactgttgtagca

SEQ ID NO: 770 - DNA, 27 bp, Homo sapiens
  source 1..27: mol_type = other DNA
tcaatgtacctccacacagagggcttc

SEQ ID NO: 771 - DNA, 27 bp, Homo sapiens
  source 1..27: mol_type = other DNA
cacattctgagtcacttgcatgaagtc

SEQ ID NO: 772 - DNA, 30 bp, Homo sapiens
  source 1..30: mol_type = other DNA
agtatctctgaagccccacttacagaggtt

SEQ ID NO: 773 - DNA, 27 bp, Homo sapiens
  source 1..27: mol_type = other DNA
caccagatcaacagtgaagccacattt

SEQ ID NO: 774 - DNA, 30 bp, Homo sapiens
  source 1..30: mol_type = other DNA
gacatcaagcaggaaggggtcggtgcattt

SEQ ID NO: 775 - DNA, 27 bp, Homo sapiens
  source 1..27: mol_type = other DNA
agcgctctgggagttactattaccaaa

SEQ ID NO: 776 - DNA, 30 bp, Homo sapiens
  source 1..30: mol_type = other DNA
acagtgaatgactttacccaaaaccccagg

SEQ ID NO: 777 - DNA, 27 bp, Homo sapiens
  source 1..27: mol_type = other DNA
tctttcatccaagcaggatcctcgtat

SEQ ID NO: 778 - DNA, 27 bp, Homo sapiens
  source 1..27: mol_type = other DNA
ggagttactattaccaaaactctcaag

SEQ ID NO: 779 - DNA, 27 bp, Homo sapiens
  source 1..27: mol_type = other DNA
gctgagaggcctcagggcctggctgtc

SEQ ID NO: 780 - DNA, 27 bp, Homo sapiens
  source 1..27: mol_type = other DNA
agggaaaagggacccgtcaaggaagtt

SEQ ID NO: 781 - DNA, 27 bp, Homo sapiens
  source 1..27: mol_type = other DNA
acctcagtgcctcccttcaacctaaga

SEQ ID NO: 782 - DNA, 27 bp, Homo sapiens
  source 1..27: mol_type = other DNA
ttatcacgccttccaagcattgccttt

SEQ ID NO: 783 - DNA, 27 bp, Homo sapiens
  source 1..27: mol_type = other DNA
ttcccagttcatgctgctgagaagttc

SEQ ID NO: 784 - DNA, 24 bp, Homo sapiens
  source 1..24: mol_type = other DNA
ggggagatctctgcatcacatgtc

SEQ ID NO: 785 - DNA, 30 bp, Homo sapiens
  source 1..30: mol_type = other DNA
gctgcagatgggggtcaacactggacgtat

SEQ ID NO: 786 - DNA, 27 bp, Homo sapiens
  source 1..27: mol_type = other DNA
gagatctctgcatcacatgtcagcagg

SEQ ID NO: 787 - DNA, 27 bp, Homo sapiens
  source 1..27: mol_type = other DNA
tcgccccggatcccgttcagcaccttc

SEQ ID NO: 788 - DNA, 33 bp, Homo sapiens
  source 1..33: mol_type = other DNA
aagctcatggacccgggctccctgccgcccctc

SEQ ID NO: 789 - DNA, 27 bp, Homo sapiens
  source 1..27: mol_type = other DNA
gaaacaaagccagagtttgaaaggagg

SEQ ID NO: 790 - DNA, 27 bp, Homo sapiens
  source 1..27: mol_type = other DNA
gagacgatcatgaaatttttgaaaatg

SEQ ID NO: 791 - DNA, 27 bp, Homo sapiens
  source 1..27: mol_type = other DNA
aggcgcgcccacctttggagcactttt

SEQ ID NO: 792 - DNA, 27 bp, Homo sapiens
  source 1..27: mol_type = other DNA
catctccaggaaaagctgcagagtttg

SEQ ID NO: 793 - DNA, 27 bp, Homo sapiens
  source 1..27: mol_type = other DNA
gaactctcagtggtcacaagtgagaga

SEQ ID NO: 794 - DNA, 30 bp, Homo sapiens
  source 1..30: mol_type = other DNA
tacttggaccagcaagtgccctacaccttc

SEQ ID NO: 795 - DNA, 27 bp, Homo sapiens
  source 1..27: mol_type = other DNA
gaagcaattcctcgaatccctcgcttg

SEQ ID NO: 796 - DNA, 33 bp, Homo sapiens
  source 1..33: mol_type = other DNA
ggatacttggaccagcaagtgccctacaccttc

SEQ ID NO: 797 - DNA, 27 bp, Homo sapiens
  source 1..27: mol_type = other DNA
agccctaaactgggcgggattggattt

SEQ ID NO: 798 - DNA, 27 bp, Homo sapiens
  source 1..27: mol_type = other DNA
tatcctgagaaatcacgtggatgcctc

SEQ ID NO: 799 - DNA, 33 bp, Homo sapiens
  source 1..33: mol_type = other DNA
accaccattcccttcagcactctacgtcagagg

SEQ ID NO: 800 - DNA, 30 bp, Homo sapiens
  source 1..30: mol_type = other DNA
agcctgggtgcccacagtcctgccctgtac

SEQ ID NO: 801 - DNA, 27 bp, Homo sapiens
  source 1..27: mol_type = other DNA
tctcaaaatggacaactaatccattac

SEQ ID NO: 802 - DNA, 33 bp, Homo sapiens
  source 1..33: mol_type = other DNA
aaagccaaggagcacaccgccaatatttcatgg

SEQ ID NO: 803 - DNA, 27 bp, Homo sapiens
  source 1..27: mol_type = other DNA
agggtatcaaagtcagattctactttc

SEQ ID NO: 804 - DNA, 27 bp, Homo sapiens
  source 1..27: mol_type = other DNA
tccctcaattctcaagtcacaggtctt

SEQ ID NO: 805 - DNA, 30 bp, Homo sapiens
  source 1..30: mol_type = other DNA
aactcagatttaagtgaaaaattggaatat

SEQ ID NO: 806 - DNA, 27 bp, Homo sapiens
  source 1..27: mol_type = other DNA
gacagttccactgttgggcttcagcga

SEQ ID NO: 807 - DNA, 27 bp, Homo sapiens
  source 1..27: mol_type = other DNA
agagcttcagctattataattcagaga

SEQ ID NO: 808 - DNA, 27 bp, Homo sapiens
  source 1..27: mol_type = other DNA
tcccaggaaacacttccactcaagatt

SEQ ID NO: 809 - DNA, 27 bp, Homo sapiens
  source 1..27: mol_type = other DNA
agcacctttgatggtttagacagaaga

SEQ ID NO: 810 - DNA, 27 bp, Homo sapiens
  source 1..27: mol_type = other DNA
cgagtgaaggagattgtaatcaacaag

SEQ ID NO: 811 - DNA, 27 bp, Homo sapiens
  source 1..27: mol_type = other DNA
gctcagccccaccatggtgtgccatat

SEQ ID NO: 812 - DNA, 33 bp, Homo sapiens
  source 1..33: mol_type = other DNA
gaagtaatacagttaaaagaacgagaccgtaag

SEQ ID NO: 813 - DNA, 24 bp, Homo sapiens
  source 1..24: mol_type = other DNA
gaggtgaaggaaaagacggaactc

SEQ ID NO: 814 - DNA, 24 bp, Homo sapiens
  source 1..24: mol_type = other DNA
actgaccctgagaccctgcactac

SEQ ID NO: 815 - DNA, 27 bp, Homo sapiens
  source 1..27: mol_type = other DNA
aaaatgaaggagaaaacacaagagctt

SEQ ID NO: 816 - DNA, 30 bp, Homo sapiens
  source 1..30: mol_type = other DNA
agcacctttgatggtttagacagaagaaga

SEQ ID NO: 817 - DNA, 27 bp, Homo sapiens
  source 1..27: mol_type = other DNA
aaaatgtcagagctgcagacctatgtt

SEQ ID NO: 818 - DNA, 27 bp, Homo sapiens
  source 1..27: mol_type = other DNA
ggcactgaccctgagaccctgcactac

SEQ ID NO: 819 - DNA, 27 bp, Homo sapiens
  source 1..27: mol_type = other DNA
acagaggaaaaggggagcattttaatt

SEQ ID NO: 820 - DNA, 27 bp, Homo sapiens
  source 1..27: mol_type = other DNA
gacactgctatgcctgcccattcaaga

SEQ ID NO: 821 - DNA, 27 bp, Homo sapiens
  source 1..27: mol_type = other DNA
aagctccaggaagcactgaagaccatc

SEQ ID NO: 822 - DNA, 30 bp, Homo sapiens
  source 1..30: mol_type = other DNA
ggcactgaccctgagaccctgcactacttc

SEQ ID NO: 823 - DNA, 27 bp, Homo sapiens
  source 1..27: mol_type = other DNA
agtttaaaaaatcgaattgaatctgta

SEQ ID NO: 824 - DNA, 27 bp, Homo sapiens
  source 1..27: mol_type = other DNA
aaccttaaagacccagttggggtatac

SEQ ID NO: 825 - DNA, 27 bp, Homo sapiens
  source 1..27: mol_type = other DNA
ttcacagagaccaacccacaagtgtac

SEQ ID NO: 826 - DNA, 33 bp, Homo sapiens
  source 1..33: mol_type = other DNA
gaaacccaggaggcagaggttgcagtgagccga

SEQ ID NO: 827 - DNA, 27 bp, Homo sapiens
  source 1..27: mol_type = other DNA
tctcccagaccagagtcgggtactgtt

SEQ ID NO: 828 - DNA, 27 bp, Homo sapiens
  source 1..27: mol_type = other DNA
tctgagtttgcgaaggatccgtccctc

SEQ ID NO: 829 - DNA, 27 bp, Homo sapiens
  source 1..27: mol_type = other DNA
aaggttatcaagaaaattggacattat

SEQ ID NO: 830 - DNA, 27 bp, Homo sapiens
  source 1..27: mol_type = other DNA
aggacgtggcaaaccagaaaacagttt

SEQ ID NO: 831 - DNA, 27 bp, Homo sapiens
  source 1..27: mol_type = other DNA
gccatggcccacaaagtaattaaagct

SEQ ID NO: 832 - DNA, 27 bp, Homo sapiens
  source 1..27: mol_type = other DNA
aaactaaaacagtttcagaaggaatat

SEQ ID NO: 833 - DNA, 27 bp, Homo sapiens
  source 1..27: mol_type = other DNA
tctccattggaagatgaagagtcatta

SEQ ID NO: 834 - DNA, 27 bp, Homo sapiens
  source 1..27: mol_type = other DNA
aactctacagactttaccactgacaga

SEQ ID NO: 835 - DNA, 30 bp, Homo sapiens
  source 1..30: mol_type = other DNA
gaaacgaaggataccaaaggtcgttatttt

SEQ ID NO: 836 - DNA, 27 bp, Homo sapiens
  source 1..27: mol_type = other DNA
cggcctcgagtatcctccacagaaatg

SEQ ID NO: 837 - DNA, 27 bp, Homo sapiens
  source 1..27: mol_type = other DNA
ttaatggacaaatcaagcactatttac

SEQ ID NO: 838 - DNA, 30 bp, Homo sapiens
  source 1..30: mol_type = other DNA
gggaccctccggccgctcaaagttcgcagg

SEQ ID NO: 839 - DNA, 27 bp, Homo sapiens
  source 1..27: mol_type = other DNA
atcctgagtgaaggatccgtccctctc

SEQ ID NO: 840 - DNA, 27 bp, Homo sapiens
  source 1..27: mol_type = other DNA
acagtctataattttaggactgaactt

SEQ ID NO: 841 - DNA, 27 bp, Homo sapiens
  source 1..27: mol_type = other DNA
ctcccctcttcagctgatgtagaattt

SEQ ID NO: 842 - DNA, 30 bp, Homo sapiens
  source 1..30: mol_type = other DNA
aggctcccctcttcagctgatgtagaattt

SEQ ID NO: 843 - DNA, 27 bp, Homo sapiens
  source 1..27: mol_type = other DNA
gccttgatggataaaagtcttcatgtt

SEQ ID NO: 844 - DNA, 27 bp, Homo sapiens
  source 1..27: mol_type = other DNA
atgcacaatgccttgcacatctatatg

SEQ ID NO: 845 - DNA, 27 bp, Homo sapiens
  source 1..27: mol_type = other DNA
atggagaaagaggattaccacagcttg

SEQ ID NO: 846 - DNA, 30 bp, Homo sapiens
  source 1..30: mol_type = other DNA
atggagaaagaggattaccacagcttgtat

SEQ ID NO: 847 - DNA, 27 bp, Homo sapiens
  source 1..27: mol_type = other DNA
taccacagcttgtatcagagccattta

SEQ ID NO: 848 - DNA, 27 bp, Homo sapiens
  source 1..27: mol_type = other DNA
tacatggttccttttataccactgtac

SEQ ID NO: 849 - DNA, 30 bp, Homo sapiens
  source 1..30: mol_type = other DNA
ctcatggagaaagaggattaccacagcttg

SEQ ID NO: 850 - DNA, 27 bp, Homo sapiens
  source 1..27: mol_type = other DNA
tttgcccatgaagcaccagcttttctg

SEQ ID NO: 851 - DNA, 27 bp, Homo sapiens
  source 1..27: mol_type = other DNA
tttgctagtccacttactgggatagcg

SEQ ID NO: 852 - DNA, 27 bp, Homo sapiens
  source 1..27: mol_type = other DNA
catgaagcaccagcttttctgccttgg

SEQ ID NO: 853 - DNA, 27 bp, Homo sapiens
  source 1..27: mol_type = other DNA
gtcctcttcaagaagtttatccagaag

SEQ ID NO: 854 - DNA, 27 bp, Homo sapiens
  source 1..27: mol_type = other DNA
gcaccagcttttctgccttggcataga

SEQ ID NO: 855 - DNA, 33 bp, Homo sapiens
  source 1..33: mol_type = other DNA
ctcctcatggagaaagaggattaccacagcttg

SEQ ID NO: 856 - DNA, 27 bp, Homo sapiens
  source 1..27: mol_type = other DNA
cattattatgtgtcaatggatgcactg

SEQ ID NO: 857 - DNA, 24 bp, Homo sapiens
  source 1..24: mol_type = other DNA
tgggaacaagaaatccagaagctg

SEQ ID NO: 858 - DNA, 33 bp, Homo sapiens
  source 1..33: mol_type = other DNA
ctcatggagaaagaggattaccacagcttgtat

SEQ ID NO: 859 - DNA, 30 bp, Homo sapiens
  source 1..30: mol_type = other DNA
ttggaacaagcgagtcggatctggtcatgg

SEQ ID NO: 860 - DNA, 30 bp, Homo sapiens
  source 1..30: mol_type = other DNA
aacatcttcgatttgagtgccccagagaag

SEQ ID NO: 861 - DNA, 30 bp, Homo sapiens
  source 1..30: mol_type = other DNA
gaagcaccagcttttctgccttggcataga

SEQ ID NO: 862 - DNA, 27 bp, Homo sapiens
  source 1..27: mol_type = other DNA
tctgaaatctggagagacattgatttt

SEQ ID NO: 863 - DNA, 24 bp, Homo sapiens
  source 1..24: mol_type = other DNA
gcggcgatggtaggggccgtcctc

SEQ ID NO: 864 - DNA, 30 bp, Homo sapiens
  source 1..30: mol_type = other DNA
actttccttggggtaggacaggaaagcttt

SEQ ID NO: 865 - DNA, 30 bp, Homo sapiens
  source 1..30: mol_type = other DNA
gagtcgtggccttccgtcttttataatagg

SEQ ID NO: 866 - DNA, 27 bp, Homo sapiens
  source 1..27: mol_type = other DNA
atttctcaggacacaactgtgggacgt

SEQ ID NO: 867 - DNA, 27 bp, Homo sapiens
  source 1..27: mol_type = other DNA
aggccctggagtggtccctacatccta

SEQ ID NO: 868 - DNA, 33 bp, Homo sapiens
  source 1..33: mol_type = other DNA
agctcagactatgtcatccccatagggacctat

SEQ ID NO: 869 - DNA, 30 bp, Homo sapiens
  source 1..30: mol_type = other DNA
gattcagacccagactcttttcaagactac

SEQ ID NO: 870 - DNA, 27 bp, Homo sapiens
  source 1..27: mol_type = other DNA
gacacaaggccctggagtggtccctac

SEQ ID NO: 871 - DNA, 27 bp, Homo sapiens
  source 1..27: mol_type = other DNA
cactattactcagtcaaaaagactttc

SEQ ID NO: 872 - DNA, 27 bp, Homo sapiens
  source 1..27: mol_type = other DNA
aatgtctgtggctctcagcaaggccgg

SEQ ID NO: 873 - DNA, 27 bp, Homo sapiens
  source 1..27: mol_type = other DNA
tatatgaatggaacaatgtcccaggta

SEQ ID NO: 874 - DNA, 27 bp, Homo sapiens
  source 1..27: mol_type = other DNA
atggcaaagcgcacaactcacccttta

SEQ ID NO: 875 - DNA, 30 bp, Homo sapiens
  source 1..30: mol_type = other DNA
aggccctggagtggtccctacatcctacga

SEQ ID NO: 876 - DNA, 27 bp, Homo sapiens
  source 1..27: mol_type = other DNA
atgacggtggacagcctagtgaacaag

SEQ ID NO: 877 - DNA, 30 bp, Homo sapiens
  source 1..30: mol_type = other DNA
acaaggccctggagtggtccctacatccta

SEQ ID NO: 878 - DNA, 33 bp, Homo sapiens
  source 1..33: mol_type = other DNA
acaactcaccctttatttgtcattgccaccagg

SEQ ID NO: 879 - DNA, 27 bp, Homo sapiens
  source 1..27: mol_type = other DNA
tttgtttggacacactattactcagtc

SEQ ID NO: 880 - DNA, 27 bp, Homo sapiens
  source 1..27: mol_type = other DNA
ttaagtaaagaagaaaagaaccacttt

SEQ ID NO: 881 - DNA, 27 bp, Homo sapiens
  source 1..27: mol_type = other DNA
agagctgcaaaccaggtctttgttttg

SEQ ID NO: 882 - DNA, 27 bp, Homo sapiens
  source 1..27: mol_type = other DNA
atggtgagaagcgctaccctcattaaa

SEQ ID NO: 883 - DNA, 27 bp, Homo sapiens
  source 1..27: mol_type = other DNA
aaccaggtctttgttttgcactcttat

SEQ ID NO: 884 - DNA, 24 bp, Homo sapiens
  source 1..24: mol_type = other DNA
cacaactccacagagtttctcatc

SEQ ID NO: 885 - DNA, 27 bp, Homo sapiens
  source 1..27: mol_type = other DNA
tctcaggatgttgcaggcacaactttc

SEQ ID NO: 886 - DNA, 27 bp, Homo sapiens
  source 1..27: mol_type = other DNA
gcacacaactccacagagtttctcatc

SEQ ID NO: 887 - DNA, 27 bp, Homo sapiens
  source 1..27: mol_type = other DNA
tccacagagtttctcatctacgaaaga

SEQ ID NO: 888 - DNA, 27 bp, Homo sapiens
  source 1..27: mol_type = other DNA
gaagggtttttcacgagacaggagcgc

SEQ ID NO: 889 - DNA, 24 bp, Homo sapiens
  source 1..24: mol_type = other DNA
tttcagaaagttttggtatcctac

SEQ ID NO: 890 - DNA, 30 bp, Homo sapiens
  source 1..30: mol_type = other DNA
ttcacagatttcatgggccagattgtgtac

SEQ ID NO: 891 - DNA, 27 bp, Homo sapiens
  source 1..27: mol_type = other DNA
tactttcagaaagttttggtatcctac

SEQ ID NO: 892 - DNA, 30 bp, Homo sapiens
  source 1..30: mol_type = other DNA
gcactggtgaacatgcctcctcactaccgc

SEQ ID NO: 893 - DNA, 30 bp, Homo sapiens
  source 1..30: mol_type = other DNA
ggtgtaatgtccagcaccctgtacactgtg

SEQ ID NO: 894 - DNA, 27 bp, Homo sapiens
  source 1..27: mol_type = other DNA
ttgctatctaatacggtctcaacacta

SEQ ID NO: 895 - DNA, 30 bp, Homo sapiens
  source 1..30: mol_type = other DNA
agggtttttcacgagacaggagcgcagaga

SEQ ID NO: 896 - DNA, 27 bp, Homo sapiens
  source 1..27: mol_type = other DNA
tcggccagcgaccactgccggtccagg

SEQ ID NO: 897 - DNA, 27 bp, Homo sapiens
  source 1..27: mol_type = other DNA
cgcagagatggaggcatcataatctat

SEQ ID NO: 898 - DNA, 27 bp, Homo sapiens
  source 1..27: mol_type = other DNA
gtagcagctttgattgctaacccaagg

SEQ ID NO: 899 - DNA, 27 bp, Homo sapiens
  source 1..27: mol_type = other DNA
agactgttgggtacagaattccaggtc

SEQ ID NO: 900 - DNA, 27 bp, Homo sapiens
  source 1..27: mol_type = other DNA
ttctccatcatcggagtttcccgaagg

SEQ ID NO: 901 - DNA, 27 bp, Homo sapiens
  source 1..27: mol_type = other DNA
ttgtcatcagatggaatgtacgagtat

SEQ ID NO: 902 - DNA, 24 bp, Homo sapiens
  source 1..24: mol_type = other DNA
gatgaatacttcctaccctccctg

SEQ ID NO: 903 - DNA, 30 bp, Homo sapiens
  source 1..30: mol_type = other DNA
tcctacattggattaaagggtctttacttc

SEQ ID NO: 904 - DNA, 30 bp, Homo sapiens
  source 1..30: mol_type = other DNA
ttcacaggttttggaggtgccctgggttac

SEQ ID NO: 905 - DNA, 30 bp, Homo sapiens
  source 1..30: mol_type = other DNA
aagacctgggaccaggtgcctttctccgtg

SEQ ID NO: 906 - DNA, 27 bp, Homo sapiens
  source 1..27: mol_type = other DNA
agggtggcggatattccaataaagcca

SEQ ID NO: 907 - DNA, 27 bp, Homo sapiens
  source 1..27: mol_type = other DNA
tctgttgccaagcacacccagagctac

SEQ ID NO: 908 - DNA, 30 bp, Homo sapiens
  source 1..30: mol_type = other DNA
cagacaaaagggggccaaacatgggcgaga

SEQ ID NO: 909 - DNA, 27 bp, Homo sapiens
  source 1..27: mol_type = other DNA
gaaacaggaagttcattcatgagtttt

SEQ ID NO: 910 - DNA, 27 bp, Homo sapiens
  source 1..27: mol_type = other DNA
tcactttattcttactttcagaaagtt

SEQ ID NO: 911 - DNA, 33 bp, Homo sapiens
  source 1..33: mol_type = other DNA
cagacaaaagggggccaaacatgggcgagaagg

SEQ ID NO: 912 - DNA, 33 bp, Homo sapiens
  source 1..33: mol_type = other DNA
actgttgtagcagctttgattgctaacccaagg

SEQ ID NO: 913 - DNA, 27 bp, Homo sapiens
  source 1..27: mol_type = other DNA
catctcatggtgaaagattggcagctg

SEQ ID NO: 914 - DNA, 27 bp, Homo sapiens
  source 1..27: mol_type = other DNA
aaccctctaagtaagctctctgtgctc

SEQ ID NO: 915 - DNA, 33 bp, Homo sapiens
  source 1..33: mol_type = other DNA
tttgtaattcctagcacgaaagactctaacagg

SEQ ID NO: 916 - DNA, 24 bp, Homo sapiens
  source 1..24: mol_type = other DNA
cactatcaggtgcagacccacctc

SEQ ID NO: 917 - DNA, 33 bp, Homo sapiens
  source 1..33: mol_type = other DNA
gcctcggacatcctgtcctttgcgcacaagtac

SEQ ID NO: 918 - DNA, 27 bp, Homo sapiens
  source 1..27: mol_type = other DNA
agactggggcagggcgtgcccctcgtg

SEQ ID NO: 919 - DNA, 30 bp, Homo sapiens
  source 1..30: mol_type = other DNA
agtgtaccagaccatgtccaaccctctaag

SEQ ID NO: 920 - DNA, 27 bp, Homo sapiens
  source 1..27: mol_type = other DNA
aggctgctggaaaagcacatctccctg

SEQ ID NO: 921 - DNA, 27 bp, Homo sapiens
  source 1..27: mol_type = other DNA
tccctctttggggtctcagagagattg

SEQ ID NO: 922 - DNA, 27 bp, Homo sapiens
  source 1..27: mol_type = other DNA
aatcactatcaggtgcagacccacctc

SEQ ID NO: 923 - DNA, 33 bp, Homo sapiens
  source 1..33: mol_type = other DNA
gacctggttggaaaggatgtaacaagagtgtac

SEQ ID NO: 924 - DNA, 27 bp, Homo sapiens
  source 1..27: mol_type = other DNA
tctctcagtcctccaacactaccaaga

SEQ ID NO: 925 - DNA, 30 bp, Homo sapiens
  source 1..30: mol_type = other DNA
ctggctgacaatggcaccctgggcaagtat

SEQ ID NO: 926 - DNA, 27 bp, Homo sapiens
  source 1..27: mol_type = other DNA
aaagccatgtatatccgtgtatcctat

SEQ ID NO: 927 - DNA, 27 bp, Homo sapiens
  source 1..27: mol_type = other DNA
atgcagcccaagctgaaacaagtcttt

SEQ ID NO: 928 - DNA, 27 bp, Homo sapiens
  source 1..27: mol_type = other DNA
gcaattattcaggatggcatcttgctg

SEQ ID NO: 929 - DNA, 24 bp, Homo sapiens
  source 1..24: mol_type = other DNA
gcgtttgacagagctgttcacttc

SEQ ID NO: 930 - DNA, 27 bp, Homo sapiens
  source 1..27: mol_type = other DNA
aatactcctaacattgtctctttaaga

SEQ ID NO: 931 - DNA, 30 bp, Homo sapiens
  source 1..30: mol_type = other DNA
agggtggcggatattccaataaagccatgg

SEQ ID NO: 932 - DNA, 27 bp, Homo sapiens
  source 1..27: mol_type = other DNA
cactatataatcccactgcaaacaata

SEQ ID NO: 933 - DNA, 27 bp, Homo sapiens
  source 1..27: mol_type = other DNA
gtcatgaagggcattaatcaaggttat

SEQ ID NO: 934 - DNA, 27 bp, Homo sapiens
  source 1..27: mol_type = other DNA
gcgacgtccccgccggcctcggtccgc

SEQ ID NO: 935 - DNA, 27 bp, Homo sapiens
  source 1..27: mol_type = other DNA
gcactggagaaggatacccccaggtat

SEQ ID NO: 936 - DNA, 30 bp, Homo sapiens
  source 1..30: mol_type = other DNA
caagtcatgaagggcattaatcaaggttat

SEQ ID NO: 937 - DNA, 27 bp, Homo sapiens
  source 1..27: mol_type = other DNA
gcagcgtttgacagagctgttcacttc

SEQ ID NO: 938 - DNA, 27 bp, Homo sapiens
  source 1..27: mol_type = other DNA
agaccgctgaggatcaaaggggttttc

SEQ ID NO: 939 - DNA, 33 bp, Homo sapiens
  source 1..33: mol_type = other DNA
aggcaagtcatgaagggcattaatcaaggttat

SEQ ID NO: 940 - DNA, 27 bp, Homo sapiens
  source 1..27: mol_type = other DNA
aaactggacaccactgtaaagaacatc

SEQ ID NO: 941 - DNA, 27 bp, Homo sapiens
  source 1..27: mol_type = other DNA
cttccgcgaactcctcgccccgacctc

SEQ ID NO: 942 - DNA, 27 bp, Homo sapiens
  source 1..27: mol_type = other DNA
ataacttacaccgaaagcaactcaagg

SEQ ID NO: 943 - DNA, 27 bp, Homo sapiens
  source 1..27: mol_type = other DNA
cgacttcagggatctgtcaccctcgtc

SEQ ID NO: 944 - DNA, 33 bp, Homo sapiens
  source 1..33: mol_type = other DNA
tctcaattcccaagtaaaaatcttcctgctttt

SEQ ID NO: 945 - DNA, 33 bp, Homo sapiens
  source 1..33: mol_type = other DNA
cttccgcgaactcctcgccccgacctcatcctt

SEQ ID NO: 946 - DNA, 27 bp, Homo sapiens
  source 1..27: mol_type = other DNA
gacactgccccacaatcccatcaagtc

SEQ ID NO: 947 - DNA, 27 bp, Homo sapiens
  source 1..27: mol_type = other DNA
atggctatcctgggcacagcggggcgg

SEQ ID NO: 948 - DNA, 27 bp, Homo sapiens
  source 1..27: mol_type = other DNA
tctgtgtcctctcacctcattcataga

SEQ ID NO: 949 - DNA, 30 bp, Homo sapiens
  source 1..30: mol_type = other DNA
tccacgtcaccagggacaggagtcaggaaa

SEQ ID NO: 950 - DNA, 30 bp, Homo sapiens
  source 1..30: mol_type = other DNA
gaagtggctgaagtccttgggagtgtgaag

SEQ ID NO: 951 - DNA, 30 bp, Homo sapiens
  source 1..30: mol_type = other DNA
gagataccacgaaccccctcagaaggaaga

SEQ ID NO: 952 - DNA, 27 bp, Homo sapiens
  source 1..27: mol_type = other DNA
gtgtttttaaaattctccagaagattt

SEQ ID NO: 953 - DNA, 30 bp, Homo sapiens
  source 1..30: mol_type = other DNA
tctgtgtcctctcacctcattcatagatac

SEQ ID NO: 954 - DNA, 27 bp, Homo sapiens
  source 1..27: mol_type = other DNA
acacatctagaagaccctcaccttatg

SEQ ID NO: 955 - DNA, 27 bp, Homo sapiens
  source 1..27: mol_type = other DNA
agtgtctttcccaggacctcctccaga

SEQ ID NO: 956 - DNA, 33 bp, Homo sapiens
  source 1..33: mol_type = other DNA
gggcagtatggacaaagtaagccttggactttt

SEQ ID NO: 957 - DNA, 30 bp, Homo sapiens
  source 1..30: mol_type = other DNA
gttccaagacctaccagcacagtaggatta

SEQ ID NO: 958 - DNA, 33 bp, Homo sapiens
  source 1..33: mol_type = other DNA
aattctgtgtcctctcacctcattcatagatac

SEQ ID NO: 959 - DNA, 30 bp, Homo sapiens
  source 1..30: mol_type = other DNA
caagaagaagacaaccttctaagatacttt

SEQ ID NO: 960 - DNA, 30 bp, Homo sapiens
  source 1..30: mol_type = other DNA
acacatctagaagaccctcaccttatgctt

SEQ ID NO: 961 - DNA, 27 bp, Homo sapiens
  source 1..27: mol_type = other DNA
gctacaacaaacatccttgaacatgta

SEQ ID NO: 962 - DNA, 27 bp, Homo sapiens
  source 1..27: mol_type = other DNA
atgactctgaggcctacacagccacgt

SEQ ID NO: 963 - DNA, 27 bp, Homo sapiens
  source 1..27: mol_type = other DNA
gaaacaagaccgaacaactccaagttc

SEQ ID NO: 964 - DNA, 27 bp, Homo sapiens
  source 1..27: mol_type = other DNA
cactcaatcacggagcctctgctctat

SEQ ID NO: 965 - DNA, 30 bp, Homo sapiens
  source 1..30: mol_type = other DNA
catttattcttacaacctccaagcaaaaga

SEQ ID NO: 966 - DNA, 27 bp, Homo sapiens
  source 1..27: mol_type = other DNA
gagacagttagatttacttcctctttt

SEQ ID NO: 967 - DNA, 30 bp, Homo sapiens
  source 1..30: mol_type = other DNA
tccccaatgcctatacccccattgtatctt

SEQ ID NO: 968 - DNA, 27 bp, Homo sapiens
  source 1..27: mol_type = other DNA
gaatcttatggtaggtatactttttac

SEQ ID NO: 969 - DNA, 27 bp, Homo sapiens
  source 1..27: mol_type = other DNA
aaatcacaaggttcatttatttccttt

SEQ ID NO: 970 - DNA, 27 bp, Homo sapiens
  source 1..27: mol_type = other DNA
ggtactatcttgcacataggtgtattt

SEQ ID NO: 971 - DNA, 27 bp, Homo sapiens
  source 1..27: mol_type = other DNA
aaagtattttctggaagcagcaagagg

SEQ ID NO: 972 - DNA, 33 bp, Homo sapiens
  source 1..33: mol_type = other DNA
tcaagcttcagcacccttgcatctctggataaa

SEQ ID NO: 973 - DNA, 27 bp, Homo sapiens
  source 1..27: mol_type = other DNA
accacccagggagctgctcctcagagg

SEQ ID NO: 974 - DNA, 30 bp, Homo sapiens
  source 1..30: mol_type = other DNA
cataccacccagggagctgctcctcagagg

SEQ ID NO: 975 - DNA, 27 bp, Homo sapiens
  source 1..27: mol_type = other DNA
aagatcttccaagagagggcagccttt

SEQ ID NO: 976 - DNA, 27 bp, Homo sapiens
  source 1..27: mol_type = other DNA
gggcaaatcacacataacacttcattc

SEQ ID NO: 977 - DNA, 30 bp, Homo sapiens
  source 1..30: mol_type = other DNA
attagtctcaagcctttgatagcacggtat

SEQ ID NO: 978 - DNA, 27 bp, Homo sapiens
  source 1..27: mol_type = other DNA
aagcattctgatttgattttattctgg

SEQ ID NO: 979 - DNA, 30 bp, Homo sapiens
  source 1..30: mol_type = other DNA
atggtatgggaggggcatcccagagaaaga

SEQ ID NO: 980 - DNA, 27 bp, Homo sapiens
  source 1..27: mol_type = other DNA
tcaactgatttgcccattcttcttaaa

SEQ ID NO: 981 - DNA, 30 bp, Homo sapiens
  source 1..30: mol_type = other DNA
gaggtaatttctatcaacactggacatttc

SEQ ID NO: 982 - DNA, 33 bp, Homo sapiens
  source 1..33: mol_type = other DNA
gagacagagcgggtcctggagaagtcttcttac

SEQ ID NO: 983 - DNA, 27 bp, Homo sapiens
  source 1..27: mol_type = other DNA
aagataaaaactataaatctgagatat

SEQ ID NO: 984 - DNA, 27 bp, Homo sapiens
  source 1..27: mol_type = other DNA
tatgtgagtcagaaagtttcagacttt

SEQ ID NO: 985 - DNA, 27 bp, Homo sapiens
  source 1..27: mol_type = other DNA
gatgcaggcatccctgtgtaccaccga

SEQ ID NO: 986 - DNA, 33 bp, Homo sapiens
  source 1..33: mol_type = other DNA
accatcttggaagcagagatccttcaccaggca

SEQ ID NO: 987 - DNA, 27 bp, Homo sapiens
  source 1..27: mol_type = other DNA
gaatatgctcagaaaacacctctcttt

SEQ ID NO: 988 - DNA, 27 bp, Homo sapiens
  source 1..27: mol_type = other DNA
acatggacaccacatggtatcagaatc

SEQ ID NO: 989 - DNA, 30 bp, Homo sapiens
  source 1..30: mol_type = other DNA
gaggtccacgaaggtggagttgctcgattt

SEQ ID NO: 990 - DNA, 27 bp, Homo sapiens
  source 1..27: mol_type = other DNA
agagaaatagaaatagagggaagatta

SEQ ID NO: 991 - DNA, 27 bp, Homo sapiens
  source 1..27: mol_type = other DNA
tcctggctgccaatcccagccaaatat

SEQ ID NO: 992 - DNA, 27 bp, Homo sapiens
  source 1..27: mol_type = other DNA
cgctactccgaaagggtctacactatc

SEQ ID NO: 993 - DNA, 30 bp, Homo sapiens
  source 1..30: mol_type = other DNA
ttcacagactcgcagggcaatgacattaaa

SEQ ID NO: 994 - DNA, 30 bp, Homo sapiens
  source 1..30: mol_type = other DNA
tcagtgccaagcgacttcctgagtctcagg

SEQ ID NO: 995 - DNA, 27 bp, Homo sapiens
  source 1..27: mol_type = other DNA
cggcagatgagtctggagttcagggca

SEQ ID NO: 996 - DNA, 27 bp, Homo sapiens
  source 1..27: mol_type = other DNA
gaaaccatctcccccaggccttcaata

SEQ ID NO: 997 - DNA, 27 bp, Homo sapiens
  source 1..27: mol_type = other DNA
gggccagcaggaggaaggggagtcatc

SEQ ID NO: 998 - DNA, 27 bp, Homo sapiens
  source 1..27: mol_type = other DNA
ctgactgaccatcacataaaagaatac

SEQ ID NO: 999 - DNA, 33 bp, Homo sapiens
  source 1..33: mol_type = other DNA
gaagcagtaccttataatggttccttgttcaaa

SEQ ID NO: 1000 - DNA, 27 bp, Homo sapiens
  source 1..27: mol_type = other DNA
aatctgctggcggagatccacggtgtg

SEQ ID NO: 1001 - DNA, 27 bp, Homo sapiens
  source 1..27: mol_type = other DNA
aaagaggggccagtgacgcgaggagca

SEQ ID NO: 1002 - DNA, 27 bp, Homo sapiens
  source 1..27: mol_type = other DNA
gtacacgtggatttgaatcttggcttc

SEQ ID NO: 1003 - DNA, 27 bp, Homo sapiens
  source 1..27: mol_type = other DNA
cacactgtacagggactagctggattt

SEQ ID NO: 1004 - DNA, 30 bp, Homo sapiens
  source 1..30: mol_type = other DNA
atgcgtatagaggacagccacactaagctt

SEQ ID NO: 1005 - DNA, 27 bp, Homo sapiens
  source 1..27: mol_type = other DNA
agtgccctgtcttcagcccagcccagg

SEQ ID NO: 1006 - DNA, 30 bp, Homo sapiens
  source 1..30: mol_type = other DNA
tccccttccatcaaaagtctactagtttat

SEQ ID NO: 1007 - DNA, 33 bp, Homo sapiens
  source 1..33: mol_type = other DNA
ccaactgatgccactttaaaccgtttgctctac

SEQ ID NO: 1008 - DNA, 30 bp, Homo sapiens
  source 1..30: mol_type = other DNA
tggacttcccagcccccaaactatgagaaa

SEQ ID NO: 1009 - DNA, 30 bp, Homo sapiens
  source 1..30: mol_type = other DNA
cccgccccccgccccgtggcacccaccctg

SEQ ID NO: 1010 - DNA, 27 bp, Homo sapiens
  source 1..27: mol_type = other DNA
gtatatctccattctaacagcacatta

SEQ ID NO: 1011 - DNA, 27 bp, Homo sapiens
  source 1..27: mol_type = other DNA
tctttgtatgcctggactcagagccac

SEQ ID NO: 1012 - protein, 16 aa, Homo sapiens
CASHPAGADMNTEAFF

SEQ ID NO: 1013 - protein, 13 aa, Homo sapiens
CASSLAGRETQYF

SEQ ID NO: 1014 - protein, 15 aa, Homo sapiens
CASSVWGEPYNEQFF

SEQ ID NO: 1015 - protein, 13 aa, Homo sapiens
CASSRTDSYEQYF

SEQ ID NO: 1016 - protein, 13 aa, Homo sapiens
CASSLQGANGYTF

SEQ ID NO: 1017 - protein, 13 aa, Homo sapiens
CASSPTNTYEQYF

SEQ ID NO: 1018 - protein, 13 aa, Homo sapiens
CASSLSYTGELFF

SEQ ID NO: 1019 - protein, 13 aa, Homo sapiens
CASSRTGNQPQHF

SEQ ID NO: 1020 - protein, 11 aa, Homo sapiens
CASSSSGEQYF

SEQ ID NO: 1021 - protein, 14 aa, Homo sapiens
CASSVEEGSYEQYF

SEQ ID NO: 1022 - protein, 10 aa, Homo sapiens
CASRDYGYTF

SEQ ID NO: 1023 - protein, 14 aa, Homo sapiens
CASSYSGDYNEQFF

SEQ ID NO: 1024 - protein, 15 aa, Homo sapiens
CASSFEAYSNQPQHF

SEQ ID NO: 1025 - protein, 14 aa, Homo sapiens
CASSGGDSPYEQYF

SEQ ID NO: 1026 - protein, 16 aa, Homo sapiens
CASSLDTGVGANVLTF

SEQ ID NO: 1027 - protein, 13 aa, Homo sapiens
CASSQLGVTEAFF

SEQ ID NO: 1028 - protein, 14 aa, Homo sapiens
CASSPGTAQETQYF

SEQ ID NO: 1029 - protein, 14 aa, Homo sapiens
CASSLDRGDYEQYF

SEQ ID NO: 1030 - protein, 13 aa, Homo sapiens
CASSLGGLYEQYF

SEQ ID NO: 1031 - protein, 15 aa, Homo sapiens
CASSQDLANTGELFF

SEQ ID NO: 1032 - protein, 15 aa, Homo sapiens
CSVEQLGEMNTEAFF

SEQ ID NO: 1033 - protein, 13 aa, Homo sapiens
CASSQGEIDGYTF

SEQ ID NO: 1034 - protein, 12 aa, Homo sapiens
CASSLTGDGYTF

SEQ ID NO: 1035 - protein, 14 aa, Homo sapiens
CASSPRDGTYEQYF

SEQ ID NO: 1036 - protein, 13 aa, Homo sapiens
CASSLDRAYEQYF

SEQ ID NO: 1037 - protein, 15 aa, Homo sapiens
CASSQDLGSSYEQYF

SEQ ID NO: 1038 - protein, 14 aa, Homo sapiens
CASGTGGSTDTQYF

SEQ ID NO: 1039 - protein, 11 aa, Homo sapiens
CASSETDTQYF

SEQ ID NO: 1040 - protein, 15 aa, Homo sapiens
CASSVGQFSNQPQHF

SEQ ID NO: 1041 - protein, 13 aa, Homo sapiens
CASSPRTNQPQHF

SEQ ID NO: 1042 - protein, 15 aa, Homo sapiens
CASSLGGTGPYEQYF

SEQ ID NO: 1043 - protein, 12 aa, Homo sapiens
CAISGDGYGYTF

SEQ ID NO: 1044 - protein, 13 aa, Homo sapiens
CASSLTGGTEAFF

SEQ ID NO: 1045 - protein, 13 aa, Homo sapiens
CASSLSDSYEQYF

SEQ ID NO: 1046 - protein, 15 aa, Homo sapiens
CASSQEGREVDGYTF

SEQ ID NO: 1047 - protein, 13 aa, Homo sapiens
CASSEVSNQPQHF

SEQ ID NO: 1048 - protein, 6 aa, Homo sapiens
GNTIYF

SEQ ID NO: 1049 - protein, 11 aa, Homo sapiens
CASQGAYEQYF

SEQ ID NO: 1050 - protein, 17 aa, Homo sapiens
CASSFKPGLKWRDEQFF

SEQ ID NO: 1051 - protein, 15 aa, Homo sapiens
CASSIGTANSYEQYF

SEQ ID NO: 1052 - protein, 13 aa, Homo sapiens
CASSIGTNTEAFF

SEQ ID NO: 1053 - protein, 14 aa, Homo sapiens
CASSVGDSGNTIYF

SEQ ID NO: 1054 - protein, 14 aa, Homo sapiens
CASSSGVTNEKLFF

SEQ ID NO: 1055 - protein, 13 aa, Homo sapiens
CASSATGDTEAFF

SEQ ID NO: 1056 - protein, 15 aa, Homo sapiens
CASSLRTGRGYGYTF

SEQ ID NO: 1057 - protein, 13 aa, Homo sapiens
CAIQRGRTGELFF

SEQ ID NO: 1058 - protein, 14 aa, Homo sapiens
CASSLATGYNEQFF

SEQ ID NO: 1059 - protein, 15 aa, Homo sapiens
CASSFTERGYEKLFF

SEQ ID NO: 1060 - protein, 15 aa, Homo sapiens
CASSPRTGMNTEAFF

SEQ ID NO: 1061 - protein, 17 aa, Homo sapiens
CASSQDSEQGLVQPQHF

SEQ ID NO: 1062 - protein, 16 aa, Homo sapiens
CASSRSTSGRADEQFF

SEQ ID NO: 1063 - protein, 15 aa, Homo sapiens
CASRASSGLSYEQYF

SEQ ID NO: 1064 - protein, 13 aa, Homo sapiens
CASSVAGSTEAFF

SEQ ID NO: 1065 - protein, 15 aa, Homo sapiens
CASSQEGGMNTEAFF

SEQ ID NO: 1066 - protein, 14 aa, Homo sapiens
CASSSSDRVGEQYF

SEQ ID NO: 1067 - protein, 15 aa, Homo sapiens
CASSLGQNSYNEQFF

SEQ ID NO: 1068 - protein, 14 aa, Homo sapiens
CASSPRSSYNEQFF

SEQ ID NO: 1069 - protein, 14 aa, Homo sapiens
CASSLLGGGQPQHF

SEQ ID NO: 1070 - protein, 16 aa, Homo sapiens
CASSRGLAVSYNEQFF

SEQ ID NO: 1071 - protein, 15 aa, Homo sapiens
CASSLLARSSYEQYF

SEQ ID NO: 1072 - protein, 13 aa, Homo sapiens
CASSPTENTEAFF

SEQ ID NO: 1073 - protein, 15 aa, Homo sapiens
CASSVGGGRNYEQYF

SEQ ID NO: 1074 - protein, 14 aa, Homo sapiens
CASSYSGGPYEQYF

SEQ ID NO: 1075 - protein, 13 aa, Homo sapiens
CASSLGDGQPQHF

SEQ ID NO: 1076 - protein, 12 aa, Homo sapiens
CASSQGDYGYTF

SEQ ID NO: 1077 - protein, 14 aa, Homo sapiens
CASSYQGTNTEAFF

SEQ ID NO: 1078 - protein, 13 aa, Homo sapiens
CASSFIGGQPQHF

SEQ ID NO: 1079 - protein, 17 aa, Homo sapiens
CASSLGLAGTNTGELFF

SEQ ID NO: 1080 - protein, 12 aa, Homo sapiens
CASSVGPETQYF

SEQ ID NO: 1081 - protein, 15 aa, Homo sapiens
CASSQGLSSGNTIYF

SEQ ID NO: 1082 - protein, 11 aa, Homo sapiens
CASSSATEAFF

SEQ ID NO: 1083 - protein, 14 aa, Homo sapiens
CASSLKGVTYEQYF

SEQ ID NO: 1084 - protein, 12 aa, Homo sapiens
CASSPDGGEQYF

SEQ ID NO: 1085 - protein, 12 aa, Homo sapiens
CASSLFPDTQYF

SEQ ID NO: 1086 - protein, 15 aa, Homo sapiens
CASSLGDSYTGELFF

SEQ ID NO: 1087 - protein, 13 aa, Homo sapiens
CASSVEGYTEAFF

SEQ ID NO: 1088 - protein, 13 aa, Homo sapiens
CASSPPESYEQYF

SEQ ID NO: 1089 - protein, 14 aa, Homo sapiens
CASSPDPEGGEQYF

SEQ ID NO: 1090 - protein, 12 aa, Homo sapiens
CASSLQGHGYTF

SEQ ID NO: 1091 - protein, 20 aa, Homo sapiens
CASNVARTGGDISGDYEQYF

SEQ ID NO: 1092 - protein, 15 aa, Homo sapiens
CASSLGLAGSYEQYF

SEQ ID NO: 1093 - protein, 14 aa, Homo sapiens
CASSDSSYNSPLHF

SEQ ID NO: 1094 - protein, 18 aa, Homo sapiens
CASSEWGQGDSNNYGYTF

SEQ ID NO: 1095 - protein, 14 aa, Homo sapiens
CASSNLAGLNEQFF

SEQ ID NO: 1096 - protein, 14 aa, Homo sapiens
CASSPPSGGFEQFF

SEQ ID NO: 1097 - protein, 14 aa, Homo sapiens
CASSFDGWNTEAFF

SEQ ID NO: 1098 - protein, 14 aa, Homo sapiens
CASSLGGQGYGYTF

SEQ ID NO: 1099 - protein, 15 aa, Homo sapiens
CASSPGQGNNEKLFF

SEQ ID NO: 1100 - protein, 16 aa, Homo sapiens
CASSIVFGSRNSPLHF

SEQ ID NO: 1101 - protein, 11 aa, Homo sapiens
CASKESTEAFF

SEQ ID NO: 1102 - protein, 14 aa, Homo sapiens
CSANTRDISYEQYF

SEQ ID NO: 1103 - protein, 14 aa, Homo sapiens
CASSFQGLNTEAFF

SEQ ID NO: 1104 - protein, 14 aa, Homo sapiens
CASSPGQGNEKLFF

SEQ ID NO: 1105 - protein, 13 aa, Homo sapiens
CASSLGSGYEQYF

SEQ ID NO: 1106 - protein, 13 aa, Homo sapiens
CASSLGLYNEQFF

SEQ ID NO: 1107 - protein, 14 aa, Homo sapiens
CASSPGTDYNEQFF

SEQ ID NO: 1108 - protein, 13 aa, Homo sapiens
CATRPGAIRGYTF

SEQ ID NO: 1109 - protein, 15 aa, Homo sapiens
CASSLIGGADTEAFF

SEQ ID NO: 1110 - protein, 14 aa, Homo sapiens
CASSLLTGNYGYTF

SEQ ID NO: 1111 - protein, 14 aa, Homo sapiens
CASYNRGERGGYTF

SEQ ID NO: 1112 - protein, 17 aa, Homo sapiens
CASSPGTSGSSYNEQFF

SEQ ID NO: 1113 - protein, 17 aa, Homo sapiens
CASSPVPRGDVPYEQYF

SEQ ID NO: 1114 - protein, 13 aa, Homo sapiens
CASSADPNTEAFF

SEQ ID NO: 1115 - protein, 14 aa, Homo sapiens
CSARDTRGGQPQHF

SEQ ID NO: 1116 - protein, 15 aa, Homo sapiens
CASSLTSGSRNEQFF

SEQ ID NO: 1117 - protein, 13 aa, Homo sapiens
CASSSGRFYEQYF

SEQ ID NO: 1118 - protein, 15 aa, Homo sapiens
CASSVLAGGSYEQYF

SEQ ID NO: 1119 - protein, 13 aa, Homo sapiens
CASSYGTGVEQYF

SEQ ID NO: 1120 - protein, 16 aa, Homo sapiens
CASSQPGTSGYNEQFF

SEQ ID NO: 1121 - protein, 15 aa, Homo sapiens
CASSRLAGGQETQYF

SEQ ID NO: 1122 - protein, 16 aa, Homo sapiens
CASRPGTGGRMDIQYF

SEQ ID NO: 1123 - protein, 11 aa, Homo sapiens
CAIRSTDTQYF

SEQ ID NO: 1124 - protein, 13 aa, Homo sapiens
CASSLGGNQPQHF

SEQ ID NO: 1125 - protein, 15 aa, Homo sapiens
CASSSGPSGGNTIYF

SEQ ID NO: 1126 - protein, 14 aa, Homo sapiens
CASSLGTGGYEQFF

SEQ ID NO: 1127 - protein, 14 aa, Homo sapiens
CASSLGGGGYGYTF

SEQ ID NO: 1128 - protein, 14 aa, Homo sapiens
CASRTGGANTEAFF

SEQ ID NO: 1129 - protein, 13 aa, Homo sapiens
CASSGDRGHEQYF

SEQ ID NO: 1130 - protein, 15 aa, Homo sapiens
CAISETGLVGEKLFF

SEQ ID NO: 1131 - protein, 14 aa, Homo sapiens
CASSLGQGSEKLFF

SEQ ID NO: 1132 - protein, 14 aa, Homo sapiens
CASSLGRMNTEAFF

SEQ ID NO: 1133 - protein, 13 aa, Homo sapiens
CASSLLGPGELFF

SEQ ID NO: 1134 - protein, 16 aa, Homo sapiens
CASSSRDRESGNTIYF

SEQ ID NO: 1135 - protein, 16 aa, Homo sapiens
CASSPEGGSSYNEQFF

SEQ ID NO: 1136 - protein, 14 aa, Homo sapiens
CASSQSGQGYGYTF

SEQ ID NO: 1137 - protein, 15 aa, Homo sapiens
CASSGTSGAYNEQFF

SEQ ID NO: 1138 - protein, 4 aa, synthetic construct
LVGS

SEQ ID NO: 1139 - protein, 4 aa, synthetic construct
ASLG

SEQ ID NO: 1140 - protein, 4 aa, synthetic construct
PIVG

SEQ ID NO: 1141 - protein, 4 aa, synthetic construct
LLSV

SEQ ID NO: 1142 - protein, 5 aa, synthetic construct
VLSVG

SEQ ID NO: 1143 - protein, 6 aa, synthetic construct
LLSVGG

(19) United States
(12) Patent Application Publication
(10) Pub. No.: US 2026/0284166 A1
(43) Pub. Date: Sep. 24, 2026

(54) NOVEL TUMOR ANTIGENS FOR MELANOMA AND USES THEREOF

(71) Applicant: UNIVERSITÉ DE MONTRÉAL, Montréal (CA)

(72) Inventors: Claude PERREAULT, Montréal, Québec (CA); Pierre THIBAULT, Montréal, Québec (CA); Marie-Pierre HARDY, Terrebonne, Québec (CA); Krystel VINCENT, Terrebonne, Québec (CA); Anca APAVALOAEI, New York, NY (US)

(21) Appl. No.: 19/474,661

(22) PCT Filed: Apr. 11, 2024
(86) PCT No.: PCT/CA2024/050460

§ 371 Date: Oct. 10, 2025

Related U.S. Application Data
(60) Provisional application No. 63/496,084, filed on Apr. 14, 2023.
(60) Provisional application No. 63/515,147, filed on Jul. 24, 2023.

(51) Int. Cl. A61K 39/00; A61K 45/06; A61P 37/04; C07K 14/47; C07K 14/705; C07K 16/30

(52) U.S. Cl. CPC A61K 39/00119; A61K 45/06; A61P 37/04; C07K 14/4748; C07K 14/7051; C07K 16/3053

(57) ABSTRACT

While immune checkpoint blockade (ICB) therapy has significantly improved the outcome of metastatic melanoma, most patients do not derive long-term benefits. Previous studies suggest that treatment failure is partly due to insufficient immune recognition of tumor antigens (TAs). Cancer vaccines could potentially provide a complementary approach to boost antitumor immunity and act synergistically with ICIs. Novel tumor antigens shared by a large proportion of melanoma cells are described herein. Several of the tumor antigens described herein derives from aberrantly expressed unmutated genomic sequences, such as intronic and intergenic sequences, which are not expressed in normal tissues. Nucleic acids, compositions, cells and vaccines derived from these tumor antigens are described. The use of the tumor antigens, nucleic acids, compositions, cells and vaccines for the treatment of melanoma is also described.

### CROSS REFERENCE TO RELATED APPLICATIONS

The present application claims the benefit of U.S. provisional patent application Ser. No. 63/496,084, filed on Apr. 14, 2023, and U.S. provisional patent application Ser. No. 63/515,147 filed on Jul. 24, 2023, which are incorporated herein by reference in their entirety.

### SEQUENCE LISTING

A sequence listing is submitted herewith as an XML file named G17971-00091_Seq Listing.xml, created on Apr. 11, 2024, and having a size of ~995 793 bytes. The content of the aforementioned file is hereby incorporated by reference in its entirety.

### TECHNICAL FIELD

The present disclosure generally relates to the field of cancer, and more particularly to the treatment of cancers such as melanoma.

### BACKGROUND ART

Cutaneous melanoma is an aggressive form of skin cancer with over 100,000 US cases in 2021 (Siegel et al., CA Cancer J Clin. 2021; 71(1):7-33); annually, melanoma causes over 7000 deaths. Tumor stage is determined by histopathologic and clinical factors, which include the Breslow depth of the tumor, ulceration status, spread of disease from the primary tumor to lymph nodes, and the presence of metastasis. These factors are summarized into an overall stage denoted by a Roman numeral from 0 to IV, with Stage 0 disease being the earliest and Stage IV being the most advanced. Patients within a stage should theoretically have similar outcomes, but there is still significant heterogeneity within each stage. For example, most patients with Stage I disease will be cured by surgery, but a significant minority (5-10%) may go on to develop metastasis at a later time.

While immune checkpoint blockade (ICB) therapy has significantly improved the outcome of metastatic melanoma, most patients do not derive long-term benefits. Previous studies suggest that treatment failure is partly due to insufficient immune recognition of tumor antigens (TAs). Notably, immune targeting of TAs in melanoma has focused on TAs derived from non-synonymous genomic mutations.

In view of this, there is a need to identify the tumor antigens that can elicit therapeutic immune responses again melanoma. Such antigens could be used as vaccines (±immune checkpoint inhibitors) or as targets for T-cell receptor-based approaches (cell therapy, bispecific biologics).

The present description refers to a number of documents, the content of which is herein incorporated by reference in their entirety.

### SUMMARY

In various aspects and embodiments, the present disclosure provides the following items 1 to 79:
1. A tumor antigen peptide (TAP) comprising or consisting of one of the amino acid sequences set forth in any one of SEQ ID NOs:1-505.
2. The TAP of item 1, wherein the TAP binds to an HLA-A*01:01 molecule and comprises or consists of the sequence of SEQ ID NO: 11, 14, 20, 27, 29, 37, 86, 87, 121, 150, 165, 173, 279, 288, 299, 308, 312, 316, 319, 331, 340, 352, 362, 363, 384, 395, 398, 411, 419, 429, 458, 474, 476, 487, 492, 500, or 501.
3. The TAP of item 1, wherein the TAP binds to an HLA-A*02:01 molecule and comprises or consists of the sequence of SEQ ID NO: 1, 2, 4, 19, 22, 25, 40, 50, 52, 54, 60, 65, 67, 76, 81, 89, 91, 103, 107, 120, 124, 128, 135, 157, 175, 179, 186, 188, 201, 208, 218, 219, 228, 232, 233, 240, 250, 257, 261, 263, 265, 266, 282, 286, 298, 311, 315, 317, 325, 333, 337, 349, 367, 373, 387, 388, 393, 399, 404, 412, 414, 415, 422, 434, 437, 455, 480, 489, or 494.
4. The TAP of item 1, wherein the TAP binds to an HLA-A*02:09 molecule and comprises or consists of the sequence of SEQ ID NO: 22, 40, 50, 62, 83, 89, 232, 265, 282, 311, 315, 317, 337, 349, 367, 399, 407, 412, or 494.
5. The TAP of item 1, wherein the TAP binds to an HLA-A*03:01 molecule and comprises or consists of the sequence of SEQ ID NO: 209, 247, 272, 347, or 505.
6. The TAP of item 1, wherein the TAP binds to an HLA-A*23:01 molecule and comprises or consists of the sequence of SEQ ID NO: 48, 84, 129, 151, 152, 171, 174, 182, 223, 237, 245, 290, 358, 397, 426, 446, 481, 482, or 486.
7. The TAP of item 1, wherein the TAP binds to an HLA-A*24:02 molecule and comprises or consists of the sequence of SEQ ID NO: 31, 244 or 481.
8. The TAP of item 1, wherein the TAP binds to an HLA-A*25:01 molecule and comprises or consists of the sequence of SEQ ID NO: 11, 31, 249, 289, 403, 425, 464, or 490.
9. The TAP of item 1, wherein the TAP binds to an HLA-A*26:01 molecule and comprises or consists of the sequence of SEQ ID NO: 11, 20, 27, 39, 41, 42, 45, 114, 116, 122, 168, 181, 268, 284, 329, 364, 398, 417, 430, 440, 447, 452, 457, 460, 462, 475, 490, or 497.
10. The TAP of item 1, wherein the TAP binds to an HLA-A*30:01 molecule and comprises or consists of the sequence of SEQ ID NO: 26, 304 or 376.
11. The TAP of item 1, wherein the TAP binds to an HLA-A*30:02 molecule and comprises or consists of the sequence of SEQ ID NO: 117, 185, 190, 193, 197, 256, 294, 323, 471, or 485.
12. The TAP of item 1, wherein the TAP binds to an HLA-A*33:01 molecule and comprises or consists of the sequence of SEQ ID NO: 248.
13. The TAP of item 1, wherein the TAP binds to an HLA-A*68:01 molecule and comprises or consists of the sequence of SEQ ID NO: 6-8, 12, 15, 23, 28, 41, 42, 47, 51, 53, 56, 59, 64, 66, 68, 70, 73-75, 77, 79, 80, 82, 85, 88, 92, 93, 96-99, 101, 102, 108-112, 118, 119, 123, 130, 131, 134, 137, 138, 141, 142, 144-147, 149, 153, 154, 156, 158, 160, 161, 166, 169, 172, 176, 184, 198, 202, 203, 212, 214, 216, 217, 220, 224, 226, 229, 234, 236, 239, 248, 254, 269, 270, 275, 280, 283, 287, 293, 300, 301, 303, 306, 310, 314, 320, 328, 332, 348, 354, 355, 359, 360, 366, 369, 370, 372, 381, 382, 386, 389, 390, 392, 394, 402, 405, 406, 409, 413, 418, 424, 428, 436, 441-445, 449, 456, 459, 465-468, 473, 479, 483, 488, 493, 499, or 502.
14. The TAP of item 1, wherein the TAP binds to an HLA-B*07:02 molecule and comprises or consists of the sequence of SEQ ID NO: 35, 36, 43, 104, 125, 127, 139, 140, 187, 191, 196, 206, 231, 243, 246, 251, 252, 260, 281, 291, 292, 321, 330, 361, 408, 432, 435, 439, 451, 461, 491, or 503.
15. The TAP of item 1, wherein the TAP binds to an HLA-B*08:01 molecule and comprises or consists of the sequence of SEQ ID NO: 21, 162, 189, 286, 307, 309, 317, 339, 343, 349, or 414.
16. The TAP of item 1, wherein the TAP binds to an HLA-B*13:02 molecule and comprises or consists of the sequence of SEQ ID NO: 207, 311, 393, 400, or 455.
17. The TAP of item 1, wherein the TAP binds to an HLA-B*14:01 molecule and comprises or consists of the sequence of SEQ ID NO: 396.
18. The TAP of item 1, wherein the TAP binds to an HLA-B*15:01 molecule and comprises or consists of the sequence of SEQ ID NO: 11, 26, 30, 63, 113, 132, 148, 192, 195, 200, 207, 210, 222, 235, 238, 258, 262, 267, 276, 295, 297, 305, 309, 318, 326, 336, 342, 374, 377, 383, 401, 421, 427, 433, 438, 450, 463, 470, or 477.
19. The TAP of item 1, wherein the TAP binds to an HLA-B*18:01 molecule and comprises or consists of the sequence of SEQ ID NO: 3, 10, 33, 225 or 396.
20. The TAP of item 1, wherein the TAP binds to an HLA-B*37:01 molecule and comprises or consists of the sequence of SEQ ID NO: 302.
21. The TAP of item 1, wherein the TAP binds to an HLA-B*38:01 molecule and comprises or consists of the sequence of SEQ ID NO: 26, 180, 338, 341, 378, 380, 416, 448, 454, 496, or 498.
22. The TAP of item 1, wherein the TAP binds to an HLA-B*40:01 molecule and comprises or consists of the sequence of SEQ ID NO: 215 or 322.
23. The TAP of item 1, wherein the TAP binds to an HLA-B*40:02 molecule and comprises or consists of the sequence of SEQ ID NO: 213, 215, 339, or 351.
24. The TAP of item 1, wherein the TAP binds to an HLA-B*44:02 molecule and comprises or consists of the sequence of SEQ ID NO: 346, 353, 356, or 453.
25. The TAP of item 1, wherein the TAP binds to an HLA-B*49:01 molecule and comprises or consists of the sequence of SEQ ID NO: 9, 57, 78, 115, 136, 167, 255, 273, 274, 278, 313, 339, 480, 484, or 495.
26. The TAP of item 1, wherein the TAP binds to an HLA-B*51:01 molecule and comprises or consists of the sequence of SEQ ID NO: 227 or 259.
27. The TAP of item 1, wherein the TAP binds to an HLA-C*03:03 molecule and comprises or consists of the sequence of SEQ ID NO: 24, 69, 204 or 388.
28. The TAP of item 1, wherein the TAP binds to an HLA-C*03:04 molecule and comprises or consists of the sequence of SEQ ID NO: 24, 49, 69, 159, 241, 242 or 334.
29. The TAP of item 1, wherein the TAP binds to an HLA-C*06:02 molecule and comprises or consists of the sequence of SEQ ID NO: 13, 34 or 72.
30. The TAP of item 1, wherein the TAP binds to an HLA-C*07:01 molecule and comprises or consists of the sequence of SEQ ID NO: 13, 75, 133, 163, 183, 199, 205, 285, 334, 391 or 472.
31. The TAP of item 1, wherein the TAP binds to an HLA-C*07:02 molecule and comprises or consists of the sequence of SEQ ID NO: 13, 32, 36, 105, 177, 339, 371 or 478.
32. The TAP of item 1, wherein the TAP binds to an HLA-C*08:02 molecule and comprises or consists of the sequence of SEQ ID NO: 18, 54 or 379.
33. The TAP of item 1, wherein the TAP binds to an HLA-C*12:03 molecule and comprises or consists of the sequence of SEQ ID NO: 24, 49, 58, 71, 94, 162, 178, 242, 289, 344, 345, 357, 367, 368, 375, 423 or 431.
34. The TAP of item 1, wherein the TAP binds to an HLA-C*14:02 molecule and comprises or consists of the sequence of SEQ ID NO: 105, 271, 350, 365, 385, 410, 423, or 504.
35. The TAP of any one of items 1-34, which is encoded by a sequence located a non-protein coding region of the genome or by a long non-coding RNA.
36. The TAP of item 35, wherein said non-protein coding region of the genome is an intergenic region.
37. The TAP of item 35, wherein said non-protein coding region of the genome is an intron.
38. The TAP of any one of items 1 to 37, wherein said TAP is conjugated to a molecule that increases protease resistance, plasma protein binding, plasma half-life and/or intracellular penetration of the TAP, for example a fatty acid, a protein (e.g., albumin), a sugar or polysaccharide, or a polymer (e.g., polyethylene glycol).
39. A combination comprising at least two of the TAPs defined in any one of items 1-38.
40. A synthetic long peptide (SLP) comprising at least one of the amino acid sequences defined in item 1.
41. A nucleic acid encoding one or more of the TAPs of items 1 to 38, the combination of item 39, or the SLP of item 40.
42. The nucleic acid of item 41, wherein the nucleic acid is an mRNA, and wherein the mRNA optionally comprises one or more 5′-end modifications, 3′-end modifications, and/or modified nucleosides to increase stability, improve translation and/or reduce immunogenicity of the mRNA.
43. The nucleic acid of item 41, wherein the nucleic acid is a DNA.
44. The nucleic acid of any one of items 41-43, wherein the nucleic acid is a component of a viral vector.
45. A vesicle or particle comprising the TAP of any one of items 1-38, the combination of item 39, the SLP of item 40, or the nucleic acid of any one of items 41-44.
46. The vesicle or particle of item 45, wherein the vesicle is a lipid nanoparticle (LNP).
47. The vesicle or particle of item 45 or 46, which comprises a cationic lipid.
48. A composition comprising the TAP of any one of items 1-38, the combination of item 39, the SLP of item 40, the nucleic acid of any one of items 41-44, or the vesicle or particle of any one of items 45-47, and a pharmaceutically acceptable carrier.
49. A vaccine comprising the TAP of any one of items 1-38, the combination of item 39, the SLP of item 40, the nucleic acid of any one of items 41-44, the vesicle or particle of any one of items 45-47, or the composition of item 47, and an adjuvant.
50. An isolated major histocompatibility complex (MHC) class I molecule comprising the TAP of any one of items 1-38 in its peptide binding groove.
51. The isolated MHC class I molecule of item 50, which is in the form of a multimer.
52. The isolated MHC class I molecule of item 51, wherein said multimer is a tetramer.
53. An isolated cell comprising (i) the TAP of any one of items 1-38; (ii) the combination of item 39; (iii) the SLP of item 40; (iv) the nucleic acid of any one of items 41-44; or (v) a vector comprising a nucleotide sequence encoding the TAP of any one of items 1-38, the combination of item 39 or the SLP of item 40.
54. An isolated cell expressing at its surface major histocompatibility complex (MHC) class I molecules comprising the TAP of any one of items 1-38 or combination of item 39 in their peptide binding groove.
55. The cell of item 53 or 54, which is an antigen-presenting cell (APC).
56. The cell of item 55, wherein said APC is a dendritic cell.
57. A T-cell receptor (TCR) that specifically recognizes the isolated MHC class I molecule of any one of items 50-52 and/or MHC class I molecules expressed at the surface of the cell of any one of items 54-56, or a nucleic acid encoding said TCR.
58. The TCR of item 57, which is a soluble TCR.
59. An antibody or an antigen-binding fragment thereof that specifically binds to the isolated MHC class I molecule of any one of items 50-52 and/or MHC class I molecules expressed at the surface of the cell of any one of items 54-56, or a nucleic acid encoding said antibody or antigen-binding fragment thereof.
60. The TCR of item 57 or 58, or the antibody or antigen-binding fragment thereof according to item 59, which is a bispecific TCR or a bispecific antibody or antigen-binding fragment thereof.
61. The TCR, antibody or antigen-binding fragment thereof according to item 60, wherein the bispecific antibody or antigen-binding fragment thereof is a single-chain diabody (scDb).
62. The TCR, antibody or antigen-binding fragment thereof according to item 60 or 61, wherein the bispecific TCR, antibody or antigen-binding fragment thereof also specifically binds to a T cell signaling molecule.
63. The TCR, antibody or antigen-binding fragment thereof according to item 62, wherein the T cell signaling molecule is a CD3 chain.
64. A chimeric antigen receptor (CAR) comprising the antibody or an antigen-binding fragment thereof of item 59, or a nucleic acid encoding said CAR.
65. An isolated cell expressing at its cell surface the TCR of item 57 or the CAR of item 64.
66. The isolated cell of item 65, which is a CD8⁺ T lymphocyte.
67. A cell population comprising at least 0.5% of the isolated cell as defined in item 65 or 66.
68. A method of treating cancer in a subject comprising administering to the subject an effective amount of:
   (a) a TAP comprising or consisting of any one of the sequences set forth in SEQ ID NOs: 1-505 or any combination thereof, or a synthetic long peptide (SLP) comprising at least one of the sequences set forth in SEQ ID NOs: 1-505;
   (b) at least one nucleic acid encoding the TAP, combination thereof or SLP defined in (a);
   (c) a vesicle or particle comprising the TAP, combination thereof or SLP defined in (a) or the at least one nucleic acid defined in (b);
   (d) a composition comprising the TAP, combination thereof or SLP defined in (a), the at least one nucleic acid defined in (b), or the vesicle or particle defined in (c), and a pharmaceutically acceptable carrier;
   (e) a vaccine comprising the TAP, combination thereof or SLP defined in (a), the at least one nucleic acid defined in (b), the vesicle or particle defined in (c), or the composition defined in (d), and an adjuvant;
   (f) a cell expressing at its surface major histocompatibility complex (MHC) class I molecules comprising the TAP or combination thereof defined in (a) in their peptide binding groove;
   (g) a cell expressing at its cell surface a T-cell receptor (TCR) or a chimeric antigen receptor (CAR) that specifically recognizes MHC class I molecules expressed at the surface of the cell defined in (f); or
   (h) a soluble TCR, an antibody or an antigen-binding fragment thereof, or a CAR, that specifically binds to the MHC class I molecules expressed at the surface of the cell defined in (f), or a nucleic acid encoding said soluble TCR, antibody, antigen-binding fragment thereof, or CAR.
69. The method of item 68, wherein the cancer is melanoma.
70. The method of item 68 or 69, further comprising administering at least one additional antitumor agent or therapy to the subject.
71. The method of item 70, wherein said at least one additional antitumor agent or therapy is a chemotherapeutic agent, immunotherapy, an immune checkpoint inhibitor, radiotherapy or surgery.
72. Use of:
   (a) a TAP comprising or consisting of any one of the sequences set forth in SEQ ID NOs: 1-505 or any combination thereof, or a synthetic long peptide (SLP) comprising at least one of the sequences set forth in SEQ ID NOs: 1-505;
   (b) at least one nucleic acid encoding the TAP, combination thereof or SLP defined in (a);
   (c) a vesicle or particle comprising the TAP, combination thereof or SLP defined in (a) or the at least one nucleic acid defined in (b);
   (d) a composition comprising the TAP, combination thereof or SLP defined in (a), the at least one nucleic acid defined in (b), or the vesicle or particle defined in (c), and a pharmaceutically acceptable carrier;
   (e) a vaccine comprising the TAP, combination thereof or SLP defined in (a), the at least one nucleic acid defined in (b), the vesicle or particle defined in (c), or the composition defined in (d), and an adjuvant;
   (f) a cell expressing at its surface major histocompatibility complex (MHC) class I molecules comprising the TAP or combination thereof defined in (a) in their peptide binding groove;
   (g) a cell expressing at its cell surface a T-cell receptor (TCR) or a chimeric antigen receptor (CAR) that specifically recognizes MHC class I molecules expressed at the surface of the cell defined in (f); or
   (h) a soluble TCR, an antibody or an antigen-binding fragment thereof, or a CAR, that specifically binds to the MHC class I molecules expressed at the surface of the cell defined in (f), or a nucleic acid encoding said soluble TCR, antibody, antigen-binding fragment thereof, or CAR;
for treating cancer in a subject, or for the manufacture of a medicament for treating cancer in a subject.
73. The use of item 72, wherein said cancer is melanoma.
74. The use of item 72 or 73, further comprising the use at least one additional antitumor agent or therapy.
75. The use of item 74, wherein said at least one additional antitumor agent or therapy is a chemotherapeutic agent, immunotherapy, an immune checkpoint inhibitor, radiotherapy or surgery.
76. An agent for use in treating cancer in a subject, wherein the agent is:
   (a) a TAP comprising or consisting of any one of the sequences set forth in SEQ ID NOs: 1-505 or any combination thereof, or a synthetic long peptide (SLP) comprising at least one of the sequences set forth in SEQ ID NOs: 1-505:
   (b) at least one nucleic acid encoding the TAP, combination thereof or SLP defined in (a);
   (c) a vesicle or particle comprising the TAP, combination thereof or SLP defined in (a) or the at least one nucleic acid defined in (b);
   (d) a composition comprising the TAP, combination thereof or SLP defined in (a), the at least one nucleic acid defined in (b), or the vesicle or particle defined in (c), and a pharmaceutically acceptable carrier;
   (e) a vaccine comprising the TAP, combination thereof or SLP defined in (a), the at least one nucleic acid defined in (b), the vesicle or particle defined in (c), or the composition defined in (d), and an adjuvant;
   (f) a cell expressing at its surface major histocompatibility complex (MHC) class I molecules comprising the TAP or combination thereof defined in (a) in their peptide binding groove;
   (g) a cell expressing at its cell surface a T-cell receptor (TCR) or a chimeric antigen receptor (CAR) that specifically recognizes MHC class I molecules expressed at the surface of the cell defined in (f); or
   (h) a soluble TCR, an antibody or an antigen-binding fragment thereof, or a CAR, that specifically binds to the MHC class I molecules expressed at the surface of the cell defined in (f), or a nucleic acid encoding said soluble TCR, antibody, antigen-binding fragment thereof, or CAR.
77. The agent for use according to item 76, wherein said cancer is melanoma.
78. The agent for use according to item 76 or 77, further comprising the use at least one additional antitumor agent or therapy to the subject.
79. The agent for use according to item 78, wherein said at least one additional antitumor agent or therapy is a chemotherapeutic agent, immunotherapy, an immune checkpoint inhibitor, radiotherapy or surgery.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIGS. 1A-E show that aeTSAs are immunogenic and can contribute to the response to ICB in melanoma. FIG. 1A: Box plots showing the number of TA-HLA pairs (i.e., the sum of the HLA alleles per sample capable of presenting each expressed TA) per pre-treatment sample (grey dots) from Riaz et al.⁸, according to the response groups from the original study. P-values from unpaired two-tailed T-test. FIG. 1B: Box plots showing the number of TA-HLA pairs in pre- and on-treatment samples from Riaz et al.⁸, according to the response groups from the original study. Grey lines connect pre- and on-treatment samples per patient; p-values from paired two-tailed T-test. FIG. 1C: Pearson's correlation between the number of expanded T-cell clones and the number of TA-HLA pairs lost on-therapy per patient (colored dots) from Riaz et al.⁸, according to the response groups from the original study. Patients with stable disease (SD) were excluded due to the low number of samples with both RNA-seq and TCR-seq data (n=2). FIG. 1D: CD8 T cell clonotypes expanded in the on-treatment sample from responder Pt44 in the Riaz et al. cohort⁸, expressed as a percentage of total clonotypes, where the IGF2BP1-derived aeTSA IMKKIRESY-specific TCRB clonotype is highlighted. The grey lines connect each expanded TCRB clonotype in pre- and on-treatment samples (left) and the RNA expression of the cognate aeTSA in the pre- and on-treatment samples from Pt44 (right).

FIGS. 2A-D show the predicted presentation of unmutated TAs in melanoma and NSCLC samples from patients receiving ICB. FIG. 2A: Box plots showing the number of TA-HLA pairs (i.e., the sum of the HLA alleles per sample capable of presenting each expressed TA) per pre-treatment sample (grey dots) from various published studies in melanoma^{11,12,19-21} according to the response groups from the original studies. P-values from unpaired two-tailed T-test; no adjustments were made for multiple testing. FIGS. 2B-C: Box plots showing the number of TA-HLA pairs in pre- and on-treatment samples from Gide et al.¹¹ (FIG. 2B) and Du et al.¹² (FIG. 2C) according to the response groups from the original studies. Grey lines connect pre- and on-treatment samples per patient, p-values from paired two-tailed T-test; no adjustments were made for multiple testing. FIG. 2D: Box plots showing the difference in purity scores from ESTIMATE between on- and pre-therapy samples, where negative values indicate a decrease in tumor purity on-therapy in samples from Riaz et al.⁸ (upper). The lower heatmap shows Pearson's correlation coefficient between the purity change (from the upper panel) and the change in the number of TA-HLA pairs in on- vs. pre-ICB samples from corresponding patients. Box plots show the median and interquartile range (IQR), and whiskers extend to the largest value no further than 1.5*IQR from the box hinges.

FIG. 3A depicts the results of a FEST assay showing the expansion of specific T cell clonotypes following in vitro stimulation with the indicated aeTSAs selected based on their complete loss of RNA expression in at least one responder on-therapy from Riaz et al.⁸

FIG. 3B shows that the immunogenicity predictor PRIME 2.0 identifies melanoma aeTSAs described herein as immunogenic targets. PRIME scores of each peptide-HLA allele pairs were calculated by PRIME 2.0 for aeTSAs (n=200 binding 27 HLA alleles) and mTSAs (n=5 binding 5 HLA alleles) identified and reported herein as well as for immunogenic (n=590 binding 53 HLA alleles) and non-immunogenic (n=5887 binding 62 HLA alleles) peptide sequences used to train PRIME2.0. For each unique peptide sequence, the score of the highest scoring HLA allele was kept and used for plotting. Groups were compared using non-parametric Mann-Whitney U tests, where ns stands for not significant; *** P value <1E-10.

FIGS. 4A-D show TA sharing and expression regulation across cancer samples. FIG. 4A: Stacked bar chart showing the proportion of TA types (and absolute TA numbers) shared between different numbers of melanoma samples analyzed. FIG. 4B: Box plots showing the proportion of TCGA samples expressing each TA (grey dots) at least two times higher than the 95^{th} percentile value for the respective TA in GTEx samples except testis for melanoma TAs. Box plots show the median and interquartile range (IQR), and whiskers extend to the largest value no further than 1.5*IQR from the box hinges. FIGS. 4C-D: Spearman's correlation between the RPHM expression of each melanoma TA and the corresponding omics values [FPKM expression, copy-number variation, methylation β-value, and tumor mutational burden (TMB)]across the analyzed SKCM samples from TCGA (FIG. 4C), and the proportion of TAs with a significant correlation (p-adj <0.05, heatmap cells with * in FIG. 4C among TAs with omics data available (non-empty cells in FIG. 4C) (FIG. 4D).

FIG. 5 shows box plots showing the stemness scores obtained using ssGSEA in the TCGA samples studied herein across the LUAD, LUSC, and SKCM cohorts. P-values from two-sided Wilcoxon test.

FIGS. 6A-B show annotation of scRNA-seq data from previous studies of melanoma. FIG. 6A: balloon plot showing the average expression and proportion of cells expressing the indicated genes used for cluster annotation in each cluster identified across cutaneous melanoma samples from Zhang et al.¹⁴. The genes used for cluster annotation were obtained from the original article. FIG. 6B: UMAPs showing the clusters identified (upper) and their cell type annotation according to the genes in (FIG. 6A) (lower) across cutaneous melanoma samples from Zhang et al.¹⁴.

FIGS. 7A-B show the expression of unmutated TAs in scRNA-seq data from melanoma. FIG. 7A: Box plots showing the read count of cancer-specific melanoma TAs from FIG. 8A across cell types from cutaneous melanoma samples from Zhang et al.¹⁴. Each grey dot represents one TA per cell. FIG. 7B: Box plots showing the read count of melanoma TAs expressed in non-cancer cell types from cutaneous melanoma samples from Zhang et al.¹⁴. Each grey dot represents one TA per cell.

FIGS. 8A-C show TA expression in scRNA-seq data from melanoma. FIG. 8A: Bar plots showing the proportion (and absolute numbers) of melanoma TAs expressed (read count above 1) in cancer cells only in cutaneous melanoma scRNA-seq data from Zhang et al.¹⁴. FIG. 8B: Proportion of cell doublets among cells expressing a TA (cells expressing TA >1 read count) vs. the TA-negative cell fraction per annotated cell type from cutaneous melanomas from Zhang et al.¹⁴. Each grey dot represents a TA expressed in at least one cell of the respective cell type. FIG. 8C: Box plots show the normalized expression of MLANA (upper panel) and PMEL (lower panel) in cell types from cutaneous melanoma samples, comparing cells expressing at least one TA (TApos) vs. cells negative for all TAs (TAneg), per TA type identified in melanoma. All box plots show the median and interquartile range (IQR), and whiskers extend to the largest value no further than 1.5*IQR from the box hinges. P-values from two-sided Wilcoxon's nonparametric test; no adjustments were made for multiple testing.

### DETAILED DISCLOSURE

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless specifically defined otherwise, all technical and scientific terms used herein shall be taken to have the same meaning as commonly understood by one of ordinary skill in the art (e.g., in stem cell biology, cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

In the studies described herein, the present inventors have identified 505 tumor antigens (TAs) discovered using comprehensive proteogenomic analyses of cutaneous melanomas. Of these 505 TAs, five (1%) derive from mutated sequences and 500 (99%) from unmutated genomic sequences. While mutated TAs were rare and patient-specific, unmutated TAs were shared among tumor samples. Unmutated TAs belong to three classes: i) 200 aberrantly-expressed tumor-specific antigens (aeTSAs) absent from normal tissues, ii) 129 tumor-associated antigens (TAAs) overexpressed in tumors relative to normal tissues, and iii) 171 lineage-specific antigens (LSAs) expressed specifically in melanoma samples, normal skin, and melanocytes. While a majority of TAAs (88%) derive from exons of annotated protein-coding genes, around half of the aeTSAs (53%) and LSAs (45%) derive from allegedly non-coding genomic regions such as ncRNAs, introns, and intergenic regions. The novel tumor antigen candidates identified herein, which includes melanocyte lineage-specific antigens (LSAs), tumor-associated antigens (TAAs) and tumor-specific antigens (TSAs), may be useful, e.g., for immunotherapies and vaccines against cancers expressing the tumor antigen candidates, such as melanoma.

The present disclosure relates to a tumor antigen peptide (TAP) (or tumor-specific peptide), such as a melanoma TAP, comprising, or consisting of, one of the amino acid sequences set forth in any one of SEQ ID NOs:1-505:

| Peptide | SEQ | Peptide | SEQ | Peptide | SEQ |
|---|---|---|---|---|---|
| sequence | ID NO: | sequence | ID NO: | sequence | ID NO: |
| TLDDAVHGV | 1 | KLGINNVSF | 170 | MEKEDYHSL | 339 |
| YQIGQVQGV | 2 | KYISQFQVF | 171 | MEKEDYHSLY | 340 |
| NEYSNITRLY | 3 | QTAAVSGVK | 172 | YHSLYQSHL | 341 |
| RLLADLTGA | 4 | LTPLNHPFSY | 173 | YMVPFIPLY | 342 |
| TPLSPDPTL | 5 | VFINKKYVF | 174 | LMEKEDYHSL | 343 |
| STYYWPRPR | 6 | NLITEEHPV | 175 | FAHEAPAFL | 344 |
| DSFSATETR | 7 | ELVKVIDGSSR | 176 | FASPLTGIA | 345 |
| ETSYVKVLH | 8 | FHSLPIHLF | 177 | HEAPAFLPW | 346 |
| AEMLGSVV | 9 | YSHDYSYY | 178 | VLFKKFIQK | 347 |
| EELSVLEVF | 10 | FQIEKLTPL | 179 | APAFLPWHR | 348 |
| FVQENYLEY | 11 | EHSVENGSI | 180 | LLMEKEDYHSL | 349 |
| IVQQPTEEK | 12 | ETIQNTHSF | 181 | HYYVSMDAL | 350 |
| SVFAHPRKL | 13 | YYSIWKTQI | 182 | WEQEIQKL | 351 |
| ASSLPTTMNY | 14 | SYYFHGLSF | 183 | LMEKEDYHSLY | 352 |
| IVQQPTEEKR | 15 | ESFQQPYIPR | 184 | LEQASRIWSW | 353 |
| LPTTMNYPL | 16 | KQVPSRVLY | 185 | NIFDLSAPEK | 354 |
| GSNPARYEF | 17 | MLFNKDLDAA | 186 | EAPAFLPWHR | 355 |
| TADDATVM | 18 | APRSAEPRAT | 187 | SEIWRDIDF | 356 |
| VLEHVVRV | 19 | YLDPGAVHAVF | 188 | AAMVGAVL | 357 |
| EVDPIGHLY | 20 | TLKKHKIV | 189 | TFLGVGQESF | 358 |
| QIMPKAGL | 21 | VGVPISTLY | 190 | ESWPSVFYNR | 359 |
| KVLEHVVRV | 22 | SPRPQPEL | 191 | ISQDTTVGR | 360 |
| QVPGSNPAR | 23 | KLSFPDSKF | 192 | RPWSGPYIL | 361 |
| FVYGEPREL | 24 | KNKDEYIHFY | 193 | SSDYVIPIGTY | 362 |
| TLPTFDPTKV | 25 | RAFEGRRRGW | 194 | DSDPDSFQDY | 363 |
| NQIMPKTGF | 26 | RLKARPSTY | 195 | DTRPWSGPY | 364 |
| EVDPIGHVY | 27 | APSLHATL | 196 | HYYSVKKTF | 365 |
| ELVRRILSR | 28 | AQKEPTGNY | 197 | NVCGSQQGR | 366 |
| MEVDPIGHLY | 29 | LSLGTTACR | 198 | YMNGTMSQV | 367 |
| SVMSSNVSF | 30 | SRNPTSYKY | 199 | MAKRTTHPL | 368 |
| TVPSSFPSW | 31 | SLNNDDHQIY | 200 | RPWSGPYILR | 369 |
| KYKDYFPVI | 32 | YVDPMITEI | 201 | MTVDSLVNK | 370 |
| MEVDPIGHVY | 33 | PSFSPYAKR | 202 | TRPWSGPYIL | 371 |
| TVYGEPRKL | 34 | HVNGSRPHAR | 203 | TTHPLFVIATR | 372 |
| GPDVREGTL | 35 | ITVRLAPPL | 204 | FVWTHYYSV | 373 |
| VYDGREHTV | 36 | SANPLSHRY | 205 | LSKEEKNHF | 374 |
| LMEVDPIGHLY | 37 | VPLAHSSSA | 206 | RAANQVFVL | 375 |
| KYKDYFPVILK | 38 | GQVPTTEVV | 207 | MVRSATLIK | 376 |
| EVFEGREDSIF | 39 | KTWGQYWQV | 208 | NQVFVLHSY | 377 |
| GVYDGREHTV | 40 | ALNFPGSQK | 209 | HNSTEFLI | 378 |
| EVVPISHLY | 41 | YVWKTWGQY | 210 | SQDVAGTTF | 379 |
| NAVGVYAGR | 42 | MPTAESTGM | 211 | AHNSTEFLI | 380 |
| RPDSRLLEL | 43 | HTMEVTVYHRR | 212 | STEFLIYER | 381 |
| SVFAHPRKLLM | 44 | EEAAGIGIL | 213 | EGFFTRQER | 382 |
| EVFEGREDSVF | 45 | EGFDHRDSK | 214 | FQKVLVSY | 383 |
| RVSHSHASF | 46 | AEEAAGIGIL | 215 | FTDFMGQIVY | 384 |
| ATFLQRKVLR | 47 | LTSSMVTPR | 216 | YFQKVLVSY | 385 |
| SYVKVLHHL | 48 | TSSMVTPRR | 217 | ALVNMPPHYR | 386 |
| FAFGEPREL | 49 | GLSNLTHVL | 218 | GVMSSTLYTV | 387 |
| KMAELVHFL | 50 | QLLALLPSL | 219 | LLSNTVSTL | 388 |
| FAHGHPLYTR | 51 | EAAQPMTKK | 220 | RVFHETGAQR | 389 |
| GVYAGREHFV | 52 | RVLGTKPSW | 221 | SASDHCRSR | 390 |
| NTAIATFLQRK | 53 | VLYPVPLESY | 222 | RRDGGIIIY | 391 |
| TLDEKVAEL | 54 | PYLGQMINL | 223 | VAALIANPR | 392 |
| RLLELHITM | 55 | ELFSYLIEK | 224 | RLLGTEFQV | 393 |
| EVPNSSPPR | 56 | EEQYIAQF | 225 | FSIIGVSRR | 394 |
| VEVTLREV | 57 | EAAQPMTKKR | 226 | LSSDGMYEY | 395 |
| ISGGPRISY | 58 | MPMQDIKMI | 227 | DEYFLPSL | 396 |
| GVPGPLLILPR | 59 | RLSEGDVMHL | 228 | SYIGLKGLYF | 397 |
| GVYDGEEHSV | 60 | HASSYISPEK | 229 | FTGFGGALGY | 398 |
| KISGGPRISY | 61 | MPMQDIKMIL | 230 | KTWDQVPFSV | 399 |
| FLYNQTNIRM | 62 | SPSVSQLSVL | 231 | RVADIPIKP | 400 |
| GQYQLVVSL | 63 | RLDQLLRHV | 232 | SVAKHTQSY | 401 |
| GVDDLVFRR | 64 | ALLERASATL | 233 | QTKGGQTWAR | 402 |
| KVLEYVIKV | 65 | TVAPTPATAR | 234 | ETGSSFMSF | 403 |
| HTLPALSRR | 66 | SQSPSVSQL | 235 | SLYSYFQKV | 404 |
| ALREEEEGV | 67 | STVAPTPATAR | 236 | QTKGGQTWARR | 405 |
| TTINFTRQR | 68 | QYIAQFTSQF | 237 | TVAALIANPR | 406 |
| SAYGEPRKL | 69 | GQHLHLETF | 238 | HLMVKDWQL | 407 |
| STANVTRAH | 70 | TIASVLVAR | 239 | NPLSKLSVL | 408 |
| SAFPTTINF | 71 | SLLQHLIGL | 240 | FVIPSTKDSNR | 409 |
| FRAVITKKV | 72 | TASPRSPVM | 241 | HYQVQTHL | 410 |
| AAAGVSSTK | 73 | AAFDGRHSQTL | 242 | ASDILSFAHKY | 411 |
| FPTTINFTR | 74 | SPRRLVEL | 243 | RLGQGVPLV | 412 |
| TTSEKIHER | 75 | LYVDSLFFL | 244 | SVPDHVQPSK | 413 |
| GLYDGMEHL | 76 | VFLREFPNF | 245 | RLLEKHISL | 414 |
| STANVTRAHGR | 77 | RPRFSTATEIA | 246 | SLFGVSERL | 415 |
| IEDPKAGLHV | 78 | ALIANPRRK | 247 | NHYQVQTHL | 416 |
| GVVGPQGLR | 79 | DFMGQIVYR | 248 | DLVGKDVTRVY | 417 |
| NVFAHATGESR | 80 | EIPDTVMGL | 249 | SLSPPTLPR | 418 |
| FLFGEPKRL | 81 | VMSSTLYTV | 250 | LADNGTLGKY | 419 |
| SAFPTTINFTR | 82 | SPQHKPRL | 251 | KAMYIRVSY | 420 |
| RLFSEDIHV | 83 | EPKPPTTGL | 252 | MQPKLKQVF | 421 |
| VYMPANTTF | 84 | IPDTIASVL | 253 | AIIQDGILL | 422 |
| EAVGFQPPR | 85 | TDFMGQIVYR | 254 | AFDRAVHF | 423 |
| EVDPTSHSY | 86 | GEDDEHRKI | 255 | NTPNIVSLR | 424 |
| LLDPAQRNLY | 87 | RLRGSIQSRY | 256 | RVADIPIKPW | 425 |
| SGISQVFQR | 88 | HLDESPAYL | 257 | HYIIPLQTI | 426 |
| RLFSEDIHVA | 89 | KMEETTVTY | 258 | VMKGINQGY | 427 |
| VFDVGKGNM | 90 | LPTDLFNSV | 259 | ATSPPASVR | 428 |
| KASEKIFYV | 91 | RPQGLAVL | 260 | ALEKDTPRY | 429 |
| EAAAAFRFPR | 92 | SLSEKSPEV | 261 | QVMKGINQGY | 430 |
| MSISMSLLK | 93 | GAFREGPPY | 262 | AAFDRAVHF | 431 |
| KASPSSLPH | 94 | YLNGATVVA | 263 | RPLRIKGVF | 432 |
| RTLQQGVLQDW | 95 | SMYLHTEGF | 264 | RQVMKGINQGY | 433 |
| TTATTFRAR | 96 | HILSHLHEV | 265 | KLDTTVKNI | 434 |
| NPSHLITPR | 97 | SISEAPLTEV | 266 | LPRTPRPDL | 435 |
| ETMETLRTSR | 98 | HQINSEATF | 267 | ITYTESNSR | 436 |
| MVWEGHPRERR | 99 | DIKQEGVGAF | 268 | RLQGSVTLV | 437 |
| LPDGSRVEL | 100 | SALGVTITK | 269 | SQFPSKNLPAF | 438 |
| DVVTGNVPLK | 101 | TVNDFTQNPR | 270 | LPRTPRPDLIL | 439 |
| ENPSHLITPR | 102 | SFIQAGSSY | 271 | DTAPQSHQV | 440 |
| SLARTILHL | 103 | GVTITKTLK | 272 | MAILGTAGR | 441 |
| KPHYGLRAI | 104 | AERPQGLAV | 273 | SVSSHLIHR | 442 |
| AFLDIHTAL | 105 | REKGPVKEV | 274 | STSPGTGVRK | 443 |
| KMSEFVYRF | 106 | TSVPPFNLR | 275 | EVAEVLGSVK | 444 |
| FLVTKTVTM | 107 | LSRLPSIAF | 276 | EIPRTPSEGR | 445 |
| HTTSVMENK | 108 | FPVHAAEKF | 277 | VFLKFSRRF | 446 |
| LVSIPIAVR | 109 | GEISASHV | 278 | SVSSHLIHRY | 447 |
| ITSGVKVKK | 110 | AADGGQHWTY | 279 | THLEDPHLM | 448 |
| VSIPIAVRR | 111 | EISASHVSR | 280 | SVFPRTSSR | 449 |
| DIIELQEGLR | 112 | SPRIPFSTF | 28 | GQYGQSKPWTF | 450 |
| HLKPPELGF | 113 | KLMDPGSLPPL | 282 | VPRPTSTVGL | 451 |
| QVIVKIIGH | 114 | ETKPEFERR | 283 | NSVSSHLIHRY | 452 |
| AEQEIMKKV | 115 | ETIMKFLKM | 284 | QEEDNLLRYF | 453 |
| EVPLKILAH | 116 | RRAHLWSTF | 285 | THLEDPHLML | 454 |
| IVKIIGHFY | 117 | HLQEKLQSL | 286 | ATTNILEHV | 455 |
| SLVSIPIAVR | 118 | ELSVVTSER | 287 | MTLRPTQPR | 456 |
| MVSPGVSLFK | 119 | YLDQQVPYTF | 288 | ETRPNNSKF | 457 |
| VLMMKLEDL | 120 | EAIPRIPRL | 289 | HSITEPLLY | 458 |
| YSDLHTPGRY | 121 | GYLDQQVPYTF | 290 | HLFLQPPSKR | 459 |
| EVARFLTYY | 122 | SPKLGGIGF | 291 | ETVRFTSSF | 460 |
| SVGDLMAPR | 123 | YPEKSRGCL | 292 | SPMPIPPLYL | 461 |
| VLWRGDSPL | 124 | TTIPFSTLRQR | 293 | ESYGRYTFY | 462 |
| QPRQGSPVAA | 125 | SLGAHSPALY | 294 | KSQGSFISF | 463 |
| KQTGIRHSY | 126 | SQNGQLIHY | 295 | GTILHIGVF | 464 |
| RPRQDAISV | 127 | KAKEHTANISW | 296 | KVFSGSSKR | 465 |
| KLITQIRTA | 128 | RVSKSDSTF | 297 | SSFSTLASLDK | 466 |
| SYLSEHSAAIF | 129 | SLNSQVTGL | 298 | TTQGAAPQR | 467 |
| TSVGDLMAPR | 130 | NSDLSEKLEY | 299 | HTTQGAAPQR | 468 |
| EIPTGVAKR | 131 | DSSTVGLQR | 300 | KIFQERAAF | 469 |
| SQINLSQIY | 132 | RASAIIIQR | 301 | GQITHNTSF | 470 |
| RRPSGGFRL | 133 | SQETLPLKI | 302 | ISLKPLIARY | 471 |
| EVFNVKNTFPK | 134 | STFDGLDRR | 303 | KHSDLILFW | 472 |
| KVLGGGWTV | 135 | RVKEIVINK | 304 | MVWEGHPRER | 473 |
| HEAPGQHSEV | 136 | AQPHHGVPY | 305 | STDLPILLK | 474 |
| EVSHPNMSGR | 137 | EVIQLKERDRK | 306 | EVISINTGHF | 475 |
| DTAPATFLR | 138 | EVKEKTEL | 307 | ETERVLEKSSY | 476 |
| KPQKDLHFI | 139 | TDPETLHY | 308 | KIKTINLRY | 477 |
| RVRGAGTHL | 140 | KMKEKTQEL | 309 | YVSQKVSDF | 478 |
| TAPIIVAQR | 141 | STFDGLDRRR | 310 | DAGIPVYHR | 479 |
| TSWDTKFYEK | 142 | KMSELQTYV | 311 | TILEAEILHQA | 480 |
| KVFAEHKISY | 143 | GTDPETLHY | 312 | EYAQKTPLF | 481 |
| DAFQGIANR | 144 | TEEKGSILI | 313 | TWTPHGIRI | 482 |
| IVADPRVPR | 145 | DTAMPAHSR | 314 | EVHEGGVARF | 483 |
| TGFSTSLGR | 146 | KLQEALKTI | 315 | REIEIEGRL | 484 |
| ATAPIIVAQR | 147 | GTDPETLHYF | 316 | SWLPIPAKY | 485 |
| GQFLVKSGY | 148 | SLKNRIESV | 317 | RYSERVYTI | 486 |
| WTSRTVQNK | 149 | NLKDPVGVY | 318 | FTDSQGNDIK | 487 |
| ATYGGSDHYI | 150 | FTETNPQVY | 319 | SVPSDFLSLR | 488 |
| VYIAELEKI | 151 | ETQEAEVAVSR | 320 | RQMSLEFRA | 489 |
| VFLDRAVSI | 152 | SPRPESGTV | 321 | ETISPRPSI | 490 |
| MPSPIDHPR | 153 | SEFAKDPSL | 322 | GPAGGRGVI | 491 |
| EAVRHILHGSR | 154 | KVIKKIGHY | 323 | LTDHHIKEY | 492 |
| YPAGNVKEL | 155 | RTWQTRKQF | 324 | EAVPYNGSLFK | 493 |
| SVPRSLASR | 156 | AMAHKVIKA | 325 | NLLAEIHGV | 494 |
| ALIKHVANA | 157 | KLKQFQKEY | 326 | KEGPVTRGA | 495 |
| YTANLVPVR | 158 | SPLEDEESL | 327 | VHVDLNLGF | 496 |
| LSSSLVTKL | 159 | NSTDFTTDR | 328 | HTVQGLAGF | 497 |
| QNSNVLAVR | 160 | ETKDTKGRYF | 329 | MRIEDSHTKL | 498 |
| TTTKNTLNK | 161 | RPRVSSTEM | 330 | SALSSAQPR | 499 |
| HNYINLHSL | 162 | LMDKSSTIY | 331 | SPSIKSLLVY | 500 |
| HLKDYFEEY | 163 | GTLRPLKVRR | 332 | PTDATLNRLLY | 501 |
| SPEYFTSYL | 164 | ILSEGSVPL | 333 | WTSQPPNYEK | 502 |
| ATDVLDHLY | 165 | TVYNFRTEL | 334 | PAPRPVAPTL | 503 |
| QTFEESLGR | 166 | LPSSADVEF | 335 | VYLHSNSTL | 504 |
| HEQPELQKV | 167 | RLPSSADVEF | 336 | SLYAWTQSH | 505 |
| MATDVLDHLY | 168 | ALMDKSLHV | 337 | | |
| TSGSLLFPR | 169 | MHNALHIYM | 338 | | |

In general, peptides such as tumor antigen peptides (TAPs) presented in the context of HLA class I vary in length from about 7 or 8 to about 15, or preferably 8 to 14 amino acid residues. In some embodiments of the methods of the disclosure, longer peptides comprising the TAP sequences defined herein are artificially loaded into cells such as antigen presenting cells (APCs), processed by the cells and the TAP is presented by MHC class I molecules at the surface of the APC. In this method, peptides/polypeptides longer than 15 amino acid residues can be loaded into APCs, are processed by proteases in the APC cytosol providing the corresponding TAP as defined herein for presentation. In some embodiments, the precursor peptide/polypeptide that is used to generate the TAP defined herein is for example 1000, 500, 400, 300, 200, 150, 100, 75, 50, 45, 40, 35, 30, 25, 20 or 15 amino acids or less. Thus, all the methods and processes using the TAPs described herein include the use of longer peptides or polypeptides (including the native protein), i.e., tumor antigen precursor peptides/polypeptides, to induce the presentation of the “final” 8-14 TAP following processing by the cell (APCs). In some embodiments, the herein-mentioned TAP is about 8 to 14, 8 to 13, or 8 to 12 amino acids long (e.g., 8, 9, 10, 11, 12 or 13 amino acids long), small enough for a direct fit in an HLA class I molecule. In an embodiment, the TAP comprises 20 amino acids or less, preferably 15 amino acids or less, more preferably 14 amino acids or less. In an embodiment, the TAP comprises at least 7 amino acids, preferably at least 8 amino acids or less, more preferably at least 9 amino acids.

The term “amino acid” as used herein includes both L- and D-isomers of the naturally occurring amino acids as well as other amino acids (e.g., naturally-occurring amino acids, non-naturally-occurring amino acids, amino acids which are not encoded by nucleic acid sequences, etc.) used in peptide chemistry to prepare synthetic analogs of TAPs. Examples of naturally occurring amino acids are glycine, alanine, valine, leucine, isoleucine, serine, threonine, etc. Other amino acids include for example non-genetically encoded forms of amino acids, amino acid analogs as well as a conservative substitution of an L-amino acid. Naturally-occurring non-genetically encoded amino acids and amino acid analogs include, for example, beta-alanine, 3-amino-propionic acid, 2,3-diaminopropionic acid, alpha-aminoisobutyric acid (Aib), 4-amino-butyric acid, N-methylglycine (sarcosine), hydroxyproline, ornithine (e.g., L-ornithine), citrulline, t-butylalanine, t-butylglycine, N-methylisoleucine, phenylglycine, cyclohexylalanine, norleucine (NIe), norvaline, 2-napthylalanine, pyridylalanine, 3-benzothienyl alanine, 4-chlorophenylalanine, 2-fluorophenylalanine, 3-fluorophenylalanine, 4-fluorophenylalanine, penicillamine, 1,2,3,4-tetrahydro-isoquinoline-3-carboxylix acid, beta-2-thienylalanine, methionine sulfoxide, L-homoarginine (Hoarg), N-acetyl lysine, 2-amino butyric acid, 2-amino butyric acid, 2,4,-diaminobutyric acid (D- or L-), p-aminophenylalanine, N-methylvaline, homocysteine, homoserine (HoSer), cysteic acid, epsilon-amino hexanoic acid, delta-amino valeric acid, benzyloxy-tyrosine, p-phenylalanine or 2,3-diaminobutyric acid (D- or L-), etc. These amino acids are well known in the art of biochemistry/peptide chemistry. Thus, one or more of the amino acids in the TAPs described herein (SEQ ID NOs:1-505) may be replaced by a non-genetically encoded amino acid and/or an amino acid analog. The TAPs may also be modified to improve the proteolytic stability of the peptides, for example by the incorporation of methyl-amino acids, p-amino acids or peptoids. In an embodiment, the TAP comprises only naturally-occurring amino acids.

In embodiments, the TAPs described herein include peptides with altered sequences containing substitutions of functionally equivalent amino acid residues, relative to the herein-mentioned sequences. For example, one or more amino acid residues within the sequence can be substituted by another amino acid of a similar polarity (having similar physico-chemical properties) which acts as a functional equivalent, resulting in a silent alteration. Substitution for an amino acid within the sequence may be selected from other members of the class to which the amino acid belongs. For example, positively charged (basic) amino acids include arginine, lysine and histidine (as well as homoarginine and ornithine). Nonpolar (hydrophobic) amino acids include leucine, isoleucine, alanine, phenylalanine, valine, proline, tryptophan and methionine. Uncharged polar amino acids include serine, threonine, cysteine, tyrosine, asparagine and glutamine. Negatively charged (acidic) amino acids include glutamic acid and aspartic acid. The amino acid glycine may be included in either the nonpolar amino acid family or the uncharged (neutral) polar amino acid family. Substitutions made within a family of amino acids are generally understood to be conservative substitutions. The herein-mentioned TAP may comprise all L-amino acids, all D-amino acids or a mixture of L- and D-amino acids. In an embodiment, the herein-mentioned TAP comprises all L-amino acids.

In an embodiment, in the sequences of the TAPs comprising or consisting of one of sequences of SEQ ID NOs: 1-505, the amino acid residues that do not substantially contribute to interactions with the T-cell receptor may be modified by replacement with other amino acid whose incorporation does not substantially affect T-cell reactivity and does not eliminate binding to the relevant MHC.

The TAP may also be modified by replacing one or more of the amide bonds of the peptide that may improve chemical stability and/or enhanced biological/pharmacological properties (e.g., half-life, absorption, potency, efficiency, etc.). Typical peptide bond replacements include esters, polyamines and derivatives thereof as well as substituted alkanes and alkenes, such as aminomethyl and ketomethylene. For example, the above-mentioned TAP may have one or more amide bonds replaced by linkages such as —CH₂NH—, —CH₂S—, —CH₂—CH₂—, —CH═CH— (cis or trans), —CH₂SO—, —CH(OH)CH₂—, or —COCH₂—.

The TAP may also be N- and/or C-terminally capped or modified to prevent degradation, increase stability, affinity and/or uptake. Thus, in another aspect, the present disclosure provides a modified TAP of the formula Z¹—X—Z², wherein X is a TAP comprising, or consisting of, one of the amino acid sequences of SEQ ID NOs: 1-505.

In an embodiment, the amino terminal residue (i.e., the free amino group at the N-terminal end) of the TAP is modified (e.g., for protection against degradation), for example by covalent attachment of a moiety/chemical group (Z¹). Z¹ may be a straight chained or branched alkyl group of one to eight carbons, or an acyl group (R—CO—), wherein R is a hydrophobic moiety (e.g., acetyl, propionyl, butanyl, iso-propionyl, or iso-butanyl), or an aroyl group (Ar—CO—), wherein Ar is an aryl group. In an embodiment, the acyl group is a C₁-C₁₆ or C₃-C₁₆ acyl group (linear or branched, saturated or unsaturated), in a further embodiment, a saturated C₁-C₆ acyl group (linear or branched) or an unsaturated C₃-C₆ acyl group (linear or branched), for example an acetyl group (CH₃—CO—, Ac). In an embodiment, Z¹ is absent. The carboxy terminal residue (i.e., the free carboxy group at the C-terminal end of the TAP) of the TAP may be modified (e.g., for protection against degradation), for example by amidation (replacement of the OH group by a NH₂ group), thus in such a case Z² is a NH₂ group. In an embodiment, Z² may be an hydroxamate group, a nitrile group, an amide (primary, secondary or tertiary) group, an aliphatic amine of one to ten carbons such as methyl amine, iso-butylamine, iso-valerylamine or cyclohexylamine, an aromatic or arylalkyl amine such as aniline, napthylamine, benzylamine, cinnamylamine, or phenylethylamine, an alcohol or CH₂OH. In an embodiment, Z² is absent. In an embodiment, the TAP comprises one of the amino acid sequences of SEQ ID NOs: 1-505. In an embodiment, the TAP consists of one of the amino acid sequences of SEQ ID NOs: 1-505, i.e., wherein Z¹ and Z² are absent.

In another aspect, the present disclosure provides a TAP binding to an HLA-A*01:01 molecule, comprising or consisting of the sequence of SEQ ID NO: 11, 14, 20, 27, 29, 37, 86, 87, 121, 150, 165, 173, 279, 288, 299, 308, 312, 316, 319, 331, 340, 352, 362, 363, 384, 395, 398, 411, 419, 429, 458, 474, 476, 487, 492, 500, or 501.

In another aspect, the present disclosure provides a TAP binding to an HLA-A*02:01 molecule, comprising or consisting of the sequence of SEQ ID NO: 1, 2, 4, 19, 22, 25, 40, 50, 52, 54, 60, 65, 67, 76, 81, 89, 91, 103, 107, 120, 124, 128, 135, 157, 175, 179, 186, 188, 201, 208, 218, 219, 228, 232, 233, 240, 250, 257, 261, 263, 265, 266, 282, 286, 298, 311, 315, 317, 325, 333, 337, 349, 367, 373, 387, 388, 393, 399, 404, 412, 414, 415, 422, 434, 437, 455, 480, 489, or 494.

In another aspect, the present disclosure provides a TAP binding to an HLA-A*02:09 molecule, comprising or consisting of the sequence of SEQ ID NO: 22, 40, 50, 62, 83, 89, 232, 265, 282, 311, 315, 317, 337, 349, 367, 399, 407, 412, or 494.

In another aspect, the present disclosure provides a TAP binding to an HLA-A*03:01 molecule, comprising or consisting of the sequence of SEQ ID NO: 209, 247, 272, 347, or 505.

In another aspect, the present disclosure provides a TAP binding to an HLA-A*23:01 molecule, comprising or consisting of the sequence of SEQ ID NO: 48, 84, 129, 151, 152, 171, 174, 182, 223, 237, 245, 290, 358, 397, 426, 446, 481, 482, or 486.

In another aspect, the present disclosure provides a TAP binding to an HLA-A*24:02 molecule, comprising or consisting of the sequence of SEQ ID NO: 31, 244 or 481.

In another aspect, the present disclosure provides a TAP binding to an HLA-A*25:01 molecule, comprising or consisting of the sequence of SEQ ID NO: 11, 31, 249, 289, 403, 425, 464, or 490.

In another aspect, the present disclosure provides a TAP binding to an HLA-A*26:01 molecule, comprising or consisting of the sequence of SEQ ID NO: 11, 20, 27, 39, 41, 42, 45, 114, 116, 122, 168, 181, 268, 284, 329, 364, 398, 417, 430, 440, 447, 452, 457, 460, 462, 475, 490, or 497.

In another aspect, the present disclosure provides a TAP binding to an HLA-A*30:01 molecule, comprising or consisting of the sequence of SEQ ID NO: 26, 304 or 376.

In another aspect, the present disclosure provides a TAP binding to an HLA-A*30:02 molecule, comprising or consisting of the sequence of SEQ ID NO: 117, 185, 190, 193, 197, 256, 294, 323, 471, or 485.

In another aspect, the present disclosure provides a TAP binding to an HLA-A*32:01 molecule, comprising or consisting of the sequence of SEQ ID NO: 17, 44, 46, 61, 95, 106, 126, 143, 170, 194, 208, 221, 264, 296, 324, 326, 401, 414, 420, 425 or 469.

In another aspect, the present disclosure provides a TAP binding to an HLA-A*33:01 molecule, comprising or consisting of the sequence of SEQ ID NO: 248.

In another aspect, the present disclosure provides a TAP binding to an HLA-A*68:01 molecule, comprising or consisting of the sequence of SEQ ID NO: 6-8, 12, 15, 23, 28, 41, 42, 47, 51, 53, 56, 59, 64, 66, 68, 70, 73-75, 77, 79, 80, 82, 85, 88, 92, 93, 96-99, 101, 102, 108-112, 118, 119, 123, 130, 131, 134, 137, 138, 141, 142, 144-147, 149, 153, 154, 156, 158, 160, 161, 166, 169, 172, 176, 184, 198, 202, 203, 212, 214, 216, 217, 220, 224, 226, 229, 234, 236, 239, 248, 254, 269, 270, 275, 280, 283, 287, 293, 300, 301, 303, 306, 310, 314, 320, 328, 332, 348, 354, 355, 359, 360, 366, 369, 370, 372, 381, 382, 386, 389, 390, 392, 394, 402, 405, 406, 409, 413, 418, 424, 428, 436, 441-445, 449, 456, 459, 465-468, 473, 479, 483, 488, 493, 499, or 502.

In another aspect, the present disclosure provides a TAP binding to an HLA-B*07:02 molecule, comprising or consisting of the sequence of SEQ ID NO: 35, 36, 43, 104, 125, 127, 139, 140, 187, 191, 196, 206, 231, 243, 246, 251, 252, 260, 281, 291, 292, 321, 330, 361, 408, 432, 435, 439, 451, 461, 491, or503.

In another aspect, the present disclosure provides a TAP binding to an HLA-B*08:01 molecule, comprising or consisting of the sequence of SEQ ID NO: 21, 162, 189, 286, 307, 309, 317, 339, 343, 349, or 414.

In another aspect, the present disclosure provides a TAP binding to an HLA-B*13:02 molecule, comprising or consisting of the sequence of SEQ ID NO: 207, 311, 393, 400, or 455.

In another aspect, the present disclosure provides a TAP binding to an HLA-B*14:01 molecule, comprising or consisting of the sequence of SEQ ID NO: 396.

In another aspect, the present disclosure provides a TAP binding to an HLA-B*15:01 molecule, comprising or consisting of the sequence of SEQ ID NO: 11, 26, 30, 63, 113, 132, 148, 192, 195, 200, 207, 210, 222, 235, 238, 258, 262, 267, 276, 295, 297, 305, 309, 318, 326, 336, 342, 374, 377, 383, 401, 421, 427, 433, 438, 450, 463, 470, or 477.

In another aspect, the present disclosure provides a TAP binding to an HLA-B*18:01 molecule, comprising or consisting of the sequence of SEQ ID NO: 3, 10, 33, 225 or 396.

In another aspect, the present disclosure provides a TAP binding to an HLA-B*35:03 molecule, comprising or consisting of the sequence of SEQ ID NO: 5, 16, 100, 155, 164, 211, 230, 253, 259, 277, 327, or 335.

In another aspect, the present disclosure provides a TAP binding to an HLA-B*37:01 molecule, comprising or consisting of the sequence of SEQ ID NO: 302.

In another aspect, the present disclosure provides a TAP binding to an HLA-B*38:01 molecule, comprising or consisting of the sequence of SEQ ID NO: 26, 180, 338, 341, 378, 380, 416, 448, 454, 496, or 498.

In another aspect, the present disclosure provides a TAP binding to an HLA-B*40:01 molecule, comprising or consisting of the sequence of SEQ ID NO: 215 or 322.

In another aspect, the present disclosure provides a TAP binding to an HLA-B*40:02 molecule, comprising or consisting of the sequence of SEQ ID NO: 213, 215, 339, or 351.

In another aspect, the present disclosure provides a TAP binding to an HLA-B*44:02 molecule, comprising or consisting of the sequence of SEQ ID NO: 346, 353, 356, or 453.

In another aspect, the present disclosure provides a TAP binding to an HLA-B*49:01 molecule, comprising or consisting of the sequence of SEQ ID NO: 9, 57, 78, 115, 136, 167, 255, 273, 274, 278, 313, 339, 480, 484, or 495.

In another aspect, the present disclosure provides a TAP binding to an HLA-B*51:01 molecule, comprising or consisting of the sequence of SEQ ID NO: 227 or 259.

In another aspect, the present disclosure provides a TAP binding to an HLA-C*03:03 molecule, comprising or consisting of the sequence of SEQ ID NO: 24, 69, 204 or 388.

In another aspect, the present disclosure provides a TAP binding to an HLA-C*03:04 molecule, comprising or consisting of the sequence of SEQ ID NO: 24, 49, 69, 159, 241, 242 or 334.

In another aspect, the present disclosure provides a TAP binding to an HLA-C*04:01 molecule, comprising or consisting of the sequence of SEQ ID NO: 90, 257, or 423.

In another aspect, the present disclosure provides a TAP binding to an HLA-C*06:02 molecule, comprising or consisting of the sequence of SEQ ID NO: 13, 34 or 72.

In another aspect, the present disclosure provides a TAP binding to an HLA-C*07:01 molecule, comprising or consisting of the sequence of SEQ ID NO: 13, 75, 133, 163, 183, 199, 205, 285, 334, 391 or 472.

In another aspect, the present disclosure provides a TAP binding to an HLA-C*07:02 molecule, comprising or consisting of the sequence of SEQ ID NO: 13, 32, 36, 105, 177, 339, 371 or 478.

In another aspect, the present disclosure provides a TAP binding to an HLA-C*08:02 molecule, comprising or consisting of the sequence of SEQ ID NO: 18, 54 or 379.

In another aspect, the present disclosure provides a TAP binding to an HLA-C*12:03 molecule, comprising or consisting of the sequence of SEQ ID NO: 24, 49, 58, 71, 94, 162, 178, 242, 289, 344, 345, 357, 367, 368, 375, 423 or 431.

In another aspect, the present disclosure provides a TAP binding to an HLA-C*14:02 molecule, comprising or consisting of the sequence of SEQ ID NO: 105, 271, 350, 365, 385, 410, 423, or 504.

The TAPs of the disclosure may be produced by expression in a host cell comprising a nucleic acid encoding the TAPs (recombinant expression) or by chemical synthesis (e.g., solid-phase peptide synthesis). Peptides can be readily synthesized by manual and/or automated solid phase procedures well known in the art. Suitable syntheses can be performed for example by utilizing “T-boc” or “Fmoc” procedures. Techniques and procedures for solid phase synthesis are described in for example Solid Phase Peptide Synthesis: A Practical Approach, by E. Atherton and R. C. Sheppard, published by IRL, Oxford University Press, 1989. Alternatively, the TAPs may be prepared by way of segment condensation, as described, for example, in Liu et al., Tetrahedron Lett. 37: 933-936, 1996; Baca et al., J. Am. Chem. Soc. 117: 1881-1887, 1995; Tam et al., Int. J. Peptide Protein Res. 45: 209-216, 1995; Schnolzer and Kent, Science 256: 221-225, 1992; Liu and Tam, J. Am. Chem. Soc. 116: 4149-4153, 1994; Liu and Tam, Proc. Natl. Acad. Sci. USA 91: 6584-6588, 1994; and Yamashiro and Li, Int. J. Peptide Protein Res. 31: 322-334, 1988). Other methods useful for synthesizing the TAPs are described in Nakagawa et al., J. Am. Chem. Soc. 107: 7087-7092, 1985. In an embodiment, the TAP is chemically synthesized (synthetic peptide). Another embodiment of the present disclosure relates to a non-naturally occurring peptide wherein said peptide consists or consists essentially of an amino acid sequences defined herein and has been synthetically produced (e.g., synthesized) as a pharmaceutically acceptable salt. The salts of the TAPs according to the present disclosure differ substantially from the peptides in their state(s) in vivo, as the peptides as generated in vivo are no salts. The non-natural salt form of the peptide may modulate the solubility of the peptide, in particular in the context of pharmaceutical compositions comprising the peptides, e.g., the peptide vaccines as disclosed herein. Preferably, the salts are pharmaceutically acceptable salts of the peptides.

In an embodiment, the herein-mentioned TAP is substantially pure. A compound is “substantially pure” when it is separated from the components that naturally accompany it. Typically, a compound is substantially pure when it is at least 60%, more generally 75%, 80% or 85%, preferably over 90% and more preferably over 95%, by weight, of the total material in a sample. Thus, for example, a polypeptide that is chemically synthesized or produced by recombinant technology will generally be substantially free from its naturally associated components, e.g., components of its source macromolecule. A nucleic acid molecule is substantially pure when it is not immediately contiguous with (i.e., covalently linked to) the coding sequences with which it is normally contiguous in the naturally occurring genome of the organism from which the nucleic acid is derived. A substantially pure compound can be obtained, for example, by extraction from a natural source; by expression of a recombinant nucleic acid molecule encoding a peptide compound; or by chemical synthesis. Purity can be measured using any appropriate method such as column chromatography, gel electrophoresis, HPLC, etc. In an embodiment, the TAP is in solution. In another embodiment, the TAP is in solid form, e.g., lyophilized.

In an embodiment, the TAP is encoded by a sequence located a non-protein coding region of the genome. In an embodiment, the TAP is encoded by a sequence located in an intergenic region. In another embodiment, the TAP is encoded by a non-coding RNA (ncRNA). In another embodiment, the TAP is encoded by a sequence located in an intron. In another embodiment, the TAP is encoded by a sequence located in an untranslated region (UTR), for example a 5′UTR. In another embodiment, the TAP is encoded by a sequence located in a non-coding exon.

In another aspect, the disclosure further provides a synthetic long peptide (SLP) comprising at least one of the TAP described herein. In an embodiment, the SLP comprises at least two TAPs, wherein at least one of the TAP is a TAP as described herein. In an embodiment, the SLP comprises at least two, three, four or five of the TAPs described herein. In an embodiment, the SLP comprises at least 10, 15, 20, 25, 30, 35 or 40 of the TAPs described herein. In an embodiment, the SLP comprises at least one of the TAPs described herein linked to one or more amino acid sequences or domains that confer desired properties to the SLP, such as sequences or domains that stabilize the SLP and/or that improve processing and presentation by MHC molecules, for example a sequence comprising a motif cleavable by cellular proteases such as cathepsins. In another embodiment, the SLP comprises at least one of the TAPs described herein, and a TAP that binds to MHC class II molecules. The TAPs may be directly attached to each other, or may be indirectly attached via a linker such as a short amino acid linker. In embodiments, the linker comprises about 4 to about 20 amino acids, or about 4 to about 15 amino acids, e.g., 4, 5, 6, 7, 8, 9, 10, 11, 12, 13, 14 or 15 amino acids. In an embodiment, the linker comprises glycine residues, serine residues, proline residues, threonine residues, or a mixture thereof. The linker may include sequences promoting the processing of the SLP to release the TAPs, such as a cathepsin-sensitive linker (e.g., a linker of 4-6 amino acids comprising the sequence LVGS (SEQ ID NO:1138), ASLG (SEQ ID NO:1139), PIVG (SEQ ID NO:1140), LLSV (SEQ ID NO:1141), VLSVG (SEQ ID NO:1142) or LLSVGG (SEQ ID NO:1143), see Rabu et al., Oncoimmunology. 2019; 8(4): e1560919). In an embodiment, the SLP has a length of 200, 150, 100, 90, 80, 70, 60 or 50 amino acids or less. In a further embodiment, the SLP has a length of 20 to 50, 45 or 40 amino acids, for example from 20 or 25 amino acids to 30, 35 or 40 amino acids.

The TAPs or SLPs described herein may further comprise one or more modifications that confer additional biological properties to the TAPs or SLPs such as protease resistance, plasma protein binding, increased plasma half-life, intracellular penetration, etc. Such modifications include, for example, covalent attachment of molecules/moiety to the TAPs or SLPs such as fatty acids (e.g., C₆-C₁₈), attachment of proteins such as albumin (see, e.g., U.S. Pat. No. 7,268,113); sugars/polysaccharides (glycosylation), biotinylation or PEGylation (see, e.g., U.S. Pat. Nos. 7,256,258 and 6,528,485). The above description of modification of the TAPs or SLPs does not limit the scope of the approaches nor the possible modifications that can be engineered. Thus, in another aspect, the present disclosure provides a conjugate comprising the TAPs or SLPs described herein and one or more additional molecules or agents (hereinafter secondary molecules or agents). The TAPs or SLPs may be conjugated to any type of synthetic or natural secondary molecules or agents, such as peptides. proteins, saccharides/polysaccharides, lipids, naturally-occurring or synthetic polymers/co-polymers, etc. to modify one or more properties of the TAPs or SLPs.

In another aspect, the disclosure further provides a nucleic acid (isolated) encoding the herein-mentioned TAPs or a tumor antigen precursor-peptide or SLP. In an embodiment, the nucleic acid comprises from about 21 nucleotides to about 45 nucleotides, from about 24 to about 45 nucleotides, for example 24, 27, 30, 33, 36, 39, 42 or 45 nucleotides. “Isolated” as used herein refers to a peptide or nucleic acid molecule separated from other components that are present in the natural environment of the molecule or a naturally occurring source macromolecule (e.g., including other nucleic acids, proteins, lipids, sugars, etc.). “Synthetic”, as used herein, refers to a peptide or nucleic molecule that is not isolated from its natural sources, e.g., which is produced through recombinant technology or using chemical synthesis. In an embodiment, the nucleic acid (DNA, RNA) encoding the TAP of the disclosure comprises or consists of any one of the sequences set forth in Table 1 below or a corresponding RNA sequence. In an embodiment, the nucleic acid encoding the TAP is an mRNA molecule. In other embodiments, the nucleic acid encoding the TAP or SLP is a self-amplifying mRNA (saRNA), a trans-amplifying mRNA (taRNA) or a circular mRNA (circRNA) (see, e.g., Liu et al., Nature Reviews Cancer, Volume 23, August 2023, pages 526-543).

TABLE 1
Nucleotide sequence of the nucleic acids encoding the
TSAs, TAAs and LSAs identified herein

| Peptide | |
|---|---|
| Sequence | |
| (SEQ ID NO:) | Encoding nucleotide sequence |
| TLDDAVHGV (1) | ACTCTCGATGACGCCGTCCACGGCGTT (506) |
| YQIGQVQGV (2) | TACCAAATTGGCCAAGTTCAAGGTGTA (507) |
| NEYSNITRLY (3) | AATGAATATTCTAACATTACCCGGCTTTAT (508) |
| RLLADLTGA (4) | CGGCTCCTGGCTGATCTCACTGGGGCC (509) |
| TPLSPDPTL (5) | ACTCCATTGTCTCCTGACCCCACCCTC (510) |
| STYYWPRPR (6) | TCGACCTATTATTGGCCTAGACCAAGG (511) |
| DSFSATETR (7) | GATTCTTTCTCCGCTACTGAGACACGG (512) |
| ETSYVKVLH (8) | GAAACCAGCTATGTGAAAGTCCTGCAC (513) |
| AEMLGSVV (9) | GCAGAAATGCTGGGGAGTGTCGTC (514) |
| EELSVLEVF (10) | GAGGAGCTGAGTGTGTTAGAGGTGTTT (515) |
| FVQENYLEY (11) | TTCGTGCAGGAAAACTACCTGGAGTAC (516) |
| IVQQPTEEK (12) | ATTGTCCAGCAGCCCACTGAGGAAAAA (517) |
| SVFAHPRKL (13) | AGTGTCTTTGCGCATCCCAGGAAGCTG (518); |
| | AGTGTCTTCGCACATCCCAGGAAGCTG (519) |
| ASSLPTTMNY (14) | GCCTCCAGCCTCCCCACTACCATGAACTAC (520) |
| IVQQPTEEKR (15) | ATTGTCCAGCAGCCCACTGAGGAAAAACGT (521) |
| LPTTMNYPL (16) | CTCCCCACTACCATGAACTACCCTCTC (522) |
| GSNPARYEF (17) | GGCAGTAATCCTGCGCGCTATGAGTTC (523) |
| TADDATVM (18) | ACCGCAGATGATGCCACTGTCATG (524) |
| VLEHVVRV (19) | GTCCTGGAGCATGTGGTCAGGGTC (525) |
| EVDPIGHLY (20) | GAAGTGGACCCCATCGGCCACTTGTAC (526) |
| QIMPKAGL (21) | CAGATCATGCCCAAGGCAGGCCTC (527) |
| KVLEHVVRV (22) | AAAGTCCTGGAGCATGTGGTCAGGGTC (528) |
| QVPGSNPAR (23) | CAGGTACCCGGCAGTAATCCTGCGCGC (529) |
| FVYGEPREL (24) | TTCGTCTATGGGGAGCCTAGGGAGCTC (530) |
| TLPTFDPTKV (25) | ACTCTGCCCACTTTTGATCCCACTAAAGTG (531) |
| NQIMPKTGF (26) | AATCAGATCATGCCCAAGACAGGCTTC (532) |
| EVDPIGHVY (27) | GAAGTGGACCCCATCGGCCACGTGTAC (533) |
| ELVRRILSR (28) | GAGCTGGTCCGCAGGATCCTGTCCCGG (534) |
| MEVDPIGHLY (29) | ATGGAAGTGGACCCCATCGGCCACTTGTAC (535) |
| SVMSSNVSF (30) | AGTGTCATGTCCAGCAACGTCTCCTTT (536) |
| TVPSSFPSW (31) | ACTGTTCCTAGTTCCTTTCCATCCTGG (537) |
| KYKDYFPVI (32) | AAGTACAAAGATTACTTTCCTGTGATA (538) |
| MEVDPIGHVY (33) | ATGGAAGTGGACCCCATCGGCCACGTGTAC (539) |
| TVYGEPRKL (34) | ACTGTCTATGGGGAGCCCAGGAAACTG (540) |
| GPDVREGTL (35) | GGTCCTGATGTCAGGGAGGGGACTCTG (541) |
| VYDGREHTV (36) | GTGTATGATGGGAGGGAGCACACTGTC (542) |
| LMEVDPIGHLY (37) | CTGATGGAAGTGGACCCCATCGGCCACTTGTAC (543) |
| KYKDYFPVILK (38) | AAGTACAAAGATTACTTTCCTGTGATACTCAAG (544) |
| EVFEGREDSIF (39) | GAGGTGTTTGAGGGGAGGGAAGACAGTATCTTC (545) |
| GVYDGREHTV (40) | GGGGTGTATGATGGGAGGGAGCACACTGTC (546) |
| EVVPISHLY (41) | GAAGTGGTCCCCATCAGCCACTTGTAC (547) |
| NAVGVYAGR (42) | AATGCAGTAGGGGTATATGCTGGGAGG (548) |
| RPDSRLLEL (43) | AGGCCGGACAGCCGCCTGCTTGAGTTG (549) |
| SVFAHPRKLLM (44) | AGTGTCTTCGCACATCCCAGGAAGCTGCTCATG (550) |
| EVFEGREDSVF (45) | GAGGTGTTTGAGGGGAGGGAGGACAGTGTCTTC (551) |
| RVSHSHASF (46) | CGAGTATCTCACTCACATGCTTCTTTT (552) |
| ATFLQRKVLR (47) | GCCACCTTTTTGCAGAGGAAGGTACTGAGG (553) |
| SYVKVLHHL (48) | AGCTATGTGAAAGTCCTGCACCATTTG (554) |
| FAFGEPREL (49) | TTTGCCTTTGGGGAGCCCAGGGAGCTC (555) |
| KMAELVHFL (50) | AAGATGGCTGAGTTGGTTCATTTTCTG (556) |
| FAHGHPLYTR (51) | TTTGCCCACGGGCACCCACTGTACACACGC (557) |
| GVYAGREHFV (52) | GGGGTATATGCTGGGAGGGAGCACTTCGTC (558) |
| NTAIATFLQRK (53) | AACACTGCTATTGCCACCTTTTTGCAGAGGAAG (559) |
| TLDEKVAEL (54) | ACACTAGATGAAAAGGTGGCCGAGTTA (560) |
| RLLELHITM (55) | CGCCTGCTTGAGTTGCACATCACGATG (561) |
| EVPNSSPPR (56) | GAGGTGCCCAACTCTTCTCCTCCTCGT (562) |
| VEVTLREV (57) | GTGGAAGTCACCCTGCGGGAGGTG (563) |
| ISGGPRISY (58) | ATCAGTGGAGGACCTCGCATTTCCTAC (564) |
| GVPGPLLILPR (59) | GGAGTCCCCGGACCCCTCCTCATCCTTCCCAGG (565) |
| GVYDGEEHSV (60) | GGAGTCTATGATGGAGAGGAGCACTCAGTC (566) |
| KISGGPRISY (61) | AAGATCAGTGGAGGACCTCGCATTTCCTAC (567) |
| FLYNQTNIRM (62) | TTTCTATATAATCAGACAAATATTAGGATG (568) |
| GQYQLVVSL (63) | GGCCAGTACCAGCTGGTGGTGAGCCTG (569) |
| GVDDLVFRR (64) | GGAGTTGATGACCTTGTTTTCAGAAGG (570) |
| KVLEYVIKV (65) | AAAGTCCTTGAGTATGTGATCAAGGTC (571) |
| HTLPALSRR (66) | CACACTTTACCAGCCCTTTCTAGAAGG (572) |
| ALREEEEGV (67) | GCTTTGAGAGAGGAGGAAGAGGGAGTC (573) |
| TTINFTRQR (68) | ACTACCATCAACTTCACTCGACAGAGG (574) |
| SAYGEPRKL (69) | AGTGCCTATGGGGAGCCCAGGAAGCTG (575) |
| STANVTRAH (70) | AGCACTGCCAATGTGACGCGGGCCCAC (576) |
| SAFPTTINF (71) | TCCGCCTTTCCCACTACCATCAACTTC (577) |
| FRAVITKKV (72) | TTCCGAGCAGTAATCACTAAGAAGGTG (578) |
| AAAGVSSTK (73) | GCGGCTGCGGGTGTTTCATCCACAAAA (579) |
| FPTTINFTR (74) | TTTCCCACTACCATCAACTTCACTCGA (580) |
| TTSEKIHER (75) | ACTACCTCTGAGAAGATTCACGAGAGA (581) |
| GLYDGMEHL (76) | GGGCTGTATGATGGGATGGAGCACCTC (582) |
| STANVTRAHGR (77) | AGCACTGCCAATGTGACGCGGGCCCACGGCCGC (583) |
| IEDPKAGLHV (78) | ATAGAGGACCCAAAGGCAGGGCTGCATGTG (584) |
| GVVGPQGLR (79) | GGTGTGGTGGGCCCCCAGGGCCTGAGG (585) |
| NVFAHATGESR (80) | AATGTATTTGCTCATGCAACTGGGGAATCAAGG (586) |
| FLFGEPKRL (81) | TTCCTCTTTGGGGAGCCCAAGAGGCTC (587) |
| SAFPTTINFTR (82) | TCCGCCTTTCCCACTACCATCAACTTCACTCGA (588) |
| RLFSEDIHV (83) | AGGCTCTTCTCAGAAGACATACATGTC (589) |
| VYMPANTTF (84) | GTTTACATGCCTGCTAATACAACATTT (590) |
| EAVGFQPPR (85) | GAGGCTGTGGGCTTTCAACCACCACGA (591) |
| EVDPTSHSY (86) | GAAGTGGACCCCACTAGCCACTCCTAT (592) |
| LLDPAQRNLY (87) | TTACTGGACCCAGCTCAGAGAAACCTGTAC (593) |
| SGISQVFQR (88) | TCTGGAATCTCGCAAGTCTTCCAGAGG (594) |
| RLFSEDIHVA (89) | AGGCTCTTCTCAGAAGACATACATGTCGCC (595) |
| VFDVGKGNM (90) | GTGTTTGATGTTGGCAAAGGAAACATG (596) |
| KASEKIFYV (91) | AAAGCCTCGGAGAAAATCTTCTATGTG (597) |
| EAAAAFRFPR (92) | GAGGCCGCCGCTGCCTTCAGGTTTCCGAGA (598) |
| MSISMSLLK (93) | ATGAGTATCTCCATGTCTTTATTGAAA (599) |
| KASPSSLPH (94) | AAGGCATCCCCCAGCTCTCTGCCTCAT (600) |
| RTLQQGVLQDW (95) | AGAACCTTGCAGCAAGGAGTTCTCCAAGACTGG (601) |
| TTATTFRAR (96) | ACTACTGCCACGACTTTTAGAGCGCGT (602) |
| NPSHLITPR (97) | AATCCTAGCCATCTCATCACACCCAGA (603) |
| ETMETLRTSR (98) | GAGACAATGGAGACTCTGAGAACATCCAGA (604) |
| MVWEGHPRERR (99) | ATGGTATGGGAGGGGCATCCCAGAGAAAGAAGA (605) |
| LPDGSRVEL (100) | CTCCCAGACGGCTCCCGCGTGGAGCTG (606) |
| DVVTGNVPLK (101) | GATGTTGTGACTGGCAACGTGCCTCTAAAA (607) |
| ENPSHLITPR (102) | GAGAATCCTAGCCATCTCATCACACCCAGA (608) |
| SLARTILHL (103) | TCACTGGCCAGGACTATACTCCATCTC (609) |
| KPHYGLRAI (104) | AAACCACATTATGGATTGAGAGCAATC (610) |
| AFLDIHTAL (105) | GCCTTTCTGGACATCCACACAGCCCTG (611) |
| KMSEFVYRF (106) | AAGATGTCAGAGTTTGTCTACAGATTT (612) |
| FLVTKTVTM (107) | TTTTTAGTGACAAAGACTGTGACTATG (613) |
| HTTSVMENK (108) | CACACCACCTCTGTGATGGAGAACAAG (614) |
| LVSIPIAVR (109) | CTTGTGAGCATTCCCATTGCAGTAAGA (615) |
| ITSGVKVKK (110) | ATTACATCAGGAGTAAAAGTGAAGAAA (616) |
| VSIPIAVRR (111) | GTGAGCATTCCCATTGCAGTAAGAAGA (617) |
| DIIELQEGLR (112) | GACATCATTGAGCTCCAGGAGGGGCTGAGA (618) |
| HLKPPELGF (113) | CATCTCAAGCCTCCAGAGCTGGGCTTC (619) |
| QVIVKIIGH (114) | CAGGTCATCGTGAAAATCATCGGACAT (620) |
| AEQEIMKKV (115) | GCCGAGCAGGAAATAATGAAGAAAGTT (621) |
| EVPLKILAH (116) | GAGGTTCCCCTGAAGATCCTGGCCCAT (622) |
| IVKIIGHFY (117) | ATCGTGAAAATCATCGGACATTTCTAT (623) |
| SLVSIPIAVR (118) | TCTCTTGTGAGCATTCCCATTGCAGTAAGA (624) |
| MVSPGVSLFK (119) | ATGGTTTCTCCTGGAGTTTCTCTATTCAAA (625) |
| VLMMKLEDL (120) | GTGTTGATGATGAAGTTGGAAGATTTG (626) |
| YSDLHTPGRY (121) | TATTCGGACTTACACACCCCTGGGAGGTAT (627) |
| EVARFLTYY (122) | GAAGTGGCCCGGTTCCTGACCTACTAC (628) |
| SVGDLMAPR (123) | AGTGTCGGAGACCTCATGGCTCCCCGG (629) |
| VLWRGDSPL (124) | GTGCTCTGGAGAGGTGACTCTCCTCTA (630) |
| QPRQGSPVAA (125) | CAGCCCCGCCAGGGCTCACCTGTGGCAGCG (631) |
| KQTGIRHSY (126) | AAACAAACAGGTATTAGGCACAGTTAT (632) |
| RPRQDAISV (127) | AGACCCAGACAAGATGCCATTTCAGTA (633) |
| KLITQIRTA (128) | AAACTGATAACACAGATACGTACAGCA (634) |
| SYLSEHSAAIF (129) | TCTTATTTGTCTGAGCACTCTGCAGCTATTTTC (635) |
| TSVGDLMAPR (130) | ACCAGTGTCGGAGACCTCATGGCTCCCCGG (636) |
| EIPTGVAKR (131) | GAGATACCAACTGGGGTGGCCAAGCGG (637) |
| SQINLSQIY (132) | AGTCAAATCAACCTTAGCCAAATTTAT (638) |
| RRPSGGFRL (133) | CGAAGACCCTCAGGCGGGTTCCGGCTC (639) |
| EVFNVKNTFPK | GAAGTATTTAATGTAAAAAATACATTTCCAAAG (640) |
| (134) | |
| KVLGGGWTV (135) | AAGGTCTTAGGAGGTGGATGGACTGTC (641) |
| HEAPGQHSEV (136) | CATGAGGCTCCTGGCCAGCACAGTGAAGTT (642) |
| EVSHPNMSGR (137) | GAAGTAAGCCACCCTAACATGAGTGGCAGA (643) |
| DTAPATFLR (138) | GACACAGCCCCAGCCACCTTCCTACGG (644) |
| KPQKDLHFI (139) | AAACCCCAAAAAGACCTGCACTTCATA (645) |
| RVRGAGTHL (140) | CGAGTGCGTGGGGCAGGAACACACCTG (646) |
| TAPIIVAQR (141) | ACGGCTCCCATTATTGTGGCTCAGAGA (647) |
| TSWDTKFYEK (142) | ACGAGCTGGGATACCAAGTTCTATGAAAAG (648) |
| KVFAEHKISY (143) | AAAGTGTTTGCGGAGCACAAGATCTCCTAC (649) |
| DAFQGIANR (144) | GACGCCTTCCAGGGCATCGCCAACAGG (650) |
| IVADPRVPR (145) | ATTGTGGCCGACCCGCGGGTGCCCCGT (651) |
| TGFSTSLGR (146) | ACAGGCTTCTCCACGAGCCTGGGCAGA (652) |
| ATAPIIVAQR (147) | GCCACGGCTCCCATTATTGTGGCTCAGAGA (653) |
| GQFLVKSGY (148) | GGCCAGTTCTTGGTCAAATCCGGCTAC (654) |
| WTSRTVQNK (149) | TGGACTTCCAGAACGGTGCAAAATAAA (655) |
| ATYGGSDHYI (150) | GCCACATATGGGGGTTCAGATCATTACATT (656) |
| VYIAELEKI (151) | GTTTACATTGCAGAGTTGGAAAAGATA (657) |
| VFLDRAVSI (152) | GTGTTCCTGGACCGCGCCGTGTCCATC (658) |
| MPSPIDHPR (153) | ATGCCCAGTCCCATCGACCACCCAAGG (659) |
| EAVRHILHGSR | GAAGCAGTAAGGCACATCTTACATGGCAGCAGG |
| (154) | (660) |
| YPAGNVKEL (155) | TACCCAGCAGGCAACGTCAAAGAGCTA (661) |
| SVPRSLASR (156) | AGCGTACCCAGAAGCCTGGCATCCCGG (662) |
| ALIKHVANA (157) | GCGCTAATTAAGCACGTTGCGAACGCA (663) |
| YTANLVPVR (158) | TACACGGCAAATCTGGTTCCAGTTAGA (664) |
| LSSSLVTKL (159) | TTGTCCTCTTCCCTTGTCACCAAACTT (665) |
| QNSNVLAVR (160) | CAGAACAGTAATGTTTTAGCAGTGAGG (666) |
| TTTKNTLNK (161) | ACGACAACAAAAAACACCTTGAACAAA (667) |
| HNYINLHSL (162) | CACAATTATATTAATTTGCATTCTCTG (668) |
| HLKDYFEEY (163) | CACCTTAAAGATTACTTTGAGGAATAT (669) |
| SPEYFTSYL (164) | TCTCCAGAATATTTTACCAGCTATCTG (670) |
| ATDVLDHLY (165) | GCCACAGATGTTTTGGACCACTTATAT (671) |
| QTFEESLGR (166) | CAGACCTTTGAGGAATCACTGGGAAGA (672) |
| HEQPELQKV (167) | CATGAACAGCCAGAACTGCAGAAGGTA (673) |
| MATDVLDHLY (168) | ATGGCCACAGATGTTTTGGACCACTTATAT (674) |
| TSGSLLFPR (169) | ACTTCTGGCAGTCTCCTGTTTCCCAGA (675) |
| KLGINNVSF (170) | AAGCTTGGAATAAATAACGTTTCCTTT (676) |
| KYISQFQVF (171) | AAATATATTTCACAATTTCAAGTATTC (677) |
| QTAAVSGVK (172) | CAGACTGCGGCTGTGTCGGGAGTAAAG (678) |
| LTPLNHPFSY (173) | TTGACACCATTAAACCATCCTTTTTCCTAC (679) |
| VFINKKYVF (174) | GTATTTATAAATAAAAAGTACGTTTTT (680) |
| NLITEEHPV (175) | AACCTCATCACTGAGGAGCATCCTGTC (681) |
| ELVKVIDGSSR | GAGTTAGTGAAGGTCATTGATGGAAGCTCCAGG |
| (176) | (682) |
| FHSLPIHLF (177) | TTTCACTCACTTCCAATTCATTTGTTC (683) |
| YSHDYSYY (178) | TATTCTCATGATTACTCTTACTAT (684) |
| FQIEKLTPL (179) | TTCCAGATAGAAAAATTGACACCCCTG (685) |
| EHSVENGSI (180) | GAGCACTCTGTTGAAAATGGCAGTATT (686) |
| ETIQNTHSF (181) | GAGACAATTCAAAACACACATTCATTT (687) |
| YYSIWKTQI (182) | TACTACAGTATTTGGAAGACACAAATT (688) |
| SYYFHGLSF (183) | TCTTATTATTTTCACGGCCTGTCATTT (689) |
| ESFQQPYIPR (184) | GAGTCTTTTCAACAACCTTATATTCCCAGG (690) |
| KQVPSRVLY (185) | AAACAGGTACCCTCTAGGGTCCTCTAC (691) |
| MLFNKDLDAA (186) | ATGTTATTTAATAAAGATTTGGATGCAGCA (692) |
| APRSAEPRAT (187) | GCCCCACGGTCAGCAGAGCCACGGGCCACC (693) |
| YLDPGAVHAVF | TATCTGGATCCTGGGGCAGTGCATGCAGTGTTT |
| (188) | (694) |
| TLKKHKIV (189) | ACCCTTAAGAAACATAAGATAGTT (695) |
| VGVPISTLY (190) | GTGGGTGTGCCAATCAGCACTCTGTAT (696) |
| SPRPQPEL (191) | AGCCCCCGCCCCCAGCCAGAGCTC (697) |
| KLSFPDSKF (192) | AAGCTTTCCTTCCCTGATTCAAAATTT (698) |
| KNKDEYIHFY (193) | AAAAACAAAGATGAATATATCCATTTTTAC (699) |
| RAFEGRRRGW (194) | AGGGCATTTGAGGGGAGAAGAAGAGGATGG (700) |
| RLKARPSTY (195) | CGTCTCAAGGCCAGGCCTTCCACCTAC (701) |
| APSLHATL (196) | GCCCCCTCTCTTCACGCCACACTC (702) |
| AQKEPTGNY (197) | GCGCAGAAAGAACCCACTGGCAATTAC (703) |
| LSLGTTACR (198) | CTCTCACTGGGTACCACAGCTTGCAGG (704) |
| SRNPTSYKY (199) | AGCAGAAACCCTACAAGCTACAAGTAT (705) |
| SLNNDDHQIY (200) | AGCCTAAATAATGACGATCACCAGATATAC (706) |
| YVDPMITEI (201) | TATGTTGATCCCATGATTACAGAGATA (707) |
| PSFSPYAKR (202) | CCCAGCTTTTCTCCATATGCAAAAAGG (708) |
| HVNGSRPHAR (203) | CACGTCAACGGCTCCCGCCCCCATGCACGG (709) |
| ITVRLAPPL (204) | ATAACAGTGAGATTGGCTCCTCCACTC (710) |
| SANPLSHRY (205) | TCAGCCAATCCCCTAAGTCATAGATAC (711) |
| VPLAHSSSA (206) | GTGCCTCTTGCTCATTCCAGCTCAGCC (712) |
| GQVPTTEVV (207) | GGCCAAGTGCCTACTACAGAAGTTGTG (713) |
| KTWGQYWQV (208) | AAGACCTGGGGCCAATACTGGCAAGTT (714) |
| ALNFPGSQK (209) | GCCTTGAACTTCCCTGGAAGCCAAAAG (715) |
| YVWKTWGQY (210) | TATGTCTGGAAGACCTGGGGCCAATAC (716) |
| MPTAESTGM (211) | ATGCCAACTGCAGAGAGCACAGGTATG (717) |
| HTMEVTVYHRR | CACACCATGGAAGTGACTGTCTACCATCGCCGG |
| (212) | (718) |
| EEAAGIGIL (213) | GAAGAGGCCGCTGGGATCGGCATCCTG (719) |
| EGFDHRDSK (214) | GAAGGGTTTGATCATCGGGACAGCAAA (720) |
| AEEAAGIGIL (215) | GCTGAAGAGGCCGCTGGGATCGGCATCCTG (721) |
| LTSSMVTPR (216) | CTCACTTCATCTATGGTTACCCCAAGA (722) |
| TSSMVTPRR (217) | ACTTCATCTATGGTTACCCCAAGAAGG (723) |
| GLSNLTHVL (218) | GGGCTGAGCAATCTGACCCACGTGCTG (724) |
| QLLALLPSL (219) | CAGCTCCTTGCCCTCCTGCCTTCCCTG (725) |
| EAAQPMTKK (220) | GAAGCAGCTCAGCCCATGACAAAGAAG (726) |
| RVLGTKPSW (221) | AGGGTTTTAGGAACAAAGCCTTCCTGG (727) |
| VLYPVPLESY (222) | GTGCTGTATCCTGTCCCCCTGGAGAGTTAT (728) |
| PYLGQMINL (223) | CCTTACCTGGGCCAGATGATTAATCTG (729) |
| ELFSYLIEK (224) | GAATTGTTCTCCTACCTCATTGAGAAA (730) |
| EEQYIAQF (225) | GAAGAGCAGTATATCGCCCAGTTC (731) |
| EAAQPMTKKR (226) | GAAGCAGCTCAGCCCATGACAAAGAAGCGA (732) |
| MPMQDIKMI (227) | ATGCCCATGCAGGATATCAAGATGATC (733) |
| RLSEGDVMHL (228) | CGGCTTTCGGAAGGGGATGTGATGCATCTG (734) |
| HASSYISPEK (229) | CATGCATCTTCCTACATTTCCCCGGAGAAG (735) |
| MPMQDIKMIL (230) | ATGCCCATGCAGGATATCAAGATGATCCTG (736) |
| SPSVSQLSVL (231) | AGTCCCAGCGTCAGTCAGCTAAGTGTCCTG (737) |
| RLDQLLRHV (232) | CGCCTGGATCAGTTGCTCAGGCACGTG (738) |
| ALLERASATL (233) | GCTCTGCTGGAGAGAGCCTCTGCCACCCTC (739) |
| TVAPTPATAR (234) | ACCGTCGCTCCTACCCCGGCCACCGCTCGG (740) |
| SQSPSVSQL (235) | TCCCAGAGTCCCAGCGTCAGTCAGCTA (741) |
| STVAPTPATAR | TCGACCGTCGCTCCTACCCCGGCCACCGCTCGG |
| (236) | (742) |
| QYIAQFTSQF (237) | CAGTATATCGCCCAGTTCACCTCTCAGTTC (743) |
| GQHLHLETF (238) | GGACAACATCTTCACCTGGAGACCTTC (744) |
| TIASVLVAR (239) | ACAATTGCAAGTGTGTTGGTTGCAAGA (745) |
| SLLQHLIGL (240) | AGTCTCCTGCAGCACCTCATCGGGCTG (746) |
| TASPRSPVM (241) | ACAGCCAGTCCCAGGAGCCCAGTAATG (747) |
| AAFDGRHSQTL | GCAGCCTTTGACGGGAGACACAGCCAGACCCTG |
| (242) | (748) |
| SPRRLVEL (243) | AGCCCACGGAGACTTGTGGAGCTG (749) |
| LYVDSLFFL (244) | CTCTATGTGGACTCTTTATTTTTCCTT (750) |
| VFLREFPNF (245) | GTCTTCCTGAGAGAATTTCCTAATTTT (751) |
| RPRFSTATEIA | AGGCCCAGATTCTCCACTGCTACCGAGATCGCC |
| (246) | (752) |
| ALIANPRRK (247) | GCTTTGATTGCTAACCCAAGGAGGAAG (753) |
| DFMGQIVYR (248) | GATTTCATGGGCCAGATTGTGTACCGC (754) |
| EIPDTVMGL (249) | GAAATTCCCGATACAGTAATGGGCCTT (755) |
| VMSSTLYTV (250) | GTAATGTCCAGCACCCTGTACACTGTG (756) |
| SPQHKPRL (251) | TCACCTCAGCACAAGCCACGACTG (757) |
| EPKPPTTGL (252) | GAGCCTAAGCCACCCACCACAGGGCTT (758) |
| IPDTIASVL (253) | ATACCAGACACAATTGCAAGTGTGTTG (759) |
| TDFMGQIVYR (254) | ACAGATTTCATGGGCCAGATTGTGTACCGC (760) |
| GEDDEHRKI (255) | GGAGAAGATGATGAACACAGAAAAATT (761) |
| RLRGSIQSRY (256) | CGTTTGCGGGGTTCCATTCAGAGCCGATAC (762) |
| HLDESPAYL (257) | CACTTGGATGAGAGCCCCGCCTACCTC (763) |
| KMEETTVTY (258) | AAGATGGAAGAAACTACGGTCACATAT (764) |
| LPTDLFNSV (259) | CTCCCAACAGATCTGTTTAATAGTGTG (765) |
| RPQGLAVL (260) | AGGCCTCAGGGCCTGGCTGTCCTG (766) |
| SLSEKSPEV (261) | AGTCTATCTGAAAAGTCTCCAGAAGTC (767) |
| GAFREGPPY (262) | GGTGCATTTCGAGAGGGGCCGCCCTAC (768) |
| YLNGATVVA (263) | TACCTCAATGGGGCTACTGTTGTAGCA (769) |
| SMYLHTEGF (264) | TCAATGTACCTCCACACAGAGGGCTTC (770) |
| HILSHLHEV (265) | CACATTCTGAGTCACTTGCATGAAGTC (771) |
| SISEAPLTEV (266) | AGTATCTCTGAAGCCCCACTTACAGAGGTT (772) |
| HQINSEATF (267) | CACCAGATCAACAGTGAAGCCACATTT (773) |
| DIKQEGVGAF (268) | GACATCAAGCAGGAAGGGGTCGGTGCATTT (774) |
| SALGVTITK (269) | AGCGCTCTGGGAGTTACTATTACCAAA (775) |
| TVNDFTQNPR (270) | ACAGTGAATGACTTTACCCAAAACCCCAGG (776) |
| SFIQAGSSY (271) | TCTTTCATCCAAGCAGGATCCTCGTAT (777) |
| GVTITKTLK (272) | GGAGTTACTATTACCAAAACTCTCAAG (778) |
| AERPQGLAV (273) | GCTGAGAGGCCTCAGGGCCTGGCTGTC (779) |
| REKGPVKEV (274) | AGGGAAAAGGGACCCGTCAAGGAAGTT (780) |
| TSVPPFNLR (275) | ACCTCAGTGCCTCCCTTCAACCTAAGA (781) |
| LSRLPSIAF (276) | TTATCACGCCTTCCAAGCATTGCCTTT (782) |
| FPVHAAEKF (277) | TTCCCAGTTCATGCTGCTGAGAAGTTC (783) |
| GEISASHV (278) | GGGGAGATCTCTGCATCACATGTC (784) |
| AADGGQHWTY (279) | GCTGCAGATGGGGGTCAACACTGGACGTAT (785) |
| EISASHVSR (280) | GAGATCTCTGCATCACATGTCAGCAGG (786) |
| SPRIPFSTF (281) | TCGCCCCGGATCCCGTTCAGCACCTTC (787) |
| KLMDPGSLPPL | AAGCTCATGGACCCGGGCTCCCTGCCGCCCCTC |
| (282) | (788) |
| ETKPEFERR (283) | GAAACAAAGCCAGAGTTTGAAAGGAGG (789) |
| ETIMKFLKM (284) | GAGACGATCATGAAATTTTTGAAAATG (790) |
| RRAHLWSTF (285) | AGGCGCGCCCACCTTTGGAGCACTTTT (791) |
| HLQEKLQSL (286) | CATCTCCAGGAAAAGCTGCAGAGTTTG (792) |
| ELSVVTSER (287) | GAACTCTCAGTGGTCACAAGTGAGAGA (793) |
| YLDQQVPYTF (288) | TACTTGGACCAGCAAGTGCCCTACACCTTC (794) |
| EAIPRIPRL (289) | GAAGCAATTCCTCGAATCCCTCGCTTG (795) |
| GYLDQQVPYTF | GGATACTTGGACCAGCAAGTGCCCTACACCTTC |
| (290) | (796) |
| SPKLGGIGF (291) | AGCCCTAAACTGGGCGGGATTGGATTT (797) |
| YPEKSRGCL (292) | TATCCTGAGAAATCACGTGGATGCCTC (798) |
| TTIPFSTLRQR (293) | ACCACCATTCCCTTCAGCACTCTACGTCAGAGG (799) |
| SLGAHSPALY (294) | AGCCTGGGTGCCCACAGTCCTGCCCTGTAC (800) |
| SQNGQLIHY (295) | TCTCAAAATGGACAACTAATCCATTAC (801) |
| KAKEHTANISW | AAAGCCAAGGAGCACACCGCCAATATTTCATGG |
| (296) | (802) |
| RVSKSDSTF (297) | AGGGTATCAAAGTCAGATTCTACTTTC (803) |
| SLNSQVTGL (298) | TCCCTCAATTCTCAAGTCACAGGTCTT (804) |
| NSDLSEKLEY (299) | AACTCAGATTTAAGTGAAAAATTGGAATAT (805) |
| DSSTVGLQR (300) | GACAGTTCCACTGTTGGGCTTCAGCGA (806) |
| RASAIIIQR (301) | AGAGCTTCAGCTATTATAATTCAGAGA (807) |
| SQETLPLKI (302) | TCCCAGGAAACACTTCCACTCAAGATT (808) |
| STFDGLDRR (303) | AGCACCTTTGATGGTTTAGACAGAAGA (809) |
| RVKEIVINK (304) | CGAGTGAAGGAGATTGTAATCAACAAG (810) |
| AQPHHGVPY (305) | GCTCAGCCCCACCATGGTGTGCCATAT (811) |
| EVIQLKERDRK (306) | GAAGTAATACAGTTAAAAGAACGAGACCGTAAG (812) |
| EVKEKTEL (307) | GAGGTGAAGGAAAAGACGGAACTC (813) |
| TDPETLHY (308) | ACTGACCCTGAGACCCTGCACTAC (814) |
| KMKEKTQEL (309) | AAAATGAAGGAGAAAACACAAGAGCTT (815) |
| STFDGLDRRR (310) | AGCACCTTTGATGGTTTAGACAGAAGAAGA (816) |
| KMSELQTYV (311) | AAAATGTCAGAGCTGCAGACCTATGTT (817) |
| GTDPETLHY (312) | GGCACTGACCCTGAGACCCTGCACTAC (818) |
| TEEKGSILI (313) | ACAGAGGAAAAGGGGAGCATTTTAATT (819) |
| DTAMPAHSR (314) | GACACTGCTATGCCTGCCCATTCAAGA (820) |
| KLQEALKTI (315) | AAGCTCCAGGAAGCACTGAAGACCATC (821) |
| GTDPETLHYF (316) | GGCACTGACCCTGAGACCCTGCACTACTTC (822) |
| SLKNRIESV (317) | AGTTTAAAAAATCGAATTGAATCTGTA (823) |
| NLKDPVGVY (318) | AACCTTAAAGACCCAGTTGGGGTATAC (824) |
| FTETNPQVY (319) | TTCACAGAGACCAACCCACAAGTGTAC (825) |
| ETQEAEVAVSR (320) | GAAACCCAGGAGGCAGAGGTTGCAGTGAGCCGA (826) |
| SPRPESGTV (321) | TCTCCCAGACCAGAGTCGGGTACTGTT (827) |
| SEFAKDPSL (322) | TCTGAGTTTGCGAAGGATCCGTCCCTC (828) |
| KVIKKIGHY (323) | AAGGTTATCAAGAAAATTGGACATTAT (829) |
| RTWQTRKQF (324) | AGGACGTGGCAAACCAGAAAACAGTTT (830) |
| AMAHKVIKA (325) | GCCATGGCCCACAAAGTAATTAAAGCT (831) |
| KLKQFQKEY (326) | AAACTAAAACAGTTTCAGAAGGAATAT (832) |
| SPLEDEESL (327) | TCTCCATTGGAAGATGAAGAGTCATTA (833) |
| NSTDFTTDR (328) | AACTCTACAGACTTTACCACTGACAGA (834) |
| ETKDTKGRYF (329) | GAAACGAAGGATACCAAAGGTCGTTATTTT (835) |
| RPRVSSTEM (330) | CGGCCTCGAGTATCCTCCACAGAAATG (836) |
| LMDKSSTIY (331) | TTAATGGACAAATCAAGCACTATTTAC (837) |
| GTLRPLKVRR (332) | GGGACCCTCCGGCCGCTCAAAGTTCGCAGG (838) |
| ILSEGSVPL (333) | ATCCTGAGTGAAGGATCCGTCCCTCTC (839) |
| TVYNFRTEL (334) | ACAGTCTATAATTTTAGGACTGAACTT (840) |
| LPSSADVEF (335) | CTCCCCTCTTCAGCTGATGTAGAATTT (841) |
| RLPSSADVEF (336) | AGGCTCCCCTCTTCAGCTGATGTAGAATTT (842) |
| ALMDKSLHV (337) | GCCTTGATGGATAAAAGTCTTCATGTT (843) |
| MHNALHIYM (338) | ATGCACAATGCCTTGCACATCTATATG (844) |
| MEKEDYHSL (339) | ATGGAGAAAGAGGATTACCACAGCTTG (845) |
| MEKEDYHSLY (340) | ATGGAGAAAGAGGATTACCACAGCTTGTAT (846) |
| YHSLYQSHL (341) | TACCACAGCTTGTATCAGAGCCATTTA (847) |
| YMVPFIPLY (342) | TACATGGTTCCTTTTATACCACTGTAC (848) |
| LMEKEDYHSL (343) | CTCATGGAGAAAGAGGATTACCACAGCTTG (849) |
| FAHEAPAFL (344) | TTTGCCCATGAAGCACCAGCTTTTCTG (850) |
| FASPLTGIA (345) | TTTGCTAGTCCACTTACTGGGATAGCG (851) |
| HEAPAFLPW (346) | CATGAAGCACCAGCTTTTCTGCCTTGG (852) |
| VLFKKFIQK (347) | GTCCTCTTCAAGAAGTTTATCCAGAAG (853) |
| APAFLPWHR (348) | GCACCAGCTTTTCTGCCTTGGCATAGA (854) |
| LLMEKEDYHSL (349) | CTCCTCATGGAGAAAGAGGATTACCACAGCTTG (855) |
| HYYVSMDAL (350) | CATTATTATGTGTCAATGGATGCACTG (856) |
| WEQEIQKL (351) | TGGGAACAAGAAATCCAGAAGCTG (857) |
| LMEKEDYHSLY (352) | CTCATGGAGAAAGAGGATTACCACAGCTTGTAT (858) |
| LEQASRIWSW (353) | TTGGAACAAGCGAGTCGGATCTGGTCATGG (859) |
| NIFDLSAPEK (354) | AACATCTTCGATTTGAGTGCCCCAGAGAAG (860) |
| EAPAFLPWHR (355) | GAAGCACCAGCTTTTCTGCCTTGGCATAGA (861) |
| SEIWRDIDF (356) | TCTGAAATCTGGAGAGACATTGATTTT (862) |
| AAMVGAVL (357) | GCGGCGATGGTAGGGGCCGTCCTC (863) |
| TFLGVGQESF (358) | ACTTTCCTTGGGGTAGGACAGGAAAGCTTT (864) |
| ESWPSVFYNR (359) | GAGTCGTGGCCTTCCGTCTTTTATAATAGG (865) |
| ISQDTTVGR (360) | ATTTCTCAGGACACAACTGTGGGACGT (866) |
| RPWSGPYIL (361) | AGGCCCTGGAGTGGTCCCTACATCCTA (867) |
| SSDYVIPIGTY (362) | AGCTCAGACTATGTCATCCCCATAGGGACCTAT (868) |
| DSDPDSFQDY (363) | GATTCAGACCCAGACTCTTTTCAAGACTAC (869) |
| DTRPWSGPY (364) | GACACAAGGCCCTGGAGTGGTCCCTAC (870) |
| HYYSVKKTF (365) | CACTATTACTCAGTCAAAAAGACTTTC (871) |
| NVCGSQQGR (366) | AATGTCTGTGGCTCTCAGCAAGGCCGG (872) |
| YMNGTMSQV (367) | TATATGAATGGAACAATGTCCCAGGTA (873) |
| MAKRTTHPL (368) | ATGGCAAAGCGCACAACTCACCCTTTA (874) |
| RPWSGPYILR (369) | AGGCCCTGGAGTGGTCCCTACATCCTACGA (875) |
| MTVDSLVNK (370) | ATGACGGTGGACAGCCTAGTGAACAAG (876) |
| TRPWSGPYIL (371) | ACAAGGCCCTGGAGTGGTCCCTACATCCTA (877) |
| TTHPLFVIATR (372) | ACAACTCACCCTTTATTTGTCATTGCCACCAGG (878) |
| FVWTHYYSV (373) | TTTGTTTGGACACACTATTACTCAGTC (879) |
| LSKEEKNHF (374) | TTAAGTAAAGAAGAAAAGAACCACTTT (880) |
| RAANQVFVL (375) | AGAGCTGCAAACCAGGTCTTTGTTTTG (881) |
| MVRSATLIK (376) | ATGGTGAGAAGCGCTACCCTCATTAAA (882) |
| NQVFVLHSY (377) | AACCAGGTCTTTGTTTTGCACTCTTAT (883) |
| HNSTEFLI (378) | CACAACTCCACAGAGTTTCTCATC (884) |
| SQDVAGTTF (379) | TCTCAGGATGTTGCAGGCACAACTTTC (885) |
| AHNSTEFLI (380) | GCACACAACTCCACAGAGTTTCTCATC (886) |
| STEFLIYER (381) | TCCACAGAGTTTCTCATCTACGAAAGA (887) |
| EGFFTRQER (382) | GAAGGGTTTTTCACGAGACAGGAGCGC (888) |
| FQKVLVSY (383) | TTTCAGAAAGTTTTGGTATCCTAC (889) |
| FTDFMGQIVY (384) | TTCACAGATTTCATGGGCCAGATTGTGTAC (890) |
| YFQKVLVSY (385) | TACTTTCAGAAAGTTTTGGTATCCTAC (891) |
| ALVNMPPHYR (386) | GCACTGGTGAACATGCCTCCTCACTACCGC (892) |
| GVMSSTLYTV (387) | GGTGTAATGTCCAGCACCCTGTACACTGTG (893) |
| LLSNTVSTL (388) | TTGCTATCTAATACGGTCTCAACACTA (894) |
| RVFHETGAQR (389) | AGGGTTTTTCACGAGACAGGAGCGCAGAGA (895) |
| SASDHCRSR (390) | TCGGCCAGCGACCACTGCCGGTCCAGG (896) |
| RRDGGIIIY (391) | CGCAGAGATGGAGGCATCATAATCTAT (897) |
| VAALIANPR (392) | GTAGCAGCTTTGATTGCTAACCCAAGG (898) |
| RLLGTEFQV (393) | AGACTGTTGGGTACAGAATTCCAGGTC (899) |
| FSIIGVSRR (394) | TTCTCCATCATCGGAGTTTCCCGAAGG (900) |
| LSSDGMYEY (395) | TTGTCATCAGATGGAATGTACGAGTAT (901) |
| DEYFLPSL (396) | GATGAATACTTCCTACCCTCCCTG (902) |
| SYIGLKGLYF (397) | TCCTACATTGGATTAAAGGGTCTTTACTTC (903) |
| FTGFGGALGY (398) | TTCACAGGTTTTGGAGGTGCCCTGGGTTAC (904) |
| KTWDQVPFSV (399) | AAGACCTGGGACCAGGTGCCTTTCTCCGTG (905) |
| RVADIPIKP (400) | AGGGTGGCGGATATTCCAATAAAGCCA (906) |
| SVAKHTQSY (401) | TCTGTTGCCAAGCACACCCAGAGCTAC (907) |
| QTKGGQTWAR (402) | CAGACAAAAGGGGGCCAAACATGGGCGAGA (908) |
| ETGSSFMSF (403) | GAAACAGGAAGTTCATTCATGAGTTTT (909) |
| SLYSYFQKV (404) | TCACTTTATTCTTACTTTCAGAAAGTT (910) |
| QTKGGQTWARR (405) | CAGACAAAAGGGGGCCAAACATGGGCGAGAAGG (911) |
| TVVAALIANPR (406) | ACTGTTGTAGCAGCTTTGATTGCTAACCCAAGG (912) |
| HLMVKDWQL (407) | CATCTCATGGTGAAAGATTGGCAGCTG (913) |
| NPLSKLSVL (408) | AACCCTCTAAGTAAGCTCTCTGTGCTC (914) |
| FVIPSTKDSNR (409) | TTTGTAATTCCTAGCACGAAAGACTCTAACAGG (915) |
| HYQVQTHL (410) | CACTATCAGGTGCAGACCCACCTC (916) |
| ASDILSFAHKY (411) | GCCTCGGACATCCTGTCCTTTGCGCACAAGTAC (917) |
| RLGQGVPLV (412) | AGACTGGGGCAGGGCGTGCCCCTCGTG (918) |
| SVPDHVQPSK (413) | AGTGTACCAGACCATGTCCAACCCTCTAAG (919) |
| RLLEKHISL (414) | AGGCTGCTGGAAAAGCACATCTCCCTG (920) |
| SLFGVSERL (415) | TCCCTCTTTGGGGTCTCAGAGAGATTG (921) |
| NHYQVQTHL (416) | AATCACTATCAGGTGCAGACCCACCTC (922) |
| DLVGKDVTRVY (417) | GACCTGGTTGGAAAGGATGTAACAAGAGTGTAC (923) |
| SLSPPTLPR (418) | TCTCTCAGTCCTCCAACACTACCAAGA (924) |
| LADNGTLGKY (419) | CTGGCTGACAATGGCACCCTGGGCAAGTAT (925) |
| KAMYIRVSY (420) | AAAGCCATGTATATCCGTGTATCCTAT (926) |
| MQPKLKQVF (421) | ATGCAGCCCAAGCTGAAACAAGTCTTT (927) |
| AIIQDGILL (422) | GCAATTATTCAGGATGGCATCTTGCTG (928) |
| AFDRAVHF (423) | GCGTTTGACAGAGCTGTTCACTTC (929) |
| NTPNIVSLR (424) | AATACTCCTAACATTGTCTCTTTAAGA (930) |
| RVADIPIKPW (425) | AGGGTGGCGGATATTCCAATAAAGCCATGG (931) |
| HYIIPLQTI (426) | CACTATATAATCCCACTGCAAACAATA (932) |
| VMKGINQGY (427) | GTCATGAAGGGCATTAATCAAGGTTAT (933) |
| ATSPPASVR (428) | GCGACGTCCCCGCCGGCCTCGGTCCGC (934) |
| ALEKDTPRY (429) | GCACTGGAGAAGGATACCCCCAGGTAT (935) |
| QVMKGINQGY (430) | CAAGTCATGAAGGGCATTAATCAAGGTTAT (936) |
| AAFDRAVHF (431) | GCAGCGTTTGACAGAGCTGTTCACTTC (937) |
| RPLRIKGVF (432) | AGACCGCTGAGGATCAAAGGGGTTTTC (938) |
| RQVMKGINQGY (433) | AGGCAAGTCATGAAGGGCATTAATCAAGGTTAT (939) |
| KLDTTVKNI (434) | AAACTGGACACCACTGTAAAGAACATC (940) |
| LPRTPRPDL (435) | CTTCCGCGAACTCCTCGCCCCGACCTC (941) |
| ITYTESNSR (436) | ATAACTTACACCGAAAGCAACTCAAGG (942) |
| RLQGSVTLV (437) | CGACTTCAGGGATCTGTCACCCTCGTC (943) |
| SQFPSKNLPAF (438) | TCTCAATTCCCAAGTAAAAATCTTCCTGCTTTT (944) |
| LPRTPRPDLIL (439) | CTTCCGCGAACTCCTCGCCCCGACCTCATCCTT (945) |
| DTAPQSHQV (440) | GACACTGCCCCACAATCCCATCAAGTC (946) |
| MAILGTAGR (441) | ATGGCTATCCTGGGCACAGCGGGGCGG (947) |
| SVSSHLIHR (442) | TCTGTGTCCTCTCACCTCATTCATAGA (948) |
| STSPGTGVRK (443) | TCCACGTCACCAGGGACAGGAGTCAGGAAA (949) |
| EVAEVLGSVK (444) | GAAGTGGCTGAAGTCCTTGGGAGTGTGAAG (950) |
| EIPRTPSEGR (445) | GAGATACCACGAACCCCCTCAGAAGGAAGA (951) |
| VFLKFSRRF (446) | GTGTTTTTAAAATTCTCCAGAAGATTT (952) |
| SVSSHLIHRY (447) | TCTGTGTCCTCTCACCTCATTCATAGATAC (953) |
| THLEDPHLM (448) | ACACATCTAGAAGACCCTCACCTTATG (954) |
| SVFPRTSSR (449) | AGTGTCTTTCCCAGGACCTCCTCCAGA (955) |
| GQYGQSKPWTF (450) | GGGCAGTATGGACAAAGTAAGCCTTGGACTTTT (956) |
| VPRPTSTVGL (451) | GTTCCAAGACCTACCAGCACAGTAGGATTA (957) |
| NSVSSHLIHRY (452) | AATTCTGTGTCCTCTCACCTCATTCATAGATAC (958) |
| QEEDNLLRYF (453) | CAAGAAGAAGACAACCTTCTAAGATACTTT (959) |
| THLEDPHLML (454) | ACACATCTAGAAGACCCTCACCTTATGCTT (960) |
| ATTNILEHV (455) | GCTACAACAAACATCCTTGAACATGTA (961) |
| MTLRPTQPR (456) | ATGACTCTGAGGCCTACACAGCCACGT (962) |
| ETRPNNSKF (457) | GAAACAAGACCGAACAACTCCAAGTTC (963) |
| HSITEPLLY (458) | CACTCAATCACGGAGCCTCTGCTCTAT (964) |
| HLFLQPPSKR (459) | CATTTATTCTTACAACCTCCAAGCAAAAGA (965) |
| ETVRFTSSF (460) | GAGACAGTTAGATTTACTTCCTCTTTT (966) |
| SPMPIPPLYL (461) | TCCCCAATGCCTATACCCCCATTGTATCTT (967) |
| ESYGRYTFY (462) | GAATCTTATGGTAGGTATACTTTTTAC (968) |
| KSQGSFISF (463) | AAATCACAAGGTTCATTTATTTCCTTT (969) |
| GTILHIGVF (464) | GGTACTATCTTGCACATAGGTGTATTT (970) |
| KVFSGSSKR (465) | AAAGTATTTTCTGGAAGCAGCAAGAGG (971) |
| SSFSTLASLDK (466) | TCAAGCTTCAGCACCCTTGCATCTCTGGATAAA (972) |
| TTQGAAPQR (467) | ACCACCCAGGGAGCTGCTCCTCAGAGG (973) |
| HTTQGAAPQR (468) | CATACCACCCAGGGAGCTGCTCCTCAGAGG (974) |
| KIFQERAAF (469) | AAGATCTTCCAAGAGAGGGCAGCCTTT (975) |
| GQITHNTSF (470) | GGGCAAATCACACATAACACTTCATTC (976) |
| ISLKPLIARY (471) | ATTAGTCTCAAGCCTTTGATAGCACGGTAT (977) |
| KHSDLILFW (472) | AAGCATTCTGATTTGATTTTATTCTGG (978) |
| MVWEGHPRER (473) | ATGGTATGGGAGGGGCATCCCAGAGAAAGA (979) |
| STDLPILLK (474) | TCAACTGATTTGCCCATTCTTCTTAAA (980) |
| EVISINTGHF (475) | GAGGTAATTTCTATCAACACTGGACATTTC (981) |
| ETERVLEKSSY (476) | GAGACAGAGCGGGTCCTGGAGAAGTCTTCTTAC (982) |
| KIKTINLRY (477) | AAGATAAAAACTATAAATCTGAGATAT (983) |
| YVSQKVSDF (478) | TATGTGAGTCAGAAAGTTTCAGACTTT (984) |
| DAGIPVYHR (479) | GATGCAGGCATCCCTGTGTACCACCGA (985) |
| TILEAEILHQA (480) | ACCATCTTGGAAGCAGAGATCCTTCACCAGGCA (986) |
| EYAQKTPLF (481) | GAATATGCTCAGAAAACACCTCTCTTT (987) |
| TWTPHGIRI (482) | ACATGGACACCACATGGTATCAGAATC (988) |
| EVHEGGVARF (483) | GAGGTCCACGAAGGTGGAGTTGCTCGATTT (989) |
| REIEIEGRL (484) | AGAGAAATAGAAATAGAGGGAAGATTA (990) |
| SWLPIPAKY (485) | TCCTGGCTGCCAATCCCAGCCAAATAT (991) |
| RYSERVYTI (486) | CGCTACTCCGAAAGGGTCTACACTATC (992) |
| FTDSQGNDIK (487) | TTCACAGACTCGCAGGGCAATGACATTAAA (993) |
| SVPSDFLSLR (488) | TCAGTGCCAAGCGACTTCCTGAGTCTCAGG (994) |
| RQMSLEFRA (489) | CGGCAGATGAGTCTGGAGTTCAGGGCA (995) |
| ETISPRPSI (490) | GAAACCATCTCCCCCAGGCCTTCAATA (996) |
| GPAGGRGVI (491) | GGGCCAGCAGGAGGAAGGGGAGTCATC (997) |
| LTDHHIKEY (492) | CTGACTGACCATCACATAAAAGAATAC (998) |
| EAVPYNGSLFK (493) | GAAGCAGTACCTTATAATGGTTCCTTGTTCAAA (999) |
| NLLAEIHGV (494) | AATCTGCTGGCGGAGATCCACGGTGTG (1000) |
| KEGPVTRGA (495) | AAAGAGGGGCCAGTGACGCGAGGAGCA (1001) |
| VHVDLNLGF (496) | GTACACGTGGATTTGAATCTTGGCTTC (1002) |
| HTVQGLAGF (497) | CACACTGTACAGGGACTAGCTGGATTT (1003) |
| MRIEDSHTKL (498) | ATGCGTATAGAGGACAGCCACACTAAGCTT (1004) |
| SALSSAQPR (499) | AGTGCCCTGTCTTCAGCCCAGCCCAGG (1005) |
| SPSIKSLLVY (500) | TCCCCTTCCATCAAAAGTCTACTAGTTTAT (1006) |
| PTDATLNRLLY (501) | CCAACTGATGCCACTTTAAACCGTTTGCTCTAC (1007) |
| WTSQPPNYEK (502) | TGGACTTCCCAGCCCCCAAACTATGAGAAA (1008) |
| PAPRPVAPTL (503) | CCCGCCCCCCGCCCCGTGGCACCCACCCTG (1009) |
| VYLHSNSTL (504) | GTATATCTCCATTCTAACAGCACATTA (1010) |
| SLYAWTQSH (505) | TCTTTGTATGCCTGGACTCAGAGCCAC (1011) |

Of course, because of the degeneracy of the genetic code, the TAPs described herein may be encoded by variants of the above-noted sequences.

A nucleic acid of the disclosure may be used for recombinant expression of the TAP or SLP of the disclosure, and may be included in a vector or plasmid, such as a cloning vector or an expression vector, which may be transfected into a host cell. In an embodiment, the disclosure provides a cloning, expression or viral vector or plasmid comprising a nucleic acid sequence encoding the TAP of the disclosure. Alternatively, a nucleic acid encoding a TAP of the disclosure may be incorporated into the genome of the host cell. In either case, the host cell expresses the TAP or protein encoded by the nucleic acid. The term “host cell” as used herein refers not only to the particular subject cell, but to the progeny or potential progeny of such a cell. A host cell can be any prokaryotic (e.g., E. coli) or eukaryotic cell (e.g., insect cells, yeast cells, plant cells, or mammalian cells) capable of expressing the TAPs described herein. The vector or plasmid contains the necessary elements for the transcription and translation of the inserted coding sequence, and may contain other components such as resistance genes, cloning sites, etc. Methods that are well known to those skilled in the art may be used to construct expression vectors containing sequences encoding peptides or polypeptides and appropriate transcriptional and translational control/regulatory elements operably linked thereto. These methods include in vitro recombinant DNA techniques, synthetic techniques, and in vivo genetic recombination. Such techniques are described in Sambrook. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y. “Operably linked” refers to a juxtaposition of components, particularly nucleotide sequences, such that the normal function of the components can be performed. Thus, a coding sequence that is operably linked to regulatory sequences refers to a configuration of nucleotide sequences wherein the coding sequences can be expressed under the regulatory control, that is, transcriptional and/or translational control, of the regulatory sequences. “Regulatory/control region” or “regulatory/control sequence”, as used herein, refers to the non-coding nucleotide sequences that are involved in the regulation of the expression of a coding nucleic acid. Thus, the term regulatory region includes promoter sequences, regulatory protein binding sites, upstream activator sequences, and the like. The vector (e.g., expression vector) may have the necessary 5′ upstream and 3′ downstream regulatory elements such as promoter sequences such as CMV, PGK and EF-1a promoters, ribosome recognition and binding TATA box, and 3′ UTR AAUAAA transcription termination sequence for the efficient gene transcription and translation in its respective host cell. Other suitable promoters include the constitutive promoter of simian vims 40 (SV40) early promoter, mouse mammary tumor virus (MMTV), HIV LTR promoter, MoMuLV promoter, avian leukemia virus promoter, EBV immediate early promoter, and Rous sarcoma vims promoter. Human gene promoters may also be used, including, but not limited to the actin promoter, the myosin promoter, the hemoglobin promoter, and the creatine kinase promoter. In certain embodiments inducible promoters are also contemplated as part of the vectors expressing the TAP. This provides a molecular switch capable of turning on expression of the polynucleotide sequence of interest or turning off expression. Examples of inducible promoters include, but are not limited to a metallothionine promoter, a glucocorticoid promoter, a progesterone promoter, or a tetracycline promoter. Examples of vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or PI-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are Lenti-X™ Bicistronic Expression System (Neo) vectors (Contech), pClneo vectors (Promega) for expression in mammalian cells; pLenti4N5-DEST™, pLenti6N5-DEST™, and pLenti6.2N5-GW/lacZ (Invitrogen) for lentivirus-mediated gene transfer and expression in mammalian cells. The coding sequences of the TAPs disclosed herein can be ligated into such expression vectors for the expression of the TAP in mammalian cells.

In certain embodiments, the nucleic acids encoding the TAP of the present disclosure are provided in a viral vector. A viral vector can be those derived from adenovirus, vaccinia virus, retrovirus, lentivirus, or foamy virus. As used herein, the term “viral vector” refers to a nucleic acid vector construct that includes at least one element of viral origin and has the capacity to be packaged into a viral vector particle. The viral vector can contain the coding sequence for the various proteins described herein in place of nonessential viral genes. In another embodiment, the nucleic acids encoding the TAP of the present disclosure are provided in a self-amplifying or self-replicating RNA (srRNA) vectors. srRNAs are derived from positive-strand RNA viruses where the structural proteins have been removed and replaced with heterologous genes of interest. srRNAs have been successfully derived from flaviviruses, nodamura viruses, nidoviruses, and alphaviruses with therapeutic versions of the technology providing the structural proteins in trans to create single cycle viral replicon particles (VRPs) (see, e.g., Aliahmad et al. Next generation self-replicating RNA vectors for vaccines and immunotherapies. Cancer Gene Ther (2022). https://doi.org/10.1038/s41417-022-00435-8). The vector and/or particle can be utilized for the purpose of transferring DNA, RNA or other nucleic acids into cells either in vitro or in vivo. Numerous forms of viral vectors are known in the art.

In embodiment, the nucleic acid (DNA, RNA) encoding the TAP of the disclosure is comprised within a vesicle or nanoparticle such as a lipid vesicle (e.g., liposome) or lipid nanoparticle (LNP), or any other suitable vehicle. Thus, in another aspect, the present disclosure provides a vesicle or nanoparticle, such as a lipid vesicle or nanoparticle, comprising a nucleic acid, such as an mRNA, encoding one or more of the TAP described herein.

The term liposome as used herein in accordance with its usual meaning, referring to microscopic lipid vesicles composed of a bilayer of phospholipids or any similar amphipathic lipids (e.g., sphingolipids) encapsulating an internal aqueous medium.

The term “lipid nanoparticle” refers to liposome-like structure that may include one or more lipid bilayer rings surrounding an internal aqueous medium similar to liposomes, or micellar-like structures that encapsulates molecules (e.g., nucleic acids) in a non-aqueous core. Lipid nanoparticles typically contain cationic lipids, such as ionizable cationic lipids. Examples of cationic lipids that may be used for LNPs include DOTMA, DOSPA, DOTAP, ePC, DLin-MC3-DMA, C12-200, ALC-0315, cKK-E12, Lipid H (SM-102), OF-Deg-Lin, A2-Iso5-2DC18, 3060,io, BAME-016B, TT3, 9A1P9, FTT5, COATSOME® SS-E, COATSOME® SS-EC, COATSOME® SS-OC and COATSOME® SS-OP (see, e.g., Hou et al., Nature Reviews Materials, volume 6, pages 1078-1094 (2021); Tenchov et al., ACS Nano, 15, 16982-17015 (2021).

Liposomes and lipid nanoparticles typically include other lipid components such as lipids, lipid-like materials, and polymers that can improve liposome or nanoparticle properties, such as stability, delivery efficacy, tolerability and biodistribution. These include phospholipids (e.g., phosphatidylcholines, phosphatidylethanolamines, phosphatidylserines, and phosphatidylglycerol) such as 1,2-distearoyl-sn-glycero-3-phosphocholine (DSPC) and DOPE, sterols (such as cholesterol and cholesterol derivatives), PEGylated lipids (PEG-lipids) such as 1,2-dimyristoyl-rac-glycero-3-methoxypolyethylene glycol-2000 (PEG₂₀₀₀-DMG) and 1,2-distearoyl-rac-glycero-3-methoxypolyethylene glycol-2000 (PEG₂₀₀₀-DSG).

In an embodiment, the lipid nanoparticle according to the present disclosure comprises one or more cationic lipids, such as ionizable cationic lipids. Examples of ionizable cationic lipids include those listed in PCT publications Nos. WO 2017/061150 and WO 2019/188867, which encompassed ionizable cationic lipids commercialized under the tradenames COATSOME® SS-E, COATSOME® SS-EC, COATSOME® SS-OC and COATSOME® SS-OP.

The nucleic acid (e.g., mRNA) encoding one or more of the TAP, may be modified, for example to increase stability and/or reduce immunogenicity. For example, the 5′ end may be capped to stabilize the molecule and decrease immunogenicity (for example, as described in U.S. Ser. No. 10/519,189 and U.S. Ser. No. 10/494,399). One or more nucleosides of the mRNA may be modified or substituted with 1-methyl pseudo-uridine to either increase stability of the molecule or reduce recognition of the molecule by the innate immune system. A form of modified nucleosides are described in U.S. Pat. No. 9,371,511. Other types of modifications that may be made to the mRNA include incorporation of anti-reverse cap analog (ARCA), 5′-methyl-cytidine triphosphate (m5CTP), N6-methyl-adenosine-5′-triphosphate (m6ATP), 2-thio-uridine triphosphate (s2UTP), pseudouridine triphosphate, N¹Methylpseudouridine triphosphate or 5-Methoxyuridine triphosphate (5moUTP). The mRNA may also include additional modifications to the 5′- and/or 3′-untranslated regions (UTRs) and polyadenylation (polyA) tail (see, for example, Kim et al., Molecular & cellular toxicology vol. 18, 1 (2022): 1-8). All these modifications and other modifications to the nucleic acid (e.g., mRNA) encoding the TAP are encompassed by the present disclosure.

In another aspect, the present disclosure provides an MHC class I molecule comprising (i.e., presenting or bound to) one or more of the TAPs comprising or consisting of the sequence of SEQ ID NOs: 1-505 defined herein.

In an embodiment, the MHC class I molecule is an HLA-A*01:01 molecule. In an embodiment, the MHC class I molecule is an HLA-A*02:01 molecule. In an embodiment, the MHC class I molecule is an HLA-A*02:09 molecule. In an embodiment, the MHC class I molecule is an HLA-A*03:01 molecule. In an embodiment, the MHC class I molecule is an HLA-A*03:01. In an embodiment, the MHC class I molecule is an HLA-A*23:01 molecule. In an embodiment, the MHC class I molecule is an HLA-A*24:02 molecule. In an embodiment, the MHC class I molecule is an HLA-A*25:01 molecule. In an embodiment, the MHC class I molecule is an HLA-A*26:01 molecule. In an embodiment, the MHC class I molecule is an HLA-A*30:01 molecule. In an embodiment, the MHC class I molecule is an HLA-A*30:02 molecule. In an embodiment, the MHC class I molecule is an HLA-A*33:01 molecule. In an embodiment, the MHC class I molecule is an HLA-A*68:01 molecule. In an embodiment, the MHC class I molecule is an HLA-B*07:02 molecule. In an embodiment, the MHC class I molecule is an HLA-B*08:01 molecule. In an embodiment, the MHC class I molecule is an HLA-B*13:02 molecule. In an embodiment, the MHC class I molecule is an HLA-B*14:01 molecule. In an embodiment, the MHC class I molecule is an HLA-B*15:01 molecule. In an embodiment, the MHC class I molecule is an HLA-B*18:01 molecule. In an embodiment, the MHC class I molecule is an HLA-B*35:03 molecule. In an embodiment, the MHC class I molecule is an HLA-B*37:01 molecule. In an embodiment, the MHC class I molecule is an HLA-B*38:01 molecule. In an embodiment, the MHC class I molecule is an HLA-B*40:01. In an embodiment, the MHC class I molecule is an HLA-B*40:02. In an embodiment, the MHC class I molecule is an HLA-B*44:02. In an embodiment, the MHC class I molecule is an HLA-B*49:01. In an embodiment, the MHC class I molecule is an HLA-B*51:01. In an embodiment, the MHC class I molecule is an HLA-C*03:03. In an embodiment, the MHC class I molecule is an HLA-C*03:04. In an embodiment, the MHC class I molecule is an HLA-C*04:01. In an embodiment, the MHC class I molecule is an HLA-C*06:02. In an embodiment, the MHC class I molecule is an HLA-C*07:01 molecule. In an embodiment, the MHC class I molecule is an HLA-C*07:02 molecule. In an embodiment, the MHC class I molecule is an HLA-C*08:02 molecule. In an embodiment, the MHC class I molecule is an HLA-C*12:03 molecule. In an embodiment, the MHC class I molecule is an HLA-C*14:02 molecule.

In an embodiment, the TAP (e.g., comprising or consisting of the sequence of SEQ ID NOs: 1-505 defined herein is non-covalently bound to the MHC class I molecule (i.e., the TAP is loaded into, or non-covalently bound to the peptide binding groove/pocket of the MHC class I molecule). In another embodiment, the TAP is covalently attached/bound to the MHC class I molecule (alpha chain). In such a construct, the TAP and the MHC class I molecule (alpha chain) are produced as a synthetic fusion protein, typically with a short (e.g., 5 to 20 residues, preferably about 8-12, e.g., 10) flexible linker or spacer (e.g., a polyglycine linker). In another aspect, the disclosure provides a nucleic acid encoding a fusion protein comprising a TAP defined herein fused to an MHC class I molecule (alpha chain). In an embodiment, the MHC class I molecule (alpha chain)—peptide complex is multimerized. Accordingly, in another aspect, the present disclosure provides a multimer of MHC class I molecule loaded (covalently or not) with the herein-mentioned TAP. Such multimers may be attached to a tag, for example a fluorescent tag, which allows the detection of the multimers. A great number of strategies have been developed for the production of MHC multimers, including MHC dimers, tetramers, pentamers, octamers, etc. (reviewed in Bakker and Schumacher, Current Opinion in Immunology 2005, 17:428-433). MHC multimers are useful, for example, for the detection and purification of antigen-specific T cells. Thus, in another aspect, the present disclosure provides a method for detecting or purifying (isolating, enriching) CD8⁺ T lymphocytes specific for a TAP defined herein, the method comprising contacting a cell population with a multimer of MHC class I molecule loaded (covalently or not) with the TAP; and detecting or isolating the CD8⁺ T lymphocytes bound by the MHC class I multimers. CD8⁺ T lymphocytes bound by the MHC class I multimers may be isolated using known methods, for example fluorescence activated cell sorting (FACS) or magnetic activated cell sorting (MACS).

In yet another aspect, the present disclosure provides a cell (e.g., a host cell), in an embodiment an isolated cell, comprising the herein-mentioned nucleic acid, vector or plasmid of the disclosure, i.e., a nucleic acid or vector encoding one or more TAPs. In another aspect, the present disclosure provides a cell expressing at its surface an MHC class I molecule (e.g., an MHC class I molecule of one of the alleles disclosed above) bound to or presenting a TAP according to the disclosure. In one embodiment, the host cell is a eukaryotic cell, such as a mammalian cell, preferably a human cell. a cell line or an immortalized cell. In another embodiment, the cell is an antigen-presenting cell (APC). In one embodiment, the host cell is a primary cell, a cell line or an immortalized cell. In another embodiment, the cell is an antigen-presenting cell (APC). Nucleic acids and vectors can be introduced into cells via conventional transformation or transfection techniques. The terms “transformation” and “transfection” refer to techniques for introducing foreign nucleic acid into a host cell, including calcium phosphate or calcium chloride co-precipitation, DEAE-dextran-mediated transfection, lipofection, electroporation, microinjection and viral-mediated transfection. Suitable methods for transforming or transfecting host cells can for example be found in Sambrook et al. (supra), and other laboratory manuals. Methods for introducing nucleic acids into mammalian cells in vivo are also known, and may be used to deliver the vector or plasmid of the disclosure to a subject for gene therapy.

Cells such as APCs can be loaded with one or more TAPs using a variety of methods known in the art. As used herein “loading a cell” with a TAP means that RNA or DNA encoding the TAP, or the TAP, is transfected into the cells or alternatively that the APC is transformed with a nucleic acid encoding the TAP. The cell can also be loaded by contacting the cell with exogenous TAPs that can bind directly to MHC class I molecule present at the cell surface (e.g., peptide-pulsed cells). The TAPs may also be fused to a domain or motif that facilitates its presentation by MHC class I molecules, for example to an endoplasmic reticulum (ER) retrieval signal, a C-terminal Lys-Asp-Glu-Leu sequence (see Wang et al., Eur J Immunol. 2004 December; 34(12):3582-94).

In another aspect, the present disclosure provides a composition or peptide combination/pool comprising any one of, or any combination of, the TAPs defined herein (or a nucleic acid encoding said peptide(s)). In an embodiment, the composition comprises any combination of the TAPs defined herein (any combination of 2, 3, 4, 5, 6, 7, 8, 9, 10 or more TAPs), or a combination of nucleic acids encoding said TAPs). Compositions comprising any combination/sub-combination of the TAPs defined herein are encompassed by the present disclosure. In another embodiment, the combination or pool may comprise one or more known tumor antigens.

Thus, in another aspect, the present disclosure provides a composition comprising any one of, or any combination of, the TAPs defined herein (e.g., comprising or consisting of the sequence of SEQ ID NOs: 1-505 defined herein) and a cell expressing an MHC class I molecule (e.g., a MHC class I molecule of one of the alleles disclosed above). APC for use in the present disclosure are not limited to a particular type of cell and include professional APCs such as dendritic cells (DCs), Langerhans cells, macrophages and B cells, which are known to present proteinaceous antigens on their cell surface so as to be recognized by CD8⁺ T lymphocytes. For example, an APC can be obtained by inducing DCs from peripheral blood monocytes and then contacting (stimulating) the TAPs, either in vitro, ex vivo or in vivo. APC can also be activated to present a TAP in vivo where one or more of the TAPs of the disclosure are administered to a subject and APCs that present a TAP are induced in the body of the subject. The phrase “inducing an APC” or “stimulating an APC” includes contacting or loading a cell with one or more TAPs, or nucleic acids encoding the TAPs such that the TAPs are presented at its surface by MHC class I molecules. As noted herein, according to the present disclosure, the TAPs may be loaded indirectly for example using longer peptides/polypeptides comprising the sequence of the TAPs (including the native protein), which is then processed (e.g., by proteases) inside the APCs to generate the TAP/MHC class I complexes at the surface of the cells. After loading APCs with TAPs and allowing the APCs to present the TAPs, the APCs can be administered to a subject as a vaccine. For example, the ex vivo administration can include the steps of: (a) collecting APCs from a first subject, (b) contacting/loading the APCs of step (a) with a TAP to form MHC class I/TAP complexes at the surface of the APCs; and (c) administering the peptide-loaded APCs to a second subject in need for treatment.

The first subject and the second subject may be the same subject (e.g., autologous vaccine), or may be different subjects (e.g., allogeneic vaccine). Alternatively, according to the present disclosure, use of a TAP described herein (or a combination thereof) for manufacturing a composition (e.g., a pharmaceutical composition) for inducing antigen-presenting cells is provided. In addition, the present disclosure provides a method or process for manufacturing a pharmaceutical composition for inducing antigen-presenting cells, wherein the method or the process includes the step of admixing or formulating the TAP, or a combination thereof, with a pharmaceutically acceptable carrier. Cells such as APCs expressing a MHC class I molecule (e.g., any of the above-noted HLA molecules) loaded with any one of, or any combination of, the TAPs defined herein, may be used for stimulating/amplifying CD8⁺ T lymphocytes, for example autologous CD8⁺ T lymphocytes. Accordingly, in another aspect, the present disclosure provides a composition comprising any one of, or any combination of, the TAPs defined herein (or a nucleic acid or vector encoding same); a cell expressing an MHC class I molecule and a T lymphocyte, more specifically a CD8⁺ T lymphocyte (e.g., a population of cells comprising CD8⁺ T lymphocytes).

In an embodiment, the composition further comprises a buffer, an excipient, a carrier, a diluent and/or a medium (e.g., a culture medium). In a further embodiment, the buffer, excipient, carrier, diluent and/or medium is/are pharmaceutically acceptable buffer(s), excipient(s), carrier(s), diluent(s) and/or medium (media). As used herein “pharmaceutically acceptable buffer, excipient, carrier, diluent and/or medium” includes any and all solvents, buffers, binders, lubricants, fillers, thickening agents, disintegrants, plasticizers, coatings, barrier layer formulations, lubricants, stabilizing agent, release-delaying agents, dispersion media, coatings, antibacterial and antifungal agents, isotonic agents, and the like that are physiologically compatible, do not interfere with effectiveness of the biological activity of the active ingredient(s) and that are not toxic to the subject. The use of such media and agents for pharmaceutically active substances is well known in the art (Rowe et al., Handbook of pharmaceutical excipients, 2003, 4^{th} edition, Pharmaceutical Press, London UK). Except insofar as any conventional media or agent is incompatible with the active compound (peptides, cells), use thereof in the compositions of the disclosure is contemplated. In an embodiment, the buffer, excipient, carrier and/or medium is a non-naturally occurring buffer, excipient, carrier and/or medium. In an embodiment, one or more of the TAPs defined herein, or the nucleic acids (e.g., mRNAs) encoding said one or more TAPs, are comprised within or complexed to a lipid vesicle or liposome, e.g., a cationic liposome (see, e.g., Vitor M T et al., Recent Pat Drug Deliv Formul. 2013 August; 7(2):99-110) or suitable other carriers.

In another aspect, the present disclosure provides a composition comprising one of more of the any one of, or any combination of, the TAPs defined herein (e.g., comprising or consisting of the sequence of SEQ ID NOs: 1-505 defined herein) (or a nucleic acid such as a mRNA encoding said peptide(s)), and a buffer, an excipient, a carrier, a diluent and/or a medium. For compositions comprising cells (e.g., APCs, T lymphocytes), the composition comprises a suitable medium that allows the maintenance of viable cells. Representative examples of such media include saline solution, Earl's Balanced Salt Solution (Life Technologies®) or PlasmaLyte® (Baxter International®). In an embodiment, the composition (e.g., pharmaceutical composition) is an “immunogenic composition”, “vaccine composition” or “vaccine”. The term “Immunogenic composition”, “vaccine composition” or “vaccine” as used herein refers to a composition or formulation comprising one or more TAPs, nucleic acids or vaccine vector and which is capable of inducing an immune response against the one or more TAPs present therein when administered to a subject. Vaccination methods for inducing an immune response in a mammal (e.g., human) comprise use of a vaccine or vaccine vector to be administered by any conventional route known in the vaccine field, e.g., via a mucosal (e.g., ocular, intranasal, pulmonary, oral, gastric, intestinal, rectal, vaginal, or urinary tract) surface, via a parenteral (e.g., subcutaneous, intradermal, intramuscular, intravenous, or intraperitoneal) route, or topical administration (e.g., via a transdermal delivery system such as a patch). In an embodiment, the TAP (or a combination thereof) is conjugated to a carrier protein (conjugate vaccine) to increase the immunogenicity of the TAP(s). The present disclosure thus provides a composition (conjugate) comprising a TAP (or a combination thereof), or a nucleic acid encoding the TAP or combination thereof, and a carrier protein. For example, the TAP(s) or nucleic acid(s) may be conjugated or complexed to a Toll-like receptor (TLR) ligand (see, e.g., Zom et al., Adv Immunol. 2012, 114: 177-201) or polymers/dendrimers (see, e.g., Liu et al., Biomacromolecules. 2013 Aug. 12; 14(8):2798-806) such as polymer-linked TLR agonists (see, e.g., Lynn et al., Nature Biotechnology 33: 1201-1210 (2015); Lynn et al., Nature Biotechnology 38: 320-332 (2020)). In an embodiment, the immunogenic composition or vaccine further comprises an adjuvant. “Adjuvant” refers to a substance which, when added to an immunogenic agent such as an antigen (TAPs, nucleic acids and/or cells according to the present disclosure), nonspecifically enhances or potentiates an immune response to the agent in the host upon exposure to the mixture. Examples of adjuvants currently used in the field of vaccines include (1) mineral salts (aluminum salts such as aluminum phosphate and aluminum hydroxide, calcium phosphate gels), squalene, (2) oil-based adjuvants such as oil emulsions and surfactant based formulations, e.g., MF59 (microfluidised detergent stabilised oil-in-water emulsion), QS21 (purified saponin), AS02 [SBAS2](oil-in-water emulsion+MPL+QS-21), (3) particulate adjuvants, e.g., virosomes (unilamellar liposomal vehicles incorporating influenza haemagglutinin), AS04 ([SBAS4]aluminum salt with MPL), ISCOMS (structured complex of saponins and lipids), polylactide co-glycolide (PLG), (4) microbial derivatives (natural and synthetic), e.g., monophosphoryl lipid A (MPL), Detox (MPL+M. phlei cell wall skeleton), AGP [RC-529](synthetic acylated monosaccharide), DC_Chol (lipoidal immunostimulators able to self-organize into liposomes), OM-174 (lipid A derivative), CpG motifs (synthetic oligonucleotides containing immunostimulatory CpG motifs), modified Cholera toxin (CT) and Escherichia coli enterotoxin (LT) (genetically modified bacterial toxins to provide non-toxic adjuvant effects), (5) endogenous human immunomodulators, e.g., hGM-CSF or hIL-12 (cytokines that can be administered either as protein or plasmid encoded), Immudaptin (C3d tandem array) and/or (6) inert vehicles, such as gold particles, and the like.

In an embodiment, the TAP(s) (e.g., comprising or consisting of the sequence of SEQ ID NOs: 1-505 defined herein) (or a nucleic acid such as a mRNA encoding said peptide(s)) or composition comprising same is/are in lyophilized form. In another embodiment, the TAP(s), nucleic acid(s) or composition comprising same is/are in a liquid composition. In a further embodiment, the TAP(s) or nucleic acid(s) is/are at a concentration of about 0.01 μg/mL to about 100 μg/mL in the composition. In further embodiments, the TAP(s) or nucleic acid(s) is/are at a concentration of about 0.2 μg/mL to about 50 μg/mL, about 0.5 μg/mL to about 10, 20, 30, 40 or 50 μg/mL, about 1 μg/mL to about 10 μg/mL, or about 2 μg/mL, in the composition.

As noted herein, cells such as APCs that express an MHC class I molecule loaded with or bound to any one of, or any combination of, the TAPs defined herein, may be used for stimulating/amplifying CD8⁺ T lymphocytes in vivo or ex vivo. Accordingly, in another aspect, the present disclosure provides T cell receptor (TCR) molecules capable of interacting with or binding the herein-mentioned MHC class I molecule/TAP complex, and nucleic acid molecules encoding such TCR molecules, and vectors comprising such nucleic acid molecules. A TCR according to the present disclosure is capable of specifically interacting with or binding a TAP loaded on, or presented by, an MHC class I molecule, preferably at the surface of a living cell in vitro or in vivo.

The term TCR as used herein refers to an immunoglobulin superfamily member having a variable binding domain, a constant domain, a transmembrane region, and a short cytoplasmic tail; see, e.g., Janeway et al, Immunobiology: The Immune System in Health and Disease, 3rd Ed., Current Biology Publications, p. 4:33, 1997) capable of specifically binding to an antigen peptide bound to a MHC receptor. A TCR can be found on the surface of a cell and generally is comprised of a heterodimer having a and p chains (also known as TCRα and TCRβ, respectively). Like immunoglobulins, the extracellular portion of TCR chains (e.g., α-chain, β-chain) contain two immunoglobulin regions, a variable region (e.g., TCR variable α region or Vα and TCR variable β region or Vβ; typically amino acids 1 to 116 based on Rabat numbering at the N-terminus), and one constant region (e.g., TCR constant domain α or Cα and typically amino acids 117 to 259 based on Rabat, TCR constant domain β or Cβ, typically amino acids 117 to 295 based on Rabat) adjacent to the cell membrane. Also, like immunoglobulins, the variable domains contain complementary determining regions (CDRs, 3 in each chain) separated by framework regions (FRs). In certain embodiments, a TCR is found on the surface of T cells (or T lymphocytes) and associates with the CD3 complex.

A TCR and in particular nucleic acids encoding a TCR of the disclosure may for instance be applied to genetically transform/modify T lymphocytes (e.g., CD8⁺ T lymphocytes) or other types of lymphocytes generating new T lymphocyte clones that specifically recognize an MHC class I/TAP complex. In a particular embodiment, T lymphocytes (e.g., CD8⁺ T lymphocytes) obtained from a patient are transformed to express one or more TCRs that recognize a TAP and the transformed cells are administered to the patient (autologous cell transfusion). In a particular embodiment, T lymphocytes (e.g., CD8⁺ T lymphocytes) obtained from a donor are transformed to express one or more TCRs that recognize a TAP and the transformed cells are administered to a recipient (allogenic cell transfusion). In another embodiment, the disclosure provides a T lymphocyte e.g., a CD8⁺ T lymphocyte transformed/transfected by a vector or plasmid encoding a TAP-specific TCR. In a further embodiment the disclosure provides a method of treating a patient with autologous or allogenic cells transformed with a TAP-specific TCR. In certain embodiments, TCRs are expressed in primary T cells (e.g., cytotoxic T cells) by replacing an endogenous locus, e.g., an endogenous TRAC and/or TRBC locus, using, e.g., CRISPR, TALEN, zinc finger nuclease, or other targeted disruption systems.

In another embodiment, the present disclosure provides a nucleic acid encoding the above-noted TCR. In a further embodiment, the nucleic acid is present in a vector, such as the vectors described above.

In yet a further embodiment the use of a tumor antigen-specific TCR in the manufacture of autologous or allogenic cells for the treatment of cancer, such as melanoma, is provided.

In some embodiments, patients treated with the compositions (e.g., pharmaceutical compositions) of the disclosure are treated prior to or following treatment with an anti-tumor agent and/or immunotherapy (e.g., CAR therapy, immune checkpoint inhibitor therapy). Compositions of the disclosure include: allogenic T lymphocytes (e.g., CD8⁺ T lymphocyte) activated ex vivo against a TAP; allogenic or autologous APC vaccines loaded with a TAP; vaccines including TAPs of nucleic acids (e.g. mRNA) encoding TAPs and allogenic or autologous T lymphocytes (e.g., CD8⁺ T lymphocyte) or lymphocytes transformed with a tumor antigen-specific TCR. The method to provide T lymphocyte clones capable of recognizing a TAP according to the disclosure may be generated for and can be specifically targeted to tumor cells expressing the TAP in a subject (e.g., graft recipient), for example an allogenic T lymphocyte and/or donor lymphocyte infusion (DLI) recipient. Hence the disclosure provides a CD8⁺ T lymphocyte encoding and expressing a T cell receptor capable of specifically recognizing or binding a TAP/MHC class I molecule complex. Said T lymphocyte (e.g., CD8⁺ T lymphocyte) may be a recombinant (engineered) or a naturally selected T lymphocyte. This specification thus provides at least two methods for producing CD8+T lymphocytes of the disclosure, comprising the step of bringing undifferentiated lymphocytes into contact with a TAP/MHC class I molecule complex (typically expressed at the surface of cells, such as APCs) under conditions conducive of triggering T cell activation and expansion, which may be done in vitro or in vivo (i.e., in a patient administered with a APC vaccine wherein the APC is loaded with a TAP or in a patient treated with a TAP vaccine). Using a combination or pool of TAPs bound to MHC class I molecules, it is possible to generate a population CD8⁺ T lymphocytes capable of recognizing a plurality of TAPs. Alternatively, tumor antigen-specific or targeted T lymphocytes may be produced/generated in vitro or ex vivo by cloning one or more nucleic acids (genes) encoding a TCR (more specifically the alpha and beta chains) that specifically binds to a MHC class I molecule/TAP complex (i.e. engineered or recombinant CD8⁺ T lymphocytes). Nucleic acids encoding a TAP-specific TCR of the disclosure, may be obtained using methods known in the art from a T lymphocyte activated against a TAP ex vivo (e.g., with an APC loaded with a TAP); or from an individual exhibiting an immune response against peptide/MHC molecule complex. TAP-specific TCRs of the disclosure may be recombinantly expressed in a host cell and/or a host lymphocyte obtained from a graft recipient or graft donor, and optionally differentiated in vitro to provide cytotoxic T lymphocytes (CTLs). The nucleic acid(s) (transgene(s)) encoding the TCR alpha and beta chains may be introduced into a T cells (e.g., from a subject to be treated or another individual) using any suitable methods such as transfection (e.g., electroporation) or transduction (e.g., using viral vector). The engineered CD8⁺ T lymphocytes expressing a TCR specific for a TAP may be expanded in vitro using well known culturing methods.

The present disclosure provides methods for making the immune effector cells which express the TCRs as described herein. In one embodiment, the method comprises transfecting or transducing immune effector cells, e.g., immune effector cells isolated from a subject, such as a subject having melanoma, such that the immune effector cells express one or more TCR as described herein. In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a TCR. In this regard, the immune effector cells may be cultured before or after being genetically modified (i.e., transduced or transfected to express a TCR as described herein).

Prior to in vitro manipulation or genetic modification of the immune effector cells described herein, the source of cells may be obtained from a subject. In particular, the immune effector cells for use with the TCRs as described herein comprise T cells. T cells can be obtained from a number of sources, including peripheral blood mononuclear cells (PBMCs), bone marrow, lymph nodes tissue, cord blood, thymus issue, tissue from a site of infection, ascites, pleural effusion, spleen tissue, and tumors. In certain embodiments, T cell can be obtained from a unit of blood collected from the subject using any number of techniques known to the skilled person, such as FICOLL™ separation. In one embodiment, cells from the circulating blood of an individual are obtained by apheresis. The apheresis product typically contains lymphocytes, including T cells, monocytes, granulocyte, B cells, other nucleated white blood cells, red blood cells, and platelets. In one embodiment, the cells collected by apheresis may be washed to remove the plasma fraction and to place the cells in an appropriate buffer or media for subsequent processing. In one embodiment of the invention, the cells are washed with PBS. In an alternative embodiment, the washed solution lacks calcium and may lack magnesium or may lack many if not all divalent cations. As would be appreciated by those of ordinary skill in the art, a washing step may be accomplished by methods known to those in the art, such as by using a semi-automated flow-through centrifuge. After washing, the cells may be resuspended in a variety of biocompatible buffers or other saline solution with or without buffer. In certain embodiments, the undesirable components of the apheresis sample may be removed in the cell directly resuspended culture media. In certain embodiments, T cells are isolated from peripheral blood mononuclear cells (PBMCs) by lysing the red blood cells and depleting the monocytes, for example, by centrifugation through a PERCOLL™ gradient. A specific subpopulation of T cells, such as CD28⁺, CD4⁺, CD8⁺, CD45RA⁺, and CD45RO⁺ T cells, can be further isolated by positive or negative selection techniques. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD8+ cells by negative selection, a monoclonal antibody cocktail typically includes antibodies to CD14, CD20, CD11b, CD16, HLA-DR, and CD4. Flow cytometry and cell sorting may also be used to isolate cell populations of interest for use in the present disclosure. PBMC may be used directly for genetic modification with the TCRs using methods as described herein. In certain embodiments, after isolation of PBMC, T lymphocytes are further isolated and in certain embodiments, both cytotoxic and helper T lymphocytes can be sorted into naive, memory, and effector T cell subpopulations either before or after genetic modification and/or expansion.

The present disclosure provides isolated immune cells such as T lymphocytes (e.g., CD8⁺ T lymphocytes) that are specifically induced, activated and/or amplified (expanded) by a TAP (i.e., a TAP bound to MHC class I molecules expressed at the surface of cell), or a combination of TAPs. The present disclosure also provides a composition comprising CD8⁺ T lymphocytes capable of recognizing a TAP, or a combination thereof, according to the disclosure (i.e., one or more TAPs bound to MHC class I molecules) and said TAP(s).

In another aspect, the present disclosure provides a cell population or cell culture (e.g., a CD8⁺ T lymphocyte population) enriched in T lymphocytes (e.g., CD8⁺ T lymphocytes) that specifically recognize one or more MHC class I molecule/TAP complex(es) as described herein. Such enriched population may be obtained by performing an ex vivo expansion of specific T lymphocytes (e.g., TILs) using cells such as APCs that express MHC class I molecules loaded with (e.g., presenting) one or more of the TAPs disclosed herein. “Enriched” as used herein means that the proportion of tumor antigen-specific T lymphocytes (e.g., CD8⁺ T lymphocytes) in the population is significantly higher relative to a native population of cells, i.e., which has not been subjected to a step of ex vivo-expansion of specific T lymphocytes. In an embodiment, the cell population is a TIL population or is derived from TILs, e.g., TILs isolated from a patient and expanded ex vivo. In a further embodiment, the proportion of TAP-specific T lymphocytes (e.g., CD8⁺ T lymphocytes) in the cell population is at least about 0.5%, for example at least about 1%, 1.5%, 2% or 3%. In some embodiments, the proportion of TAP-specific T lymphocytes (e.g., CD8+T lymphocytes) in the cell population is about 0.5 to about 10%, about 0.5 to about 8%, about 0.5 to about 5%, about 0.5 to about 4%, about 0.5 to about 3%, about 1% to about 5%, about 1% to about 4%, about 1% to about 3%, about 2% to about 5%, about 2% to about 4%, about 2% to about 3%, about 3% to about 5% or about 3% to about 4%. Such cell population or culture (e.g., a CD8⁺ T lymphocyte population) enriched in T lymphocytes (e.g., CD8⁺ T lymphocytes) that specifically recognizes one or more MHC class I molecule/peptide (TAP) complex(es) of interest may be used in tumor antigen-based cancer immunotherapy, as detailed below. In some embodiments, the population of TAP-specific T lymphocytes (e.g., CD8⁺ T lymphocytes) is further enriched, for example using affinity-based systems such as multimers of MHC class I molecule loaded (covalently or not) with the TAP(s) defined herein. Thus, the present disclosure provides a purified or isolated population of TAP-specific T lymphocytes (e.g., CD8⁺ T lymphocytes), e.g., in which the proportion of TAP-specific T lymphocytes (e.g., CD8⁺ T lymphocytes) is at least about 50%, 60%, 70%, 80%, 85%, 90%, 95%, 96%, 97%, 98%, 99% or 100%.

In another aspect, the present disclosure provides an antibody or an antigen-binding fragment thereof (e.g., a TCR mimic or TCR-like antibody), or a soluble TCR, that specifically binds to a complex comprising a TAP as described herein bound to an HLA molecule, such as the HLA molecules defined herein. The term “antibody or antigen-binding fragment thereof” as used herein refers to any type of antibody/antibody fragment including monoclonal antibodies (including full-length monoclonal antibodies), polyclonal antibodies, multispecific antibodies, humanized antibodies, CDR-grafted antibodies, chimeric antibodies and antibody fragments so long as they exhibit the desired antigenic specificity/binding activity. Antibody fragments comprise a portion of a full-length antibody, generally an antigen binding or variable region thereof. Examples of antibody fragments include Fab, Fab′, F(ab′)₂, and Fv fragments, diabodies, linear antibodies, single-chain antibody molecules (e.g., single-chain Fv, scFv), single domain antibodies (e.g., from camelids), shark NAR single domain antibodies, and multispecific antibodies formed from antibody fragments, single-chain diabodies (scDbs), bispecific T cell engagers (BiTEs), dual affinity retargeting molecules (DARTs), bivalent scFv-Fcs, and trivalent scFv-Fcs. Antibody fragments can also refer to binding moieties comprising CDRs or antigen binding domains including, but not limited to, V_{H} regions (V_{H}, V_{H}-V_{H}), anticalins, PepBodies, antibody-T-cell epitope fusions (Troybodies) or Peptibodies. In an embodiment, the antibody or antigen-binding fragment thereof is a single-chain antibody, preferably a single-chain Fv (scFv). In an embodiment, the antibody or antigen-binding fragment thereof comprises at least one constant domain, e.g., a constant domain of a light and/or heavy chain, or a fragment thereof. In a further embodiment, the antibody or antigen-binding fragment thereof comprises a Fragment crystallizable (Fc) fragment of the constant heavy chain of an antibody. In an embodiment, the antibody or antigen-binding fragment is a scFv comprising a Fc fragment (scFV-Fc). In an embodiment, the scFv component is connected to the Fc fragment by a linker, for example a hinge. The presence of an Fc region is useful to induce a complement-dependent cytotoxicity (CDC), antibody-dependent cellular phagocytosis (ADCP), or antibody-dependent cellular cytotoxicity (ADCC) response against a tumor cell.

In an embodiment, the antibody or antigen-binding fragment thereof is a multispecific antibody or an antigen-binding fragment thereof, such as a bispecific antibody or an antigen-binding fragment thereof, wherein at least one of the antigen-binding domains of the multispecific antibody or antibody fragment recognize(s) a complex comprising a TAP as described herein bound to an HLA molecule. In an embodiment, at least one of the antigen-binding domains of the multispecific antibody or antibody fragment recognize(s) an immune cell effector molecule. The term “immune cell effector molecule” refers to a molecule (e.g., protein) expressed by an immune cell and whose engagement by the multispecific antibody or antibody fragment leads to activation of the immune cells. Examples of immune cell effector molecules include the CD3 signaling complex in T cells such as CD8 T cells and the various activating receptors on NK cells (NKG2D, KIR2DS, NKp44, etc.). In a further embodiment, at least one of the antigen-binding domains of the multispecific antibody or antibody fragment recognize(s) and engage(s) the CD3 signaling complex in T cells (e.g., anti-CD3). In a further embodiment, the multispecific antibody or antibody fragment is a single-chain diabody (scDb). In a further embodiment, the scDb comprises a first antibody fragment (e.g., scFv) that binds to a complex comprising a TAP as described herein bound to an HLA molecule and a second antibody fragment (e.g., scFv) that binds to and engages an immune cell effector molecule, such as the CD3 signaling complex in T cells (e.g., anti-CD3 scFv). Such constructs may be used for example to induce the cytotoxic T cell-mediated killing of tumor cells expressing the tumor antigen/MHC complex recognized by the multispecific antibody or antibody fragment. Antibodies or antigen-binding fragments thereof may also be used as a chimeric antigen receptor (CAR) to produce CAR T cells, CAR NK cells, etc. CAR combines a ligand-binding domain (e.g., antibody or antibody fragment) that provides specificity for a desired antigen (e.g., MHC/TAP complex) with an activating intracellular domain (or signal transducing domain) portion, such as a T cell or NK cell activating domain, providing a primary activation signal. Antigen-binding fragments of antibodies, and more particularly scFv, capable of binding to molecules expressed by tumor cells are commonly used as ligand-binding domains in CAR.

In an embodiment, the soluble TCR is a soluble therapeutic bispecific TCR (see, e.g., Robinson et al., FEBS J. 2021 November; 288(21):6159-6173; Dilchert et al., Antibodies (Basel). 2022 May 10; 11(2):34).

In an embodiment, the TCR or soluble TCR comprises a TCR beta (β) chain comprising a complementary determining region 3 (CDR3) comprising one of the amino acid sequences set forth in Table 3 (right column).

In an embodiment, the soluble TCR, antibody or antibody fragment (e.g., TCR-mimic) is attached to an antitumor agent to form an antibody-drug conjugate (ADC). Such ADC permits to target the antitumor agent to tumor cells expressing one or more of the TAPs described herein (see, e.g., Shen et al., Asian J Pharm Sci. 2020 November; 15(6):777-785).

The present disclosure also provides a nucleic acid such as an mRNA encoding the soluble TCR, antibody, antibody fragment or CAR described herein. Such nucleic acids may be formulated into suitable vehicles such as lipid nanoparticles are described above, and may be used in the treatment of cancers such as melanoma, as described below.

Thus, in another aspect, the present disclosure provides a host cell, preferably an immune cell such as a T cell or NK cell, expressing the antibody or antibody fragment (e.g., scFv) described herein, i.e., a CAR T cell or NK cell.

The present disclosure further relates to a pharmaceutical composition or vaccine comprising the above-noted immune cell (CD8⁺ T lymphocytes, CAR T cell, CAR NK cell) or population of TAP-specific CD8⁺ T lymphocytes. Such pharmaceutical composition or vaccine may comprise one or more pharmaceutically acceptable excipients and/or adjuvants, as described above.

In another aspect, the present disclosure further relates to the use of any of the TAP comprising or consisting of any of the sequences of SEQ ID NOs:1-505 defined herein, nucleic acid, expression vector, T cell receptor, antibody/antibody fragment, cell (e.g., T lymphocyte, APC, CAR T cell, CAR NK cell), and/or composition according to the present disclosure, or any combination thereof, as a medicament or in the manufacture of a medicament for the treatment of cancer (e.g., melanoma). The present disclosure relates to any TAP, nucleic acid, expression vector, T cell receptor, antibody/antibody fragment, cell (e.g., T lymphocyte, APC), and/or composition (e.g., vaccine composition) according to the present disclosure, or any combination thereof, for use in the treatment of cancer (e.g., melanoma), e.g., as a melanoma cancer vaccine. The TAP sequences identified herein may be used for the production of synthetic peptides to be used i) for in vitro priming and expansion of tumor antigen-specific T cells to be injected into tumor patients and/or ii) as vaccines to induce or boost the anti-tumor T cell response in cancer (e.g., melanoma) patients.

In another aspect, the present disclosure provides the use of a TAP described herein (e.g., comprising or consisting of any of the sequences of SEQ ID NOs:1-505 defined herein), or a combination thereof (e.g., a peptide pool), or of one or more nucleic acid(s) encoding the TAP(s), as a vaccine for treating cancer (e.g., melanoma) in a subject. The present disclosure also provides the TAP described herein, or a combination thereof (e.g., a peptide pool), or of one or more nucleic acid(s) encoding the TAP(s), for use as a vaccine for treating cancer (e.g., melanoma) in a subject. In an embodiment, the subject is a recipient of TAP-specific T lymphocytes (e.g., CD8⁺ T lymphocytes). Accordingly, in another aspect, the present disclosure provides a method of treating cancer, and more particularly melanoma (e.g., of reducing the number of tumor cells, killing tumor cells), said method comprising administering (infusing) to a subject in need thereof an effective amount of T lymphocytes (e.g., CD8⁺ T lymphocytes) recognizing (i.e., expressing a TCR that binds) one or more MHC class I molecule/TAP complexes (expressed at the surface of a cell such as an APC). In an embodiment, the method further comprises administering an effective amount of the TAP, or a combination thereof, or of one or more nucleic acid(s) encoding the TAP(s), and/or a cell (e.g., an APC such as a dendritic cell) expressing MHC class I molecule(s) loaded with the TAP(s), to said subject after administration/infusion of said CD8⁺ T lymphocytes. In yet a further embodiment, the method comprises administering to a subject in need thereof a therapeutically effective amount of a dendritic cell loaded with one or more TAPs. In yet a further embodiment the method comprises administering to a patient in need thereof a therapeutically effective amount of an allogenic or autologous cell that expresses a recombinant TCR that binds to a TAP presented by an MHC class I molecule.

In another aspect, the present disclosure provides the use of T lymphocytes (e.g., CD8⁺ T lymphocytes) that recognize one or more MHC class I molecules loaded with (presenting) a TAP, or a combination thereof, for treating cancer, and more particularly melanoma (e.g., of reducing the number of tumor cells, killing tumor cells) in a subject. In another aspect, the present disclosure provides the use of T lymphocytes (e.g., CD8⁺ T lymphocytes) that recognize one or more MHC class I molecules loaded with (presenting) a TAP, or a combination thereof, for the preparation/manufacture of a medicament for treating cancer, and more particularly melanoma (e.g., for reducing the number of tumor cells, killing tumor cells) in a subject. In another aspect, the present disclosure provides T lymphocytes (e.g., CD8⁺ T lymphocytes) that recognize one or more MHC class I molecule(s) loaded with (presenting) a TAP, or a combination thereof, for use in the treatment of cancer, and more particularly melanoma (e.g., for reducing the number of tumor cells, killing tumor cells), in a subject. In a further embodiment, the use further comprises the use of an effective amount of a TAP (or a combination thereof), or of one or more nucleic acid(s) encoding the TAP(s), and/or of a cell (e.g., an APC) that expresses one or more MHC class I molecule(s) loaded with (presenting) a TAP, after the use of said TAP-specific T lymphocytes.

The present disclosure also provides a method of generating an immune response against tumor cells (e.g., melanoma cells) expressing human class I MHC molecules loaded with any of the TAP disclosed herein (e.g., comprising or consisting of any of the sequences of SEQ ID NO:1-505 defined herein) or combination thereof in a subject, the method comprising administering cytotoxic T lymphocytes that specifically recognizes the class I MHC molecules loaded with the TAP or combination of TAPs. The present disclosure also provides the use of cytotoxic T lymphocytes that specifically recognizes class I MHC molecules loaded with any of the TAP or combination of TAPs disclosed herein for generating an immune response against tumor cells expressing the human class I MHC molecules loaded with the TAP or combination thereof.

The TAP, combination thereof (e.g., a peptide pool), nucleic acid(s) encoding the TAP(s), as well as antibodies, TCR, cells (e.g., CD8 T cells, APC), vaccines and compositions disclosed herein may be used of the prevention or treatment of any cancer that expresses the TAP, such as melanoma.

TAP, combination thereof (e.g., a peptide pool), nucleic acid(s) encoding the TAP(s), as well as antibodies/antibody fragments, TCR, cell (e.g., T lymphocyte, CAR T or NK cell, APC), vaccines and compositions disclosed herein may be used for inducing or stimulating an immune response against cancer cells expressing the TAP. In an embodiment, the cancer is melanoma. In an embodiment, the melanoma is cutaneous melanoma. In an embodiment, the melanoma is of the superficial spreading subtype. In an embodiment, the melanoma is of the nodular subtype. In an embodiment, the melanoma is of the lentigo maligna subtype. In an embodiment, the melanoma is of the nodular subtype. In an embodiment, the melanoma is of the acral lentiginous subtype. In an embodiment, the melanoma is ocular melanoma (e.g., choroidal melanoma, iris melanoma or uveal melanoma). In an embodiment, the melanoma is mucosal melanoma. In an embodiment, the melanoma is a stage 0 melanoma. In an embodiment, the melanoma is a stage 1 melanoma. In an embodiment, the melanoma is a stage 2 melanoma. In an embodiment, the melanoma is a stage 3 melanoma. In an embodiment, the melanoma is a stage 4 melanoma. In an embodiment, the melanoma is a relapsing melanoma. In an embodiment, the melanoma is a chemotherapy-resistant melanoma.

In an embodiment, the methods or uses described herein further comprise determining the HLA class I alleles expressed by the patient prior to the treatment/use, and administering or using TAPs that bind to one or more of the HLA class I alleles expressed by the patient. For example, if it is determined that the patient expresses HLA-01*01 and HLA-B40*01, any combinations of (i) the TAPs of SEQ ID NO: 11, 14, 20, 27, 29, 37, 86, 87, 121, 150, 165, 173, 279, 288, 299, 308, 312, 316, 319, 331, 340, 352, 362, 363, 384, 395, 398, 411, 419, 429, 458, 474, 476, 487, 492, 500, and/or 501 (that bind to HLA-A01*01) and (ii) the TAPs of SEQ ID NO:215 and/or 322 (that bind to HLA-B40*01) may be administered or used in the patient.

In some embodiments, patients treated with the compositions (e.g., pharmaceutical compositions) of the disclosure are treated prior to or following treatment with allogenic stem cell transplant (ASCL), allogenic lymphocyte infusion or autologous lymphocyte infusion.

In an embodiment, the TAP, nucleic acid, expression vector, T cell receptor, antibody/antibody fragment, cell (e.g., T lymphocyte, CAR T or NK cell, APC), and/or composition according to the present disclosure, or any combination thereof, may be used in combination with one or more additional active agents or therapies to treat melanoma, such as chemotherapy (e.g., vinca alkaloids, agents that disrupt microtubule formation (such as colchicines and its derivatives, monomethyl auristatin E (MMAE)), anti-angiogenic agents, therapeutic antibodies, EGFR targeting agents, tyrosine kinase targeting agent (such as tyrosine kinase inhibitors), transitional metal complexes, proteasome inhibitors, antimetabolites (such as nucleoside analogs), alkylating agents, platinum-based agents, anthracycline antibiotics, topoisomerase inhibitors, macrolides, retinoids (such as all-trans retinoic acids or a derivatives thereof), geldanamycin or a derivative thereof (such as 17-AAG), inhibitors of CDK4/6, TGF-β, WNT-β-catenin, MYC or PI3K, surgery, immune checkpoint inhibitors or immunotherapeutic agents (e.g., PD-1/PD-L1 inhibitors such as anti-PD-1/PD-L1 antibodies, CTLA-4 inhibitors such as anti-CTLA-4 antibodies, B7-1/B7-2 inhibitors such as anti-B7-1/B7-2 antibodies, TIM3 inhibitors such as anti-TIM3 antibodies, BTLA inhibitors such as anti-BTLA antibodies, CD47 inhibitors such as anti-CD47 antibodies, GITR inhibitors such as anti-GITR antibodies), antibodies against tumor antigens (e.g., anti-CD19, anti-CD22 antibodies), cell-based therapies (e.g., CAR T cells, CAR NK cells), and cytokines such as IL-2, IL-7, IL-21, and IL-15. In an embodiment, the TAP, nucleic acid, expression vector, T cell receptor, cell (e.g., T lymphocyte, APC), and/or composition according to the present disclosure is administered/used in combination with an immune checkpoint inhibitor. In an embodiment, the TAP, nucleic acid, expression vector, T cell receptor, cell (e.g., T lymphocyte, APC), and/or composition according to the present disclosure is administered/used in combination with radiotherapy. In an embodiment, the TAP, nucleic acid, expression vector, T cell receptor, antibody/antibody fragment, cell (e.g., T lymphocyte, CAR T or NK cell, APC), and/or composition according to the present disclosure is administered/used in combination with surgery. In an embodiment, the TAP, nucleic acid, expression vector, T cell receptor, antibody/antibody fragment, cell (e.g., T lymphocyte, CAR T or NK cell, APC), and/or composition according to the present disclosure is administered/used in combination with inhibitors of CDK4/6, TGF-β and/or WNT-β-catenin. Several CDK4/6 inhibitors are in clinical trials including Palbociclib (PD-0332991, Ibrance), Ribociclib (LEE-011, Kisqali), Abemaciclib (LY2835219, Verzenios), SHR6390 and Trilaciclib (G1T28). Inhibitors of TGF-β include antisense inhibitors such as AP12009 (Trabedersen) and ISTH0036, antibodies and ligand traps such as GC1008 (Fresolimumab), LY2382770, and P144, vaccines targeting the TGF-β pathway such as Belagenpumatucel-L (Lucanix™), and FANG™ or vigil (Gemogenovatucel-T), as well as small molecule inhibitors such as LY2157299 (Galunisertib) and TEW-7197. Inhibitors of the WNT-β-catenin pathway include amino acid starvators (asparaginase), GSK3 inhibitors, C2

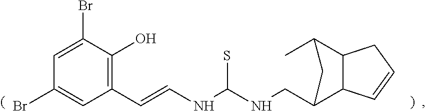 [SMILES: CC1CC(CNC(S)N/C=C/c2cc(Br)cc(Br)c2O)C2C=CCC12]

WNT974, ETC-1922159, RXC004, CGX1321, OTSA101-DTPA-90Y, Vantictumab (OMP-18R5), Ipafricept (OMP-54F28), PRI-724, SM08502, secreted frizzled-related proteins/peptides and Tankyrase inhibitors (XAV939, JW-55, RK-287107, and G007-LK).

The additional therapy may be administered prior to, concurrent with, or after the administration of the TAP, nucleic acid, expression vector, T cell receptor, antibody/antibody fragment, cell (e.g., T lymphocyte, CAR T or NK cell, APC), and/or composition according to the present disclosure.

### EXAMPLES

The present disclosure is illustrated in further details by the following non-limiting example.

### Example 1: Identification of Melanoma Tumor Antigen Candidates

### Acquisition of Samples.

The 12 flash-frozen cutaneous melanoma specimens used in this study were purchased from Tissue Solutions. The project was approved by the Research Ethics Boards of the Université de Montreal. Between 700 mg and 1.4 g per tumor were used for mass spectrometry analyses and between 30 mg and 70 mg were used for RNA-Sequencing. The immunopeptidomic and RNA-Sequencing data for the 7 patient-derived melanoma cell lines from Chong et al.¹ were downloaded from the European Genome-phenome Archive (EGA), accession number EGAS00001003724.

### Immunoprecipitation of MHC I.

The W6/32 antibodies (BioXcell) were incubated in PBS for 60 min at room temperature with PureProteome™ protein A magnetic beads (Millipore) at a ratio of 1 mg of antibody per mL of slurry. Antibodies were covalently cross-linked to magnetic beads using dimethylpimelidate, as described previously². The beads were stored at 4° C. in PBS pH 7.2 and 0.02% NaN₃. Primary tumor tissue samples were cut into small pieces (cubes, approx. 3 mm in size) and 6 ml of ice-cold PBS containing protein inhibitor cocktail (Sigma, cat #P8340-5 ml) was added. Tissues were homogenized twice for 20 seconds using an Ultra Turrax T25 homogenizer (IKA-Labortechnik) set at a speed of 20,000 rpm. Then, 700 μl of ice-cold 1OX lysis buffer (5% w/v CHAPS) was added to each sample. After 60-minute incubation with tumbling at 4° C., tissue samples were spun at 16,000 g for 20 minutes at 4° C. Supernatants were transferred into new tubes containing 1 mg of W6/32 antibody covalently-cross-linked protein A magnetic beads. Samples were incubated with tumbling for 20 hrs at 4° C. and placed on a magnet to recover bound MHC I complexes to magnetic beads. Magnetic beads were first washed with 8×1 mL PBS, then with 1×1 mL of 0.1×PBS and finally with 1×1 mL of water. MHC I complexes were eluted from the magnetic beads by acidic treatment using 1% trifluoroacetic acid (TFA). To remove any residual magnetic beads, eluates were transferred into 2 mL Costar mL Spin-X centrifuge tube filters (0.45 pm, Corning) and spun 5 min at 1500 g. Filtrates containing peptides were separated from MHC I subunits (HLA molecules and p-2 macroglobulin) using home-made stage tips packed with two 1 mm diameter octadecyl (C-18) solid-phase extraction disks (EMPORE). Stage tips were pre-washed first with methanol then with 80% acetonitrile (ACN) in 0.1% TFA, followed by 0.1% TFA and finally with 1% TFA. Samples were loaded onto the stage tips and the peptides were retained on the stage tips while the HLA molecules and p-2 macroglobulin were found in the flow through. Stage tips were washed with 1% TFA and then with 0.1% TFA, and peptides were eluted with 30% ACN in 0.1% TFA. The peptides were dried using vacuum centrifugation and then stored at −20° C. until MS analysis.

### TMT Labeling.

TMT labeling was performed for the 12 primary melanoma samples used in the study. The samples were reconstituted in 20 μL of 200 mM HEPES buffer, pH 8.2. The TMT reagents (Thermo Fisher Scientific) were dissolved in 40 μL of anhydrous ACN (Sigma-Aldrich) and 50 or 100 μg of reagent was added to the peptides. The solution was gently mixed and incubated for 90 min without agitation at RT before the reaction was quenched by hydroxylamine (Thermo Fisher Scientific). Samples were desalted on Silica C18 UltraMicroSpin™ Column (The Nest Group), dried down and reconstituted in 4% FA (EMD Millipore).

### Liquid Chromatography-Tandem MS Analyses.

Dried peptide extracts were resuspended in 4% FA and loaded on a homemade C18 analytical column (20 cm×150 m i.d. packed with C18 Jupiter Phenomenex) with a 106-minute gradient from 0% to 30% ACN (0.2% FA) and a 600 nL/min flow rate on an EASY-nLC II system. 2 samples were analyzed with a Q-Exactive HF and 10 samples with an Orbitrap Fusion mass spectrometer (Thermo Fisher Scientific). For Q-Exactive HF, analyses were done in positive ion mode with Nanospray 2 source at 1.6 kV. Each full MS spectrum, acquired with a 60,000 resolution was followed by 20 MS/MS spectra, where the most abundant multiply charged ions were selected for MS/MS sequencing with a resolution of 60,000, an automatic gain control target of 2×104, an injection time of 800 ms and collisional energy of 28%. Analyses with Orbitrap Fusion mass spectrometer were done in positive ion mode with Nanoflex source at 2.8 kV. Each full MS spectrum, acquired with a 120,000 resolution was followed by 20 MS/MS spectra, where the most abundant multiply charged ions were selected for MS/MS sequencing with a resolution of 50,000, an automatic gain control target of 2×10⁴, an injection time of 1000 ms, and collisional energy of 35%.

### Construction of MS Databases.

Cancer-specific proteomes were assembled using k-mer profiling as described³⁴. k-mers (33-nucleotide-long) present more than once in the mTECs k-mer database were removed from each cancer sample database, and the remaining k-mers were assembled into contigs. Finally, the contigs were 3-frame translated, and the different polypeptides were linked with “JJ” linkers. Each of these databases was concatenated with the corresponding sample's canonical proteome and used for TA identification.

### MAP Identification.

LC-MS/MS data were searched against the relevant database using Peaks X Pro (Bioinformatics Solution Inc.). For peptide identification, tolerance was set at 10 ppm and 0.01 Da for precursor and fragment ions, respectively. Oxidation (M) and deamidation were set as variable modifications. In addition, for the TMT labeling, the occurrences of K and N-term were set as fixed modifications, and the occurrence of STY was set as a variable modification. Following peptide identification, the modified target-decoy approach built-in PEAKS was used to apply a sample-specific threshold on the PEAKS scores to ensure a false discovery rate (FDR) of 5%, calculated as the ratio between the number of decoy hits and the number of target hits above the score threshold. PEAKS scores corresponding to a 5% FDR for each sample were determined, and peptides that passed the threshold were further filtered to match the following criteria: peptide length between 8 and 11 amino acids, eluted ligand likelihood prediction rank to any of the sample's HLA alleles <2% based on NetMHCpan-4.1b⁵. These filtering steps were performed with the use of MAPDP⁶.

### Selection of TSAs, TAAs and LSAs.

TA candidates were selected as previously described^{3,4} based on their source RNA expression in the cancer sample of origin and mTECs (FC≥10). The expression of the TA candidates' coding sequences was then evaluated in normal tissues from GTEx (n=up to 50 samples per tissue, 50 tissues), mTECs (n=11), purified blood and bone marrow samples (n=115), purified melanocytes (n=11), melanoma samples from TCGA (n=150, TCGA_SKCM), and melanoma cell lines from Chong et al.¹ (n=7) using BamQuery^{4,7}. TAs listed herein are the peptides meeting the following criteria:
1. mTSAs are derived from mutated genomic sequences supported by at least 5 reads and 5% reads at locus; the mutation is not reported in dbSNP version 155 (except if annotated as ‘pathogenic’);
2. For unmutated TAs, the 95^{th} percentile RNA expression value in TCGA_SKCM is at least 2 times higher than the 95^{th} percentile RNA expression value in GTEx (except testis and skin) and mTEC samples;
   A) for aeTSAs: The aeTSA's source RNA is expressed below 8.55 reads per hundred million (rphm) in more than 90% normal samples from mTECs, melanocytes, blood and bone marrow cells, and each GTEx tissue except the testis; the mean expression is at least 2 times higher in TCGA_SKCM compared to normal GTEx skin; and the mean expression is at least 2 times higher in melanoma cell lines compared to purified melanocytes;
   B) for TAAs: The TAA's source RNA can be expressed above 8.55 reads per hundred million (rphm) in >10% of samples from any normal tissues (GTEx, melanocytes, mTECs and/or blood and bone marrow cells), but the mean expression is at least 2 times higher in TCGA_SKCM compared to mTECs and each GTEx tissue except testis, and the mean expression in melanoma cell lines is at least 2 times higher compared to purified melanocytes.
   C) For LSAs: The LSA's source RNA is expressed above 8.55 rphm in at least 10% of GTEx skin samples or purified melanocytes, and the mean expression in GTEx skin is higher compared to all other GTEx tissues; and is expressed below 8.55 rphm in more than 90% normal samples from mTECs, blood and bone marrow cells, and each GTEx tissue except the testis and skin.

### TCGA Analyses.

TCGA RNA-seq gene expression data for hg38 were retrieved as upper quartile-normalized fragments per kilobase of transcript per million mapped reads (FPKM-UQ) using the R package TCGAbiolinks. For genes with duplicate entries, we calculated the average expression across entries. FPKM-UQ values were then used for correlations with each TA's RPHM expression from BamQuery, or to perform single-sample gene set enrichment analyses (ssGSEA) for the stemness gene set reported by Miranda et al.¹⁸ using the R package GSVA. Processed level 3 methylation data (HM450) from the Liftover Pipeline for TCGA samples were previously retrieved using the TCGAbiolinks package. Only probes within 2 kb of the transcription start site of a given TA-source gene were kept. The mean beta value was used for genes associated with multiple probes. For focal CNV correlations, processed hg38 gene-level copy number scores were previously retrieved using the TCGAbiolinks package. Mean DNA copy-number values were used for multiple segments associated with a TA-coding region. Mutation rate data for TCGA samples were retrieved from Firebrowse (http://firebrowse.org/) as the number of non-synonymous mutations per base (rate_non column). All correlations with TA RPHM expression were performed with the rcorr function in R. P-values were adjusted using the Benjamini-Hochberg method.

The characteristics of the TSAs, TAAs and LSAs identified herein are described in Tables 2A-2C.

TABLE 2A
characteristics of the TSAs, TAAs and LSAs identified herein

| Peptide Sequence | | | | |
|---|---|---|---|---|
| (SEQ ID NO:) | Categ. | HLA Allele | Sample | Biotype |
| TLDDAVHGV (1) | aeTSA | HLA-A02:01 | T1185B_Melanoma_ctrl | Intergenic |
| YQIGQVQGV (2) | aeTSA | HLA-A02:01 | Me290_Melanoma | ncRNA |
| NEYSNITRLY³ (3) | mTSA | HLA-B18:01 | 0MM745_Melanoma_ctrl | annotated_ORF |
| RLLADLTGA⁴ (4) | mTSA | HLA-A02:01 | Me275_Melanoma | annotated_ORF |
| TPLSPDPTL⁵ (5) | mTSA | HLA-B35:03 | T1015A_Melanoma | 5UTR |
| STYYWPRPR (6) | aeTSA | HLA-A68:01 | T1185B_Melanoma_ctrl | annotated_ORF |
| DSFSATETR (7) | aeTSA | HLA-A68:01 | T1185B_Melanoma_ctrl | ncRNA |
| ETSYVKVLH (8) | aeTSA | HLA-A68:01 | T1185B_Melanoma_ctrl | annotated_ORF |
| AEMLGSVV (9) | aeTSA | HLA-B49:01 | T1015A_Melanoma | annotated_ORF |
| EELSVLEVF (10) | aeTSA | HLA-B18:01 | 258_20T | annotated_ORF |
| FVQENYLEY (11) | aeTSA | HLA-A25:01, | 258_20T,0NVC_Melanoma_ctrl, | annotated_ORF |
| | | HLA-A26:01, | Me290_Melanoma, | |
| | | HLA-A01:01, | 1496_16T_Melanoma | |
| | | HLA-B15:01 | | |
| IVQQPTEEK (12) | aeTSA | HLA-A68:01 | T1185B_Melanoma_ctrl | annotated_ORF |
| SVFAHPRKL (13) | aeTSA | HLA-C06:02, | T1185B_Melanoma_ctrl, | annotated_ORF |
| | | HLA-C07:01, | Me290_Melanoma, | |
| | | HLA-C07:02 | Me275_Melanoma | |
| ASSLPTTMNY (14) | aeTSA | HLA-A01:01 | Me290_Melanoma | annotated_ORF |
| IVQQPTEEKR (15) | aeTSA | HLA-A68:01 | T1185B_Melanoma_ctrl | annotated_ORF |
| LPTTMNYPL (16) | aeTSA | HLA-B35:03 | T1015A_Melanoma | annotated_ORF |
| GSNPARYEF (17) | aeTSA | HLA-A32:01 | T1015A_Melanoma | annotated_ORF |
| TADDATVM (18) | aeTSA | HLA-C08:02 | 738_20T | annotated_ORF |
| VLEHVVRV (19) | aeTSA | HLA-A02:01 | Me275_Melanoma | annotated_ORF |
| EVDPIGHLY (20) | aeTSA | HLA-A01:01, | 1836_18T, | annotated_ORF |
| | | HLA-A26:01 | 0NVC_Melanoma_ctrl | |
| | | | 0MM745_Melanoma_ctrl, | |
| | | | Me290_Melanoma | |
| QIMPKAGL (21) | aeTSA | HLA-B08:01 | 0MM745_Melanoma_ctrl | annotated_ORF |
| KVLEHVVRV (22) | aeTSA | HLA-A02:09, | T1015A_Melanoma, | annotated_ORF |
| | | HLA-A02:01 | T1185B_Melanoma_ctrl, | |
| | | | Me275_Melanoma | |
| QVPGSNPAR (23) | aeTSA | HLA-A68:01 | T1185B_Melanoma_ctrl | annotated_ORF |
| FVYGEPREL (24) | aeTSA | HLA-C12:03, | 258_20T, 1367_16T, 542_20T, | annotated_ORF |
| | | HLA-C03:03, | 738_20T, | |
| | | HLA-C03:04 | T1185B_Melanoma_ctrl | |
| TLPTFDPTKV (25) | aeTSA | HLA-A02:01 | 542_20T, Me275_Melanoma | annotated_ORF |
| NQIMPKTGF (26) | aeTSA | HLA-B38:01, | 0NVC_Melanoma_ctrl | annotated_ORF |
| | | HLA-B15:01 | | |
| EVDPIGHVY (27) | aeTSA | HLA-A26:01, | Me290_Melanoma, | annotated_ORF |
| | | HLA-A01:01 | 0D5P_Melanoma_ctrl | |
| ELVRRILSR (28) | aeTSA | HLA-A68:01 | T1185B_Melanoma_ctrl | annotated_ORF |
| MEVDPIGHLY (29) | aeTSA | HLA-A01:01 | 0MM745_Melanoma_ctrl | annotated_ORF |
| SVMSSNVSF (30) | aeTSA | HLA-B15:01 | 1367_16T | annotated_ORF |
| TVPSSFPSW (31) | aeTSA | HLA-A25:01, | 258_20T, 1496_16T_Melanoma | annotated_ORF |
| | | HLA-A24:02 | | |
| KYKDYFPVI (32) | aeTSA | HLA-C07:02 | 542_20T | annotated_ORF |
| MEVDPIGHVY (33) | aeTSA | HLA-B18:01 | 0MM745_Melanoma_ctrl | annotated_ORF |
| TVYGEPRKL (34) | aeTSA | HLA-C06:02 | T1185B_Melanoma_ctrl | annotated_ORF |
| GPDVREGTL (35) | aeTSA | HLA-B07:02 | Me275_Melanoma | annotated_ORF |
| VYDGREHTV (36) | aeTSA | HLA-C07:02 | Me275_Melanoma | annotated_ORF |
| LMEVDPIGHLY (37) | aeTSA | HLA-A01:01 | Me290_Melanoma | annotated_ORF |
| KYKDYFPVILK (38) | aeTSA | HLA-A30:01 | 542_20T | annotated_ORF |
| EVFEGREDSIF (39) | aeTSA | HLA-A26:01 | 0NVC_Melanoma_ctrl | annotated_ORF |
| GVYDGREHTV (40) | aeTSA | HLA-A02:09, | T1015A_Melanoma, | annotated_ORF |
| | | HLA-A02:01 | T1185B_Melanoma_ctrl, | |
| | | | Me275_Melanoma, | |
| | | | 1496_16T_Melanoma | |
| EVVPISHLY (41) | aeTSA | HLA-A26:01, | 2883_18T, | annotated_ORF |
| | | HLA-A68:01 | 0NVC_Melanoma_ctrl, | |
| | | | T1185B_Melanoma_ctrl | |
| NAVGVYAGR (42) | aeTSA | HLA-A26:01, | 0NVC_Melanoma_ctrl, T1185B | annotated_ORF |
| | | HLA-A68:01 | Melanoma_ctrl | |
| RPDSRLLEL (43) | aeTSA | HLA-B07:02 | Me275_Melanoma | annotated_ORF |
| SVFAHPRKLLM (44) | aeTSA | HLA-A32:01 | T1015A_Melanoma | annotated_ORF |
| EVFEGREDSVF (45) | aeTSA | HLA-A26:01 | 2883_18T,0NVC_Melanoma_ctrl | annotated_ORF |
| RVSHSHASF (46) | aeTSA | HLA-A32:01 | T1015A_Melanoma | ncRNA |
| ATFLQRKVLR (47) | aeTSA | HLA-A68:01 | T1185B_Melanoma_ctrl | ncRNA |
| SYVKVLHHL (48) | aeTSA | HLA-A23:01 | Me275_Melanoma | annotated_ORF |
| FAFGEPREL (49) | aeTSA | HLA-C12:03, | 258_20T,738_20T,0NVC_Melan | annotated_ORF |
| | | HLA-C03:04 | oma_ctrl, T1185B_Melanoma_ctrl | |
| KMAELVHFL (50) | aeTSA | HLA-A02:09, | T1015A_Melanoma, | annotated_ORF |
| | | HLA-A02:01 | T1185B_Melanoma_ctrl, | |
| | | | Me290_Melanoma, | |
| | | | Me275_Melanoma | |
| FAHGHPLYTR (51) | aeTSA | HLA-A68:01 | T1185B_Melanoma_ctrl | annotated_ORF |
| GVYAGREHFV (52) | aeTSA | HLA-A02:01 | 542_20T | annotated_ORF |
| NTAIATFLQRK (53) | aeTSA | HLA-A68:01 | T1185B_Melanoma_ctrl | ncRNA |
| TLDEKVAEL (54) | aeTSA | HLA-C08:02, | 1367_16T,542_20T, 738_20T | annotated_ORF |
| | | HLA-A02:01 | | |
| RLLELHITM (55) | aeTSA | HLA-A02:01 | Me275_Melanoma | annotated_ORF |
| EVPNSSPPR (56) | aeTSA | HLA-A68:01 | T1185B_Melanoma_ctrl | annotated_ORF |
| VEVTLREV (57) | aeTSA | HLA-B49:01 | T1015A_Melanoma | annotated_ORF |
| ISGGPRISY (58) | aeTSA | HLA-C12:03 | 0NVC_Melanoma_ctrl | annotated_ORF |
| GVPGPLLILPR (59) | aeTSA | HLA-A68:01 | T1185B_Melanoma_ctrl | Intergenic |
| GVYDGEEHSV (60) | aeTSA | HLA-A02:01 | T1185B_Melanoma_ctrl, | annotated_ORF |
| | | | Me275_Melanoma | |
| KISGGPRISY (61) | aeTSA | HLA-A32:01 | T1015A_Melanoma | annotated_ORF |
| FLYNQTNIRM (62) | aeTSA | HLA-A02:09 | T1015A_Melanoma | ncRNA |
| GQYQLVVSL (63) | aeTSA | HLA-B15:01 | 1367_16T | annotated_ORF |
| GVDDLVFRR (64) | aeTSA | HLA-A68:01 | T1185B_Melanoma_ctrl | 5UTR |
| KVLEYVIKV (65) | aeTSA | HLA-A02:01 | T1185B_Melanoma_ctrl, | annotated_ORF |
| | | | Me275_Melanoma, | |
| | | | 1496_16T_Melanoma | |
| HTLPALSRR (66) | aeTSA | HLA-A68:01 | T1185B_Melanoma_ctrl | Intergenic |
| ALREEEEGV (67) | aeTSA | HLA-A02:01 | T1185B_Melanoma_ctrl, | annotated_ORF |
| | | | Me275_Melanoma | |
| TTINFTRQR (68) | aeTSA | HLA-A68:01 | T1185B_Melanoma_ctrl | annotated_ORF |
| SAYGEPRKL (69) | aeTSA | HLA-C03:04, | T1185B_Melanoma_ctrl, | annotated_ORF |
| | | HLA-C03:03 | 1496_16T_Melanoma | |
| STANVTRAH (70) | aeTSA | HLA-A68:01 | T1185B_Melanoma_ctrl | annotated_ORF |
| SAFPTTINF (71) | aeTSA | HLA-C12:03 | 542_20T | annotated_ORF |
| FRAVITKKV (72) | aeTSA | HLA-C06:02 | T1185B_Melanoma_ctrl | annotated_ORF |
| AAAGVSSTK (73) | aeTSA | HLA-A68:01 | T1185B_Melanoma_ctrl | annotated_ORF |
| FPTTINFTR (74) | aeTSA | HLA-A68:01 | T1185B_Melanoma_ctrl | annotated_ORF |
| TTSEKIHER (75) | aeTSA | HLA-C07:01, | T1015A_Melanoma, | annotated_ORF |
| | | HLA-A68:01 | T1185B_Melanoma_ctrl | |
| GLYDGMEHL (76) | aeTSA | HLA-A02:01 | Me275_Melanoma | annotated_ORF |
| STANVTRAHGR (77) | aeTSA | HLA-A68:01 | T1185B_Melanoma_ctrl | annotated_ORF |
| IEDPKAGLHV (78) | aeTSA | HLA-B49:01 | T1015A_Melanoma | annotated_ORF |
| GVVGPQGLR (79) | aeTSA | HLA-A68:01 | T1185B_Melanoma_ctrl | annotated_ORF |
| NVFAHATGESR (80) | aeTSA | HLA-A68:01 | T1185B_Melanoma_ctrl | ncRNA |
| FLFGEPKRL (81) | aeTSA | HLA-A02:01 | T1185B_Melanoma_ctrl | annotated_ORF |
| SAFPTTINFTR (82) | aeTSA | HLA-A68:01 | T1185B_Melanoma_ctrl | annotated_ORF |
| RLFSEDIHV (83) | aeTSA | HLA-A02:09 | T1015A_Melanoma | Intergenic |
| VYMPANTTF (84) | aeTSA | HLA-A23:01 | Me275_Melanoma | Introns |
| EAVGFQPPR (85) | aeTSA | HLA-A68:01 | T1185B_Melanoma_ctrl | ncRNA |
| EVDPTSHSY (86) | aeTSA | HLA-A01:01 | 0MM745_Melanoma_ctrl | annotated_ORF |
| LLDPAQRNLY (87) | aeTSA | HLA-A01:01 | 0D5P_Melanoma_ctrl | annotated_ORF |
| SGISQVFQR (88) | aeTSA | HLA-A68:01 | T1185B_Melanoma_ctrl | annotated_ORF |
| RLFSEDIHVA (89) | aeTSA | HLA-A02:09, | T1015A_Melanoma, | Intergenic |
| | | HLA-A02:01 | Me290_Melanoma, | |
| | | | Me275_Melanoma | |
| VFDVGKGNM (90) | aeTSA | HLA-C04:01 | T1015A_Melanoma | ncRNA |
| KASEKIFYV (91) | aeTSA | HLA-A02:01 | Me275_Melanoma | annotated_ORF |
| EAAAAFRFPR (92) | aeTSA | HLA-A68:01 | T1185B_Melanoma_ctrl | ncRNA |
| MSISMSLLK (93) | aeTSA | HLA-A68:01 | T1185B_Melanoma_ctrl | Intergenic |
| KASPSSLPH (94) | aeTSA | HLA-C12:03 | 0D5P_Melanoma_ctrl | Introns |
| RTLQQGVLQDW (95) | aeTSA | HLA-A32:01 | T1015A_Melanoma | annotated_ORF |
| TTATTFRAR (96) | aeTSA | HLA-A68:01 | T1185B_Melanoma_ctrl | annotated_ORF |
| NPSHLITPR (97) | aeTSA | HLA-A68:01 | T1185B_Melanoma_ctrl | Intergenic |
| ETMETLRTSR (98) | aeTSA | HLA-A68:01 | T1185B_Melanoma_ctrl | ncRNA |
| MVWEGHPRERR (99) | aeTSA | HLA-A68:01 | T1185B_Melanoma_ctrl | ncRNA |
| LPDGSRVEL (100) | aeTSA | HLA-B35:03 | T1015A_Melanoma | annotated_ORF |
| DVVTGNVPLK (101) | aeTSA | HLA-A68:01 | T1185B_Melanoma_ctrl | annotated_ORF |
| ENPSHLITPR (102) | aeTSA | HLA-A68:01 | T1185B_Melanoma_ctrl | Intergenic |
| SLARTILHL (103) | aeTSA | HLA-A02:01 | 542_20T | Introns |
| KPHYGLRAI (104) | aeTSA | HLA-B07:02 | Me275_Melanoma | annotated_ORF |
| AFLDIHTAL (105) | aeTSA | HLA-C07:02, | 0D5P_Melanoma_ctrl | Intergenic |
| | | HLA-C14:02 | | |
| KMSEFVYRF (106) | aeTSA | HLA-A32:01 | T1015A_Melanoma | 5UTR |
| FLVTKTVTM (107) | aeTSA | HLA-A02:01 | Me275_Melanoma | ncRNA |
| HTTSVMENK (108) | aeTSA | HLA-A68:01 | T1185B_Melanoma_ctrl | ncRNA |
| LVSIPIAVR (109) | aeTSA | HLA-A68:01 | T1185B_Melanoma_ctrl | Intergenic |
| ITSGVKVKK (110) | aeTSA | HLA-A68:01 | T1185B_Melanoma_ctrl | Intergenic |
| VSIPIAVRR (111) | aeTSA | HLA-A68:01 | T1185B_Melanoma_ctrl | Intergenic |
| DIIELQEGLR (112) | aeTSA | HLA-A68:01 | T1185B_Melanoma_ctrl | ncRNA |
| HLKPPELGF (113) | aeTSA | HLA-B15:01 | 0D5P_Melanoma_ctrl | Intergenic |
| QVIVKIIGH (114) | aeTSA | HLA-A26:01 | 0NVC_Melanoma_ctrl | annotated_ORF |
| AEQEIMKKV (115) | aeTSA | HLA-B49:01 | T1015A_Melanoma | annotated_ORF |
| EVPLKILAH (116) | aeTSA | HLA-A26:01 | 0NVC_Melanoma_ctrl | annotated_ORF |
| IVKIIGHFY (117) | aeTSA | HLA-A30:02 | 0MM745_Melanoma_ctrl | annotated_ORF |
| SLVSIPIAVR (118) | aeTSA | HLA-A68:01 | T1185B_Melanoma_ctrl | Intergenic |
| MVSPGVSLFK (119) | aeTSA | HLA-A68:01 | T1185B_Melanoma_ctrl | Intergenic |
| VLMMKLEDL (120) | aeTSA | HLA-A02:01 | Me275_Melanoma | Introns |
| YSDLHTPGRY (121) | aeTSA | HLA-A01:01 | 0MM745_Melanoma_ctrl, | annotated_ORF |
| | | | Me290_Melanoma, | |
| | | | 0D5P_Melanoma_ctrl | |
| EVARFLTYY (122) | aeTSA | HLA-A26:01 | 0NVC_Melanoma_ctrl | annotated_ORF |
| SVGDLMAPR (123) | aeTSA | HLA-A68:01 | T1185B_Melanoma_ctrl | ncRNA |
| VLWRGDSPL (124) | aeTSA | HLA-A02:01 | Me275_Melanoma | ncRNA |
| QPRQGSPVAA (125) | aeTSA | HLA-B07:02 | 0D5P_Melanoma_ctrl | annotated_ORF |
| KQTGIRHSY (126) | aeTSA | HLA-A32:01 | T1015A_Melanoma | Introns |
| RPRQDAISV (127) | aeTSA | HLA-B07:02 | 0D5P_Melanoma_ctrl | Intergenic |
| KLITQIRTA (128) | aeTSA | HLA-A02:01 | Me275_Melanoma | ncRNA |
| SYLSEHSAAIF (129) | aeTSA | HLA-A23:01 | Me275_Melanoma | Introns |
| TSVGDLMAPR (130) | aeTSA | HLA-A68:01 | T1185B_Melanoma_ctrl | ncRNA |
| EIPTGVAKR (131) | aeTSA | HLA-A68:01 | T1185B_Melanoma_ctrl | ncRNA |
| SQINLSQIY (132) | aeTSA | HLA-B15:01 | 0D5P_Melanoma_ctrl | 3UTR |
| RRPSGGFRL (133) | aeTSA | HLA-C07:01 | Me290_Melanoma | Intergenic |
| EVFNVKNTFPK (134) | aeTSA | HLA-A68:01 | T1185B_Melanoma_ctrl | Intergenic |
| KVLGGGWTV (135) | aeTSA | HLA-A02:01 | Me290_Melanoma | ncRNA |
| HEAPGQHSEV (136) | aeTSA | HLA-B49:01 | T1015A_Melanoma | ncRNA |
| EVSHPNMSGR (137) | aeTSA | HLA-A68:01 | T1185B_Melanoma_ctrl | ncRNA |
| DTAPATFLR (138) | aeTSA | HLA-A68:01 | T1185B_Melanoma_ctrl | ncRNA |
| KPQKDLHFI (139) | aeTSA | HLA-B07:02 | 0D5P_Melanoma_ctrl | ncRNA |
| RVRGAGTHL (140) | aeTSA | HLA-B07:02 | 0D5P_Melanoma_ctrl | ncRNA |
| TAPIIVAQR (141) | aeTSA | HLA-A68:01 | T1185B_Melanoma_ctrl | Intergenic |
| TSWDTKFYEK (142) | aeTSA | HLA-A68:01 | T1185B_Melanoma_ctrl | annotated_ORF |
| KVFAEHKISY (143) | aeTSA | HLA-A32:01 | T1015A_Melanoma | annotated_ORF |
| DAFQGIANR (144) | aeTSA | HLA-A68:01 | T1185B_Melanoma_ctrl | Intergenic |
| IVADPRVPR (145) | aeTSA | HLA-A68:01 | T1185B_Melanoma_ctrl | annotated_ORF |
| TGFSTSLGR (146) | aeTSA | HLA-A68:01 | T1185B_Melanoma_ctrl | Introns |
| ATAPIIVAQR (147) | aeTSA | HLA-A68:01 | T1185B_Melanoma_ctrl | Intergenic |
| GQFLVKSGY (148) | aeTSA | HLA-B15:01 | 0D5P_Melanoma_ctrl | annotated_ORF |
| WTSRTVQNK (149) | aeTSA | HLA-A68:01 | T1185B_Melanoma_ctrl | Intergenic |
| ATYGGSDHYI¹ (150) | mTSA | HLA-A01:01 | Me290_Melanoma | annotated_ORF |
| VYIAELEKI (151) | aeTSA | HLA-A23:01 | Me275_Melanoma | annotated_ORF |
| VFLDRAVSI⁶ (152) | aeTSA | HLA-A23:01 | Me275_Melanoma | annotated_ORF |
| MPSPIDHPR (153) | aeTSA | HLA-A68:01 | T1185B_Melanoma_ctrl | ncRNA |
| EAVRHILHGSR (154) | aeTSA | HLA-A68:01 | T1185B_Melanoma_ctrl | Intergenic |
| YPAGNVKEL (155) | aeTSA | HLA-B35:03 | T1015A_Melanoma | annotated_ORF |
| SVPRSLASR (156) | aeTSA | HLA-A68:01 | T1185B_Melanoma_ctrl | ncRNA |
| ALIKHVANA (157) | aeTSA | HLA-A02:01 | Me275_Melanoma | Introns |
| YTANLVPVR (158) | aeTSA | HLA-A68:01 | T1185B_Melanoma_ctrl | ncRNA |
| LSSSLVTKL (159) | aeTSA | HLA-C03:04 | T1185B_Melanoma_ctrl | Intergenic |
| QNSNVLAVR (160) | aeTSA | HLA-A68:01 | T1185B_Melanoma_ctrl | Introns |
| TTTKNTLNK (161) | aeTSA | HLA-A68:01 | T1185B_Melanoma_ctrl | ncRNA |
| HNYINLHSL (162) | aeTSA | HLA-B08:01, | 1713_19T, | Intergenic |
| | | HLA-C12:03 | 0NVC_Melanoma_ctrl | |
| HLKDYFEEY² (163) | mTSA | HLA-C07:01 | 1772_19T | annotated_ORF |
| SPEYFTSYL (164) | aeTSA | HLA-B35:03 | T1015A_Melanoma | Intergenic |
| ATDVLDHLY (165) | aeTSA | HLA-A01:01 | 1713_19T,534_20T, | ncRNA |
| | | | Me290_Melanoma, | |
| | | | 0D5P_Melanoma_ctrl | |
| QTFEESLGR (166) | aeTSA | HLA-A68:01 | T1185B_Melanoma_ctrl | Intergenic |
| HEQPELQKV (167) | aeTSA | HLA-B49:01 | T1015A_Melanoma | retained_intron |
| MATDVLDHLY (168) | aeTSA | HLA-A26:01 | 0NVC_Melanoma_ctrl | ncRNA |
| TSGSLLFPR (169) | aeTSA | HLA-A68:01 | T1185B_Melanoma_ctrl | Intergenic |
| KLGINNVSF (170) | aeTSA | HLA-A32:01 | T1015A_Melanoma | annotated_ORF |
| KYISQFQVF (171) | aeTSA | HLA-A23:01 | Me275_Melanoma | Intergenic |
| QTAAVSGVK (172) | aeTSA | HLA-A68:01 | T1185B_Melanoma_ctrl | Intergenic |
| LTPLNHPFSY (173) | aeTSA | HLA-A01:01 | 0D5P_Melanoma_ctrl | Intergenic |
| VFINKKYVF (174) | aeTSA | HLA-A23:01 | Me275_Melanoma | Intergenic |
| NLITEEHPV (175) | aeTSA | HLA-A02:01 | Me275_Melanoma | Intergenic |
| ELVKVIDGSSR (176) | aeTSA | HLA-A68:01 | T1185B_Melanoma_ctrl | Introns |
| FHSLPIHLF (177) | aeTSA | HLA-C07:02 | Me275_Melanoma | Intergenic |
| YSHDYSYY (178) | aeTSA | HLA-C12:03 | 0D5P_Melanoma_ctrl | Intergenic |
| FQIEKLTPL (179) | aeTSA | HLA-A02:01 | T1185B_Melanoma_ctrl | ncRNA |
| EHSVENGSI (180) | aeTSA | HLA-B38:01 | 0NVC_Melanoma_ctrl | ncRNA |
| ETIQNTHSF (181) | aeTSA | HLA-A26:01 | 0NVC_Melanoma_ctrl | Introns |
| YYSIWKTQI (182) | aeTSA | HLA-A23:01 | Me275_Melanoma | ncRNA |
| SYYFHGLSF (183) | aeTSA | HLA-C07:01 | 0MM745_Melanoma_ctrl | Introns |
| ESFQQPYIPR (184) | aeTSA | HLA-A68:01 | T1185B_Melanoma_ctrl | Introns |
| KQVPSRVLY (185) | aeTSA | HLA-A30:02 | 0MM745_Melanoma_ctrl | retained intron |
| MLFNKDLDAA (186) | aeTSA | HLA-A02:01 | Me275_Melanoma | Introns |
| APRSAEPRAT (187) | aeTSA | HLA-B07:02 | Me275_Melanoma | Intergenic |
| YLDPGAVHAVF (188) | aeTSA | HLA-A02:01 | Me275_Melanoma | Intergenic |
| TLKKHKIV (189) | aeTSA | HLA-B08:01 | 0MM745_Melanoma_ctrl | annotated_ORF |
| VGVPISTLY (190) | aeTSA | HLA-A30:02 | 0MM745_Melanoma_ctrl | Intergenic |
| SPRPQPEL (191) | aeTSA | HLA-B07:02 | 0D5P_Melanoma_ctrl | Intergenic |
| KLSFPDSKF (192) | aeTSA | HLA-B15:01 | 0D5P_Melanoma_ctrl | Intergenic |
| KNKDEYIHFY (193) | aeTSA | HLA-A30:02 | 0MM745_Melanoma_ctrl | Introns |
| RAFEGRRRGW (194) | aeTSA | HLA-A32:01 | T1015A_Melanoma | ncRNA |
| RLKARPSTY⁷ (195) | aeTSA | HLA-B15:01 | 0D5P_Melanoma_ctrl | ncRNA |
| APSLHATL (196) | aeTSA | HLA-B07:02 | 0D5P_Melanoma_ctrl | Introns |
| AQKEPTGNY (197) | aeTSA | HLA-A30:02 | 0MM745_Melanoma_ctrl | Intergenic |
| LSLGTTACR (198) | aeTSA | HLA-A68:01 | T1185B_Melanoma_ctrl | Intergenic |
| SRNPTSYKY (199) | aeTSA | HLA-C07:01 | 0MM745_Melanoma_ctrl | Intergenic |
| SLNNDDHQIY (200) | aeTSA | HLA-B15:01 | 0D5P_Melanoma_ctrl | annotated_ORF |
| YVDPMITEI (201) | aeTSA | HLA-A02:01 | Me290_Melanoma | annotated_ORF |
| PSFSPYAKR (202) | aeTSA | HLA-A68:01 | T1185B_Melanoma_ctrl | Intergenic |
| HVNGSRPHAR (203) | aeTSA | HLA-A68:01 | T1185B_Melanoma_ctrl | Frameshift |
| ITVRLAPPL (204) | aeTSA | HLA-C03:03 | 1496_16T_Melanoma | ncRNA |
| SANPLSHRY (205) | aeTSA | HLA-C07:01 | T1015A_Melanoma | Introns |
| VPLAHSSSA (206) | TAA | HLA-B07:02 | 0D5P_Melanoma_ctrl | annotated_ORF |
| GQVPTTEVV (207) | TAA | HLA-B13:02, | 1496_16T_Melanoma | annotated_ORF |
| | | HLA-B15:01 | | |
| KTWGQYWQV (208) | TAA | HLA-A02:01, | 1758_19T, 542_20T, 534_20T, | annotated_ORF |
| | | HLA-A32:01 | T1015A_Melanoma, | |
| | | | Me290_Melanoma | |
| ALNFPGSQK (209) | TAA | HLA-A03:01 | 1836_18T | annotated_ORF |
| YVWKTWGQY (210) | TAA | HLA-B15:01 | 1682_16T_Melanoma | annotated_ORF |
| MPTAESTGM (211) | TAA | HLA-B35:03 | 1836_18T, T1015A_Melanoma | annotated_ORF |
| HTMEVTVYHRR (212) | TAA | HLA-A68:01 | T1185B_Melanoma_ctrl | annotated_ORF |
| EEAAGIGIL (213) | TAA | HLA-B40:02 | 1758_19T | annotated_ORF |
| EGFDHRDSK (214) | TAA | HLA-A68:01 | T1185B_Melanoma_ctrl | annotated_ORF |
| AEEAAGIGIL (215) | TAA | HLA-B40:02, | 1758_19T | annotated_ORF |
| | | HLA-B40:01 | | |
| LTSSMVTPR (216) | TAA | HLA-A68:01 | T1185B_Melanoma_ctrl | Frameshift |
| TSSMVTPRR (217) | TAA | HLA-A68:01 | T1185B_Melanoma_ctrl | Frameshift |
| GLSNLTHVL (218) | TAA | HLA-A02:01 | Me290_Melanoma, | annotated_ORF |
| | | | Me275_Melanoma | |
| QLLALLPSL (219) | TAA | HLA-A02:01 | 542_20T, 738_20T, 534_20T | annotated_ORF |
| EAAQPMTKK (220) | TAA | HLA-A68:01 | T1185B_Melanoma_ctrl | annotated_ORF |
| RVLGTKPSW (221) | TAA | HLA-A32:01 | T1015A_Melanoma | annotated_ORF |
| VLYPVPLESY (222) | TAA | HLA-B15:01 | 1367_16T, 0D5P_Melanoma_ctrl | annotated_ORF |
| PYLGQMINL (223) | TAA | HLA-A23:01 | Me275_Melanoma, | annotated_ORF |
| | | | 0D5P_Melanoma_ctrl | |
| ELFSYLIEK (224) | TAA | HLA-A68:01 | T1185B_Melanoma_ctrl | annotated_ORF |
| EEQYIAQF (225) | TAA | HLA-B18:01 | 258_20T | annotated_ORF |
| EAAQPMTKKR (226) | TAA | HLA-A68:01 | T1185B_Melanoma_ctrl | annotated_ORF |
| MPMQDIKMI (227) | TAA | HLA-B51:01 | 1758_19T | annotated_ORF |
| RLSEGDVMHL (228) | TAA | HLA-A02:01 | Me290_Melanoma, | annotated_ORF |
| | | | Me275_Melanoma | |
| HASSYISPEK (229) | TAA | HLA-A68:01 | T1185B_Melanoma_ctrl | annotated_ORF |
| MPMQDIKMIL (230) | TAA | HLA-B35:03 | 1836_18T | annotated_ORF |
| SPSVSQLSVL (231) | TAA | HLA-B07:02 | 542_20T, Me275_Melanoma, | annotated_ORF |
| | | | 0D5P_Melanoma_ctrl | |
| RLDQLLRHV (232) | TAA | HLA-A02:09, | T1015A_Melanoma, | annotated_ORF |
| | | HLA-A02:01 | Me290_Melanoma | |
| ALLERASATL (233) | TAA | HLA-A02:01 | Me290_Melanoma | annotated_ORF |
| TVAPTPATAR (234) | TAA | HLA-A68:01 | T1185B_Melanoma_ctrl | 5UTR |
| SQSPSVSQL (235) | TAA | HLA-B15:01 | 0D5P_Melanoma_ctrl | annotated_ORF |
| STVAPTPATAR (236) | TAA | HLA-A68:01 | T1185B_Melanoma_ctrl | 5UTR |
| QYIAQFTSQF (237) | TAA | HLA-A23:01 | Me275_Melanoma, | annotated_ORF |
| | | | 0D5P_Melanoma_ctrl | |
| GQHLHLETF (238) | TAA | HLA-B15:01 | 1367_16T, 0D5P_Melanoma_ctrl | annotated_ORF |
| TIASVLVAR (239) | TAA | HLA-A68:01 | T1185B_Melanoma_ctrl | annotated_ORF |
| SLLQHLIGL (240) | TAA | HLA-A02:01 | 1758_19T, 542_20T, 738_20T, | annotated_ORF |
| | | | 534_20T | |
| TASPRSPVM (241) | TAA | HLA-C03:04 | T1185B_Melanoma_ctrl | annotated_ORF |
| AAFDGRHSQTL (242) | TAA | HLA-C12:03, | 542_20T, 0NVC_Melanoma_ctrl, | annotated_ORF |
| | | HLA-C03:04 | 0D5P_Melanoma_ctrl | |
| SPRRLVEL (243) | TAA | HLA-B07:02 | 0D5P_Melanoma_ctrl | annotated_ORF |
| LYVDSLFFL (244) | TAA | HLA-A24:02 | 1772_19T, 1758_19T | annotated_ORF |
| VFLREFPNF (245) | TAA | HLA-A23:01 | Me275_Melanoma | 3UTR |
| RPRFSTATEIA (246) | TAA | HLA-B07:02 | 0D5P_Melanoma_ctrl | annotated_ORF |
| ALIANPRRK (247) | TAA | HLA-A03:01 | 1836_18T | annotated_ORF |
| DFMGQIVYR (248) | TAA | HLA-A68:01, | T1185B_Melanoma_ctrl, | annotated_ORF |
| | | HLA-A33:01 | 1682_16T_Melanoma | |
| EIPDTVMGL (249) | TAA | HLA-A25:01 | 1713_19T | annotated_ORF |
| VMSSTLYTV (250) | TAA | HLA-A02:01 | 534_20T, | annotated_ORF |
| | | | T1185B_Melanoma_ctrl, | |
| | | | Me275_Melanoma | |
| SPQHKPRL (251) | TAA | HLA-B07:02 | 0D5P_Melanoma_ctrl | annotated_ORF |
| EPKPPTTGL (252) | TAA | HLA-B07:02 | 0D5P_Melanoma_ctrl | Introns |
| IPDTIASVL (253) | TAA | HLA-B35:03 | 1836_18T | annotated_ORF |
| TDFMGQIVYR (254) | TAA | HLA-A68:01 | T1185B_Melanoma_ctrl | annotated_ORF |
| GEDDEHRKI (255) | TAA | HLA-B49:01 | T1015A_Melanoma | ncRNA |
| RLRGSIQSRY⁸ (256) | TAA | HLA-A30:02 | 0MM745_Melanoma_ctrl | annotated_ORF |
| HLDESPAYL (257) | TAA | HLA-C04:01, | T1015A_Melanoma | annotated_ORF |
| | | HLA-A02:01 | | |
| KMEETTVTY (258) | TAA | HLA-B15:01 | 0D5P_Melanoma_ctrl | ncRNA |
| LPTDLFNSV (259) | TAA | HLA-B51:01, | 1758_19T, T1015A_Melanoma | annotated_ORF |
| | | HLA-B35:03 | | |
| RPQGLAVL (260) | TAA | HLA-B07:02 | 0D5P_Melanoma_ctrl | annotated_ORF |
| SLSEKSPEV (261) | TAA | HLA-A02:01 | T1185B_Melanoma_ctrl | 3UTR |
| GAFREGPPY (262) | TAA | HLA-B15:01 | 0D5P_Melanoma_ctrl | annotated_ORF |
| YLNGATVVA (263) | TAA | HLA-A02:01 | Me290_Melanoma, | annotated_ORF |
| | | | Me275_Melanoma | |
| SMYLHTEGF (264) | TAA | HLA-A32:01 | T1015A_Melanoma | annotated_ORF |
| HILSHLHEV (265) | TAA | HLA-A02:01, | 1758_19T, T1015A_Melanoma, | annotated_ORF |
| | | HLA-A02:09 | T1185B_Melanoma_ctrl, | |
| | | | Me290_Melanoma, | |
| | | | Me275_Melanoma, | |
| | | | 1496_16T_Melanoma | |
| SISEAPLTEV (266) | TAA | HLA-A02:01 | Me290_Melanoma | annotated_ORF |
| HQINSEATF (267) | TAA | HLA-B15:01 | 0D5P_Melanoma_ctrl | annotated_ORF |
| DIKQEGVGAF (268) | TAA | HLA-A26:01 | 0NVC_Melanoma_ctrl | annotated_ORF |
| SALGVTITK (269) | TAA | HLA-A68:01 | T1185B_Melanoma_ctrl | annotated_ORF |
| TVNDFTQNPR (270) | TAA | HLA-A68:01 | T1185B_Melanoma_ctrl | annotated_ORF |
| SFIQAGSSY (271) | TAA | HLA-C14:02 | 0D5P_Melanoma_ctrl | annotated_ORF |
| GVTITKTLK (272) | TAA | HLA-A03:01 | 1836_18T | annotated_ORF |
| AERPQGLAV (273) | TAA | HLA-B49:01 | T1015A_Melanoma | annotated_ORF |
| REKGPVKEV (274) | TAA | HLA-B49:01 | T1015A_Melanoma | annotated_ORF |
| TSVPPFNLR (275) | TAA | HLA-A68:01 | T1185B_Melanoma_ctrl | annotated_ORF |
| LSRLPSIAF (276) | TAA | HLA-B15:01 | 0D5P_Melanoma_ctrl | ncRNA |
| FPVHAAEKF (277) | TAA | HLA-B35:03 | T1015A_Melanoma | ncRNA |
| GEISASHV (278) | TAA | HLA-B49:01 | T1015A_Melanoma | annotated_ORF |
| AADGGQHWTY (279) | TAA | HLA-A01:01 | Me290_Melanoma, | annotated_ORF |
| | | | 0D5P_Melanoma_ctrl | |
| EISASHVSR (280) | TAA | HLA-A68:01 | T1185B_Melanoma_ctrl | annotated_ORF |
| SPRIPFSTF (281) | TAA | HLA-B07:02 | 0D5P_Melanoma_ctrl | annotated_ORF |
| KLMDPGSLPPL (282) | TAA | HLA-A02:09, | T1015A_Melanoma, | annotated_ORF |
| | | HLA-A02:01 | Me290_Melanoma, | |
| | | | Me275_Melanoma | |
| ETKPEFERR (283) | TAA | HLA-A68:01 | T1185B_Melanoma_ctrl | annotated_ORF |
| ETIMKFLKM (284) | TAA | HLA-A26:01 | 0NVC_Melanoma_ctrl | annotated_ORF |
| RRAHLWSTF (285) | TAA | HLA-C07:01 | T1015A_Melanoma | Intergenic |
| HLQEKLQSL (286) | TAA | HLA-A02:01, | 1758_19T, Me290_Melanoma, | annotated_ORF |
| | | HLA-B08:01 | Me275_Melanoma | |
| ELSVVTSER (287) | TAA | HLA-A68:01 | T1185B_Melanoma_ctrl | annotated_ORF |
| YLDQQVPYTF (288) | TAA | HLA-A01:01 | Me290_Melanoma | annotated_ORF |
| EAIPRIPRL (289) | TAA | HLA-A25:01, | 1713_19T, | annotated_ORF |
| | | HLA-C12:03 | 0NVC_Melanoma_ctrl | |
| GYLDQQVPYTF (290) | TAA | HLA-A23:01 | 0D5P_Melanoma_ctrl | annotated_ORF |
| SPKLGGIGF (291) | TAA | HLA-B07:02 | Me275_Melanoma, | annotated_ORF |
| | | | 0D5P_Melanoma_ctrl | |
| YPEKSRGCL (292) | TAA | HLA-B07:02 | 0D5P_Melanoma_ctrl | annotated_ORF |
| TTIPFSTLRQR (293) | TAA | HLA-A68:01 | T1185B_Melanoma_ctrl | 3UTR |
| SLGAHSPALY (294) | TAA | HLA-A30:02 | 0MM745_Melanoma_ctrl | annotated_ORF |
| SQNGQLIHY (295) | TAA | HLA-B15:01 | 1367_16T, 0D5P_Melanoma_ctrl | annotated_ORF |
| KAKEHTANISW (296) | TAA | HLA-A32:01 | T1015A_Melanoma | annotated_ORF |
| RVSKSDSTF (297) | TAA | HLA-B15:01 | 0D5P_Melanoma_ctrl | annotated_ORF |
| SLNSQVTGL (298) | TAA | HLA-A02:01 | Me275_Melanoma | annotated_ORF |
| NSDLSEKLEY (299) | TAA | HLA-A01:01 | 1836_18T, | annotated_ORF |
| | | | 0MM745_Melanoma_ctrl, | |
| | | | Me290_Melanoma, | |
| | | | 0D5P_Melanoma_ctrl | |
| DSSTVGLQR (300) | TAA | HLA-A68:01 | T1185B_Melanoma_ctrl | annotated_ORF |
| RASAIIIQR (301) | TAA | HLA-A68:01 | T1185B_Melanoma_ctrl | annotated_ORF |
| SQETLPLKI (302) | TAA | HLA-B37:01 | 1496_16T_Melanoma | annotated_ORF |
| STFDGLDRR (303) | TAA | HLA-A68:01 | T1185B_Melanoma_ctrl | annotated_ORF |
| RVKEIVINK (304) | TAA | HLA-A30:01 | 542_20T | annotated_ORF |
| AQPHHGVPY (305) | TAA | HLA-B15:01 | 0D5P_Melanoma_ctrl | annotated_ORF |
| EVIQLKERDRK (306) | TAA | HLA-A68:01 | T1185B_Melanoma_ctrl | annotated_ORF |
| EVKEKTEL (307) | TAA | HLA-B08:01 | 0MM745_Melanoma_ctrl | annotated_ORF |
| TDPETLHY (308) | TAA | HLA-A01:01 | 0D5P_Melanoma_ctrl | annotated_ORF |
| KMKEKTQEL (309) | TAA | HLA-B08:01, | 0D5P_Melanoma_ctrl | annotated_ORF |
| | | HLA-B15:01 | | |
| STFDGLDRRR (310) | TAA | HLA-A68:01 | T1185B_Melanoma_ctrl | annotated_ORF |
| KMSELQTYV (311) | TAA | HLA-A02:01, | 1758_19T, 542_20T, | annotated_ORF |
| | | HLA-A02:09, | T1015A_Melanoma, | |
| | | HLA-B13:02 | T1185B_Melanoma_ctrl, | |
| | | | Me290_Melanoma, | |
| | | | Me275_Melanoma, | |
| | | | 1496_16T_Melanoma | |
| GTDPETLHY (312) | TAA | HLA-A01:01 | 0D5P_Melanoma_ctrl | annotated_ORF |
| TEEKGSILI (313) | TAA | HLA-B49:01 | T1015A_Melanoma | annotated_ORF |
| DTAMPAHSR (314) | TAA | HLA-A68:01 | T1185B_Melanoma_ctrl | annotated_ORF |
| KLQEALKTI (315) | TAA | HLA-A02:09, | T1015A_Melanoma, | annotated_ORF |
| | | HLA-A02:01 | Me290_Melanoma, | |
| | | | Me275_Melanoma | |
| GTDPETLHYF (316) | TAA | HLA-A01:01 | 0D5P_Melanoma_ctrl | annotated_ORF |
| SLKNRIESV (317) | TAA | HLA-B08:01, | 0MM745_Melanoma_ctrl, | annotated_ORF |
| | | HLA-A02:09, | T1015A_Melanoma, | |
| | | HLA-A02:01 | Me290_Melanoma, | |
| | | | Me275_Melanoma | |
| NLKDPVGVY (318) | TAA | HLA-B15:01 | 0D5P_Melanoma_ctrl | annotated_ORF |
| FTETNPQVY (319) | TAA | HLA-A01:01 | 1836_18T, 0D5P_Melanoma_ctrl | annotated_ORF |
| ETQEAEVAVSR (320) | TAA | HLA-A68:01 | T1185B_Melanoma_ctrl | Intergenic |
| SPRPESGTV (321) | TAA | HLA-B07:02 | Me275_Melanoma | annotated_ORF |
| SEFAKDPSL (322) | TAA | HLA-B40:01 | T1185B_Melanoma_ctrl | annotated_ORF |
| KVIKKIGHY (323) | TAA | HLA-A30:02 | 0MM745_Melanoma_ctrl | annotated_ORF |
| RTWQTRKQF (324) | TAA | HLA-A32:01 | T1015A_Melanoma | annotated_ORF |
| AMAHKVIKA (325) | TAA | HLA-A02:01 | Me290_Melanoma, | annotated_ORF |
| | | | Me275_Melanoma | |
| KLKQFQKEY (326) | TAA | HLA-A32:01, | T1015A_Melanoma, | annotated_ORF |
| | | HLA-B15:01 | 0D5P_Melanoma_ctrl | |
| SPLEDEESL (327) | TAA | HLA-B35:03 | 1836_18T | annotated_ORF |
| NSTDFTTDR (328) | TAA | HLA-A68:01 | T1185B_Melanoma_ctrl | annotated_ORF |
| ETKDTKGRYF (329) | TAA | HLA-A26:01 | 0NVC_Melanoma_ctrl | annotated_ORF |
| RPRVSSTEM (330) | TAA | HLA-B07:02 | 0D5P_Melanoma_ctrl | annotated_ORF |
| LMDKSSTIY (331) | TAA | HLA-A01:01 | 0D5P_Melanoma_ctrl | annotated_ORF |
| GTLRPLKVRR (332) | TAA | HLA-A68:01 | T1185B_Melanoma_ctrl | annotated_ORF |
| ILSEGSVPL (333) | TAA | HLA-A02:01 | 1758_19T | annotated_ORF |
| TVYNFRTEL (334) | TAA | HLA-C03:04, | Me290_Melanoma | Intergenic |
| | | HLA-C07:01 | | |
| LPSSADVEF (335) | LSA | HLA-B35:03 | 1836_18T | annotated_ORF |
| RLPSSADVEF (336) | LSA | HLA-B15:01 | 0D5P_Melanoma_ctrl | annotated_ORF |
| ALMDKSLHV (337) | LSA | HLA-A02:01, | 1758_19T,542_20T,T1015A_Mel | annotated_ORF |
| | | HLA-A02:09 | anoma, T1185B_Melanoma_ctrl | |
| MHNALHIYM (338) | LSA | HLA-B38:01 | 542_20T | annotated_ORF |
| MEKEDYHSL (339) | LSA | HLA-B40:02, | 1758_19T, T1015A_Melanoma, | annotated_ORF |
| | | HLA-B49:01, | Me290_Melanoma | |
| | | HLA-B08:01, | | |
| | | HLA-C07:02 | | |
| MEKEDYHSLY (340) | LSA | HLA-A01:01 | Me290_Melanoma | annotated_ORF |
| YHSLYQSHL (341) | LSA | HLA-B38:01 | 542_20T, 0NVC_Melanoma_ctrl | annotated_ORF |
| YMVPFIPLY (342) | LSA | HLA-B15:01 | 1367_1 | annotated_ORF |
| LMEKEDYHSL (343) | LSA | HLA-B08:01 | Me290_Melanoma | annotated_ORF |
| FAHEAPAFL (344) | LSA | HLA-C12:03 | 0D5P_Melanoma_ctrl | annotated_ORF |
| FASPLTGIA (345) | LSA | HLA-C12:03 | 542_20T | annotated_ORF |
| HEAPAFLPW (346) | LSA | HLA-B44:02 | 1836_18T | annotated_ORF |
| VLFKKFIQK (347) | LSA | HLA-A03:01 | 1836_18T | Frameshift |
| APAFLPWHR (348) | LSA | HLA-A68:01 | T1185B_Melanoma_ctrl | annotated_ORF |
| LLMEKEDYHSL (349) | LSA | HLA-A02:01, | 1758_19T, 542_20T, 738_20T, | annotated_ORF |
| | | HLA-B08:01, | 534_20T, T1015A_Melanoma, | |
| | | HLA-A02:09 | T1185B_Melanoma_ctrl, | |
| | | | Me290_Melanoma | |
| HYYVSMDAL (350) | LSA | HLA-C14:02 | 0D5P_Melanoma_ctrl | annotated_ORF |
| WEQEIQKL (351) | LSA | HLA-B40:02 | 1758_19T | annotated_ORF |
| LMEKEDYHSLY (352) | LSA | HLA-A01:01 | 1713_19T, 1836_18T, 534_20T, | annotated_ORF |
| | | | Me290_Melanoma, | |
| | | | 0D5P_Melanoma_ctrl | |
| LEQASRIWSW (353) | LSA | HLA-B44:02 | 1836_18T | annotated_ORF |
| NIFDLSAPEK (354) | LSA | HLA-A68:01 | T1185B_Melanoma_ctrl | annotated_ORF |
| EAPAFLPWHR (355) | LSA | HLA-A68:01 | T1185B_Melanoma_ctrl | annotated_ORF |
| SEIWRDIDF (356) | LSA | HLA-B44:02 | 1836_18T | annotated_ORF |
| AAMVGAVL (357) | LSA | HLA-C12:03 | 258_20T,542_20T | annotated_ORF |
| TFLGVGQESF (358) | LSA | HLA-A23:01 | 0D5P_Melanoma_ctrl | annotated_ORF |
| ESWPSVFYNR (359) | LSA | HLA-A68:01 | T1185B_Melanoma_ctrl | annotated_ORF |
| ISQDTTVGR (360) | LSA | HLA-A68:01 | T1185B_Melanoma_ctrl | Frameshift |
| RPWSGPYIL (361) | LSA | HLA-B07:02 | 542_20T, Me275_Melanoma, | annotated_ORF |
| | | | 0D5P_Melanoma_ctrl | |
| SSDYVIPIGTY (362) | LSA | HLA-A01:01 | 0D5P_Melanoma_ctrl | annotated_ORF |
| DSDPDSFQDY (363) | LSA | HLA-A01:01 | 1713_19T, 1367_16T, | annotated_ORF |
| | | | Me290_Melanoma | |
| DTRPWSGPY (364) | LSA | HLA-A26:01 | 0NVC_Melanoma_ctrl | annotated_ORF |
| HYYSVKKTF (365) | LSA | HLA-C14:02 | 0D5P_Melanoma_ctrl | annotated_ORF |
| NVCGSQQGR (366) | LSA | HLA-A68:01 | T1185B_Melanoma_ctrl | annotated_ORF |
| YMNGTMSQV (367) | LSA | HLA-C01:02, | 2883_18T, 1758_19T, | annotated_ORF |
| | | HLA-A02:01, | 1713_19T, 542_20T, 738_20T, | |
| | | HLA-C12:03, | 534_20T, T1015A_Melanoma, | |
| | | HLA-A02:09 | T1185B_Melanoma_ctrl, | |
| | | | Me290_Melanoma, | |
| | | | Me275_Melanoma, | |
| | | | 0D5P_Melanoma_ctrl, | |
| | | | 1496_16T_Melanoma, | |
| | | | 1682_16T_Melanoma | |
| MAKRTTHPL (368) | LSA | HLA-C12:03 | 0D5P_Melanoma_ctrl | annotated_ORF |
| RPWSGPYILR (369) | LSA | HLA-A68:01 | T1185B_Melanoma_ctrl | annotated_ORF |
| MTVDSLVNK (370) | LSA | HLA-A68:01 | T1185B_Melanoma_ctrl | annotated_ORF |
| TRPWSGPYIL (371) | LSA | HLA-C07:02 | Me275_Melanoma | annotated_ORF |
| TTHPLFVIATR (372) | LSA | HLA-A68:01 | T1185B_Melanoma_ctrl | annotated_ORF |
| FVWTHYYSV (373) | LSA | HLA-A02:01 | Me290_Melanoma | annotated_ORF |
| LSKEEKNHF (374) | LSA | HLA-B15:01 | 0D5P_Melanoma_ctrl | annotated_ORF |
| RAANQVFVL (375) | LSA | HLA-C12:03 | 0D5P_Melanoma_ctrl | 5UTR |
| MVRSATLIK (376) | LSA | HLA-A30:01 | 542_20T | 5UTR |
| NQVFVLHSY (377) | LSA | HLA-B15:01 | 0D5P_Melanoma_ctrl | 5UTR |
| HNSTEFLI (378) | LSA | HLA-B38:01 | 542_20T | annotated_ORF |
| SQDVAGTTF (379) | LSA | HLA-C08:02 | 1367_16T | annotated_ORF |
| AHNSTEFLI (380) | LSA | HLA-B38:01 | 542_20T | annotated_ORF |
| STEFLIYER (381) | LSA | HLA-A68:01 | T1185B_Melanoma_ctrl | annotated_ORF |
| EGFFTRQER (382) | LSA | HLA-A68:01 | T1185B_Melanoma_ctrl | annotated_ORF |
| FQKVLVSY (383) | LSA | HLA-B15:01 | 0D5P_Melanoma_ctrl | annotated_ORF |
| FTDFMGQIVY (384) | LSA | HLA-A01:01 | 1713_19T, 1836_18T, 534_20T, | annotated_ORF |
| | | | Me290_Melanoma, | |
| | | | 0D5P_Melanoma_ctrl | |
| YFQKVLVSY (385) | LSA | HLA-C14:02 | 0D5P_Melanoma_ctrl | annotated_ORF |
| ALVNMPPHYR (386) | LSA | HLA-A68:01 | T1185B_Melanoma_ctrl | annotated_ORF |
| GVMSSTLYTV (387) | LSA | HLA-A02:01 | T1185B_Melanoma_ctrl | annotated_ORF |
| LLSNTVSTL (388) | LSA | HLA-C03:03, | 1367_16T, | annotated_ORF |
| | | HLA-A02:01 | T1185B_Melanoma_ctrl | |
| RVFHETGAQR (389) | LSA | HLA-A68:01 | T1185B_Melanoma_ctrl | Frameshift |
| SASDHCRSR (390) | LSA | HLA-A68:01 | T1185B_Melanoma_ctrl | annotated_ORF |
| RRDGGIIIY (391) | LSA | HLA-C07:01 | T1015A_Melanoma | annotated_ORF |
| VAALIANPR (392) | LSA | HLA-A68:01 | T1185B_Melanoma_ctrl | annotated_ORF |
| RLLGTEFQV (393) | LSA | HLA-A02:01, | 1758_19T, 542_20T, 534_20T, | annotated_ORF |
| | | HLA-B13:02 | T1185B_Melanoma_ctrl, | |
| | | | Me290_Melanoma, | |
| | | | Me275_Melanoma | |
| FSIIGVSRR (394) | LSA | HLA-A68:01 | T1185B_Melanoma_ctrl | Frameshift |
| LSSDGMYEY (395) | LSA | HLA-A01:01 | Me290_Melanoma | annotated_ORF |
| DEYFLPSL (396) | LSA | HLA-B18:01, | 1713_19T, 258_20T, 738_20T | annotated_ORF |
| | | HLA-B14:01 | | |
| SYIGLKGLYF (397) | LSA | HLA-A23:01 | Me275_Melanoma | annotated_ORF |
| FTGFGGALGY (398) | LSA | HLA-A26:01, | Me290_Melanoma, | annotated_ORF |
| | | HLA-A01:01 | 0D5P_Melanoma_ctrl | |
| KTWDQVPFSV (399) | LSA | HLA-A02:01, | 542_20T, 534_20T, | annotated_ORF |
| | | HLA-A02:09 | T1015A_Melanoma, | |
| | | | T1185B_Melanoma_ctrl, | |
| | | | Me290_Melanoma, | |
| | | | Me275_Melanoma | |
| RVADIPIKP (400) | LSA | HLA-B13:02 | T1185B_Melanoma_ctrl | Frameshift |
| SVAKHTQSY (401) | LSA | HLA-A32:01, | T1015A_Melanoma, | annotated_ORF |
| | | HLA-B15:01 | 0D5P_Melanoma_ctrl | |
| QTKGGQTWAR (402) | LSA | HLA-A68:01 | T1185B_Melanoma_ctrl | annotated_ORF |
| ETGSSFMSF (403) | LSA | HLA-A25:01 | 1713_19T | ncRNA |
| SLYSYFQKV (404) | LSA | HLA-A02:01 | 534_20T, | annotated_ORF |
| | | | T1185B_Melanoma_ctrl, | |
| | | | Me290_Melanoma, | |
| | | | Me275_Melanoma | |
| QTKGGQTWARR (405) | LSA | HLA-A68:01 | T1185B_Melanoma_ctrl | annotated_ORF |
| TVVAALIANPR (406) | LSA | HLA-A68:01 | T1185B_Melanoma_ctrl | annotated_ORF |
| HLMVKDWQL (407) | LSA | HLA-A02:09 | T1015A_Melanoma | annotated_ORF |
| NPLSKLSVL (408) | LSA | HLA-B07:02 | 0D5P_Melanoma_ctrl | annotated_ORF |
| FVIPSTKDSNR⁹ (409) | LSA | HLA-A68:01 | T1185B_Melanoma_ctrl | annotated_ORF |
| HYQVQTHL (410) | LSA | HLA-C14:02 | 0D5P_Melanoma_ctrl | annotated_ORF |
| ASDILSFAHKY (411) | LSA | HLA-A01:01 | Me290_Melanoma, | annotated_ORF |
| | | | 0D5P_Melanoma_ctrl | |
| RLGQGVPLV (412) | LSA | HLA-A02:09, | T1185B_Melanoma_ctrl, | annotated_ORF |
| | | HLA-A02:01 | Me290_Melanoma, | |
| | | | Me275_Melanoma | |
| SVPDHVQPSK (413) | LSA | HLA-A68:01 | T1185B_Melanoma_ctrl | Frameshift |
| RLLEKHISL (414) | LSA | HLA-A32:01, | T1015A_Melanoma, | annotated_ORF |
| | | HLA-A02:01, | T1185B_Melanoma_ctrl, | |
| | | HLA-B08:01 | Me290_Melanoma, | |
| | | | Me275_Melanoma | |
| SLFGVSERL (415) | LSA | HLA-A02:01 | Me275_Melanoma | ncRNA |
| NHYQVQTHL (416) | LSA | HLA-B38:01 | 542_20T, 0NVC_Melanoma_ctrl | annotated_ORF |
| DLVGKDVTRVY (417) | LSA | HLA-A26:01 | 0NVC_Melanoma_ctrl | annotated_ORF |
| SLSPPTLPR (418) | LSA | HLA-A68:01 | T1185B_Melanoma_ctrl | 5UTR |
| LADNGTLGKY (419) | LSA | HLA-A01:01 | Me290_Melanoma, | annotated_ORF |
| | | | 0D5P_Melanoma_ctrl | |
| KAMYIRVSY (420) | LSA | HLA-A32:01 | T1015A_Melanoma | annotated_ORF |
| MQPKLKQVF (421) | LSA | HLA-B15:01 | 0D5P_Melanoma_ctrl | annotated_ORF |
| AIIQDGILL (422) | LSA | HLA-A02:01 | 534_20T | 5UTR |
| AFDRAVHF (423) | LSA | HLA-C12:03, | 0NVC_Melanoma_ctrl, | ncRNA |
| | | HLA-C04:01, | T1015A_Melanoma, | |
| | | HLA-C14:02 | 0D5P_Melanoma_ctrl | |
| NTPNIVSLR (424) | LSA | HLA-A68:01 | T1185B_Melanoma_ctrl | ncRNA |
| RVADIPIKPW (425) | LSA | HLA-A25:01, | 258_20T, T1015A_Melanoma | Frameshift |
| | | HLA-A32:01 | | |
| HYIIPLQTI (426) | LSA | HLA-A23:01 | 0D5P_Melanoma_ctrl | 5UTR |
| VMKGINQGY (427) | LSA | HLA-B15:01 | 0D5P_Melanoma_ctrl | ncRNA |
| ATSPPASVR (428) | LSA | HLA-A68:01 | T1185B_Melanoma_ctrl | annotated_ORF |
| ALEKDTPRY (429) | LSA | HLA-A01:01 | Me290_Melanoma, | annotated_ORF |
| | | | 0D5P_Melanoma_ctrl | |
| QVMKGINQGY (430) | LSA | HLA-A26:01 | 0NVC_Melanoma_ctrl | ncRNA |
| AAFDRAVHF (431) | LSA | HLA-C12:03 | 1713_19T, 542_20T, | ncRNA |
| | | | 0NVC_Melanoma_ctrl, | |
| | | | 0D5P_Melanoma_ctrl | |
| RPLRIKGVF (432) | LSA | HLA-B07:02 | 542_20T, Me275_Melanoma, | ncRNA |
| | | | 0D5P_Melanoma_ctrl | |
| RQVMKGINQGY (433) | LSA | HLA-B15:01 | 0D5P_Melanoma_ctrl | ncRNA |
| KLDTTVKNI (434) | LSA | HLA-A02:01 | Me290_Melanoma, | annotated_ORF_ |
| | | | Me275_Melanoma | frameshift- |
| | | | | 3UTR |
| LPRTPRPDL (435) | LSA | HLA-B07:02 | 0D5P_Melanoma_ctrl | ncRNA |
| ITYTESNSR (436) | LSA | HLA-A68:01 | T1185B_Melanoma_ctrl | Intergenic |
| RLQGSVTLV (437) | LSA | HLA-A02:01 | Me290_Melanoma | ncRNA |
| SQFPSKNLPAF (438) | LSA | HLA-B15:01 | 0D5P_Melanoma_ctrl | ncRNA |
| LPRTPRPDLIL (439) | LSA | HLA-B07:02 | 0D5P_Melanoma_ctrl | ncRNA |
| DTAPQSHQV (440) | LSA | HLA-A26:01 | 0NVC_Melanoma_ctrl | ncRNA |
| MAILGTAGR (441) | LSA | HLA-A68:01 | T1185B_Melanoma_ctrl | Introns |
| SVSSHLIHR (442) | LSA | HLA-A68:01 | T1185B_Melanoma_ctrl | ncRNA |
| STSPGTGVRK (443) | LSA | HLA-A68:01 | T1185B_Melanoma_ctrl | annotated_ORF |
| EVAEVLGSVK (444) | LSA | HLA-A68:01 | T1185B_Melanoma_ctrl | ncRNA |
| EIPRTPSEGR (445) | LSA | HLA-A68:01 | T1185B_Melanoma_ctrl | Intergenic |
| VFLKFSRRF (446) | LSA | HLA-A23:01 | Me275_Melanoma, | ncRNA |
| | | | 0D5P_Melanoma_ctrl | |
| SVSSHLIHRY (447) | LSA | HLA-A26:01 | 0NVC_Melanoma_ctrl | ncRNA |
| THLEDPHLM (448) | LSA | HLA-B38:01 | 0NVC_Melanoma_ctrl | ncRNA |
| SVFPRTSSR (449) | LSA | HLA-A68:01 | T1185B_Melanoma_ctrl | annotated_ORF |
| GQYGQSKPWTF (450) | LSA | HLA-B15:01 | 0D5P_Melanoma_ctrl | annotated_ORF |
| VPRPTSTVGL (451) | LSA | HLA-B07:02 | Me275_Melanoma, | annotated_ORF |
| | | | 0D5P_Melanoma_ctrl | |
| NSVSSHLIHRY (452) | LSA | HLA-A26:01 | 0NVC_Melanoma_ctrl | ncRNA |
| QEEDNLLRYF (453) | LSA | HLA-B44:02 | 1836_18T | ncRNA |
| THLEDPHLML (454) | LSA | HLA-B38:01 | 0NVC_Melanoma_ctrl | ncRNA |
| ATTNILEHV (455) | LSA | HLA-B13:02, | Me290_Melanoma | annotated_ORF |
| | | HLA-A02:01 | | |
| MTLRPTQPR (456) | LSA | HLA-A68:01 | T1185B_Melanoma_ctrl | ncRNA |
| ETRPNNSKF (457) | LSA | HLA-A26:01 | 0NVC_Melanoma_ctrl | ncRNA |
| HSITEPLLY¹⁰ (458) | LSA | HLA-A01:01 | 0D5P_Melanoma_ctrl | Introns |
| HLFLQPPSKR (459) | LSA | HLA-A68:01 | T1185B_Melanoma_ctrl | Intergenic |
| ETVRFTSSF¹¹ (460) | LSA | HLA-A26:01 | 0NVC_Melanoma_ctrl | 5UTR |
| SPMPIPPLYL (461) | LSA | HLA-B07:02 | 0D5P_Melanoma_ctrl | ncRNA |
| ESYGRYTFY (462) | LSA | HLA-A26:01 | 0NVC_Melanoma_ctrl | Introns |
| KSQGSFISF (463) | LSA | HLA-B15:01 | 0D5P_Melanoma_ctrl | Introns |
| GTILHIGVF (464) | LSA | HLA-A25:01 | 1713_19T | retained_intron |
| KVFSGSSKR (465) | LSA | HLA-A68:01 | T1185B_Melanoma_ctrl | annotated_ORF |
| SSFSTLASLDK (466) | LSA | HLA-A68:01 | T1185B_Melanoma_ctrl | ncRNA |
| TTQGAAPQR (467) | LSA | HLA-A68:01 | T1185B_Melanoma_ctrl | Introns |
| HTTQGAAPQR (468) | LSA | HLA-A68:01 | T1185B_Melanoma_ctrl | Introns |
| KIFQERAAF (469) | LSA | HLA-A32:01 | T1015A_Melanoma | 5UTR |
| GQITHNTSF (470) | LSA | HLA-B15:01 | 0D5P_Melanoma_ctrl | annotated_ORF |
| ISLKPLIARY (471) | LSA | HLA-A30:02 | 0MM745_Melanoma_ctrl | Introns |
| KHSDLILFW (472) | LSA | HLA-C07:01 | T1015A_Melanoma | 3UTR |
| MVWEGHPRER (473) | LSA | HLA-A68:01 | T1185B_Melanoma_ctrl | ncRNA |
| STDLPILLK (474) | LSA | HLA-A01:01 | Me290_Melanoma | ncRNA |
| EVISINTGHF (475) | LSA | HLA-A26:01 | 0NVC_Melanoma_ctrl | Intergenic |
| ETERVLEKSSY (476) | LSA | HLA-A01:01 | 0D5P_Melanoma_ctrl | annotated_ORF |
| KIKTINLRY (477) | LSA | HLA-B15:01 | 0D5P_Melanoma_ctrl | Introns |
| YVSQKVSDF (478) | LSA | HLA-C07:02 | Me275_Melanoma | Introns |
| DAGIPVYHR (479) | LSA | HLA-A68:01 | T1185B_Melanoma_ctrl | annotated_ORF |
| TILEAEILHQA (480) | LSA | HLA-B49:01, | T1015A_Melanoma, | Introns |
| | | HLA-A02:01 | Me275_Melanoma | |
| EYAQKTPLF¹² (481) | LSA | HLA-A24:02, | 1758_19T, Me275_Melanoma, | Introns |
| | | HLA-A23:01 | 0D5P_Melanoma_ctrl | |
| TWTPHGIRI (482) | LSA | HLA-A23:01 | 0D5P_Melanoma_ctrl | Introns |
| EVHEGGVARF (483) | LSA | HLA-A68:01 | T1185B_Melanoma_ctrl | annotated_ORF |
| REIEIEGRL (484) | LSA | HLA-B49:01 | T1015A_Melanoma | ncRNA |
| SWLPIPAKY (485) | LSA | HLA-A30:02 | 0MM745_Melanoma_ctrl | annotated_ORF |
| RYSERVYTI (486) | LSA | HLA-A23:01 | 0D5P_Melanoma_ctrl | annotated_ORF |
| FTDSQGNDIK (487) | LSA | HLA-A01:01 | Me290_Melanoma | annotated_ORF |
| SVPSDFLSLR (488) | LSA | HLA-A68:01 | T1185B_Melanoma_ctrl | Intergenic |
| RQMSLEFRA (489) | LSA | HLA-A02:01 | Me275_Melanoma | Introns |
| ETISPRPSI (490) | LSA | HLA-A25:01, | 1713_19T, | ncRNA |
| | | HLA-A26:01 | 0NVC_Melanoma_ctrl | |
| GPAGGRGVI (491) | LSA | HLA-B07:02 | 0D5P_Melanoma_ctrl | annotated_ORF |
| LTDHHIKEY (492) | LSA | HLA-A01:01 | Me290_Melanoma | Introns |
| EAVPYNGSLFK (493) | LSA | HLA-A68:01 | T1185B_Melanoma_ctrl | ncRNA |
| NLLAEIHGV (494) | LSA | HLA-A02:09, | T1015A_Melanoma, | annotated_ORF |
| | | HLA-A02:01 | Me290_Melanoma, | |
| | | | Me275_Melanoma | |
| KEGPVTRGA (495) | LSA | HLA-B49:01 | T1015A_Melanoma | 5UTR |
| VHVDLNLGF (496) | LSA | HLA-B38:01 | 0NVC_Melanoma_ctrl | Introns |
| HTVQGLAGF (497) | LSA | HLA-A26:01 | 0NVC_Melanoma_ctrl | Introns |
| MRIEDSHTKL¹³ (498) | LSA | HLA-B38:01 | 0NVC_Melanoma_ctrl | ncRNA |
| SALSSAQPR (499) | LSA | HLA-A68:01 | T1185B_Melanoma_ctrl | annotated_ORF |
| SPSIKSLLVY (500) | LSA | HLA-A01:01 | Me290_Melanoma | Introns |
| PTDATLNRLLY (501) | LSA | HLA-A01:01 | 0D5P_Melanoma_ctrl | annotated_ORF |
| WTSQPPNYEK (502) | LSA | HLA-A68:01 | T1185B_Melanoma_ctrl | Introns |
| PAPRPVAPTL (503) | LSA | HLA-B07:02 | 0D5P_Melanoma_ctrl | Introns |
| VYLHSNSTL (504) | LSA | HLA-C14:02 | 0D5P_Melanoma_ctrl | Intergenic |
| SLYAWTQSH (505) | LSA | HLA-A03:01 | 0NVC_Melanoma_ctrl | Intergenic |

¹Reference peptide: ATYGGSDDYI
²Reference peptide: HLRDYFEEY
³Reference peptide: NEYPNITRLY
⁴Reference peptide: RLLADPTGA
⁵Reference peptide: TPLSPGPTL
⁶Reference peptide: VCLDRAVSI
⁷Reference peptide: RLKATPSTY
⁸Reference peptide: RLWGSIQSRY
⁹Reference peptide: FVIPSMKDSNR
¹⁰Reference peptide: HSIMEPLLY
¹¹Reference peptide: ETVKFTSSF
¹²Reference peptide: EYAQKTLLF
¹³Reference peptide: MHIEDSHTKL

TABLE 2B
characteristics of the TSAs, TAAs and LSAs identified herein (continued)

| Peptide | | | |
|---|---|---|---|
| (SEQ ID | | | |
| NO:) | Gene | Gene ID | Nucleotide (SEQ ID NO:) |
| 1 | NA | NA | ACTCTCGATGACGCCGTCCACGGCGTT (506) |
| 2 | ZYG11AP1 | ENSG00000232242 | TACCAAATTGGCCAAGTTCAAGGTGTA (507) |
| 3 | CPD | ENSG00000108582 | AATGAATATTCTAACATTACCCGGCTTTAT (508) |
| 4 | PRDX5 | ENSG00000126432 | CGGCTCCTGGCTGATCTCACTGGGGCC (509) |
| 5 | KIAA0895L | ENSG00000196123 | ACTCCATTGTCTCCTGACCCCACCCTC (510) |
| 6 | GAGE12C; | ENSG00000237671; | TCGACCTATTATTGGCCTAGACCAAGG (511) |
| | GAGE12G | ENSG00000215269 | |
| 7 | XAGE1B | ENSG00000204382 | GATTCTTTCTCCGCTACTGAGACACGG (512) |
| 8 | MAGEA6 | ENSG00000197172 | GAAACCAGCTATGTGAAAGTCCTGCAC (513) |
| 9 | MAGEA6 | ENSG00000197172 | GCAGAAATGCTGGGGAGTGTCGTC (514) |
| 10 | MAGEA3 | ENSG00000221867 | GAGGAGCTGAGTGTGTTAGAGGTGTTT (515) |
| 11 | MAGEA6; | ENSG00000197172; | TTCGTGCAGGAAAACTACCTGGAGTAC (516) |
| | MAGEA3 | ENSG00000221867 | |
| 12 | PAGE5 | ENSG00000158639 | ATTGTCCAGCAGCCCACTGAGGAAAAA (517) |
| 13 | MAGEA12; | ENSG00000213401; | AGTGTCTTTGCGCATCCCAGGAAGCTG (518); |
| | MAGEA2 | ENSG00000268606 | AGTGTCTTCGCACATCCCAGGAAGCTG (519) |
| 14 | MAGEA3 | ENSG00000221867 | GCCTCCAGCCTCCCCACTACCATGAACTAC (520) |
| 15 | PAGE5 | ENSG00000158639 | ATTGTCCAGCAGCCCACTGAGGAAAAACGT (521) |
| 16 | MAGEA3 | ENSG00000221867 | CTCCCCACTACCATGAACTACCCTCTC (522) |
| 17 | MAGEA4 | ENSG00000147381 | GGCAGTAATCCTGCGCGCTATGAGTTC (523) |
| 18 | MAGEC2 | ENSG00000046774 | ACCGCAGATGATGCCACTGTCATG (524) |
| 19 | MAGEA4 | ENSG00000147381 | GTCCTGGAGCATGTGGTCAGGGTC (525) |
| 20 | MAGEA3 | ENSG00000221867 | GAAGTGGACCCCATCGGCCACTTGTAC (526) |
| 21 | MAGEA3 | ENSG00000221867 | CAGATCATGCCCAAGGCAGGCCTC (527) |
| 22 | MAGEA4 | ENSG00000147381 | AAAGTCCTGGAGCATGTGGTCAGGGTC (528) |
| 23 | MAGEA4 | ENSG00000147381 | CAGGTACCCGGCAGTAATCCTGCGCGC (529) |
| 24 | MAGEC2 | ENSG00000046774 | TTCGTCTATGGGGAGCCTAGGGAGCTC (530) |
| 25 | PAGE5 | ENSG00000158639 | ACTCTGCCCACTTTTGATCCCACTAAAGTG (531) |
| 26 | MAGEA6 | ENSG00000197172 | AATCAGATCATGCCCAAGACAGGCTTC (532) |
| 27 | MAGEA6 | ENSG00000197172 | GAAGTGGACCCCATCGGCCACGTGTAC (533) |
| 28 | CTAG2 | ENSG00000126890 | GAGCTGGTCCGCAGGATCCTGTCCCGG (534) |
| 29 | MAGEA3 | ENSG00000221867 | ATGGAAGTGGACCCCATCGGCCACTTGTAC (535) |
| 30 | MAGEC2 | ENSG00000046774 | AGTGTCATGTCCAGCAACGTCTCCTTT (536) |
| 31 | MAGEC2 | ENSG00000046774 | ACTGTTCCTAGTTCCTTTCCATCCTGG (537) |
| 32 | MAGEC2 | ENSG00000046774 | AAGTACAAAGATTACTTTCCTGTGATA (538) |
| 33 | MAGEA6 | ENSG00000197172 | ATGGAAGTGGACCCCATCGGCCACGTGTAC (539) |
| 34 | MAGEA4 | ENSG00000147381 | ACTGTCTATGGGGAGCCCAGGAAACTG (540) |
| 35 | PAGE5 | ENSG00000158639 | GGTCCTGATGTCAGGGAGGGGACTCTG (541) |
| 36 | MAGEA4 | ENSG00000147381 | GTGTATGATGGGAGGGAGCACACTGTC (542) |
| 37 | MAGEA3 | ENSG00000221867 | CTGATGGAAGTGGACCCCATCGGCCACTTGTAC |
| | | | (543) |
| 38 | MAGEC2 | ENSG00000046774 | AAGTACAAAGATTACTTTCCTGTGATACTCAAG |
| | | | (544) |
| 39 | MAGEA6 | ENSG00000197172 | GAGGTGTTTGAGGGGAGGGAAGACAGTATCTTC |
| | | | (545) |
| 40 | MAGEA4 | ENSG00000147381 | GGGGTGTATGATGGGAGGGAGCACACTGTC |
| | | | (546) |
| 41 | MAGEA2; | ENSG00000268606; | GAAGTGGTCCCCATCAGCCACTTGTAC (547) |
| | MAGEA2B | ENSG00000183305 | |
| 42 | MAGEC2 | ENSG00000046774 | AATGCAGTAGGGGTATATGCTGGGAGG (548) |
| 43 | CTAG2 | ENSG00000126890 | AGGCCGGACAGCCGCCTGCTTGAGTTG (549) |
| 44 | MAGEA2 | ENSG00000268606 | AGTGTCTTCGCACATCCCAGGAAGCTGCTCATG |
| | | | (550) |
| 45 | MAGEA2; | ENSG00000268606; | GAGGTGTTTGAGGGGAGGGAGGACAGTGTCTTC |
| | MAGEA2B | ENSG00000183305 | (551) |
| 46 | FRG2DP | ENSG00000261711 | CGAGTATCTCACTCACATGCTTCTTTT (552) |
| 47 | LINC00578 | ENSG00000236385 | GCCACCTTTTTGCAGAGGAAGGTACTGAGG (553) |
| 48 | MAGEA12 | ENSG00000213401 | AGCTATGTGAAAGTCCTGCACCATTTG (554) |
| 49 | MAGEC1 | ENSG00000155495 | TTTGCCTTTGGGGAGCCCAGGGAGCTC (555) |
| 50 | MAGEA12 | ENSG00000213401 | AAGATGGCTGAGTTGGTTCATTTTCTG (556) |
| 51 | PRSS56 | ENSG00000237412 | TTTGCCCACGGGCACCCACTGTACACACGC (557) |
| 52 | MAGEC2 | ENSG00000046774 | GGGGTATATGCTGGGAGGGAGCACTTCGTC |
| | | | (558) |
| 53 | AC026355.3 | ENSG00000236385 | AACACTGCTATTGCCACCTTTTTGCAGAGGAAG |
| | | | (559) |
| 54 | MAGEC2 | ENSG00000046774 | ACACTAGATGAAAAGGTGGCCGAGTTA (560) |
| 55 | CTAG2 | ENSG00000126890 | CGCCTGCTTGAGTTGCACATCACGATG (561) |
| 56 | MAGEC1 | ENSG00000155495 | GAGGTGCCCAACTCTTCTCCTCCTCGT (562) |
| 57 | MAGEA12 | ENSG00000213401 | GTGGAAGTCACCCTGCGGGAGGTG (563) |
| 58 | MAGEA6 | ENSG00000197172 | ATCAGTGGAGGACCTCGCATTTCCTAC (564) |
| 59 | NA | NA | GGAGTCCCCGGACCCCTCCTCATCCTTCCCAGG |
| | | | (565) |
| 60 | MAGEB2 | ENSG00000099399 | GGAGTCTATGATGGAGAGGAGCACTCAGTC (566) |
| 61 | MAGEA6 | ENSG00000197172 | AAGATCAGTGGAGGACCTCGCATTTCCTAC (567) |
| 62 | FRG2DP | ENSG00000261711 | TTTCTATATAATCAGACAAATATTAGGATG (568) |
| 63 | ITIH6 | ENSG00000102313 | GGCCAGTACCAGCTGGTGGTGAGCCTG (569) |
| 64 | MAGEA2 | ENSG00000268606 | GGAGTTGATGACCTTGTTTTCAGAAGG (570) |
| 65 | MAGEA1 | ENSG00000198681 | AAAGTCCTTGAGTATGTGATCAAGGTC (571) |
| 66 | NA | NA | CACACTTTACCAGCCCTTTCTAGAAGG (572) |
| 67 | MAGEA1 | ENSG00000198681 | GCTTTGAGAGAGGAGGAAGAGGGAGTC (573) |
| 68 | MAGEA1 | ENSG00000198681 | ACTACCATCAACTTCACTCGACAGAGG (574) |
| 69 | MAGEA1 | ENSG00000198681 | AGTGCCTATGGGGAGCCCAGGAAGCTG (575) |
| 70 | PRSS56 | ENSG00000237412 | AGCACTGCCAATGTGACGCGGGCCCAC (576) |
| 71 | MAGEA1 | ENSG00000198681 | TCCGCCTTTCCCACTACCATCAACTTC (577) |
| 72 | MAGEA1 | ENSG00000198681 | TTCCGAGCAGTAATCACTAAGAAGGTG (578) |
| 73 | MAGEB2 | ENSG00000099399 | GCGGCTGCGGGTGTTTCATCCACAAAA (579) |
| 74 | MAGEA1 | ENSG00000198681 | TTTCCCACTACCATCAACTTCACTCGA (580) |
| 75 | SSX2 | ENSG00000241476 | ACTACCTCTGAGAAGATTCACGAGAGA (581) |
| 76 | MAGEA10 | ENSG00000124260 | GGGCTGTATGATGGGATGGAGCACCTC (582) |
| 77 | PRSS56 | ENSG00000237412 | AGCACTGCCAATGTGACGCGGGCCCACGGCCGC |
| | | | (583) |
| 78 | ITIH6 | ENSG00000102313 | ATAGAGGACCCAAAGGCAGGGCTGCATGTG |
| | | | (584) |
| 79 | FAM178B | ENSG00000168754 | GGTGTGGTGGGCCCCCAGGGCCTGAGG (585) |
| 80 | AL035541.1 | ENSG00000227297 | AATGTATTTGCTCATGCAACTGGGGAATCAAGG |
| | | | (586) |
| 81 | MAGEA11 | ENSG00000185247 | TTCCTCTTTGGGGAGCCCAAGAGGCTC (587) |
| 82 | MAGEA1 | ENSG00000198681 | TCCGCCTTTCCCACTACCATCAACTTCACTCGA |
| | | | (588) |
| 83 | NA | NA | AGGCTCTTCTCAGAAGACATACATGTC (589) |
| 84 | AL390957.1 | ENSG00000285280 | GTTTACATGCCTGCTAATACAACATTT (590) |
| 85 | LINC00221 | ENSG00000270816 | GAGGCTGTGGGCTTTCAACCACCACGA (591) |
| 86 | MAGEA11 | ENSG00000185247 | GAAGTGGACCCCACTAGCCACTCCTAT (592) |
| 87 | ZNF560 | ENSG00000198028 | TTACTGGACCCAGCTCAGAGAAACCTGTAC (593) |
| 88 | TRIM48 | ENSG00000150244 | TCTGGAATCTCGCAAGTCTTCCAGAGG (594) |
| 89 | NA | NA | AGGCTCTTCTCAGAAGACATACATGTCGCC (595) |
| 90 | LINC00326 | ENSG00000231023 | GTGTTTGATGTTGGCAAAGGAAACATG (596) |
| 91 | SSX2 | ENSG00000241476 | AAAGCCTCGGAGAAAATCTTCTATGTG (597) |
| 92 | LINC00221 | ENSG00000270816 | GAGGCCGCCGCTGCCTTCAGGTTTCCGAGA |
| | | | (598) |
| 93 | NA | NA | ATGAGTATCTCCATGTCTTTATTGAAA (599) |
| 94 | AL162457.2 | ENSG00000225106 | AAGGCATCCCCCAGCTCTCTGCCTCAT (600) |
| 95 | ZNF560 | ENSG00000198028 | AGAACCTTGCAGCAAGGAGTTCTCCAAGACTGG |
| | | | (601) |
| 96 | MAGEB1 | ENSG00000214107 | ACTACTGCCACGACTTTTAGAGCGCGT (602) |
| 97 | NA | NA | AATCCTAGCCATCTCATCACACCCAGA (603) |
| 98 | LINC00221 | ENSG00000270816 | GAGACAATGGAGACTCTGAGAACATCCAGA (604) |
| 99 | AC011294.1 | ENSG00000233539 | ATGGTATGGGAGGGGCATCCCAGAGAAAGAAGA |
| | | | (605) |
| 100 | ACTL8 | ENSG00000117148 | CTCCCAGACGGCTCCCGCGTGGAGCTG (606) |
| 101 | CT55 | ENSG00000169551 | GATGTTGTGACTGGCAACGTGCCTCTAAAA (607) |
| 102 | NA | NA | GAGAATCCTAGCCATCTCATCACACCCAGA (608) |
| 103 | LHFPL3 | ENSG00000187416 | TCACTGGCCAGGACTATACTCCATCTC (609) |
| 104 | CT55 | ENSG00000169551 | AAACCACATTATGGATTGAGAGCAATC (610) |
| 105 | NA | NA | GCCTTTCTGGACATCCACACAGCCCTG (611) |
| 106 | PACSIN2 | ENSG00000100266 | AAGATGTCAGAGTTTGTCTACAGATTT (612) |
| 107 | CNIH3 | ENSG00000143786 | TTTTTAGTGACAAAGACTGTGACTATG (613) |
| 108 | AC023824.3 | ENSG00000260958 | CACACCACCTCTGTGATGGAGAACAAG (614) |
| 109 | NA | NA | CTTGTGAGCATTCCCATTGCAGTAAGA (615) |
| 110 | NA | NA | ATTACATCAGGAGTAAAAGTGAAGAAA (616) |
| 111 | NA | NA | GTGAGCATTCCCATTGCAGTAAGAAGA (617) |
| 112 | SLC25A24P1 | ENSG00000241361 | GACATCATTGAGCTCCAGGAGGGGCTGAGA (618) |
| 113 | NA | NA | CATCTCAAGCCTCCAGAGCTGGGCTTC (619) |
| 114 | IGF2BP1 | ENSG00000159217 | CAGGTCATCGTGAAAATCATCGGACAT (620) |
| 115 | IGF2BP1 | ENSG00000159217 | GCCGAGCAGGAAATAATGAAGAAAGTT (621) |
| 116 | IGF2BP1 | ENSG00000159217 | GAGGTTCCCCTGAAGATCCTGGCCCAT (622) |
| 117 | IGF2BP1 | ENSG00000159217 | ATCGTGAAAATCATCGGACATTTCTAT (623) |
| 118 | NA | NA | TCTCTTGTGAGCATTCCCATTGCAGTAAGA (624) |
| 119 | NA | NA | ATGGTTTCTCCTGGAGTTTCTCTATTCAAA (625) |
| 120 | AKAP6 | ENSG00000151320 | GTGTTGATGATGAAGTTGGAAGATTTG (626) |
| 121 | PTCHD4 | ENSG00000244694 | TATTCGGACTTACACACCCCTGGGAGGTAT (627) |
| 122 | ACCSL | ENSG00000205126 | GAAGTGGCCCGGTTCCTGACCTACTAC (628) |
| 123 | FAM230C | ENSG00000279516 | AGTGTCGGAGACCTCATGGCTCCCCGG (629) |
| 124 | LINC02434 | ENSG00000248370 | GTGCTCTGGAGAGGTGACTCTCCTCTA (630) |
| 125 | IGF2BP1 | ENSG00000159217 | CAGCCCCGCCAGGGCTCACCTGTGGCAGCG |
| | | | (631) |
| 126 | LINC01320 | ENSG00000228262 | AAACAAACAGGTATTAGGCACAGTTAT (632) |
| 127 | NA | NA | AGACCCAGACAAGATGCCATTTCAGTA (633) |
| 128 | AC093791.1 | ENSG00000250038 | AAACTGATAACACAGATACGTACAGCA (634) |
| 129 | SUGCT-AS1 | ENSG00000203446 | TCTTATTTGTCTGAGCACTCTGCAGCTATTTTC |
| | | | (635) |
| 130 | FAM230C | ENSG00000279516 | ACCAGTGTCGGAGACCTCATGGCTCCCCGG |
| | | | (636) |
| 131 | MAGEA6-DT | ENSG00000286051 | GAGATACCAACTGGGGTGGCCAAGCGG (637) |
| 132 | PTCHD4 | ENSG00000244694 | AGTCAAATCAACCTTAGCCAAATTTAT (638) |
| 133 | NA | NA | CGAAGACCCTCAGGCGGGTTCCGGCTC (639) |
| 134 | NA | NA | GAAGTATTTAATGTAAAAAATACATTTCCAAAG |
| | | | (640) |
| 135 | L3MBTL4-AS1 | ENSG00000264707 | AAGGTCTTAGGAGGTGGATGGACTGTC (641) |
| 136 | LAD1 | ENSG00000267389 | CATGAGGCTCCTGGCCAGCACAGTGAAGTT (642) |
| | pseudogene | | |
| 137 | AC116424.1 | ENSG00000250129 | GAAGTAAGCCACCCTAACATGAGTGGCAGA (643) |
| 138 | LINC02109 | ENSG00000259786 | GACACAGCCCCAGCCACCTTCCTACGG (644) |
| 139 | LINC02199 | ENSG00000250198 | AAACCCCAAAAAGACCTGCACTTCATA (645) |
| 140 | novel_gene | ENSG00000289352 | CGAGTGCGTGGGGCAGGAACACACCTG (646) |
| 141 | NA | NA | ACGGCTCCCATTATTGTGGCTCAGAGA (647) |
| 142 | METTL9 | ENSG00000197006 | ACGAGCTGGGATACCAAGTTCTATGAAAAG (648) |
| 143 | IGF2BP1 | ENSG00000159217 | AAAGTGTTTGCGGAGCACAAGATCTCCTAC (649) |
| 144 | NA | NA | GACGCCTTCCAGGGCATCGCCAACAGG (650) |
| 145 | TEX13C | ENSG00000282815 | ATTGTGGCCGACCCGCGGGTGCCCCGT (651) |
| 146 | CCL17 | ENSG00000102970 | ACAGGCTTCTCCACGAGCCTGGGCAGA (652) |
| 147 | NA | NA | GCCACGGCTCCCATTATTGTGGCTCAGAGA (653) |
| 148 | IGF2BP1 | ENSG00000159217 | GGCCAGTTCTTGGTCAAATCCGGCTAC (654) |
| 149 | NA | NA | TGGACTTCCAGAACGGTGCAAAATAAA (655) |
| 150 | RICTOR | ENSG00000164327 | GCCACATATGGGGGTTCAGATCATTACATT (656) |
| 151 | SMC1B | ENSG00000077935 | GTTTACATTGCAGAGTTGGAAAAGATA (657) |
| 152 | BIRC7 | ENSG00000101197 | GTGTTCCTGGACCGCGCCGTGTCCATC (658) |
| 153 | AC106771.1 | ENSG00000248783 | ATGCCCAGTCCCATCGACCACCCAAGG (659) |
| 154 | NA | NA | GAAGCAGTAAGGCACATCTTACATGGCAGCAGG |
| | | | (660) |
| 155 | NLRP4 | ENSG00000160505 | TACCCAGCAGGCAACGTCAAAGAGCTA (661) |
| 156 | LINC02260 | ENSG00000250456 | AGCGTACCCAGAAGCCTGGCATCCCGG (662) |
| 157 | GBX2-AS1 | ENSG00000233611 | GCGCTAATTAAGCACGTTGCGAACGCA (663) |
| 158 | MAGEA4-AS1 | ENSG00000229967 | TACACGGCAAATCTGGTTCCAGTTAGA (664) |
| 159 | NA | NA | TTGTCCTCTTCCCTTGTCACCAAACTT (665) |
| 160 | WIPF1 | ENSG00000115935 | CAGAACAGTAATGTTTTAGCAGTGAGG (666) |
| 161 | AP003900.1 | ENSG00000277693 | ACGACAACAAAAAACACCTTGAACAAA (667) |
| 162 | NA | NA | CACAATTATATTAATTTGCATTCTCTG (668) |
| 163 | HNRNPA2B1 | ENSG00000122566 | CACCTTAAAGATTACTTTGAGGAATAT (669) |
| 164 | NA | NA | TCTCCAGAATATTTTACCAGCTATCTG (670) |
| 165 | novel_gene | ENSG00000287550 | GCCACAGATGTTTTGGACCACTTATAT (671) |
| 166 | NA | NA | CAGACCTTTGAGGAATCACTGGGAAGA (672) |
| 167 | CST1 | ENSG00000170373 | CATGAACAGCCAGAACTGCAGAAGGTA (673) |
| 168 | novel gene | ENSG00000287550 | ATGGCCACAGATGTTTTGGACCACTTATAT (674) |
| 169 | NA | NA | ACTTCTGGCAGTCTCCTGTTTCCCAGA (675) |
| 170 | NLRP4 | ENSG00000160505 | AAGCTTGGAATAAATAACGTTTCCTTT (676) |
| 171 | NA | NA | AAATATATTTCACAATTTCAAGTATTC (677) |
| 172 | NA | NA | CAGACTGCGGCTGTGTCGGGAGTAAAG (678) |
| 173 | NA | NA | TTGACACCATTAAACCATCCTTTTTCCTAC (679) |
| 174 | NA | NA | GTATTTATAAATAAAAAGTACGTTTTT (680) |
| 175 | NA | NA | AACCTCATCACTGAGGAGCATCCTGTC (681) |
| 176 | AL807740.1 | ENSG00000283400 | GAGTTAGTGAAGGTCATTGATGGAAGCTCCAGG |
| | | | (682) |
| 177 | NA | NA | TTTCACTCACTTCCAATTCATTTGTTC (683) |
| 178 | NA | NA | TATTCTCATGATTACTCTTACTAT (684) |
| 179 | novel_gene | ENSG00000286099 | TTCCAGATAGAAAAATTGACACCCCTG (685) |
| 180 | AL137220.1 | ENSG00000225775 | GAGCACTCTGTTGAAAATGGCAGTATT (686) |
| 181 | LINC00466 | ENSG00000224209 | GAGACAATTCAAAACACACATTCATTT (687) |
| 182 | AC018558.1 | ENSG00000260857 | TACTACAGTATTTGGAAGACACAAATT (688) |
| 183 | AC003092.1 | ENSG00000236453 | TCTTATTATTTTCACGGCCTGTCATTT (689) |
| 184 | novel_gene | ENSG00000287308 | GAGTCTTTTCAACAACCTTATATTCCCAGG (690) |
| 185 | RUVBL1 | ENSG00000175792 | AAACAGGTACCCTCTAGGGTCCTCTAC (691) |
| 186 | GPR158 | ENSG00000151025 | ATGTTATTTAATAAAGATTTGGATGCAGCA (692) |
| 187 | NA | NA | GCCCCACGGTCAGCAGAGCCACGGGCCACC |
| | | | (693) |
| 188 | NA | NA | TATCTGGATCCTGGGGCAGTGCATGCAGTGTTT |
| | | | (694) |
| 189 | ZNF716 | ENSG00000182111 | ACCCTTAAGAAACATAAGATAGTT (695) |
| 190 | NA | NA | GTGGGTGTGCCAATCAGCACTCTGTAT (696) |
| 191 | NA | NA | AGCCCCCGCCCCCAGCCAGAGCTC (697) |
| 192 | NA | NA | AAGCTTTCCTTCCCTGATTCAAAATTT (698) |
| 193 | SEMA3A | ENSG00000075213 | AAAAACAAAGATGAATATATCCATTTTTAC (699) |
| 194 | LINC00511 | ENSG00000227036 | AGGGCATTTGAGGGGAGAAGAAGAGGATGG |
| | | | (700) |
| 195 | PRRC2A | ENSG00000204469 | CGTCTCAAGGCCAGGCCTTCCACCTAC (701) |
| 196 | novel_gene | ENSG00000287308 | GCCCCCTCTCTTCACGCCACACTC (702) |
| 197 | NA | NA | GCGCAGAAAGAACCCACTGGCAATTAC (703) |
| 198 | NA | NA | CTCTCACTGGGTACCACAGCTTGCAGG (704) |
| 199 | NA | NA | AGCAGAAACCCTACAAGCTACAAGTAT (705) |
| 200 | TEX15 | ENSG00000133863 | AGCCTAAATAATGACGATCACCAGATATAC (706) |
| 201 | IL23R | ENSG00000162594 | TATGTTGATCCCATGATTACAGAGATA (707) |
| 202 | NA | NA | CCCAGCTTTTCTCCATATGCAAAAAGG (708) |
| 203 | CTSB | ENSG00000164733 | CACGTCAACGGCTCCCGCCCCCATGCACGG |
| | | | (709) |
| 204 | MIR4307HG | ENSG00000257612 | ATAACAGTGAGATTGGCTCCTCCACTC (710) |
| 205 | LINC02941 | ENSG00000236013 | TCAGCCAATCCCCTAAGTCATAGATAC (711) |
| 206 | PMEL | ENSG00000185664 | GTGCCTCTTGCTCATTCCAGCTCAGCC (712) |
| 207 | PMEL | ENSG00000185664 | GGCCAAGTGCCTACTACAGAAGTTGTG (713) |
| 208 | PMEL | ENSG00000185664 | AAGACCTGGGGCCAATACTGGCAAGTT (714) |
| 209 | PMEL | ENSG00000185664 | GCCTTGAACTTCCCTGGAAGCCAAAAG (715) |
| 210 | PMEL | ENSG00000185664 | TATGTCTGGAAGACCTGGGGCCAATAC (716) |
| 211 | PMEL | ENSG00000185664 | ATGCCAACTGCAGAGAGCACAGGTATG (717) |
| 212 | PMEL | ENSG00000185664 | CACACCATGGAAGTGACTGTCTACCATCGCCGG |
| | | | (718) |
| 213 | MLANA | ENSG00000120215 | GAAGAGGCCGCTGGGATCGGCATCCTG (719) |
| 214 | MLANA | ENSG00000120215 | GAAGGGTTTGATCATCGGGACAGCAAA (720) |
| 215 | MLANA | ENSG00000120215 | GCTGAAGAGGCCGCTGGGATCGGCATCCTG |
| | | | (721) |
| 216 | MLANA | ENSG00000120215 | CTCACTTCATCTATGGTTACCCCAAGA (722) |
| 217 | MLANA | ENSG00000120215 | ACTTCATCTATGGTTACCCCAAGAAGG (723) |
| 218 | PRAME | ENSG00000185686 | GGGCTGAGCAATCTGACCCACGTGCTG (724) |
| 219 | PRAME | ENSG00000185686 | CAGCTCCTTGCCCTCCTGCCTTCCCTG (725) |
| 220 | PRAME | ENSG00000185686 | GAAGCAGCTCAGCCCATGACAAAGAAG (726) |
| 221 | PLA1A | ENSG00000144837 | AGGGTTTTAGGAACAAAGCCTTCCTGG (727) |
| 222 | PRAME | ENSG00000185686 | GTGCTGTATCCTGTCCCCCTGGAGAGTTAT (728) |
| 223 | PRAME | ENSG00000185686 | CCTTACCTGGGCCAGATGATTAATCTG (729) |
| 224 | PRAME | ENSG00000185686 | GAATTGTTCTCCTACCTCATTGAGAAA (730) |
| 225 | PRAME | ENSG00000185686 | GAAGAGCAGTATATCGCCCAGTTC (731) |
| 226 | PRAME | ENSG00000185686 | GAAGCAGCTCAGCCCATGACAAAGAAGCGA (732) |
| 227 | PRAME | ENSG00000185686 | ATGCCCATGCAGGATATCAAGATGATC (733) |
| 228 | PRAME | ENSG00000185686 | CGGCTTTCGGAAGGGGATGTGATGCATCTG (734) |
| 229 | PRAME | ENSG00000185686 | CATGCATCTTCCTACATTTCCCCGGAGAAG (735) |
| 230 | PRAME | ENSG00000185686 | ATGCCCATGCAGGATATCAAGATGATCCTG (736) |
| 231 | PRAME | ENSG00000185686 | AGTCCCAGCGTCAGTCAGCTAAGTGTCCTG (737) |
| 232 | PRAME | ENSG00000185686 | CGCCTGGATCAGTTGCTCAGGCACGTG (738) |
| 233 | PRAME | ENSG00000185686 | GCTCTGCTGGAGAGAGCCTCTGCCACCCTC (739) |
| 234 | VGF | ENSG00000128564 | ACCGTCGCTCCTACCCCGGCCACCGCTCGG |
| | | | (740) |
| 235 | PRAME | ENSG00000185686 | TCCCAGAGTCCCAGCGTCAGTCAGCTA (741) |
| 236 | VGF | ENSG00000128564 | TCGACCGTCGCTCCTACCCCGGCCACCGCTCGG |
| | | | (742) |
| 237 | PRAME | ENSG00000185686 | CAGTATATCGCCCAGTTCACCTCTCAGTTC (743) |
| 238 | PRAME | ENSG00000185686 | GGACAACATCTTCACCTGGAGACCTTC (744) |
| 239 | SLC24A5 | ENSG00000188467 | ACAATTGCAAGTGTGTTGGTTGCAAGA (745) |
| 240 | PRAME | ENSG00000185686 | AGTCTCCTGCAGCACCTCATCGGGCTG (746) |
| 241 | XAGE1B | ENSG00000204382 | ACAGCCAGTCCCAGGAGCCCAGTAATG (747) |
| 242 | PRAME | ENSG00000185686 | GCAGCCTTTGACGGGAGACACAGCCAGACCCTG |
| | | | (748) |
| 243 | PRAME | ENSG00000185686 | AGCCCACGGAGACTTGTGGAGCTG (749) |
| 244 | PRAME | ENSG00000185686 | CTCTATGTGGACTCTTTATTTTTCCTT (750) |
| 245 | DCT | ENSG00000080166 | GTCTTCCTGAGAGAATTTCCTAATTTT (751) |
| 246 | CHCHD6 | ENSG00000159685 | AGGCCCAGATTCTCCACTGCTACCGAGATCGCC |
| | | | (752) |
| 247 | SLC45A2 | ENSG00000164175 | GCTTTGATTGCTAACCCAAGGAGGAAG (753) |
| 248 | SLC45A2 | ENSG00000164175 | GATTTCATGGGCCAGATTGTGTACCGC (754) |
| 249 | SLC24A5 | ENSG00000188467 | GAAATTCCCGATACAGTAATGGGCCTT (755) |
| 250 | SLC45A2 | ENSG00000164175 | GTAATGTCCAGCACCCTGTACACTGTG (756) |
| 251 | CA14 | ENSG00000118298 | TCACCTCAGCACAAGCCACGACTG (757) |
| 252 | CHCHD6 | ENSG00000159685 | GAGCCTAAGCCACCCACCACAGGGCTT (758) |
| 253 | SLC24A5 | ENSG00000188467 | ATACCAGACACAATTGCAAGTGTGTTG (759) |
| 254 | SLC45A2 | ENSG00000164175 | ACAGATTTCATGGGCCAGATTGTGTACCGC (760) |
| 255 | LHFPL3-AS1 | ENSG00000226869 | GGAGAAGATGATGAACACAGAAAAATT (761) |
| 256 | PRAME | ENSG00000185686 | CGTTTGCGGGGTTCCATTCAGAGCCGATAC (762) |
| 257 | ETV4 | ENSG00000175832 | CACTTGGATGAGAGCCCCGCCTACCTC (763) |
| 258 | LHFPL3-AS1 | ENSG00000226869 | AAGATGGAAGAAACTACGGTCACATAT (764) |
| 259 | ROPN1B | ENSG00000114547 | CTCCCAACAGATCTGTTTAATAGTGTG (765) |
| 260 | CA14 | ENSG00000118298 | AGGCCTCAGGGCCTGGCTGTCCTG (766) |
| 261 | CSAG1 | ENSG00000198930 | AGTCTATCTGAAAAGTCTCCAGAAGTC (767) |
| 262 | ETV4 | ENSG00000175832 | GGTGCATTTCGAGAGGGGCCGCCCTAC (768) |
| 263 | SLC45A2 | ENSG00000164175 | TACCTCAATGGGGCTACTGTTGTAGCA (769) |
| 264 | ETV4 | ENSG00000175832 | TCAATGTACCTCCACACAGAGGGCTTC (770) |
| 265 | CA14 | ENSG00000118298 | CACATTCTGAGTCACTTGCATGAAGTC (771) |
| 266 | SLC45A2 | ENSG00000164175 | AGTATCTCTGAAGCCCCACTTACAGAGGTT (772) |
| 267 | CA14 | ENSG00000118298 | CACCAGATCAACAGTGAAGCCACATTT (773) |
| 268 | ETV4 | ENSG00000175832 | GACATCAAGCAGGAAGGGGTCGGTGCATTT (774) |
| 269 | ROPN1B | ENSG00000114547 | AGCGCTCTGGGAGTTACTATTACCAAA (775) |
| 270 | ROPN1 | ENSG00000065371 | ACAGTGAATGACTTTACCCAAAACCCCAGG (776) |
| 271 | CA14 | ENSG00000118298 | TCTTTCATCCAAGCAGGATCCTCGTAT (777) |
| 272 | ROPN1B | ENSG00000114547 | GGAGTTACTATTACCAAAACTCTCAAG (778) |
| 273 | CA14 | ENSG00000118298 | GCTGAGAGGCCTCAGGGCCTGGCTGTC (779) |
| 274 | CSAG1 | ENSG00000198930 | AGGGAAAAGGGACCCGTCAAGGAAGTT (780) |
| 275 | CA14 | ENSG00000118298 | ACCTCAGTGCCTCCCTTCAACCTAAGA (781) |
| 276 | LINC00520 | ENSG00000258791 | TTATCACGCCTTCCAAGCATTGCCTTT (782) |
| 277 | BANCR | ENSG00000278910 | TTCCCAGTTCATGCTGCTGAGAAGTTC (783) |
| 278 | ROPN1 | ENSG00000065371 | GGGGAGATCTCTGCATCACATGTC (784) |
| 279 | CA14 | ENSG00000118298 | GCTGCAGATGGGGGTCAACACTGGACGTAT (785) |
| 280 | ROPN1 | ENSG00000065371 | GAGATCTCTGCATCACATGTCAGCAGG (786) |
| 281 | ROPN1 | ENSG00000065371 | TCGCCCCGGATCCCGTTCAGCACCTTC (787) |
| 282 | ETV4 | ENSG00000175832 | AAGCTCATGGACCCGGGCTCCCTGCCGCCCCTC |
| | | | (788) |
| 283 | ALX1 | ENSG00000180318 | GAAACAAAGCCAGAGTTTGAAAGGAGG (789) |
| 284 | SLC6A15 | ENSG00000072041 | GAGACGATCATGAAATTTTTGAAAATG (790) |
| 285 | NA | NA | AGGCGCGCCCACCTTTGGAGCACTTTT (791) |
| 286 | CENPF | ENSG00000117724 | CATCTCCAGGAAAAGCTGCAGAGTTTG (792) |
| 287 | CENPF | ENSG00000117724 | GAACTCTCAGTGGTCACAAGTGAGAGA (793) |
| 288 | ETV4 | ENSG00000175832 | TACTTGGACCAGCAAGTGCCCTACACCTTC (794) |
| 289 | TRPM1 | ENSG00000134160 | GAAGCAATTCCTCGAATCCCTCGCTTG (795) |
| 290 | ETV4 | ENSG00000175832 | GGATACTTGGACCAGCAAGTGCCCTACACCTTC |
| | | | (796) |
| 291 | SLC6A15 | ENSG00000072041 | AGCCCTAAACTGGGCGGGATTGGATTT (797) |
| 292 | SLC5A4 | ENSG00000100191 | TATCCTGAGAAATCACGTGGATGCCTC (798) |
| 293 | ZNF595 | ENSG00000272602 | ACCACCATTCCCTTCAGCACTCTACGTCAGAGG |
| | | | (799) |
| 294 | FAM178B | ENSG00000168754 | AGCCTGGGTGCCCACAGTCCTGCCCTGTAC (800) |
| 295 | SLC5A4 | ENSG00000100191 | TCTCAAAATGGACAACTAATCCATTAC (801) |
| 296 | ALX1 | ENSG00000180318 | AAAGCCAAGGAGCACACCGCCAATATTTCATGG |
| | | | (802) |
| 297 | ENTHD1 | ENSG00000176177 | AGGGTATCAAAGTCAGATTCTACTTTC (803) |
| 298 | IL12RB2 | ENSG00000081985 | TCCCTCAATTCTCAAGTCACAGGTCTT (804) |
| 299 | CENPF | ENSG00000117724 | AACTCAGATTTAAGTGAAAAATTGGAATAT (805) |
| 300 | PRC1 | ENSG00000198901 | GACAGTTCCACTGTTGGGCTTCAGCGA (806) |
| 301 | ASPM | ENSG00000066279 | AGAGCTTCAGCTATTATAATTCAGAGA (807) |
| 302 | ENTHD1 | ENSG00000176177 | TCCCAGGAAACACTTCCACTCAAGATT (808) |
| 303 | LRP2 | ENSG00000081479 | AGCACCTTTGATGGTTTAGACAGAAGA (809) |
| 304 | ENTHD1 | ENSG00000176177 | CGAGTGAAGGAGATTGTAATCAACAAG (810) |
| 305 | DLGAP5 | ENSG00000126787 | GCTCAGCCCCACCATGGTGTGCCATAT (811) |
| 306 | KIF4A | ENSG00000090889 | GAAGTAATACAGTTAAAAGAACGAGACCGTAAG |
| | | | (812) |
| 307 | CENPF | ENSG00000117724 | GAGGTGAAGGAAAAGACGGAACTC (813) |
| 308 | RDH8 | ENSG00000080511 | ACTGACCCTGAGACCCTGCACTAC (814) |
| 309 | CENPF | ENSG00000117724 | AAAATGAAGGAGAAAACACAAGAGCTT (815) |
| 310 | LRP2 | ENSG00000081479 | AGCACCTTTGATGGTTTAGACAGAAGAAGA (816) |
| 311 | CENPF | ENSG00000117724 | AAAATGTCAGAGCTGCAGACCTATGTT (817) |
| 312 | RDH8 | ENSG00000080511 | GGCACTGACCCTGAGACCCTGCACTAC (818) |
| 313 | IL12RB2 | ENSG00000081985 | ACAGAGGAAAAGGGGAGCATTTTAATT (819) |
| 314 | EXO1 | ENSG00000174371 | GACACTGCTATGCCTGCCCATTCAAGA (820) |
| 315 | PRR11 | ENSG00000068489 | AAGCTCCAGGAAGCACTGAAGACCATC (821) |
| 316 | RDH8 | ENSG00000080511 | GGCACTGACCCTGAGACCCTGCACTACTTC (822) |
| 317 | ARHGAP11A | ENSG00000198826 | AGTTTAAAAAATCGAATTGAATCTGTA (823) |
| 318 | KIF23 | ENSG00000137807 | AACCTTAAAGACCCAGTTGGGGTATAC (824) |
| 319 | LRP2 | ENSG00000081479 | TTCACAGAGACCAACCCACAAGTGTAC (825) |
| 320 | NA | NA | GAAACCCAGGAGGCAGAGGTTGCAGTGAGCCGA |
| | | | (826) |
| 321 | EXO1 | ENSG00000174371 | TCTCCCAGACCAGAGTCGGGTACTGTT (827) |
| 322 | PRC1 | ENSG00000198901 | TCTGAGTTTGCGAAGGATCCGTCCCTC (828) |
| 323 | EXO1 | ENSG00000174371 | AAGGTTATCAAGAAAATTGGACATTAT (829) |
| 324 | ASPM | ENSG00000066279 | AGGACGTGGCAAACCAGAAAACAGTTT (830) |
| 325 | EXO1 | ENSG00000174371 | GCCATGGCCCACAAAGTAATTAAAGCT (831) |
| 326 | DEPDC1 | ENSG00000024526 | AAACTAAAACAGTTTCAGAAGGAATAT (832) |
| 327 | CLSPN | ENSG00000092853 | TCTCCATTGGAAGATGAAGAGTCATTA (833) |
| 328 | CLSPN | ENSG00000092853 | AACTCTACAGACTTTACCACTGACAGA (834) |
| 329 | SKA1 | ENSG00000154839 | GAAACGAAGGATACCAAAGGTCGTTATTTT (835) |
| 330 | MCM10 | ENSG00000065328 | CGGCCTCGAGTATCCTCCACAGAAATG (836) |
| 331 | KIF14 | ENSG00000118193 | TTAATGGACAAATCAAGCACTATTTAC (837) |
| 332 | SLC24A4 | ENSG00000140090 | GGGACCCTCCGGCCGCTCAAAGTTCGCAGG |
| | | | (838) |
| 333 | PRC1 | ENSG00000198901 | ATCCTGAGTGAAGGATCCGTCCCTCTC (839) |
| 334 | NA | NA | ACAGTCTATAATTTTAGGACTGAACTT (840) |
| 335 | TYR | ENSG00000077498 | CTCCCCTCTTCAGCTGATGTAGAATTT (841) |
| 336 | TYR | ENSG00000077498 | AGGCTCCCCTCTTCAGCTGATGTAGAATTT (842) |
| 337 | MLANA | ENSG00000120215 | GCCTTGATGGATAAAAGTCTTCATGTT (843) |
| 338 | TYR | ENSG00000077498 | ATGCACAATGCCTTGCACATCTATATG (844) |
| 339 | TYR | ENSG00000077498 | ATGGAGAAAGAGGATTACCACAGCTTG (845) |
| 340 | TYR | ENSG00000077498 | ATGGAGAAAGAGGATTACCACAGCTTGTAT (846) |
| 341 | TYR | ENSG00000077498 | TACCACAGCTTGTATCAGAGCCATTTA (847) |
| 342 | TYR | ENSG00000077498 | TACATGGTTCCTTTTATACCACTGTAC (848) |
| 343 | TYR | ENSG00000077498 | CTCATGGAGAAAGAGGATTACCACAGCTTG (849) |
| 344 | TYR | ENSG00000077498 | TTTGCCCATGAAGCACCAGCTTTTCTG (850) |
| 345 | TYR | ENSG00000077498 | TTTGCTAGTCCACTTACTGGGATAGCG (851) |
| 346 | TYR | ENSG00000077498 | CATGAAGCACCAGCTTTTCTGCCTTGG (852) |
| 347 | TYR | ENSG00000077498 | GTCCTCTTCAAGAAGTTTATCCAGAAG (853) |
| 348 | TYR | ENSG00000077498 | GCACCAGCTTTTCTGCCTTGGCATAGA (854) |
| 349 | TYR | ENSG00000077498 | CTCCTCATGGAGAAAGAGGATTACCACAGCTTG |
| | | | (855) |
| 350 | TYR | ENSG00000077498 | CATTATTATGTGTCAATGGATGCACTG (856) |
| 351 | TYR | ENSG00000077498 | TGGGAACAAGAAATCCAGAAGCTG (857) |
| 352 | TYR | ENSG00000077498 | CTCATGGAGAAAGAGGATTACCACAGCTTGTAT |
| | | | (858) |
| 353 | TYR | ENSG00000077498 | TTGGAACAAGCGAGTCGGATCTGGTCATGG (859) |
| 354 | TYR | ENSG00000077498 | AACATCTTCGATTTGAGTGCCCCAGAGAAG (860) |
| 355 | TYR | ENSG00000077498 | GAAGCACCAGCTTTTCTGCCTTGGCATAGA (861) |
| 356 | TYR | ENSG00000077498 | TCTGAAATCTGGAGAGACATTGATTTT (862) |
| 357 | TYR | ENSG00000077498 | GCGGCGATGGTAGGGGCCGTCCTC (863) |
| 358 | TYRP1 | ENSG00000107165 | ACTTTCCTTGGGGTAGGACAGGAAAGCTTT (864) |
| 359 | TYR | ENSG00000077498 | GAGTCGTGGCCTTCCGTCTTTTATAATAGG (865) |
| 360 | TYRP1 | ENSG00000107165 | ATTTCTCAGGACACAACTGTGGGACGT (866) |
| 361 | DCT | ENSG00000080166 | AGGCCCTGGAGTGGTCCCTACATCCTA (867) |
| 362 | TYR | ENSG00000077498 | AGCTCAGACTATGTCATCCCCATAGGGACCTAT |
| | | | (868) |
| 363 | TYR | ENSG00000077498 | GATTCAGACCCAGACTCTTTTCAAGACTAC (869) |
| 364 | DCT | ENSG00000080166 | GACACAAGGCCCTGGAGTGGTCCCTAC (870) |
| 365 | TYRP1 | ENSG00000107165 | CACTATTACTCAGTCAAAAAGACTTTC (871) |
| 366 | DCT | ENSG00000080166 | AATGTCTGTGGCTCTCAGCAAGGCCGG (872) |
| 367 | TYR | ENSG00000077498 | TATATGAATGGAACAATGTCCCAGGTA (873) |
| 368 | TYRP1 | ENSG00000107165 | ATGGCAAAGCGCACAACTCACCCTTTA (874) |
| 369 | DCT | ENSG00000080166 | AGGCCCTGGAGTGGTCCCTACATCCTACGA (875) |
| 370 | DCT | ENSG00000080166 | ATGACGGTGGACAGCCTAGTGAACAAG (876) |
| 371 | DCT | ENSG00000080166 | ACAAGGCCCTGGAGTGGTCCCTACATCCTA (877) |
| 372 | TYRP1 | ENSG00000107165 | ACAACTCACCCTTTATTTGTCATTGCCACCAGG |
| | | | (878) |
| 373 | TYRP1 | ENSG00000107165 | TTTGTTTGGACACACTATTACTCAGTC (879) |
| 374 | TYRP1 | ENSG00000107165 | TTAAGTAAAGAAGAAAAGAACCACTTT (880) |
| 375 | TYRP1 | ENSG00000107165 | AGAGCTGCAAACCAGGTCTTTGTTTTG (881) |
| 376 | DCT | ENSG00000080166 | ATGGTGAGAAGCGCTACCCTCATTAAA (882) |
| 377 | TYRP1 | ENSG00000107165 | AACCAGGTCTTTGTTTTGCACTCTTAT (883) |
| 378 | SLC45A2 | ENSG00000164175 | CACAACTCCACAGAGTTTCTCATC (884) |
| 379 | SLC24A5 | ENSG00000188467 | TCTCAGGATGTTGCAGGCACAACTTTC (885) |
| 380 | SLC45A2 | ENSG00000164175 | GCACACAACTCCACAGAGTTTCTCATC (886) |
| 381 | SLC45A2 | ENSG00000164175 | TCCACAGAGTTTCTCATCTACGAAAGA (887) |
| 382 | SLC24A5 | ENSG00000188467 | GAAGGGTTTTTCACGAGACAGGAGCGC (888) |
| 383 | SLC45A2 | ENSG00000164175 | TTTCAGAAAGTTTTGGTATCCTAC (889) |
| 384 | SLC45A2 | ENSG00000164175 | TTCACAGATTTCATGGGCCAGATTGTGTAC (890) |
| 385 | SLC45A2 | ENSG00000164175 | TACTTTCAGAAAGTTTTGGTATCCTAC (891) |
| 386 | SLC45A2 | ENSG00000164175 | GCACTGGTGAACATGCCTCCTCACTACCGC (892) |
| 387 | SLC45A2 | ENSG00000164175 | GGTGTAATGTCCAGCACCCTGTACACTGTG (893) |
| 388 | SLC24A5 | ENSG00000188467 | TTGCTATCTAATACGGTCTCAACACTA (894) |
| 389 | SLC24A5 | ENSG00000188467 | AGGGTTTTTCACGAGACAGGAGCGCAGAGA (895) |
| 390 | SLC45A2 | ENSG00000164175 | TCGGCCAGCGACCACTGCCGGTCCAGG (896) |
| 391 | SLC24A5 | ENSG00000188467 | CGCAGAGATGGAGGCATCATAATCTAT (897) |
| 392 | SLC45A2 | ENSG00000164175 | GTAGCAGCTTTGATTGCTAACCCAAGG (898) |
| 393 | SLC45A2 | ENSG00000164175 | AGACTGTTGGGTACAGAATTCCAGGTC (899) |
| 394 | SLC24A5 | ENSG00000188467 | TTCTCCATCATCGGAGTTTCCCGAAGG (900) |
| 395 | SLC45A2 | ENSG00000164175 | TTGTCATCAGATGGAATGTACGAGTAT (901) |
| 396 | SLC24A5 | ENSG00000188467 | GATGAATACTTCCTACCCTCCCTG (902) |
| 397 | SLC45A2 | ENSG00000164175 | TCCTACATTGGATTAAAGGGTCTTTACTTC (903) |
| 398 | SLC45A2 | ENSG00000164175 | TTCACAGGTTTTGGAGGTGCCCTGGGTTAC (904) |
| 399 | PMEL | ENSG00000185664 | AAGACCTGGGACCAGGTGCCTTTCTCCGTG (905) |
| 400 | TRPM1 | ENSG00000134160 | AGGGTGGCGGATATTCCAATAAAGCCA (906) |
| 401 | TRPM1 | ENSG00000134160 | TCTGTTGCCAAGCACACCCAGAGCTAC (907) |
| 402 | SLC24A5 | ENSG00000188467 | CAGACAAAAGGGGGCCAAACATGGGCGAGA |
| | | | (908) |
| 403 | LINC00518 | ENSG00000183674 | GAAACAGGAAGTTCATTCATGAGTTTT (909) |
| 404 | SLC45A2 | ENSG00000164175 | TCACTTTATTCTTACTTTCAGAAAGTT (910) |
| 405 | SLC24A5 | ENSG00000188467 | CAGACAAAAGGGGGCCAAACATGGGCGAGAAGG |
| | | | (911) |
| 406 | SLC45A2 | ENSG00000164175 | ACTGTTGTAGCAGCTTTGATTGCTAACCCAAGG |
| | | | (912) |
| 407 | TRPM1 | ENSG00000134160 | CATCTCATGGTGAAAGATTGGCAGCTG (913) |
| 408 | TRPM1 | ENSG00000134160 | AACCCTCTAAGTAAGCTCTCTGTGCTC (914) |
| 409 | TRPM1 | ENSG00000134160 | TTTGTAATTCCTAGCACGAAAGACTCTAACAGG |
| | | | (915) |
| 410 | MITF | ENSG00000187098 | CACTATCAGGTGCAGACCCACCTC (916) |
| 411 | TRPM1 | ENSG00000134160 | GCCTCGGACATCCTGTCCTTTGCGCACAAGTAC |
| | | | (917) |
| 412 | TRPM1 | ENSG00000134160 | AGACTGGGGCAGGGCGTGCCCCTCGTG (918) |
| 413 | TRPM1 | ENSG00000134160 | AGTGTACCAGACCATGTCCAACCCTCTAAG (919) |
| 414 | TRPM1 | ENSG00000134160 | AGGCTGCTGGAAAAGCACATCTCCCTG (920) |
| 415 | LINC00520 | ENSG00000258791 | TCCCTCTTTGGGGTCTCAGAGAGATTG (921) |
| 416 | MITF | ENSG00000187098 | AATCACTATCAGGTGCAGACCCACCTC (922) |
| 417 | TRPM1 | ENSG00000134160 | GACCTGGTTGGAAAGGATGTAACAAGAGTGTAC |
| | | | (923) |
| 418 | MLANA | ENSG00000120215 | TCTCTCAGTCCTCCAACACTACCAAGA (924) |
| 419 | TRPM1 | ENSG00000134160 | CTGGCTGACAATGGCACCCTGGGCAAGTAT (925) |
| 420 | TRPM1 | ENSG00000134160 | AAAGCCATGTATATCCGTGTATCCTAT (926) |
| 421 | TRPM1 | ENSG00000134160 | ATGCAGCCCAAGCTGAAACAAGTCTTT (927) |
| 422 | MLANA | ENSG00000120215 | GCAATTATTCAGGATGGCATCTTGCTG (928) |
| 423 | LINC00518 | ENSG00000183674 | GCGTTTGACAGAGCTGTTCACTTC (929) |
| 424 | LINC00518 | ENSG00000183674 | AATACTCCTAACATTGTCTCTTTAAGA (930) |
| 425 | TRPM1 | ENSG00000134160 | AGGGTGGCGGATATTCCAATAAAGCCATGG (931) |
| 426 | MOK | ENSG00000080823 | CACTATATAATCCCACTGCAAACAATA (932) |
| 427 | LHFPL3-AS1 | ENSG00000226869 | GTCATGAAGGGCATTAATCAAGGTTAT (933) |
| 428 | GPR143 | ENSG00000101850 | GCGACGTCCCCGCCGGCCTCGGTCCGC (934) |
| 429 | ABCB5 | ENSG00000004846 | GCACTGGAGAAGGATACCCCCAGGTAT (935) |
| 430 | LHFPL3-AS1 | ENSG00000226869 | CAAGTCATGAAGGGCATTAATCAAGGTTAT (936) |
| 431 | LINC00518 | ENSG00000183674 | GCAGCGTTTGACAGAGCTGTTCACTTC (937) |
| 432 | LINC00518 | ENSG00000183674 | AGACCGCTGAGGATCAAAGGGGTTTTC (938) |
| 433 | LHFPL3-AS1 | ENSG00000226869 | AGGCAAGTCATGAAGGGCATTAATCAAGGTTAT |
| | | | (939) |
| 434 | TRPM1 | ENSG00000134160 | AAACTGGACACCACTGTAAAGAACATC (940) |
| 435 | KU-MEL-3 | ENSG00000205444 | CTTCCGCGAACTCCTCGCCCCGACCTC (941) |
| 436 | NA | NA | ATAACTTACACCGAAAGCAACTCAAGG (942) |
| 437 | AL162457.2 | ENSG00000225106 | CGACTTCAGGGATCTGTCACCCTCGTC (943) |
| 438 | LHFPL3-AS1 | ENSG00000226869 | TCTCAATTCCCAAGTAAAAATCTTCCTGCTTTT |
| | | | (944) |
| 439 | KU-MEL-3 | ENSG00000205444 | CTTCCGCGAACTCCTCGCCCCGACCTCATCCTT |
| | | | (945) |
| 440 | LINC00520 | ENSG00000258791 | GACACTGCCCCACAATCCCATCAAGTC (946) |
| 441 | CHCHD6 | ENSG00000159685 | ATGGCTATCCTGGGCACAGCGGGGCGG (947) |
| 442 | SUGCT-AS1 | ENSG00000203446 | TCTGTGTCCTCTCACCTCATTCATAGA (948) |
| 443 | TRPM1 | ENSG00000134160 | TCCACGTCACCAGGGACAGGAGTCAGGAAA (949) |
| 444 | LHFPL3-AS1 | ENSG00000226869 | GAAGTGGCTGAAGTCCTTGGGAGTGTGAAG (950) |
| 445 | NA | NA | GAGATACCACGAACCCCCTCAGAAGGAAGA (951) |
| 446 | LINC00520 | ENSG00000258791 | GTGTTTTTAAAATTCTCCAGAAGATTT (952) |
| 447 | SUGCT-AS1 | ENSG00000203446 | TCTGTGTCCTCTCACCTCATTCATAGATAC (953) |
| 448 | LINC01320 | ENSG00000228262 | ACACATCTAGAAGACCCTCACCTTATG (954) |
| 449 | KIAA1549L | ENSG00000110427 | AGTGTCTTTCCCAGGACCTCCTCCAGA (955) |
| 450 | PAX3 | ENSG00000135903 | GGGCAGTATGGACAAAGTAAGCCTTGGACTTTT |
| | | | (956) |
| 451 | TRIM51 | ENSG00000124900 | GTTCCAAGACCTACCAGCACAGTAGGATTA (957) |
| 452 | SUGCT-AS1 | ENSG00000203446 | AATTCTGTGTCCTCTCACCTCATTCATAGATAC |
| | | | (958) |
| 453 | LINC00520 | ENSG00000258791 | CAAGAAGAAGACAACCTTCTAAGATACTTT (959) |
| 454 | LINC01320 | ENSG00000228262 | ACACATCTAGAAGACCCTCACCTTATGCTT (960) |
| 455 | DCT | ENSG00000080166 | GCTACAACAAACATCCTTGAACATGTA (961) |
| 456 | LINC00504 | ENSG00000248360 | ATGACTCTGAGGCCTACACAGCCACGT (962) |
| 457 | MSRB3-AS1 | ENSG00000250748 | GAAACAAGACCGAACAACTCCAAGTTC (963) |
| 458 | TYR | ENSG00000077498 | CACTCAATCACGGAGCCTCTGCTCTAT (964) |
| 459 | NA | NA | CATTTATTCTTACAACCTCCAAGCAAAAGA (965) |
| 460 | FCRLA | ENSG00000132185 | GAGACAGTTAGATTTACTTCCTCTTTT (966) |
| 461 | AC011294.1 | ENSG00000233539 | TCCCCAATGCCTATACCCCCATTGTATCTT (967) |
| 462 | ABCB5 | ENSG00000004846 | GAATCTTATGGTAGGTATACTTTTTAC (968) |
| 463 | LHFPL3-AS1 | ENSG00000226869 | AAATCACAAGGTTCATTTATTTCCTTT (969) |
| 464 | SLC24A5 | ENSG00000188467 | GGTACTATCTTGCACATAGGTGTATTT (970) |
| 465 | C10orf90 | ENSG00000154493 | AAAGTATTTTCTGGAAGCAGCAAGAGG (971) |
| 466 | LINC02099 | ENSG00000253490 | TCAAGCTTCAGCACCCTTGCATCTCTGGATAAA |
| | | | (972) |
| 467 | CA14 | ENSG00000118298 | ACCACCCAGGGAGCTGCTCCTCAGAGG (973) |
| 468 | CA14 | ENSG00000118298 | CATACCACCCAGGGAGCTGCTCCTCAGAGG (974) |
| 469 | SHC4 | ENSG00000185634 | AAGATCTTCCAAGAGAGGGCAGCCTTT (975) |
| 470 | SLC6A15 | ENSG00000072041 | GGGCAAATCACACATAACACTTCATTC (976) |
| 471 | PAX3 | ENSG00000135903 | ATTAGTCTCAAGCCTTTGATAGCACGGTAT (977) |
| 472 | NLGN1 | ENSG00000169760 | AAGCATTCTGATTTGATTTTATTCTGG (978) |
| 473 | AC011294.1 | ENSG00000233539 | ATGGTATGGGAGGGGCATCCCAGAGAAAGA |
| | | | (979) |
| 474 | C5orf22 | ENSG00000082213 | TCAACTGATTTGCCCATTCTTCTTAAA (980) |
| 475 | NA | NA | GAGGTAATTTCTATCAACACTGGACATTTC (981) |
| 476 | BFSP1 | ENSG00000125864 | GAGACAGAGCGGGTCCTGGAGAAGTCTTCTTAC |
| | | | (982) |
| 477 | LHFPL3-AS1 | ENSG00000226869 | AAGATAAAAACTATAAATCTGAGATAT (983) |
| 478 | TRPM1 | ENSG00000134160 | TATGTGAGTCAGAAAGTTTCAGACTTT (984) |
| 479 | ANO4 | ENSG00000151572 | GATGCAGGCATCCCTGTGTACCACCGA (985) |
| 480 | MIR3681HG | ENSG00000224184 | ACCATCTTGGAAGCAGAGATCCTTCACCAGGCA |
| | | | (986) |
| 48 | VPS53 | ENSG00000141252 | GAATATGCTCAGAAAACACCTCTCTTT (987) |
| 482 | LHFPL3-AS1 | ENSG00000226869 | ACATGGACACCACATGGTATCAGAATC (988) |
| 483 | PRTG | ENSG00000166450 | GAGGTCCACGAAGGTGGAGTTGCTCGATTT (989) |
| 484 | MIR3681HG | ENSG00000224184 | AGAGAAATAGAAATAGAGGGAAGATTA (990) |
| 485 | PRTG | ENSG00000166450 | TCCTGGCTGCCAATCCCAGCCAAATAT (991) |
| 486 | MDGA2 | ENSG00000139915 | CGCTACTCCGAAAGGGTCTACACTATC (992) |
| 487 | SLC45A2 | ENSG00000164175 | TTCACAGACTCGCAGGGCAATGACATTAAA (993) |
| 488 | NA | NA | TCAGTGCCAAGCGACTTCCTGAGTCTCAGG (994) |
| 489 | DLGAP1 | ENSG00000170579 | CGGCAGATGAGTCTGGAGTTCAGGGCA (995) |
| 490 | AL138885.2 | ENSG00000228478 | GAAACCATCTCCCCCAGGCCTTCAATA (996) |
| 491 | IGSF11 | ENSG00000144847 | GGGCCAGCAGGAGGAAGGGGAGTCATC (997) |
| 492 | AC010894.3 | ENSG00000236449 | CTGACTGACCATCACATAAAAGAATAC (998) |
| 493 | TEX41 | ENSG00000226674 | GAAGCAGTACCTTATAATGGTTCCTTGTTCAAA |
| | | | (999) |
| 494 | DPYSL4 | ENSG00000151640 | AATCTGCTGGCGGAGATCCACGGTGTG (1000) |
| 495 | CCDC140 | ENSG00000163081 | AAAGAGGGGCCAGTGACGCGAGGAGCA (1001) |
| 496 | LHFPL3-AS1 | ENSG00000226869 | GTACACGTGGATTTGAATCTTGGCTTC (1002) |
| 497 | DCT | ENSG00000080166 | CACACTGTACAGGGACTAGCTGGATTT (1003) |
| 498 | novel_gene | ENSG00000286873 | ATGCGTATAGAGGACAGCCACACTAAGCTT |
| | | | (1004) |
| 499 | IGSF11 | ENSG00000144847 | AGTGCCCTGTCTTCAGCCCAGCCCAGG (1005) |
| 500 | PRDM7 | ENSG00000126856 | TCCCCTTCCATCAAAAGTCTACTAGTTTAT (1006) |
| 501 | TICRR | ENSG00000140534 | CCAACTGATGCCACTTTAAACCGTTTGCTCTAC |
| | | | (1007) |
| 502 | MIR3681HG | ENSG00000224184 | TGGACTTCCCAGCCCCCAAACTATGAGAAA |
| | | | (1008) |
| 503 | MLANA | ENSG00000120215 | CCCGCCCCCCGCCCCGTGGCACCCACCCTG |
| | | | (1009) |
| 504 | NA | NA | GTATATCTCCATTCTAACAGCACATTA (1010) |
| 505 | NA | NA | TCTTTGTATGCCTGGACTCAGAGCCAC (1011) |

TABLE 2C
characteristics of the TSAs, TAAs and LSAs identified herein (continued)

| Peptide | | | |
|---|---|---|---|
| (SEQ ID | | | |
| NO:) | ERE | MCS_strand | MCS_alignment |
| 1 | no | + | chr13: 18232805-18232831 |
| 2 | no | − | chr9: 102611399-102611425 |
| 3 | no | + | chr17: 30422918-30422947 |
| 4 | no | + | chr11: 64320868-64320894 |
| 5 | no | − | chr16: 67183758-67183784 |
| 6 | no | +; + | chrX: 49533350-49533376; chrX: 49571573-49571599 |
| 7 | no | − | chrX: 52516983-52517009 |
| 8 | no | − | chrX: 152766787-152766813 |
| 9 | no | − | chrX: 152767231-152767254 |
| 10 | no | + | chrX: 152701499-152701525 |
| 11 | no | −; + | chrX: 152766877-152766903; chrX: 152701580-152701606 |
| 12 | no | + | chrX: 55221461-55221463\|55221767-55221790 |
| 13 | no | +; − | chrX: 152736870-152736896; chrX: 152750629-152750655 |
| 14 | no | + | chrX: 152701034-152701063 |
| 15 | no | + | chrX: 55221461-55221463\|55221767-55221793 |
| 16 | no | + | chrX: 152701043-152701069 |
| 17 | no | + | chrX: 151924454-151924480 |
| 18 | no | − | chrX: 142202917-142202940 |
| 19 | no | + | chrX: 151924523-151924546 |
| 20 | no | + | chrX: 152701334-152701360 |
| 21 | no | + | chrX: 152701412-152701435 |
| 22 | no | + | chrX: 151924520-151924546 |
| 23 | no | + | chrX: 151924445-151924471 |
| 24 | no | − | chrX: 142203169-142203195 |
| 25 | no | + | chrX: 55222707-55222736 |
| 26 | no | − | chrX: 152767048-152767074 |
| 27 | no | − | chrX: 152767123-152767149 |
| 28 | no | − | chrX: 154652568-154652594 |
| 29 | no | + | chrX: 152701331-152701360 |
| 30 | no | − | chrX: 142202875-142202901 |
| 31 | no | − | chrX: 142202992-142203018 |
| 32 | no | − | chrX: 142203445-142203471 |
| 33 | no | − | chrX: 152767123-152767152 |
| 34 | no | + | chrX: 151924376-151924402 |
| 35 | no | + | chrX: 55222686-55222712 |
| 36 | no | + | chrX: 151924355-151924381 |
| 37 | no | + | chrX: 152701328-152701360 |
| 38 | no | − | chrX: 142203439-142203471 |
| 39 | no | − | chrX: 152766934-152766966 |
| 40 | no | + | chrX: 151924352-151924381 |
| 41 | no | −; + | chrX: 152750836-152750862; chrX: 152717608-152717634 |
| 42 | no | − | chrX: 142203202-142203228 |
| 43 | no | − | chrX: 154652631-154652631\|154653247-154653272 |
| 44 | no | − | chrX: 152750623-152750655 |
| 45 | no | −; + | chrX: 152750647-152750679; chrX: 152717791-152717823 |
| 46 | no | − | chr16: 35478345-35478371 |
| 47 | no | − | chr3: 177622656-177622685 |
| 48 | no | + | chrX: 152737005-152737031 |
| 49 | no | + | chrX: 141908507-141908533 |
| 50 | no | + | chrX: 152736495-152736521 |
| 51 | no | + | chr2: 232520644-232520673 |
| 52 | no | − | chrX: 142203190-142203219 |
| 53 | no | − | chr3: 177622665-177622697 |
| 54 | no | − | chrX: 142203544-142203570 |
| 55 | no | − | chrX: 154652619-154652631\|154653247-154653260 |
| 56 | no | + | chrX: 141908576-141908602 |
| 57 | no | + | chrX: 152736297-152736320 |
| 58 | no | − | chrX: 152766748-152766774 |
| 59 | no | − | chr9: 65485652-65485684 |
| 60 | no | + | chrX: 30219271-30219300 |
| 61 | no | − | chrX: 152766748-152766777 |
| 62 | no | − | chr16: 35477621-35477650 |
| 63 | no | − | chrX: 54790958-54790984 |
| 64 | no | − | chrX: 152752480-152752506 |
| 65 | no | + | chrX: 153183221-153183247 |
| 66 | no | − | chr14: 106400961-106400987 |
| 67 | no | + | chrX: 153183290-153183316 |
| 68 | no | + | chrX: 153182585-153182611 |
| 69 | no | + | chrX: 153183077-153183103 |
| 70 | no | + | chr2: 232521994-232522020 |
| 71 | no | + | chrX: 153182573-153182599 |
| 72 | no | + | chrX: 153182681-153182707 |
| 73 | no | + | chrX: 30218794-30218820 |
| 74 | no | + | chrX: 153182579-153182605 |
| 75 | no | − | chrX: 52700447-52700473 |
| 76 | no | − | chrX: 152134835-152134861 |
| 77 | no | + | chr2: 232521994-232522026 |
| 78 | no | − | chrX: 54751370-54751380\|54753651-54753669 |
| 79 | no | − | chr2: 96972033-96972059 |
| 80 | no | − | chr20: 57600026-57600058 |
| 81 | no | + | chrX: 149716534-149716560 |
| 82 | no | + | chrX: 153182573-153182605 |
| 83 | yes | − | chr9: 102553540-102553566 |
| 84 | no | − | chr1: 192558595-192558621 |
| 85 | no | + | chr14: 106488813-106488839 |
| 86 | no | + | chrX: 149716327-149716353 |
| 87 | no | − | chr19: 9470435-9470464 |
| 88 | no | + | chr11: 55264910-55264936 |
| 89 | yes | − | chr9: 102553537-102553566 |
| 90 | no | + | chr6: 133106274-133106300 |
| 91 | no | − | chrX: 52705165-52705191 |
| 92 | no | + | chr14: 106482609-106482637\|106488768-106488768 |
| 93 | no | + | chr4: 17371117-17371143 |
| 94 | no | − | chr20: 61754043-61754069 |
| 95 | no | − | chr19: 9471374-9471378\|9473179-9473206 |
| 96 | no | + | chrX: 30251472-30251498 |
| 97 | no | + | chr4: 17371222-17371248 |
| 98 | no | + | chr14: 106482635-106482637\|106488768-106488794 |
| 99 | no | − | chr7: 46689286-46689318 |
| 100 | no | + | chr1: 17826136-17826162 |
| 101 | no | − | chrX: 135169660-135169689 |
| 102 | no | + | chr4: 17371219-17371248 |
| 103 | no | + | chr7: 104807995-104808021 |
| 104 | no | − | chrX: 135169597-135169623 |
| 105 | no | + | chr2: 105616087-105616113 |
| 106 | no | − | chr22: 42912142-42912157\|42959572-42959582 |
| 107 | no | + | chr1: 224546913-224546939 |
| 108 | no | + | chr16: 35253070-35253096 |
| 109 | no | + | chr4: 17371189-17371215 |
| 110 | no | − | chr13: 89025188-89025214 |
| 111 | no | + | chr4: 17371192-17371218 |
| 112 | no | + | chr1: 108273256-108273285 |
| 113 | no | − | chr5: 170297884-170297910 |
| 114 | no | + | chr17: 49046332-49046358 |
| 115 | no | + | chr17: 49042315-49042341 |
| 116 | no | + | chr17: 49041385-49041411 |
| 117 | no | + | chr17: 49046338-49046364 |
| 118 | no | + | chr4: 17371186-17371215 |
| 119 | no | − | chr2: 105591292-105591321 |
| 120 | no | + | chr14: 32494283-32494309 |
| 121 | no | − | chr6: 48068323-48068352 |
| 122 | no | + | chr11: 44051345-44051371 |
| 123 | no | + | chr13: 18222528-18222554 |
| 124 | no | + | chr4: 188160305-188160331 |
| 125 | no | + | chr17: 49038292-49038321 |
| 126 | yes | + | chr2: 33757656-33757682 |
| 127 | yes | + | chr1: 226768974-226769000 |
| 128 | no | − | chr4: 28370749-28370775 |
| 129 | no | − | chr7: 40544786-40544818 |
| 130 | no | + | chr13: 18222525-18222554 |
| 131 | yes | + | chrX: 152792059-152792085 |
| 132 | no | − | chr6: 47877659-47877685 |
| 133 | no | − | chr11: 735228-735254 |
| 134 | no | + | chr6: 155509717-155509749 |
| 135 | no | + | chr18: 6258746-6258772 |
| 136 | no | − | chr19: 27893530-27893559 |
| 137 | yes | − | chr3: 131072241-131072270 |
| 138 | no | + | chr5: 28810113-28810139 |
| 139 | no | + | chr5: 8848686-8848712 |
| 140 | no | − | chr7: 2614767-2614793 |
| 141 | yes | + | chr12: 127119844-127119870 |
| 142 | no | + | chr16: 21597271-21597300 |
| 143 | no | + | chr17: 48997803-48997832 |
| 144 | no | + | chr13: 18233526-18233552 |
| 145 | no | + | chrX: 125320276-125320302 |
| 146 | no | + | chr16: 57410172-57410198 |
| 147 | yes | + | chr12: 127119841-127119870 |
| 148 | no | + | chr17: 48997836-48997862 |
| 149 | yes | + | chr4: 17391991-17392017 |
| 150 | no | − | chr5: 38947303-38947332 |
| 151 | no | − | chr22: 45406792-45406818 |
| 152 | no | + | chr20: 63239468-63239494 |
| 153 | yes | − | chr5: 12574525-12574551 |
| 154 | no | + | chr2: 193867429-193867461 |
| 155 | no | + | chr19: 55862131-55862157 |
| 156 | yes | − | chr4: 54603955-54603981 |
| 157 | no | + | chr2: 236169233-236169259 |
| 158 | no | − | chrX: 151909715-151909741 |
| 159 | no | − | chr21: 41705358-41705384 |
| 160 | no | − | chr2: 174585743-174585769 |
| 161 | yes | − | chr21: 10328984-10329010 |
| 162 | no | + | chr11: 89359134-89359160 |
| 163 | no | − | chr7: 26196913-26196939 |
| 164 | no | + | chr9: 31401937-31401963 |
| 165 | no | − | chr6: 164806073-164806099 |
| 166 | yes | − | chr6: 137599711-137599737 |
| 167 | no | − | chr20: 23749013-23749039 |
| 168 | no | − | chr6: 164806073-164806102 |
| 169 | no | + | chr4: 16542003-16542029 |
| 170 | no | + | chr19: 55861540-55861547\|55861992-55862010 |
| 171 | no | − | chr9: 102610490-102610516 |
| 172 | no | − | chr4: 81215362-81215388 |
| 173 | yes | + | chr1: 112831490-112831519 |
| 174 | no | + | chr1: 248307033-248307059 |
| 175 | no | + | chr20: 58628154-58628180 |
| 176 | no | + | chrX: 20476512-20476544 |
| 177 | no | − | chr14: 30402385-30402411 |
| 178 | no | − | chr1: 9436539-9436562 |
| 179 | yes | + | chr14: 40854984-40855010 |
| 180 | no | − | chr6: 9141095-9141121 |
| 181 | no | − | chr1: 63258011-63258037 |
| 182 | no | − | chr16: 35505262-35505288 |
| 183 | no | + | chr7: 94065599-94065625 |
| 184 | no | + | chr2: 105626131-105626160 |
| 185 | no | − | chr3: 128085111-128085131\|128087706-128087711 |
| 186 | no | + | chr10: 25475459-25475488 |
| 187 | no | + | chr6: 168825608-168825637 |
| 188 | no | + | chr1: 222101131-222101163 |
| 189 | no | + | chr7: 57469473-57469496 |
| 190 | yes | + | chr4: 188179511-188179537 |
| 191 | no | − | chr1: 9442054-9442077 |
| 192 | no | + | chr8: 29823441-29823467 |
| 193 | no | − | chr7: 84350595-84350624 |
| 194 | no | − | chr17: 72599127-72599144\|72603103-72603114 |
| 195 | no | + | chr6: 31637256-31637282 |
| 196 | no | + | chr2: 105608245-105608268 |
| 197 | yes | − | chr11: 104694495-104694521 |
| 198 | no | + | chr4: 114508557-114508583 |
| 199 | yes | − | chrX: 29599351-29599377 |
| 200 | no | − | chr8: 30847152-30847181 |
| 201 | no | + | chr1: 67258523-67258549 |
| 202 | no | − | chr15: 47183734-47183760 |
| 203 | no | − | chr8: 11847757-11847769\|11847771-11847787 |
| 204 | yes | + | chr14: 26901058-26901084 |
| 205 | no | + | chr6: 140080426-140080452 |
| 206 | no | − | chr12: 55957936-55957962 |
| 207 | no | − | chr12: 55957304-55957330 |
| 208 | no | − | chr12: 55958068-55958084\|55958473-55958482 |
| 209 | no | − | chr12: 55961366-55961392 |
| 210 | no | − | chr12: 55958077-55958084\|55958473-55958491 |
| 211 | no | − | chr12: 55957181-55957207 |
| 212 | no | − | chr12: 55957978-55958010 |
| 213 | no | + | chr9: 5892547-5892551\|5897557-5897578 |
| 214 | no | + | chr9: 5906939-5906965 |
| 215 | no | + | chr9: 5892544-5892551\|5897557-5897578 |
| 216 | no | + | chr9: 5892491-5892517 |
| 217 | no | + | chr9: 5892494-5892520 |
| 218 | no | − | chr22: 22548277-22548303 |
| 219 | no | − | chr22: 22548391-22548417 |
| 220 | no | − | chr22: 22550205-22550231 |
| 221 | no | + | chr3: 119606974-119606975\|119608770-119608794 |
| 222 | no | − | chr22: 22548253-22548282 |
| 223 | no | − | chr22: 22549893-22549919 |
| 224 | no | − | chr22: 22550085-22550111 |
| 225 | no | − | chr22: 22549815-22549838 |
| 226 | no | − | chr22: 22550202-22550231 |
| 227 | no | − | chr22: 22550001-22550027 |
| 228 | no | − | chr22: 22548571-22548600 |
| 229 | no | − | chr22: 22549839-22549868 |
| 230 | no | − | chr22: 22549998-22550027 |
| 231 | no | − | chr22: 22548535-22548564 |
| 232 | no | − | chr22: 22548637-22548643\|22549726-22549745 |
| 233 | no | − | chr22: 22548457-22548486 |
| 234 | no | − | chr7: 101165459-101165488 |
| 235 | no | − | chr22: 22548544-22548570 |
| 236 | no | − | chr7: 101165459-101165491 |
| 237 | no | − | chr22: 22549803-22549832 |
| 238 | no | − | chr22: 22550820-22550846 |
| 239 | no | + | chr15: 48141184-48141210 |
| 240 | no | − | chr22: 22548298-22548324 |
| 241 | no | − | chrX: 52515525-52515551 |
| 242 | no | − | chr22: 22550904-22550936 |
| 243 | no | − | chr22: 22551024-22551047 |
| 244 | no | − | chr22: 22549752-22549778 |
| 245 | no | − | chr13: 94437812-94437838 |
| 246 | no | + | chr3: 126957451-126957483 |
| 247 | no | − | chr5: 33982387-33982413 |
| 248 | no | − | chr5: 33951666-33951677\|33954361-33954375 |
| 249 | no | + | chr15: 48141124-48141150 |
| 250 | no | − | chr5: 33947208-33947234 |
| 251 | no | + | chr1: 150264628-150264651 |
| 252 | no | + | chr3: 126957199-126957225 |
| 253 | no | + | chr15: 48141175-48141201 |
| 254 | no | − | chr5: 33951666-33951677\|33954361-33954378 |
| 255 | no | − | chr7: 104738893-104738919 |
| 256 | no | − | chr22: 22551069-22551089\|22556812-22556820 |
| 257 | no | − | chr17: 43528576-43528602 |
| 258 | no | − | chr7: 104738926-104738952 |
| 259 | no | + | chr3: 125977064-125977090 |
| 260 | no | + | chr1: 150262582-150262605 |
| 261 | no | − | chrX: 152727583-152727609 |
| 262 | no | − | chr17: 43529627-43529653 |
| 263 | no | − | chr5: 33984200-33984226 |
| 264 | no | − | chr17: 43530138-43530164 |
| 265 | no | + | chr1: 150262834-150262860 |
| 266 | no | − | chr5: 33963844-33963873 |
| 267 | no | + | chr1: 150262268-150262294 |
| 268 | no | − | chr17: 43529645-43529674 |
| 269 | no | + | chr3: 125977151-125977165\|125982270-125982281 |
| 270 | no | − | chr3: 123969170-123969199 |
| 271 | no | + | chr1: 150263407-150263419\|150263659-150263672 |
| 272 | no | + | chr3: 125977160-125977165\|125982270-125982290 |
| 273 | no | + | chr1: 150262576-150262602 |
| 274 | no | − | chrX: 152727818-152727844 |
| 275 | no | + | chr1: 150263050-150263076 |
| 276 | yes | − | chr14: 55781253-55781279 |
| 277 | no | − | chr9: 69296854-69296867\|69304936-69304948 |
| 278 | no | − | chr3: 123970074-123970097 |
| 279 | no | + | chr1: 150258171-150258183\|150260151-150260167 |
| 280 | no | − | chr3: 123970068-123970094 |
| 281 | no | − | chr3: 123970131-123970157 |
| 282 | no | − | chr17: 43545284-43545316 |
| 283 | no | + | chr12: 85301380-85301406 |
| 284 | no | − | chr12: 84872761-84872787 |
| 285 | no | − | chr9: 65485549-65485575 |
| 286 | no | + | chr1: 214645664-214645690 |
| 287 | no | + | chr1: 214645424-214645450 |
| 288 | no | − | chr17: 43545564-43545593 |
| 289 | no | − | chr15: 31002071-31002097 |
| 290 | no | − | chr17: 43545564-43545596 |
| 291 | no | − | chr12: 84885923-84885949 |
| 292 | no | − | chr22: 32218697-32218723 |
| 293 | no | + | chr4: 68217-68249 |
| 294 | no | − | chr2: 96929235-96929264 |
| 295 | no | − | chr22: 32225739-32225765 |
| 296 | no | + | chr12: 85301434-85301466 |
| 297 | no | − | chr22: 39765374-39765400 |
| 298 | no | + | chr1: 67321775-67321801 |
| 299 | no | + | chr1: 214645085-214645114 |
| 300 | no | − | chr15: 90967200-90967202\|90969079-90969102 |
| 301 | no | − | chr1: 197103026-197103052 |
| 302 | no | − | chr22: 39835861-39835887 |
| 303 | no | − | chr2: 169256161-169256187 |
| 304 | no | − | chr22: 39765290-39765316 |
| 305 | no | − | chr14: 55175351-55175377 |
| 306 | no | + | chrX: 70376187-70376210\|70386618-70386626 |
| 307 | no | + | chr1: 214645592-214645615 |
| 308 | no | + | chr19: 10021340-10021363 |
| 309 | no | + | chr1: 214645517-214645543 |
| 310 | no | − | chr2: 169256158-169256187 |
| 311 | no | + | chr1: 214642629-214642655 |
| 312 | no | + | chr19: 10021337-10021363 |
| 313 | no | + | chr1: 67372657-67372683 |
| 314 | no | + | chr1: 241861491-241861502\|241866830-241866844 |
| 315 | no | + | chr17: 59185542-59185562\|59193492-59193497 |
| 316 | no | + | chr19: 10021337-10021366 |
| 317 | no | + | chr15: 32635837-32635863 |
| 318 | no | + | chr15: 69416043-69416063\|69417383-69417388 |
| 319 | no | − | chr2: 169275097-169275123 |
| 320 | yes | − | chr10: 28895486-28895518 |
| 321 | no | + | chr1: 241866914-241866940 |
| 322 | no | − | chr15: 90969106-90969120\|90969447-90969458 |
| 323 | no | + | chr1: 241858716-241858718\|241860517-241860540 |
| 324 | no | − | chr1: 197100614-197100640 |
| 325 | no | + | chr1: 241853458-241853481\|241857345-241857347 |
| 326 | no | − | chr1: 68477055-68477069\|68477787-68477798 |
| 327 | no | − | chr1: 35763205-35763231 |
| 328 | no | − | chr1: 35764404-35764430 |
| 329 | no | + | chr18: 50391275-50391293\|50392099-50392109 |
| 330 | no | + | chr10: 13180443-13180469 |
| 331 | no | − | chr1: 200565641-200565667 |
| 332 | no | + | chr14: 92323843-92323872 |
| 333 | no | − | chr15: 90969104-90969120\|90969524-90969533 |
| 334 | no | + | chr6: 80042709-80042735 |
| 335 | no | + | chr11: 89191316-89191342 |
| 336 | no | + | chr11: 89191313-89191342 |
| 337 | no | + | chr9: 5897645-5897653\|5906885-5906902 |
| 338 | no | + | chr11: 89227870-89227896 |
| 339 | no | + | chr11: 89295322-89295348 |
| 340 | no | + | chr11: 89295322-89295351 |
| 341 | no | + | chr11: 89295337-89295363 |
| 342 | no | + | chr11: 89284861-89284887 |
| 343 | no | + | chr11: 89295319-89295348 |
| 344 | no | + | chr11: 89178551-89178577 |
| 345 | no | + | chr11: 89227825-89227851 |
| 346 | no | + | chr11: 89178557-89178583 |
| 347 | no | + | chr11: 89284802-89284828 |
| 348 | no | + | chr11: 89178563-89178589 |
| 349 | no | + | chr11: 89295316-89295348 |
| 350 | no | + | chr11: 89178491-89178517 |
| 351 | no | + | chr11: 89178605-89178628 |
| 352 | no | + | chr11: 89295319-89295351 |
| 353 | no | + | chr11: 89295178-89295207 |
| 354 | no | + | chr11: 89178317-89178346 |
| 355 | no | + | chr11: 89178560-89178589 |
| 356 | no | + | chr11: 89178527-89178553 |
| 357 | no | + | chr11: 89295217-89295240 |
| 358 | no | + | chr9: 12695724-12695753 |
| 359 | no | + | chr11: 89178185-89178214 |
| 360 | no | + | chr9: 12694307-12694333 |
| 361 | no | − | chr13: 94479025-94479051 |
| 362 | no | + | chr11: 89178389-89178421 |
| 363 | no | + | chr11: 89284948-89284954\|89295143-89295165 |
| 364 | no | − | chr13: 94479031-94479057 |
| 365 | no | + | chr9: 12695703-12695729 |
| 366 | no | − | chr13: 94479082-94479108 |
| 367 | no | + | chr11: 89227891-89227917 |
| 368 | no | + | chr9: 12695577-12695603 |
| 369 | no | − | chr13: 94479022-94479051 |
| 370 | no | − | chr13: 94479142-94479168 |
| 371 | no | − | chr13: 94479025-94479054 |
| 372 | no | + | chr9: 12695589-12695621 |
| 373 | no | + | chr9: 12695691-12695717 |
| 374 | no | + | chr9: 12695535-12695561 |
| 375 | no | + | chr9: 12693951-12693977 |
| 376 | no | − | chr13: 94479521-94479547 |
| 377 | no | + | chr9: 12693960-12693986 |
| 378 | no | − | chr5: 33951624-33951647 |
| 379 | no | + | chr15: 48134269-48134295 |
| 380 | no | − | chr5: 33951624-33951650 |
| 381 | no | − | chr5: 33951615-33951641 |
| 382 | no | + | chr15: 48121901-48121927 |
| 383 | no | − | chr5: 33947349-33947372 |
| 384 | no | − | chr5: 33951669-33951677\|33954361-33954381 |
| 385 | no | − | chr5: 33947349-33947374\|33951554-33951554 |
| 386 | no | − | chr5: 33954439-33954468 |
| 387 | no | − | chr5: 33947208-33947237 |
| 388 | no | + | chr15: 48134524-48134538\|48134884-48134895 |
| 389 | no | + | chr15: 48121903-48121932 |
| 390 | no | − | chr5: 33984290-33984316 |
| 391 | no | + | chr15: 48121925-48121951 |
| 392 | no | − | chr5: 33982393-33982412\|33984199-33984205 |
| 393 | no | − | chr5: 33963928-33963954 |
| 394 | no | + | chr15: 48121879-48121905 |
| 395 | no | − | chr5: 33963775-33963801 |
| 396 | no | + | chr15: 48121997-48122020 |
| 397 | no | − | chr5: 33947325-33947354 |
| 398 | no | − | chr5: 33963994-33964016\|33982236-33982242 |
| 399 | no | − | chr12: 55957652-55957671\|55958473-55958482 |
| 400 | no | − | chr15: 31070033-31070059 |
| 401 | no | − | chr15: 31070091-31070117 |
| 402 | no | + | chr15: 48121048-48121077 |
| 403 | no | − | chr6: 10430213-10430239 |
| 404 | no | − | chr5: 33947361-33947374\|33951554-33951566 |
| 405 | no | + | chr15: 48121048-48121080 |
| 406 | no | − | chr5: 33982393-33982412\|33984199-33984211 |
| 407 | no | − | chr15: 31068024-31068050 |
| 408 | no | − | chr15: 31066194-31066220 |
| 409 | no | − | chr15: 31070226-31070226\|31076905-31076936 |
| 410 | no | + | chr3: 69936747-69936755\|69937822-69937836 |
| 411 | no | − | chr15: 31063132-31063164 |
| 412 | no | − | chr15: 31063267-31063292\|31066076-31066076 |
| 413 | no | − | chr15: 31066210-31066239 |
| 414 | no | − | chr15: 31066092-31066118 |
| 415 | yes | − | chr14: 55793418-55793444 |
| 416 | no | + | chr3: 69936744-69936755\|69937822-69937836 |
| 417 | no | − | chr15: 31066233-31066247\|31067063-31067080 |
| 418 | no | + | chr9: 5891119-5891145 |
| 419 | no | − | chr15: 31066140-31066169 |
| 420 | no | − | chr15: 31068075-31068092\|31070031-31070039 |
| 421 | no | − | chr15: 31067946-31067972 |
| 422 | no | + | chr9: 5891152-5891178 |
| 423 | yes | − | chr6: 10434321-10434344 |
| 424 | no | − | chr6: 10434579-10434605 |
| 425 | no | − | chr15: 31070030-31070059 |
| 426 | no | − | chr14: 102246059-102246085 |
| 427 | no | − | chr7: 104798403-104798407\|104799818-104799839 |
| 428 | no | − | chrX: 9765611-9765637 |
| 429 | no | + | chr7: 20658665-20658676\|20659066-20659080 |
| 430 | no | − | chr7: 104798403-104798407\|104799818-104799842 |
| 431 | yes | − | chr6: 10434321-10434347 |
| 432 | no | − | chr6: 10434421-10434426\|10434513-10434533 |
| 433 | no | − | chr7: 104798403-104798407\|104799818-104799845 |
| 434 | no | − | chr15: 31069825-31069851 |
| 435 | no | + | chr6: 4610754-4610780 |
| 436 | no | + | chrX: 130835850-130835876 |
| 437 | no | − | chr20: 61738753-61738779 |
| 438 | no | − | chr7: 104738602-104738634 |
| 439 | no | + | chr6: 4610754-4610786 |
| 440 | no | − | chr14: 55782028-55782054 |
| 441 | no | + | chr3: 126957031-126957057 |
| 442 | yes | − | chr7: 40539400-40539426 |
| 443 | no | − | chr15: 31002737-31002766 |
| 444 | no | − | chr7: 104785267-104785296 |
| 445 | yes | − | chr10: 28895886-28895915 |
| 446 | no | − | chr14: 55782521-55782547 |
| 447 | yes | − | chr7: 40539397-40539426 |
| 448 | yes | + | chr2: 33855692-33855718 |
| 449 | no | + | chr11: 33544273-33544299 |
| 450 | no | − | chr2: 222201927-222201959 |
| 451 | no | + | chr11: 55891477-55891506 |
| 452 | yes | − | chr7: 40539397-40539429 |
| 453 | no | − | chr14: 55782118-55782147 |
| 454 | yes | + | chr2: 33855692-33855721 |
| 455 | no | − | chr13: 94452609-94452635 |
| 456 | yes | − | chr4: 14888103-14888129 |
| 457 | no | − | chr12: 65642085-65642111 |
| 458 | no | + | chr11: 89242935-89242961 |
| 459 | no | + | chrX: 130827948-130827977 |
| 460 | no | + | chr1: 161707170-161707196 |
| 461 | yes | − | chr7: 46688406-46688435 |
| 462 | no | + | chr7: 20671857-20671883 |
| 463 | no | − | chr7: 104767903-104767929 |
| 464 | no | + | chr15: 48134416-48134442 |
| 465 | no | − | chr10: 126464952-126464978 |
| 466 | no | + | chr8: 29790336-29790368 |
| 467 | no | + | chr1: 150257481-150257507 |
| 468 | no | + | chr1: 150257478-150257507 |
| 469 | no | − | chr15: 48963604-48963630 |
| 470 | no | − | chr12: 84883748-84883774 |
| 471 | no | − | chr2: 222201534-222201563 |
| 472 | no | + | chr3: 174285640-174285666 |
| 473 | no | − | chr7: 46689289-46689318 |
| 474 | no | + | chr5: 31538210-31538236 |
| 475 | no | − | chr6: 10428732-10428761 |
| 476 | no | − | chr20: 17498906-17498938 |
| 477 | no | − | chr7: 104768197-104768223 |
| 478 | no | − | chr15: 31063812-31063838 |
| 479 | no | + | chr12: 100739961-100739987 |
| 480 | yes | + | chr2: 12315544-12315576 |
| 481 | no | − | chr17: 601915-601941 |
| 482 | no | − | chr7: 104743312-104743338 |
| 483 | no | − | chr15: 55683867-55683896 |
| 484 | no | + | chr2: 12397807-12397833 |
| 485 | no | − | chr15: 55673591-55673617 |
| 486 | no | − | chr14: 47301468-47301494 |
| 487 | no | − | chr5: 33954482-33954504\|33982236-33982242 |
| 488 | no | − | chr6: 164900690-164900719 |
| 489 | no | − | chr18: 4248611-4248637 |
| 490 | no | − | chr6: 10424768-10424794 |
| 491 | no | − | chr3: 118981885-118981911 |
| 492 | no | + | chr2: 174649757-174649783 |
| 493 | no | + | chr2: 145070829-145070861 |
| 494 | no | + | chr10: 132202111-132202116\|132203762-132203782 |
| 495 | no | + | chr2: 222303863-222303889 |
| 496 | no | − | chr7: 104783468-104783494 |
| 497 | no | − | chr13: 94443041-94443067 |
| 498 | no | + | chr2: 127892353-127892382 |
| 499 | no | − | chr3: 118904788-118904798\|118905668-118905683 |
| 500 | no | − | chr16: 90064099-90064128 |
| 501 | no | + | chr15: 89582869-89582901 |
| 502 | yes | + | chr2: 12315601-12315630 |
| 503 | no | + | chr9: 5893905-5893934 |
| 504 | no | − | chr2: 37379139-37379165 |
| 505 | no | − | chr15: 47991164-47991190 |

* ERE = endogenous retroviral element

### Example 2: Evidence that aeTSAs Contribute to ICB Response

### Method

### Predicted TA Presentation.

HLA alleles for melanoma samples from previous studies on ICB were inferred from RNA-seq using Optitype. Promiscuous binders for a given MAP (all HLA alleles capable of presenting the MAP) were obtained using NetMHCpan-4.1b. They corresponded to HLA alleles for which the given MAP had an eluted ligand likelihood prediction rank <2%. A given TA was considered as presented in a sample if it had an expression >0 RPHM and at least one of the patient's HLA allotypes was a potential binder. If the patient expressed more than one HLA allele capable of presenting a TA, the TA was counted multiple times. Only cutaneous melanoma samples from previous studies were analyzed.

Number of T Cell Clones Expanded On-Therapy in Patients from Riaz Et Al⁸.

The TCRB clonotypes from Riaz et al.⁸ were downloaded from (https://www.github.com/riazn/bms038_analysis). Then, the TCRB clones expanded per patient were determined using the FEST web tool (www.stat-apps.onc.jhmi.edu/FEST) with the following parameters per patient: 1) pre-therapy sample as a reference, 2) nTemplates_threshold=1, 3) Ignore_baseline_threshold=TRUE, 4) Nucleotide_level=FALSE, 5) FDR 5% and OR 5.

### Results

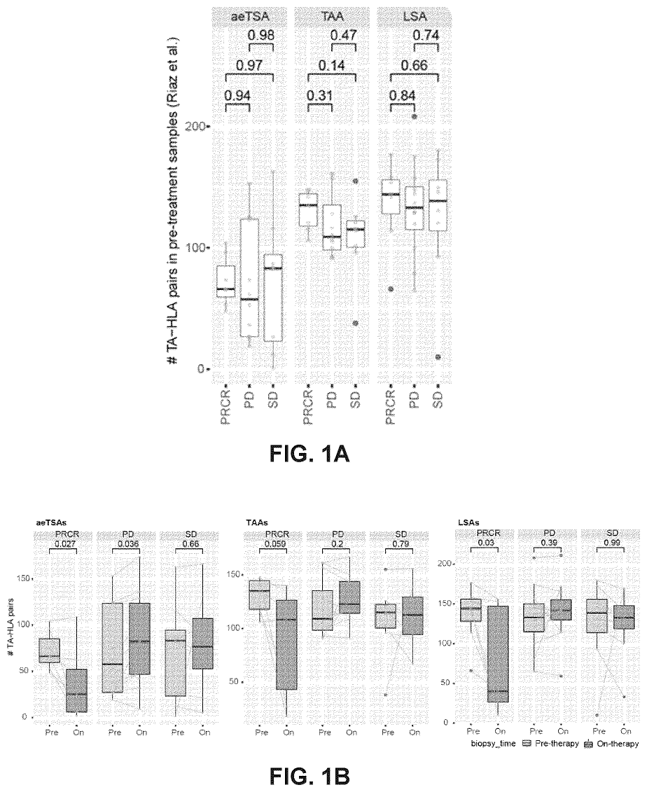

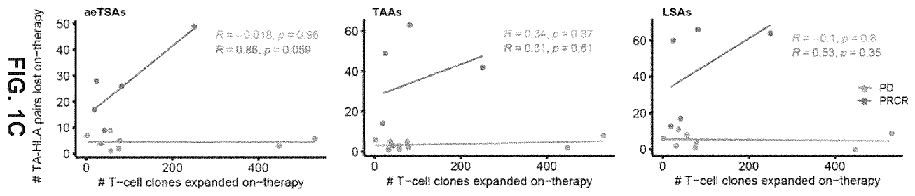

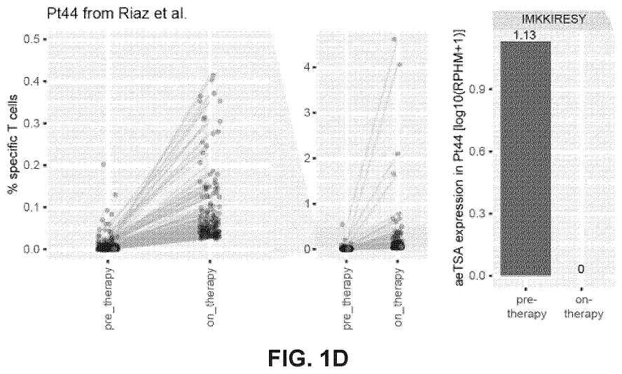

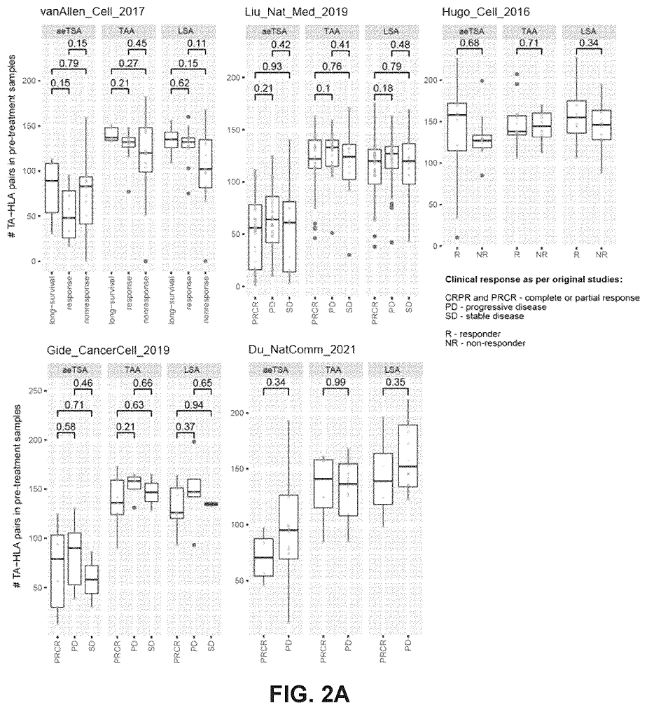

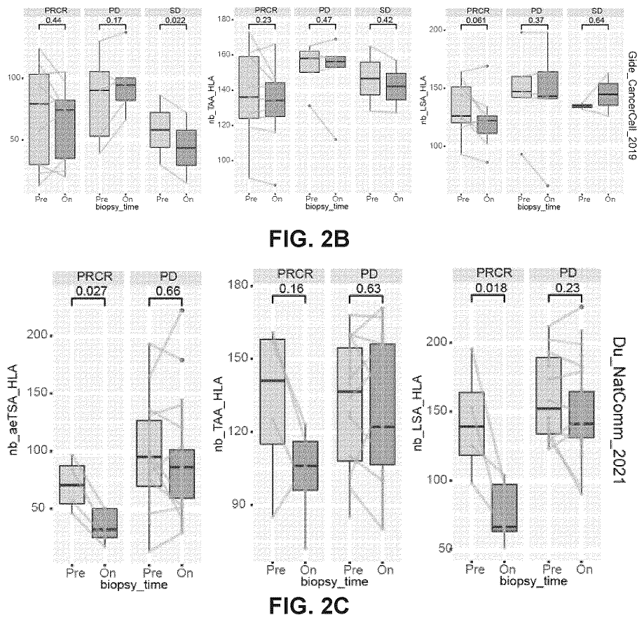

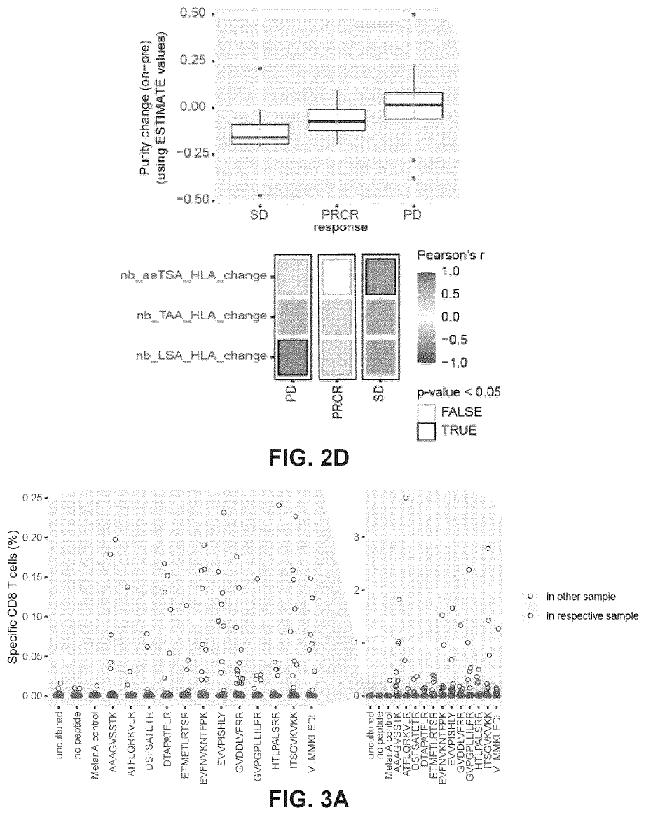

Despite their high abundance in cancer cells, little is known about the role of unmutated TAs in spontaneous or treatment-induced anti-tumor immunity in vivo. To evaluate this, the number of unmutated TAs in pre-treatment biopsies from melanoma patients treated with ICB (anti-PD-1, anti-PD-L1, and/or anti-CTLA) was predicted. TA presence in individual tumors was inferred based on two criteria: expression at the RNA level of both the TA and a cognate HLA allotype (i.e., presence of TA-HLA pairs)⁴. In all datasets tested, patients presented high numbers of TA-HLA pairs. Still, no significant difference in the number of TA-HLA pairs was seen in pre-treatment samples between responders and non-responders (FIGS. 1A, 2A, 2B). A recent meta-analysis suggests that the lack of correlation between TA load and response to ICB commonly results from a threshold effect: when one or a few TAs are immunogenic, having more TAs does not confer any advantage¹⁰. For now, the lack of immunopeptidomic data and complete sets of unmutated TAs from these patients prevents drawing definitive conclusions on the value of unmutated TA numbers in predicting response to ICB.

In their previous study of anti-PD-1 therapy in melanoma, Riaz and colleagues reported a linear correlation between the number of expanded T-cell clones and the number of predicted mTSAs lost on-therapy in responders (partial and complete response patients, PRCR) but not in non-responders (stable or progressive disease patients, SD or PD)⁸. Notably, using the Riaz et al. data⁸, it was found that responders, but not non-responders, showed a marked decrease in the number of unmutated TA-HLA pairs on-therapy (FIGS. 1B, 2C, 2D), which could not be explained solely by decreased tumor purity (FIG. 2E). The positive correlation between unmutated TA disappearance and response to ICB was validated in one out of two additional datasets with pre- and on-ICB samples^{11,12} (FIGS. 2C, 2D). Furthermore, the loss of aeTSA-HLA numbers in responders had a strong linear correlation with the number of T cell clones expanded on-therapy, whereas a positive but non-significant correlation with T cell expansion was observed for TAAs and LSAs (FIG. 1C).

To determine whether the loss of aeTSAs in responders was directly due to their recognition and elimination by certain T cell clonotypes, experiments were performed to identify the cognate antigens for the expanded T cell receptor Vβ (TCRB) sequences. To this end, the overlap between the expanded TCRB sequences from Riaz et al.⁸ and the database of aeTSA-specific CD8 TCRB clonotypes obtained using functional expansion of specific T cells (FEST) assays⁹ in previous studies was first checked^{4,15,16}. In the FEST assays, TCRB sequencing is performed on T cells isolated from healthy donor PBMCs and stimulated or not with individual synthetic aeTSAs. TCR clonotypes responsive to aeTSAs are then identified based on their significant expansion in the aeTSA-stimulated condition compared to the control. Remarkably, it was found that one TCRB clone expanded in a responder from Riaz et al.⁸ was specific to an exonic aeTSA derived from oncofetal IGF2BP1¹⁷, which was previously identified in ovarian cancer¹⁶. The expansion of this CD8 T-cell clonotype was associated with a complete loss of the IGF2BP1-derived aeTSA on-therapy, strongly indicating direct recognition and elimination (FIG. 1D).

### Example 3: Immunogenicity of Selected Melanoma Tumor Antigen Candidates

A functional expansion of specific T cells (FEST) assay was performed on melanoma aeTSAs identified herein.

### Method

aeTSA selection for Immunogenicity assay. Melanoma aeTSAs to be tested in T cell-based immunogenicity assays were selected based on the following criteria: 1) complete loss of expression (RPHM) on-therapy in at least one responder from the Riaz et al.⁸ cohort capable to present the respective aeTSAs (at least one HLA allele with rank elution <2% in NetMHCpan4.1b⁵), 2) elevated mean expression in TCGA-SKCM, 3) the HLA allotype presenting the aeTSA (HLA-A*02:01 or HLA-B*68:01 shared by the melanoma samples and PBMCs donors), 4) the aeTSA biotype (25% canonical, 75% non-canonical), 5) novel MAP status.

### Peptide-Specific T Cell Functional Expansion (FEST).

T cells were cultured as previously described⁹, with minor modifications. Briefly, on day 0, thawed PBMCs from D36 (Miltenyi Biotec) were T cell-enriched using the Human Pan T cell isolation kit (Miltenyi Biotec). T cells were resuspended at 2×10⁶/mL in AIM V media supplemented with 50 μg/mL gentamicin (ThermoFisher Scientific) and 1% HEPES. The T cell-negative fraction was irradiated at 30 Gγ, washed, and resuspended at 2×10⁶/mL in AIM V media supplemented with 50 μg/mL gentamicin and 1% HEPES. 2.5 ml per well of both T cells and irradiated T cell-depleted cells were added to a 12-well plate, along with each peptide (GLS Biochem) (1 μg/mL final concentration) or without peptide. Cells were cultured for 10 days at 37° C., 5% CO₂. On day 3 and 7, half the culture media was replaced with fresh culture media containing 100 IU/mL IL-2, 50 ng/mL IL-7, and 50 ng/mL IL-15 (day 3) and 200 IU/mL IL-2, 50 ng/mL IL-7, and 50 ng/mL IL-15 (day 7). On day 10, thawed PBMCs from the same donor were used to generate a new batch of T cell-depleted cells. These cells were irradiated at 30 Gγ and loaded with 1 g/mL of relevant peptide or without peptide during 2 h at 37° C. These cells were washed and added to cultures at a 1:1 T cell:non-T cell ratio. On day 13 and 17, at least half the culture media was replaced with fresh culture media (final concentrations: 100 IU/mL IL-2, 25 ng/mL IL-7, and 25 ng/mL IL-15). On day 20, cells were harvested to perform FEST assays.

### Functional Expansion of Specific T Cells (FEST) Assay.

For FEST assays, CD8⁺ cells were further isolated using the Human CD8⁺ T Cell Isolation Kit (Miltenyi Biotec). As a negative control, CD8⁺ T cells were also isolated from freshly thawed uncultured PBMCs of the same healthy donor. DNA was extracted from CD8⁺ T cells using a QIAGEN DNA blood mini kit (QIAGEN). TCR Vβ CDR3 sequencing was performed using the ultradeep resolution of the immunoSEQ platform (Adaptive Biotechnologies). Raw data exported from the immunoSEQ portal were processed with the FEST web tool⁹ (www.stat-apps.onc.ihmi.edu/FEST).

### Results

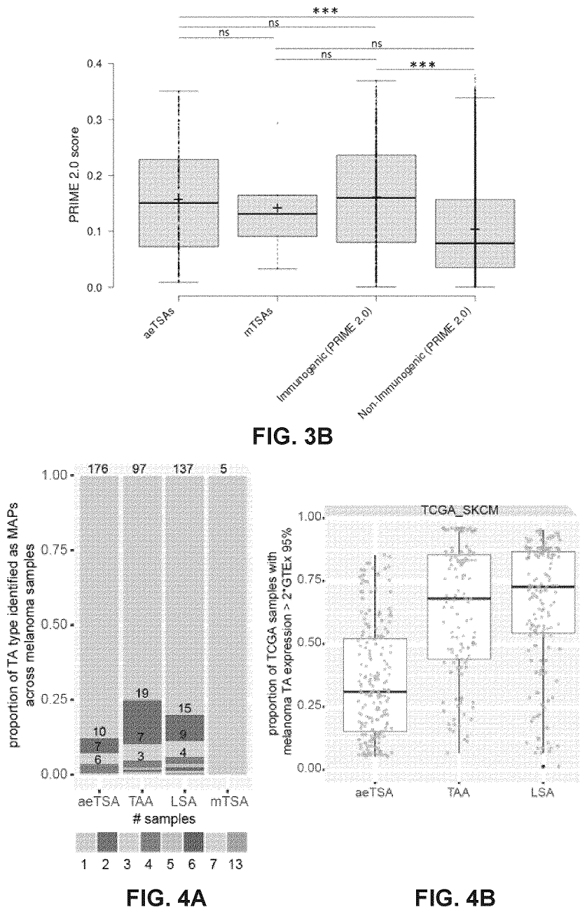

The ability of 12 selected aeTSAs to stimulate the expansion of specific T cell clonotypes in vitro was assessed in a FEST assay. The aeTSAs were able to induce specific T cell expansion (FIG. 3). The TCRβ clonotypes expanded by each of the aeTSAs are depicted in Table 3.

TABLE 3

| | Amino acid sequences of the CDR3 of the TCRβ |
|---|---|
| aeTSA | chain of the clonotypes expanded |
| AAAGVSSTK (SEQ ID NO: 73) | CASHPAGADMNTEAFF (SEQ ID NO: 1012) |
| | CASSLAGRETQYF (SEQ ID NO: 1013) |
| | CASSVWGEPYNEQFF (SEQ ID NO: 1014) |
| | CASSRTDSYEQYF (SEQ ID NO: 1015) |
| | CASSLQGANGYTF (SEQ ID NO: 1016) |
| | CASSPTNTYEQYF (SEQ ID NO: 1017) |
| ATFLQRKVLR (SEQ ID NO: 47) | CASSLSYTGELFF (SEQ ID NO: 1018) |
| | CASSRTGNQPQHF (SEQ ID NO: 1019) |
| | CASSSSGEQYF (SEQ ID NO: 1020) |
| | CASSVEEGSYEQYF (SEQ ID NO: 1021) |
| | CASRDYGYTF (SEQ ID NO: 1022) |
| | CASSYSGDYNEQFF (SEQ ID NO: 1023) |
| | CASSFEAYSNQPQHF (SEQ ID NO: 1024) |
| | CASSGGDSPYEQYF (SEQ ID NO: 1025) |
| | CASSLDTGVGANVLTF (SEQ ID NO: 1026) |
| | CASSQLGVTEAFF (SEQ ID NO: 1027) |
| | CASSPGTAQETQYF (SEQ ID NO: 1028) |
| | CASSLDRGDYEQYF (SEQ ID NO: 1029) |
| DSFSATETR (SEQ ID NO: 7) | CASSLGGLYEQYF (SEQ ID NO: 1030) |
| | CASSQDLANTGELFF (SEQ ID NO: 1031) |
| | CSVEQLGEMNTEAFF (SEQ ID NO: 1032) |
| | CASSQGEIDGYTF (SEQ ID NO: 1033) |
| | CASSLTGDGYTF (SEQ ID NO: 1034) |
| | CASSPRDGTYEQYF (SEQ ID NO: 1035) |
| | CASSLDRAYEQYF (SEQ ID NO: 1036) |
| | CASSQDLGSSYEQYF (SEQ ID NO: 1037) |
| | CASGTGGSTDTQYF (SEQ ID NO: 1038) |
| | CASSETDTQYF (SEQ ID NO: 1039) |
| | CASSVGQFSNQPQHF (SEQ ID NO: 1040) |
| | CASSPRTNQPQHF (SEQ ID NO: 1041) |
| | CASSLGGTGPYEQYF (SEQ ID NO: 1042) |
| DTAPATFLR (SEQ ID NO: 138) | CAISGDGYGYTF (SEQ ID NO: 1043) |
| | CASSLTGGTEAFF (SEQ ID NO: 1044) |
| | CASSLSDSYEQYF (SEQ ID NO: 1045) |
| | CASSQEGREVDGYTF (SEQ ID NO: 1046) |
| | CASSEVSNQPQHF (SEQ ID NO: 1047) |
| | GNTIYF (SEQ ID NO: 1048) |
| | CASQGAYEQYF (SEQ ID NO: 1049) |
| | CASSFKPGLKWRDEQFF (SEQ ID NO: 1050) |
| | CASSIGTANSYEQYF (SEQ ID NO: 1051) |
| | CASSIGTNTEAFF (SEQ ID NO: 1052) |
| | CASSVGDSGNTIYF (SEQ ID NO: 1053) |
| | CASSSGVTNEKLFF (SEQ ID NO: 1054) |
| | CASSATGDTEAFF (SEQ ID NO: 1055) |
| ETMETLRTSR (SEQ ID NO: 98) | CASSLRTGRGYGYTF (SEQ ID NO: 1056) |
| | CAIQRGRTGELFF (SEQ ID NO: 1057) |
| | CASSLATGYNEQFF (SEQ ID NO: 1058) |
| | CASSFTERGYEKLFF (SEQ ID NO: 1059) |
| | CASSPRTGMNTEAFF (SEQ ID NO: 1060) |
| | CASSQDSEQGLVQPQHF (SEQ ID NO: 1061) |
| | CASSRSTSGRADEQFF (SEQ ID NO: 1062) |
| | CASRASSGLSYEQYF (SEQ ID NO: 1063) |
| | CASSVAGSTEAFF (SEQ ID NO: 1064) |
| | CASSQEGGMNTEAFF (SEQ ID NO: 1065) |
| | CASSSSDRVGEQYF (SEQ ID NO: 1066) |
| | CASSLGQNSYNEQFF (SEQ ID NO: 1067) |
| | CASSPRSSYNEQFF (SEQ ID NO: 1068) |
| | CASSLLGGGQPQHF (SEQ ID NO: 1069) |
| | CASSRGLAVSYNEQFF (SEQ ID NO: 1070) |
| EVFNVKNTFPK (SEQ ID NO: 134) | CASSLLARSSYEQYF (SEQ ID NO: 1071) |
| | CASSPTENTEAFF (SEQ ID NO: 1072) |
| | CASSVGGGRNYEQYF (SEQ ID NO: 1073) |
| | CASSYSGGPYEQYF (SEQ ID NO: 1074) |
| | CASSLGDGQPQHF (SEQ ID NO: 1075) |
| | CASSQGDYGYTF (SEQ ID NO: 1076) |
| | CASSYQGTNTEAFF (SEQ ID NO: 1077) |
| | CASSFIGGQPQHF (SEQ ID NO: 1078) |
| | CASSLGLAGTNTGELFF (SEQ ID NO: 1079) |
| EWPISHLY (SEQ ID NO: 41) | CASSVGPETQYF (SEQ ID NO: 1080) |
| | CASSQGLSSGNTIYF (SEQ ID NO: 1081) |
| | CASSSATEAFF (SEQ ID NO: 1082) |
| | CASSLKGVTYEQYF (SEQ ID NO: 1083) |
| | CASSPDGGEQYF (SEQ ID NO: 1084) |
| | CASSLFPDTQYF (SEQ ID NO: 1085) |
| | CASSLGDSYTGELFF (SEQ ID NO: 1086) |
| | CASSVEGYTEAFF (SEQ ID NO: 1087) |
| | CASSPPESYEQYF (SEQ ID NO: 1088) |
| GVDDLVFRR (SEQ ID NO: 64) | CASSPDPEGGEQYF (SEQ ID NO: 1089) |
| | CASSLQGHGYTF (SEQ ID NO: 1090) |
| | CASNVARTGGDISGDYEQYF (SEQ ID NO: 1091) |
| | CASSLGLAGSYEQYF (SEQ ID NO: 1092) |
| | CASSDSSYNSPLHF (SEQ ID NO: 1093) |
| GVPGPLLILPR (SEQ ID NO: 59) | CASSEWGQGDSNNYGYTF (SEQ ID NO: 1094) |
| | CASSNLAGLNEQFF (SEQ ID NO: 1095) |
| | CASSPPSGGFEQFF (SEQ ID NO: 1096) |
| | CASSFDGWNTEAFF (SEQ ID NO: 1097) |
| | CASSLGGQGYGYTF (SEQ ID NO: 1098) |
| | CASSPGQGNNEKLFF (SEQ ID NO: 1099) |
| | CASSIVFGSRNSPLHF (SEQ ID NO: 1100) |
| | CASKESTEAFF (SEQ ID NO: 1101) |
| | CSANTRDISYEQYF (SEQ ID NO: 1102) |
| HTLPALSRR (SEQ ID NO: 66) | CASSFQGLNTEAFF (SEQ ID NO: 1103) |
| | CASSPGQGNEKLFF (SEQ ID NO: 1104) |
| | CASSLGSGYEQYF (SEQ ID NO: 1105) |
| | CASSLGLYNEQFF (SEQ ID NO: 1106) |
| | CASSPGTDYNEQFF (SEQ ID NO: 1107) |
| | CATRPGAIRGYTF (SEQ ID NO: 1108) |
| | CASSLIGGADTEAFF (SEQ ID NO: 1109) |
| ITSGVKVKK (SEQ ID NO: 110) | CASSLLTGNYGYTF (SEQ ID NO: 1110) |
| | CASYNRGERGGYTF (SEQ ID NO: 1111) |
| | CASSPGTSGSSYNEQFF (SEQ ID NO: 1112) |
| | CASSPVPRGDVPYEQYF (SEQ ID NO: 1113) |
| | CASSADPNTEAFF (SEQ ID NO: 1114) |
| | CSARDTRGGQPQHF (SEQ ID NO: 1115) |
| | CASSLTSGSRNEQFF (SEQ ID NO: 1116) |
| | CASSSGRFYEQYF (SEQ ID NO: 1117) |
| | CASSVLAGGSYEQYF (SEQ ID NO: 1118) |
| | CASSYGTGVEQYF (SEQ ID NO: 1119) |
| | CASSQPGTSGYNEQFF (SEQ ID NO: 1120) |
| | CASSRLAGGQETQYF (SEQ ID NO: 1121) |
| | CASRPGTGGRMDIQYF (SEQ ID NO: 1122) |
| | CAIRSTDTQYF (SEQ ID NO: 1123) |
| VLMMKLEDL (SEQ ID NO: 120) | CASSLGGNQPQHF (SEQ ID NO: 1124) |
| | CASSSGPSGGNTIYF (SEQ ID NO: 1125) |
| | CASSLGTGGYEQFF (SEQ ID NO: 1126) |
| | CASSLGGGGYGYTF (SEQ ID NO: 1127) |
| | CASRTGGANTEAFF (SEQ ID NO: 1128) |
| | CASSGDRGHEQYF (SEQ ID NO: 1129) |
| | CAISETGLVGEKLFF (SEQ ID NO: 1130) |
| | CASSLGQGSEKLFF (SEQ ID NO: 1131) |
| | CASSLGRMNTEAFF (SEQ ID NO: 1132) |
| | CASSLLGPGELFF (SEQ ID NO: 1133) |
| | CASSSRDRESGNTIYF (SEQ ID NO: 1134) |
| | CASSPEGGSSYNEQFF (SEQ ID NO: 1135) |
| | CASSQSGQGYGYTF (SEQ ID NO: 1136) |
| | CASSGTSGAYNEQFF (SEQ ID NO: 1137) |

These results show that the representative aeTSAs tested induce polyclonal T cell expansion following in vitro stimulation, providing evidence that they may be useful for immunotherapy against melanoma.

The TCR recognition propensity of 200 representative aeTSAs and 5 mTSAs described herein was also assessed using the PRedictor of Immunogenic Epitope PRIME2.0 web tool (http://ec2-18-188-210-66.us-east-2.compute.amazonaws.com:3000, Gfeller, D., et al., Improved predictions of antigen presentation and TCR recognition with MixMHCpred2.2 and PRIME2.0 reveal potent SARS-CoV-2 CD8+ T-cell epitopes. Cell Systems, 2023. 14(1): p. 72-83.e5). PRIME2.0 is based on a neural network and uses as input features (1) the predicted HLA-I presentation score (−log(% rank) of MixMHCpred2.2), (2) the amino acid frequency at positions with minimal impact on binding to HLA-I and more likely to face the TCR, and (3) the length of the peptide. The PRIME2.0 scores of the aeTSAs and mTSAs was compared to that of 590 known immunogenic peptides (binding 53 HLA alleles) and 5887 non-immunogenic peptides (binding 62 HLA alleles) used to train PRIME2.0. The results reported in FIG. 3B show that the tested aeTSAs have a PRIME2.0 score that is (1) comparable (and even better) to that of mTSAs; (2) comparable to that of a set of known immunogenic control peptides (neoantigens) used to train the PRIME 2.0 algorithm; and (3) significantly higher than that of a set of non-immunogenic control peptides used to train the PRIME 2.0 algorithm. The aeTSA of SEQ ID NO:1 described herein was shown to have a very high PRIME 2.0 immunogenicity score of 0.31991. This date provides further evidence that the aeTSAs described herein are immunogenic and may be useful for immunotherapy against melanoma.

### Example 4: Further Evidence that aeTSAs are Good Candidates for Immunotherapy

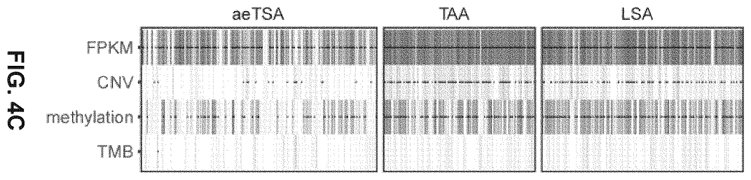

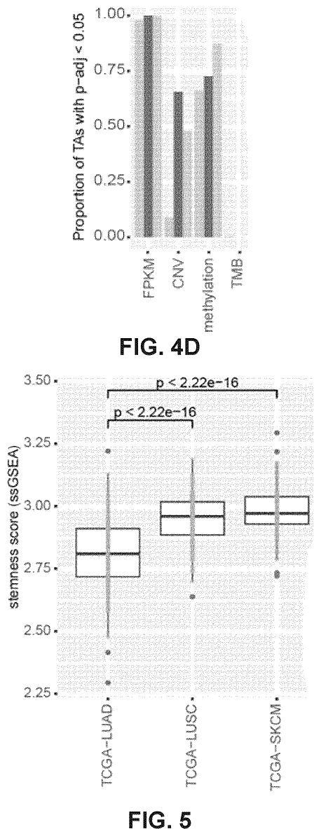

A therapeutically attractive feature of unmutated TAs is their sharing between patients. In contrast to mTSAs which were patient-specific, aeTSAs from melanoma were abundant and shared at the peptide and RNA levels (FIGS. 4A-B). Nevertheless, TAs showed a TA type-, cancer type- and cancer subtype-specific expression pattern and regulation. Specifically, it was found that aeTSAs coded by oncofetal (or cancer-germline) genes and TAAs coded by cell cycle genes were enriched in melanoma samples from TCGA, consistent with an increased stemness of these tumors (FIGS. 4B, 5).

Using TCGA multi-omics data, a pronounced correlation between the expression of TAs and their corresponding source genes was found, suggesting a gene-level regulation (FIG. 4C). Expression of aeTSAs was often correlated with hypomethylation of the source gene promoters in melanoma. By contrast, TAAs and LSAs showed a prevalent association with both the focal DNA copy number (CNV) and the hypomethylation of source-gene promoters, whereas no TA type was correlated with the TMB (FIG. 4D). In summary, these results indicate that, in agreement with the role of MAPs in mirroring the internal cell state, TA expression reflects cancer cell programs shared between patients and regulated, at least in part, at the mutational and/or epigenetic level. The association with cancer (sub)type suggests that unmutated TA prioritization is possible and likely important for effective therapy.

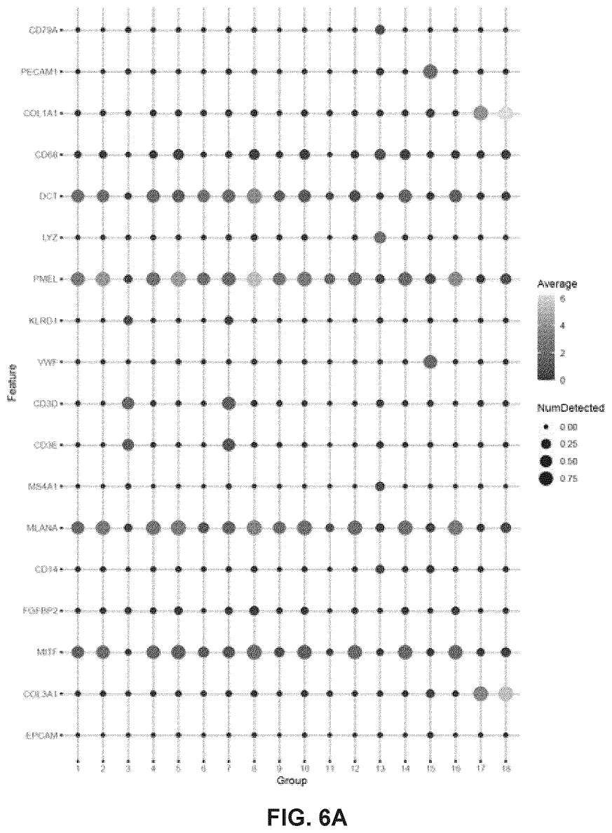

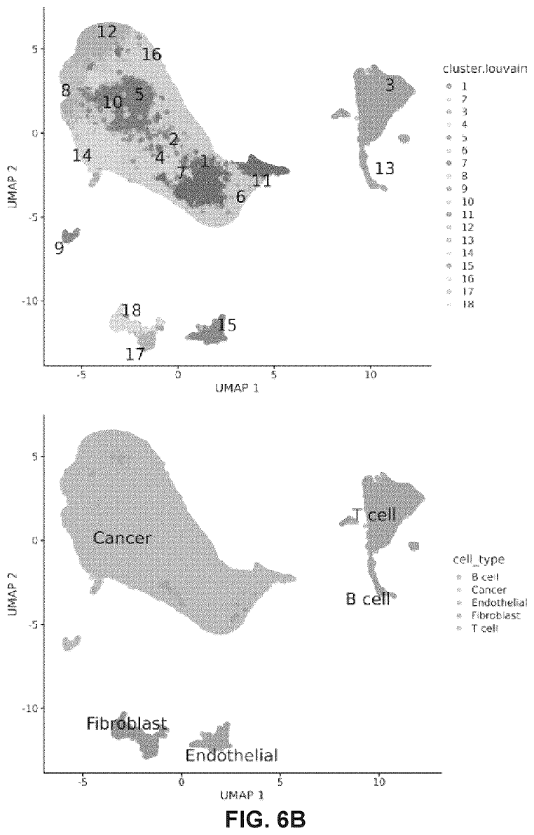

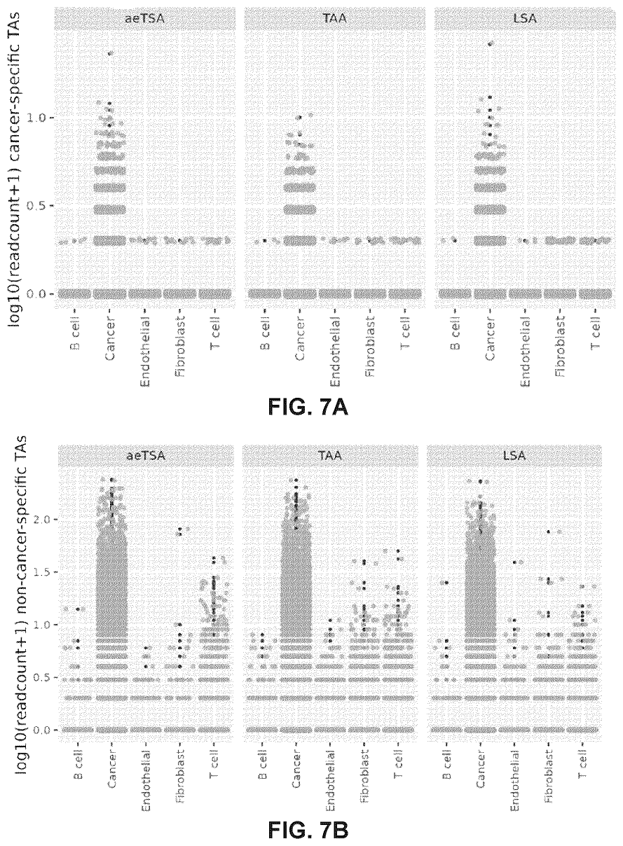

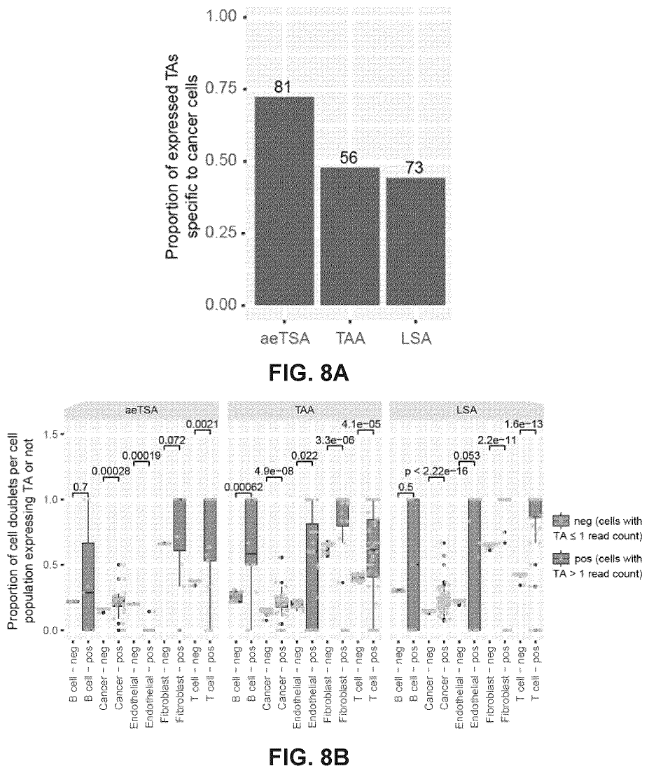

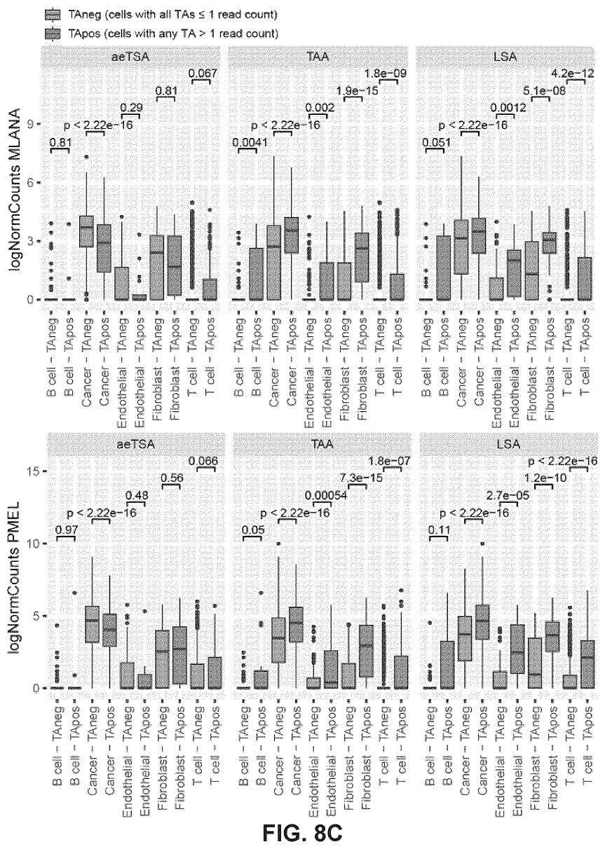

MAPs obtained from MHC I immunoprecipitation of bulk tumor lysates are “contaminated” by peptides from tumor-infiltrating immune cells and other stromal cells in the microenvironment¹³. Hence it was aimed to validate that the unmutated TA expression is associated with malignant cells (or with the cell lineage of origin for LSAs) using published single-cell RNA-seq datasets from melanoma¹⁴ (FIGS. 6A, B). It was found that TAs were highly and primarily expressed by cancer cells (FIGS. 7A, B). Among the aeTSAs detected, most were expressed in cancer cells only (72%). When detected in annotated non-cancer cells, TA expression was associated with up to 100% cell doublet formation between non-cancer and cancer cells (FIG. 8A). Indeed, melanoma TA-positive non-cancer cell populations showed increased expression of melanoma (and melanocyte) markers MLANA (FIG. 8B, upper panel) and PMEL (FIG. 8B, lower panel) relative to TA-negative non-cancer cells. Hence, aeTSAs were cancer cell-specific and their detection in other cell populations resulted from technical limitations in the single-cell sample preparation.

Altogether, the results presented in this study strongly support the immune targeting of unmutated TAs across cancers with varying TMB levels. aeTSAs are particularly attractive targets for immunotherapy considering their cancer specificity, immunogenicity, high abundance, and sharing between patients.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

1. Chong, C. et al. Integrated proteogenomic deep sequencing and analytics accurately identify non-canonical peptides in tumor immunopeptidomes. Nat. Commun. 11, 1293 (2020).
2. Lamoliatte, F., McManus, F. P., Maarifi, G., Chelbi-Alix, M. K. & Thibault, P. Uncovering the SUMOylation and ubiquitylation crosstalk in human cells using sequential peptide immunopurification. Nat. Commun. 8, 14109 (2017).
3. Laumont, C. M. et al. Noncoding regions are the main source of targetable tumor-specific antigens. Sci. Transl. Med. 10, eaau5516 (2018).
4. Ehx, G. et al. Atypical acute myeloid leukemia-specific transcripts generate shared and immunogenic MHC class-I-associated epitopes Article Atypical acute myeloid leukemia-specific transcripts generate shared and immunogenic MHC class-I-associated epitopes. Immunity 54, 737-752 (2021).
5. Reynisson, B., Alvarez, B., Paul, S., Peters, B. & Nielsen, M. NetMHCpan-4.1 and NetMHCllpan-4.0: improved predictions of MHC antigen presentation by concurrent motif deconvolution and integration of MS MHC eluted ligand data. Nucleic Acids Res. 48, W449-W454 (2020).
6. Courcelles, M. et al. MAPDP: A Cloud-Based Computational Platform for Immunopeptidomics Analyses. J. Proteome Res. 19, 1873-1881 (2020).
7. Ruiz Cuevas, M. V. et al. BamQuery: a proteogenomic tool for the genome-wide exploration of the immunopeptidome. bioRxiv (2022). doi:10.1101/2022.10.07.510944.
8. Riaz, N. et al. Tumor and Microenvironment Evolution during Immunotherapy with Nivolumab. Cell 171, 934-949.e15 (2017).
9. Danilova, L. et al. The mutation-associated neoantigen functional expansion of specific T cells (MANAFEST) assay: A sensitive platform for monitoring antitumor immunity. Cancer Immunol. Res. 6, 888-899 (2018).
10. Gurjao, C., Tsukrov, D., Imakaev, M., Luquette, L. J. & Mirny, L. A. Is tumor mutational burden predictive of response to immunotherapy? Elife (2023). doi:10.7554/eLife.87465.1.
11. Gide, T. N. et al. Distinct Immune Cell Populations Define Response to Anti-PD-1 Monotherapy and Anti-PD-1/Anti-CTLA-4 Combined Therapy. Cancer Cell 35, 238-255.e6 (2019).
12. Du, K. et al. Pathway signatures derived from on-treatment tumor specimens predict response to anti-PD1 blockade in metastatic melanoma. Nat. Commun. 12, 6023 (2021).
13. Jaeger, A. M. et al. Deciphering the immunopeptidome in vivo reveals new tumour antigens. Nature 607, 149-155 (2022).
14. Zhang, C. et al. A single-cell analysis reveals tumor heterogeneity and immune environment of acral melanoma. Nat. Commun. 13, 7250 (2022).
15. Apavaloaei, A. et al. Induced pluripotent stem cells display a distinct set of MHC I-associated peptides shared by human cancers. Cell Rep. 40, 111241 (2022).
16. Zhao, Q. et al. Proteogenomics Uncovers a Vast Repertoire of Shared Tumor-Specific Antigens in Ovarian Cancer. Cancer Immunol. Res. 8, 544-555 (2020).
17. Huang, X. et al. Insulin-like growth factor 2 mRNA-binding protein 1 (IGF2BP1) in cancer. J. Hematol. Oncol. 11, 88 (2018).
18. Miranda, A. et al. Cancer stemness, intratumoral heterogeneity, and immune response across cancers. Proc. Natl. Acad. Sci. U.S.A 116, 9020-9029 (2019).
19. Van Allen, E. M. et al. Genomic correlates of response to CTLA-4 blockade in metastatic melanoma. Science 352, 207-212 (2015).
20. Hugo, W. et al. Genomic and Transcriptomic Features of Response to Anti-PD-1 Therapy in Metastatic Melanoma. Cell 165, 35-44 (2016).
21. Liu, D. et al. Integrative molecular and clinical modeling of clinical outcomes to PD1 blockade in patients with metastatic melanoma. Nat. Med. 25, 1916-1927 (2019).

1.-79. (canceled)

80. A tumor antigen peptide (TAP) comprising or consisting of one of the amino acid sequences set forth in SEQ ID NOs:1-505.

81. The TAP of claim 80, wherein the TAP binds to (a) an HLA-A*01:01 molecule and comprises or consists of the sequence of SEQ ID NO: 11, 14, 20, 27, 29, 37, 86, 87, 121, 150, 165, 173, 279, 288, 299, 308, 312, 316, 319, 331, 340, 352, 362, 363, 384, 395, 398, 411, 419, 429, 458, 474, 476, 487, 492, 500, or 501; (b) an HLA-A*02:01 molecule and comprises or consists of the sequence of SEQ ID NO: 1, 2, 4, 19, 22, 25, 40, 50, 52, 54, 60, 65, 67, 76, 81, 89, 91, 103, 107, 120, 124, 128, 135, 157, 175, 179, 186, 188, 201, 208, 218, 219, 228, 232, 233, 240, 250, 257, 261, 263, 265, 266, 282, 286, 298, 311, 315, 317, 325, 333, 337, 349, 367, 373, 387, 388, 393, 399, 404, 412, 414, 415, 422, 434, 437, 455, 480, 489, or 494; (c) an HLA-A*02:09 molecule and comprises or consists of the sequence of SEQ ID NO: 22, 40, 50, 62, 83, 89, 232, 265, 282, 311, 315, 317, 337, 349, 367, 399, 407, 412, or 494; (d) an HLA-A*03:01 molecule and comprises or consists of the sequence of SEQ ID NO: 209, 247, 272, 347, or 505; (e) an HLA-A*23:01 molecule and comprises or consists of the sequence of SEQ ID NO: 48, 84, 129, 151, 152, 171, 174, 182, 223, 237, 245, 290, 358, 397, 426, 446, 481, 482, or 486; (f) an HLA-A*24:02 molecule and comprises or consists of the sequence of SEQ ID NO: 31, 244 or 481; (g) an HLA-A*25:01 molecule and comprises or consists of the sequence of SEQ ID NO: 11, 31, 249, 289, 403, 425, 464, or 490; (h) an HLA-A*26:01 molecule and comprises or consists of the sequence of SEQ ID NO: 11, 20, 27, 39, 41, 42, 45, 114, 116, 122, 168, 181, 268, 284, 329, 364, 398, 417, 430, 440, 447, 452, 457, 460, 462, 475, 490, or 497; (i) an HLA-A*30:01 molecule and comprises or consists of the sequence of SEQ ID NO: 26, 304 or 376; (j) an HLA-A*30:02 molecule and comprises or consists of the sequence of SEQ ID NO: 117, 185, 190, 193, 197, 256, 294, 323, 471, or 485; (k) an HLA-A*33:01 molecule and comprises or consists of the sequence of SEQ ID NO: 248; (1) an HLA-A*68:01 molecule and comprises or consists of the sequence of SEQ ID NO: 6-8, 12, 15, 23, 28, 41, 42, 47, 51, 53, 56, 59, 64, 66, 68, 70, 73-75, 77, 79, 80, 82, 85, 88, 92, 93, 96-99, 101, 102,108-112,118,119,123,130,131, 134, 137, 138, 141, 142,144-147,149,153,154,156,158, 160, 161, 166, 169, 172, 176, 184, 198, 202, 203, 212, 214, 216, 217, 220, 224, 226, 229, 234, 236, 239, 248, 254, 269, 270, 275, 280, 283, 287, 293, 300, 301, 303, 306, 310, 314, 320, 328, 332, 348, 354, 355, 359, 360, 366, 369, 370, 372, 381, 382, 386, 389, 390, 392, 394, 402, 405, 406, 409, 413, 418, 424, 428, 436, 441-445, 449, 456, 459, 465-468, 473, 479, 483, 488, 493, 499, or 502; (m) an HLA-B*07:02 molecule and comprises or consists of the sequence of SEQ ID NO: 35, 36, 43, 104, 125, 127, 139, 140, 187, 191, 196, 206, 231, 243, 246, 251, 252, 260, 281, 291, 292, 321, 330, 361, 408, 432, 435, 439, 451, 461, 491, or 503; (n) an HLA-B*08:01 molecule and comprises or consists of the sequence of SEQ ID NO: 21, 162, 189, 286, 307, 309, 317, 339, 343, 349, or 414; (o) an HLA-B*13:02 molecule and comprises or consists of the sequence of SEQ ID NO: 207, 311, 393, 400, or 455; (p) an HLA-B*14:01 molecule and comprises or consists of the sequence of SEQ ID NO: 396; (q) an HLA-B*15:01 molecule and comprises or consists of the sequence of SEQ ID NO: 11, 26, 30, 63, 113, 132, 148, 192, 195, 200, 207, 210, 222, 235, 238, 258, 262, 267, 276, 295, 297, 305, 309, 318, 326, 336, 342, 374, 377, 383, 401, 421, 427, 433, 438, 450, 463, 470, or 477; (r) an HLA-B*18:01 molecule and comprises or consists of the sequence of SEQ ID NO: 3, 10, 33, 225 or 396; (s) an HLA-B*37:01 molecule and comprises or consists of the sequence of SEQ ID NO: 302; (t) an HLA-B*38:01 molecule and comprises or consists of the sequence of SEQ ID NO: 26, 180, 338, 341, 378, 380, 416, 448, 454, 496, or 498; (u) an HLA-B*40:01 molecule and comprises or consists of the sequence of SEQ ID NO: 215 or 322; (v) an HLA-B*40:02 molecule and comprises or consists of the sequence of SEQ ID NO: 213, 215, 339, or 351; (w) an HLA-B*44:02 molecule and comprises or consists of the sequence of SEQ ID NO: 346, 353, 356, or 453; (x) an HLA-B*49:01 molecule and comprises or consists of the sequence of SEQ ID NO: 9, 57, 78, 115, 136, 167, 255, 273, 274, 278, 313, 339, 480, 484, or 495; (y) an HLA-B*51:01 molecule and comprises or consists of the sequence of SEQ ID NO: 227 or 259; (z) an HLA-C*03:03 molecule and comprises or consists of the sequence of SEQ ID NO: 24, 69, 204 or 388; (aa) an HLA-C*03:04 molecule and comprises or consists of the sequence of SEQ ID NO: 24, 49, 69, 159, 241, 242 or 334; (bb) an HLA-C*06:02 molecule and comprises or consists of the sequence of SEQ ID NO: 13, 34 or 72; (cc) an HLA-C*07:01 molecule and comprises or consists of the sequence of SEQ ID NO: 13, 75, 133, 163, 183, 199, 205, 285, 334, 391 or 472; (dd) an HLA-C*07:02 molecule and comprises or consists of the sequence of SEQ ID NO: 13, 32, 36, 105, 177, 339, 371 or 478; (ee) an HLA-C*08:02 molecule and comprises or consists of the sequence of SEQ ID NO: 18, 54 or 379; (ff) an HLA-C*12:03 molecule and comprises or consists of the sequence of SEQ ID NO: 24, 49, 58, 71, 94, 162, 178, 242, 289, 344, 345, 357, 367, 368, 375, 423 or 431; or (gg) an HLA-C*14:02 molecule and comprises or consists of the sequence of SEQ ID NO: 105, 271, 350, 365, 385, 410, 423, or 504.

82. The TAP of claim 80, which is encoded by a sequence located a non-protein coding region of the genome or by a long non-coding RNA.

83. The TAP of claim 82, wherein said non-protein coding region of the genome is an intergenic region or an intron.

84. A combination comprising at least two of the TAPs defined in claim 80.

85. A synthetic long peptide (SLP) comprising at least one of the amino acid sequences defined in claim 80.

86. A nucleic acid encoding (i) the TAP of claim 80, (ii) a combination comprising at least two of the TAPs defined in claim 80; or (iii) a synthetic long peptide (SLP) comprising at least one of the amino acid sequences defined in claim 80.

87. The nucleic acid of claim 86, wherein the nucleic acid is an mRNA, and wherein the mRNA optionally comprises one or more 5′-end modifications, 3′-end modifications, and/or modified nucleosides to increase stability, improve translation and/or reduce immunogenicity of the mRNA.

88. A vesicle or particle comprising the nucleic acid of claim 86.

89. The vesicle or particle of claim 88, wherein the vesicle is a lipid nanoparticle (LNP).

90. A vaccine comprising (a) (i) the TAP of claim 80, (ii) a combination comprising at least two of the TAPs defined in claim 80; (iii) a synthetic long peptide (SLP) comprising at least one of the amino acid sequences defined in claim 80, or a nucleic acid encoding the TAP, combination, or SLP defined in (i)-(iii), and (b) an adjuvant.

91. A T-cell receptor (TCR), an antibody or an antigen-binding fragment thereof, that specifically recognizes MHC class I molecule comprising the TAP of claim 80 in its peptide binding groove.

92. An isolated cell expressing at its cell surface the TCR, antibody or antigen-binding fragment thereof of claim 91.

93. The isolated cell of claim 92, which is a CD8⁺ T lymphocyte or a chimeric antigen receptor (CAR) cell.

94. A cell population comprising at least 0.5% of the isolated cell as defined in claim 93.

95. A method of treating cancer in a subject comprising administering to the subject an effective amount of:
(a) a TAP comprising or consisting of any one of the sequences set forth in SEQ ID NOs: 1-505 or any combination thereof, or a synthetic long peptide (SLP) comprising at least one of the sequences set forth in SEQ ID NOs: 1-505;
(b) at least one nucleic acid encoding the TAP, combination thereof or SLP defined in (a);
(c) a vesicle or particle comprising the TAP, combination thereof or SLP defined in (a) or the at least one nucleic acid defined in (b);
(d) a composition comprising the TAP, combination thereof or SLP defined in (a), the at least one nucleic acid defined in (b), or the vesicle or particle defined in (c), and a pharmaceutically acceptable carrier;
(e) a vaccine comprising the TAP, combination thereof or SLP defined in (a), the at least one nucleic acid defined in (b), the vesicle or particle defined in (c), or the composition defined in (d), and an adjuvant;
(f) a cell expressing at its surface major histocompatibility complex (MHC) class I molecules comprising the TAP or combination thereof defined in (a) in their peptide binding groove;
(g) a cell expressing at its cell surface a T-cell receptor (TCR) or a chimeric antigen receptor (CAR) that specifically recognizes MHC class I molecules expressed at the surface of the cell defined in (f); or
(h) a soluble TCR, an antibody or an antigen-binding fragment thereof, or a CAR, that specifically binds to the MHC class I molecules expressed at the surface of the cell defined in (f), or a nucleic acid encoding said soluble TCR, antibody, antigen-binding fragment thereof, or CAR.

96. The method of claim 95, wherein the cancer is melanoma.

97. The method of claim 95, further comprising administering at least one additional antitumor agent or therapy to the subject.

98. The method of claim 97, wherein said at least one additional antitumor agent or therapy is a chemotherapeutic agent, immunotherapy, an immune checkpoint inhibitor, radiotherapy or surgery.
